SEQUENCE LISTING (266 sequences)

SEQ ID NO: 1 - protein, 18 aa, synthetic construct
  REGION 1..18: note = SLN-6068(C2-11) CDR3
RMSRLLGMAPLLPEHYDS

SEQ ID NO: 2 - protein, 17 aa, synthetic construct
  REGION 1..17: note = SLN-6068(C2-11) CDR2
AAYAGGGGTVYADSVKG

SEQ ID NO: 3 - protein, 10 aa, synthetic construct
  REGION 1..10: note = SLN-6068(C2-11) CDR1
VYTSSTYYMA

SEQ ID NO: 4 - protein, 18 aa, synthetic construct
  REGION 1..18: note = SLN-6043 (F6-1) CDR3
KWPCTYRRGGSAYRPAVV

SEQ ID NO: 5 - protein, 16 aa, synthetic construct
  REGION 1..16: note = SLN-6043 (F6-1) CDR2
SISSDDSTRYADSVKG

SEQ ID NO: 6 - protein, 10 aa, synthetic construct
  REGION 1..10: note = SLN-6043 (F6-1) CDR1
GFTSDNCAVH

SEQ ID NO: 7 - protein, 18 aa, synthetic construct
  REGION 1..18: note = SLN-6062 (C8-9) CDR3
TWSWYSFTCPGDQNAYIH

SEQ ID NO: 8 - protein, 17 aa, synthetic construct
  REGION 1..17: note = SLN-6062 (C8-9) CDR2
TLNRNSGRTSYVGSVKG

SEQ ID NO: 9 - protein, 8 aa, synthetic construct
  REGION 1..8: note = SLN-6062 (C8-9) CDR1
VYTASCMG

SEQ ID NO: 10 - protein, 15 aa, synthetic construct
  REGION 1..15: note = SLN-6065 (D2-9) CDR3
GWRGGSFWTPSKYSY

SEQ ID NO: 11 - protein, 17 aa, synthetic construct
  REGION 1..17: note = SLN-6065 (D2-9) CDR2
SIATNTGNDYYADSVKG

SEQ ID NO: 12 - protein, 10 aa, synthetic construct
  REGION 1..10: note = SLN-6065 (D2-9) CDR1
RDAYFNNYMA

SEQ ID NO: 13 - protein, 127 aa, synthetic construct
  REGION 1..127: note = SLN-6068(C2-11)
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSS

SEQ ID NO: 14 - protein, 126 aa, synthetic construct
  REGION 1..126: note = SLN-6043 (F6-1)
EVQLVESGGGVVQPGGSLRLSCAASGFTSDNCAVHWVRQAPGKGLEWVSSISSDDSTRYADSVKGRFTISRDNSKNSLYLQMNSLRTEDTALYYCQTKWPCTYRRGGSAYRPAVVWGQGTLVTVSS

SEQ ID NO: 15 - protein, 125 aa, synthetic construct
  REGION 1..125: note = SLN-6062 (C8-9)
EVQLVESGGGLVQPGGSLRLSCAASVYTASCMGWFRQAPGKGLELVSTLNRNSGRTSYVGSVKGRFTISQDNSKKTLYLQMNSLRAEDTAVYYCAATWSWYSFTCPGDQNAYIHWGQGTLVTVSS

SEQ ID NO: 16 - protein, 124 aa, synthetic construct
  REGION 1..124: note = SLN-6065 (D2-9)
EVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSS

SEQ ID NO: 17 - protein, 268 aa, synthetic construct
  REGION 1..268: note = (SLN6071, bi-paratopic VHH-1)C2_F6
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGVVQPGGSLRLSCAASGFTSDNCAVHWVRQAPGKGLEWVSSISSDDSTRYADSVKGRFTISRDNSKNSLYLQMNSLRTEDTALYYCQTKWPCTYRRGGSAYRPAVVWGQGTLVTVSS

SEQ ID NO: 18 - protein, 267 aa, synthetic construct
  REGION 1..267: note = (SLN6075, , bi-paratopic VHH-2)C2_C8
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASVYTASCMGWFRQAPGKGLELVSTLNRNSGRTSYVGSVKGRFTISQDNSKKTLYLQMNSLRAEDTAVYYCAATWSWYSFTCPGDQNAYIHWGQGTLVTVSS

SEQ ID NO: 19 - protein, 266 aa, synthetic construct
  REGION 1..266: note = (SLN6079, bi-paratopic VHH-3)C2_D2
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSS

SEQ ID NO: 20 - protein, 229 aa, synthetic construct
  REGION 1..229: note = Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 495 aa, synthetic construct
  REGION 1..495: note = (SLN6073, C2_D2-Fc fusion)
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 373 aa, synthetic construct
  REGION 1..373: note = CFH
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEESTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 23 - protein, 873 aa, synthetic construct
  REGION 1..873: note = (SLN6074, C2_D2-Fc-CFH fusion)
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEESTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 24 - protein, 25 aa, synthetic construct [sequence omitted]

SEQ ID NO: 25 - protein, 14 aa, synthetic construct
  REGION 1..14: note = SLN-6068(C2-11) FR2
WFRQAPGKGREGVA

SEQ ID NO: 26 - protein, 32 aa, synthetic construct
  REGION 1..32: note = SLN-6068(C2-11) FR3
RFTISQDNSKNTLYLQMNSLRAEDTAVYYCAA

SEQ ID NO: 27 - protein, 11 aa, synthetic construct
  REGION 1..11: note = SLN-6068(C2-11) /SLN-6062(C8-9) /SLN-6065(D2-9) /SLN6043(F6-1) FR4
WGQGTLVTVSS

SEQ ID NO: 28 - protein, 25 aa, synthetic construct
  REGION 1..25: note = SLN-6043 (F6-1) FR1
EVQLVESGGGVVQPGGSLRLSCAAS

SEQ ID NO: 29 - protein, 14 aa, synthetic construct
  REGION 1..14: note = SLN-6043 (F6-1) FR2
WVRQAPGKGLEWVS

SEQ ID NO: 30 - protein, 32 aa, synthetic construct
  REGION 1..32: note = SLN-6043 (F6-1) FR3
RFTISRDNSKNSLYLQMNSLRTEDTALYYCQT

SEQ ID NO: 31 - protein, 14 aa, synthetic construct
  REGION 1..14: note = SLN-6062 (C8-9) FR2
WFRQAPGKGLELVS

SEQ ID NO: 32 - protein, 32 aa, synthetic construct
  REGION 1..32: note = SLN-6062 (C8-9) FR3
RFTISQDNSKKTLYLQMNSLRAEDTAVYYCAA

SEQ ID NO: 33 - protein, 14 aa, synthetic construct
  REGION 1..14: note = SLN-6065 (D2-9) FR2
WFRQAPGKGLEGVS

SEQ ID NO: 34 - protein, 32 aa, synthetic construct
  REGION 1..32: note = SLN-6065 (D2-9) FR3
RFTISRDNSKNTIYLQMNSLRAEDTAVYYCAA

SEQ ID NO: 35 - protein, 25 aa, synthetic construct
  REGION 1..25: note = SLN-6068(C2-11) FR1; SLN-6062 (C8-9) FR1; SLN-6065 (D2-9) FR1; SLN-6043 (F6-1) FR1
  VAR_SEQ 11: note = X = L or V
EVQLVESGGGXVQPGGSLRLSCAAS

SEQ ID NO: 36 - protein, 14 aa, synthetic construct
  REGION 1..14: note = SLN-6068(C2-11) FR2; SLN-6043 (F6-1) FR2; SLN-6062 (C8-9) FR2; SLN-6065 (D2-9) FR2
  VAR_SEQ 2: note = X = F or V
  VAR_SEQ 10: note = X = L or R
  VAR_SEQ 12: note = X = G, L or W
  VAR_SEQ 14: note = X = A or S
WXRQAPGKGXEXVX

SEQ ID NO: 37 - protein, 32 aa, synthetic construct
  REGION 1..32: note = SLN-6068(C2-11) FR3; SLN-6043 (F6-1) FR3; SLN-6062 (C8-9) FR3; SLN-6065 (D2-9) FR3
  VAR_SEQ 6: note = X = Q or R
  VAR_SEQ 11: note = X = K or N
  VAR_SEQ 12: note = X = S or T
  VAR_SEQ 13: note = X = I or L
  VAR_SEQ 22: note = X = A or T
  VAR_SEQ 27: note = X = L or V
  VAR_SEQ 31: note = X = A or Q
  VAR_SEQ 32: note = X = A or T
RFTISXDNSKXXXYLQMNSLRXEDTAXYYCXX

SEQ ID NO: 38 - protein, 429 aa, synthetic construct
  REGION 1..429: note = SLN-6066
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 5 aa, synthetic construct
  REGION 1..5: note = SLN9056/SLN0056/SLN9015/SLN9079/SLN0104/SLN0100/SLN 0097 HCDR1
GNYMG

SEQ ID NO: 40 - protein, 5 aa, synthetic construct
  REGION 1..5: note = SLN9057/SLN0072/SLN9060/SLN9061/SLN9062/SLN9077/SLN 9058/SLN0105 HCDR1
SSYMG

SEQ ID NO: 41 - protein, 6 aa, synthetic construct
  REGION 1..6: note = SLN0042 HCDR1
SFTCMG

SEQ ID NO: 42 - protein, 5 aa, synthetic construct
  REGION 1..5: note = SLN0043 HCDR1
RDWMA

SEQ ID NO: 43 - protein, 5 aa, synthetic construct
  REGION 1..5: note = SLN0044 HCDR1
TACMA

SEQ ID NO: 44 - protein, 5 aa, synthetic construct
  REGION 1..5: note = SLN0045/SLN0046 HCDR1
RACMA

SEQ ID NO: 45 - protein, 5 aa, synthetic construct
  REGION 1..5: note = SLN0049 HCDR1
RYSMG

SEQ ID NO: 46 - protein, 5 aa, synthetic construct
  REGION 1..5: note = SLN0057 HCDR1
IYYMA

SEQ ID NO: 47 - protein, 5 aa, synthetic construct
  REGION 1..5: note = SLN0059/SLN9014 HCDR1
NYYMT

SEQ ID NO: 48 - protein, 5 aa, synthetic construct
  REGION 1..5: note = SLN0062 HCDR1
RNCMG

SEQ ID NO: 49 - protein, 5 aa, synthetic construct
  REGION 1..5: note = SLN0064 HCDR1
WFCMG

SEQ ID NO: 50 - protein, 5 aa, synthetic construct
  REGION 1..5: note = SLN0065 HCDR1
LFWMG

SEQ ID NO: 51 - protein, 5 aa, synthetic construct
  REGION 1..5: note = SLN0071 HCDR1
RCCMG

SEQ ID NO: 52 - protein, 5 aa, synthetic construct
  REGION 1..5: note = SLN0058 HCDR1
SDCMG

SEQ ID NO: 53 - protein, 5 aa, synthetic construct
  REGION 1..5: note = SLN9008 HCDR1
GHCMA

SEQ ID NO: 54 - protein, 5 aa, synthetic construct
  REGION 1..5: note = SLN9013 HCDR1
RGCMG

SEQ ID NO: 55 - protein, 5 aa, synthetic construct
  REGION 1..5: note = SLN9025 HCDR1
QHCMG

SEQ ID NO: 56 - protein, 5 aa, synthetic construct
  REGION 1..5: note = SLN9026 HCDR1
SKCMA

SEQ ID NO: 57 - protein, 5 aa, synthetic construct
  REGION 1..5: note = 1902-2-1-mm-F7 HCDR1
RACMG

SEQ ID NO: 58 - protein, 5 aa, synthetic construct
  REGION 1..5: note = 1902-2-1-hh-B12 HCDR1
KYCMG

SEQ ID NO: 59 - protein, 5 aa, synthetic construct
  REGION 1..5: note = 1902-2-1-hm-D12 HCDR1
NYCMA

SEQ ID NO: 60 - protein, 17 aa, synthetic construct
  REGION 1..17: note = SLN9056/SLN0056/SLN9015/SLN9079/SLN0104/SLN0100/SLN 0097 HCDR2
VLYTGGGSTYYADSVKG

SEQ ID NO: 61 - protein, 17 aa, synthetic construct
  REGION 1..17: note = SLN9057/SLN0072/SLN9062/SLN9077/SLN9058/SLN0105 HCDR2
QINSRSGSTYYADSVKG

SEQ ID NO: 62 - protein, 17 aa, synthetic construct
  REGION 1..17: note = SLN0042 HCDR2
TINPGTGSTYYADSVKG

SEQ ID NO: 63 - protein, 17 aa, synthetic construct
  REGION 1..17: note = SLN0043 HCDR2
AFVPSIGSTFYVDSVKG

SEQ ID NO: 64 - protein, 16 aa, synthetic construct
  REGION 1..16: note = SLN0044 HCDR2
AIARYGDTTYTDSVKG

SEQ ID NO: 65 - protein, 16 aa, synthetic construct
  REGION 1..16: note = SLN0045/SLN0046 HCDR2
WIIADGSTGYADSVKG

SEQ ID NO: 66 - protein, 16 aa, synthetic construct
  REGION 1..16: note = SLN0049 HCDR2
AIDSIGRTSYADSVKG

SEQ ID NO: 67 - protein, 16 aa, synthetic construct
  REGION 1..16: note = SLN0057 HCDR2
VIYRGGSTTYADSAKG

SEQ ID NO: 68 - protein, 16 aa, synthetic construct
  REGION 1..16: note = SLN0059 HCDR2
GLTRADVTLYADSVKG

SEQ ID NO: 69 - protein, 17 aa, synthetic construct
  REGION 1..17: note = SLN0062 HCDR2
TVYPGGGSTYYADSVKG

SEQ ID NO: 70 - protein, 16 aa, synthetic construct
  REGION 1..16: note = SLN0064 HCDR2
VIDVDSIARYGDSVKG

SEQ ID NO: 71 - protein, 16 aa, synthetic construct
  REGION 1..16: note = SLN0065 HCDR2
VTGRDGSTIYADSVQG

SEQ ID NO: 72 - protein, 16 aa, synthetic construct
  REGION 1..16: note = SLN0071 HCDR2
SINVLGGTSYADSVKG

SEQ ID NO: 73 - protein, 17 aa, synthetic construct
  REGION 1..17: note = SLN0058 HCDR2
TIATAGGSTGYADSVKG

SEQ ID NO: 74 - protein, 18 aa, synthetic construct
  REGION 1..18: note = SLN9008 HCDR2
AIYTGTGSTYYADESVKG

SEQ ID NO: 75 - protein, 17 aa, synthetic construct
  REGION 1..17: note = SLN9013 HCDR2
SFLHGAASADYADSVKG

SEQ ID NO: 76 - protein, 17 aa, synthetic construct
  REGION 1..17: note = SLN9025 HCDR2
YTYVSFNVTHYADSVKG

SEQ ID NO: 77 - protein, 17 aa, synthetic construct
  REGION 1..17: note = SLN9026 HCDR2
AIDTSRGRAYLTDSVKG

SEQ ID NO: 78 - protein, 17 aa, synthetic construct
  REGION 1..17: note = SLN9060 HCDR2
QIQSRSGSTYYADSVKG

SEQ ID NO: 79 - protein, 17 aa, synthetic construct
  REGION 1..17: note = SLN9061 HCDR2
QINARSGSTYYADSVKG

SEQ ID NO: 80 - protein, 17 aa, synthetic construct
  REGION 1..17: note = 1902-2-1-mm-F7 HCDR2
RVYSDGSQSYYPDSVKG

SEQ ID NO: 81 - protein, 16 aa, synthetic construct
  REGION 1..16: note = 1902-2-1-hh-B12 HCDR2
AIDSDGTTRYADSVQG

SEQ ID NO: 82 - protein, 16 aa, synthetic construct
  REGION 1..16: note = 1902-2-1-hm-D12 HCDR2
NMNSYDWTDYDDSVKG

SEQ ID NO: 83 - protein, 19 aa, synthetic construct
  REGION 1..19: note = SLN9056/SLN0056/SLN9079/SLN0104/SLN0100/SLN0097 HCDR3
ALGSARWYTSSLDARAYNI

SEQ ID NO: 84 - protein, 18 aa, synthetic construct
  REGION 1..18: note = SLN9057 HCDR3
RFGPTFYAINLGSNLYNY

SEQ ID NO: 85 - protein, 16 aa, synthetic construct
  REGION 1..16: note = SLN0042 HCDR3
TQLFGCGSLAKSLFGY

SEQ ID NO: 86 - protein, 20 aa, synthetic construct
  REGION 1..20: note = SLN0043 HCDR3
DPLWGRKYGGSWSDPSEYNY

SEQ ID NO: 87 - protein, 15 aa, synthetic construct
  REGION 1..15: note = SLN0044 HCDR3
KSPNSGCDEYAHYET

SEQ ID NO: 88 - protein, 17 aa, synthetic construct
  REGION 1..17: note = SLN0045 HCDR3
AYNGGDRCYTLIGLYNH

SEQ ID NO: 89 - protein, 16 aa, synthetic construct
  REGION 1..16: note = SLN0049 HCDR3
TAWRDWATLREYEYGY

SEQ ID NO: 90 - protein, 16 aa, synthetic construct
  REGION 1..16: note = SLN0057 HCDR3
AHVLHVSSLLPGGYPY

SEQ ID NO: 91 - protein, 15 aa, synthetic construct
  REGION 1..15: note = SLN0059 HCDR3
ADRFRVGLREADFSA

SEQ ID NO: 92 - protein, 16 aa, synthetic construct
  REGION 1..16: note = SLN0062 HCDR3
SRRIWSCGSGAGSYDY

SEQ ID NO: 93 - protein, 20 aa, synthetic construct
  REGION 1..20: note = SLN0064 HCDR3
KTERQCRWNWMDWRTYDYPH

SEQ ID NO: 94 - protein, 18 aa, synthetic construct
  REGION 1..18: note = SLN0065 HCDR3
DQTRYSSLRLLAPNRSAS

SEQ ID NO: 95 - protein, 19 aa, synthetic construct
  REGION 1..19: note = SLN0071 HCDR3
GLASAPWRPCGTTTEEYKY

SEQ ID NO: 96 - protein, 18 aa, synthetic construct
  REGION 1..18: note = SLN0072/SLN9060/SLN9061/SLN9062/SLN9058/SLN0105 HCDR3
RFGPTFYPVPLGSNLYNY

SEQ ID NO: 97 - protein, 16 aa, synthetic construct
  REGION 1..16: note = SLN0046 HCDR3
YNGGDRCYTLIGLYNH

SEQ ID NO: 98 - protein, 18 aa, synthetic construct
  REGION 1..18: note = SLN0058 HCDR3
WGGAWWYPWCEFVFSNGY

SEQ ID NO: 99 - protein, 16 aa, synthetic construct
  REGION 1..16: note = SLN9008 HCDR3
TLLYGCGAWSPRLFGY

SEQ ID NO: 100 - protein, 19 aa, synthetic construct
  REGION 1..19: note = SLN9013 HCDR3
NPVVASCHYRLRSKYAYNY

SEQ ID NO: 101 - protein, 19 aa, synthetic construct
  REGION 1..19: note = SLN9015 HCDR3
VLGSARWYTSSLDARAYNI

SEQ ID NO: 102 - protein, 14 aa, synthetic construct
  REGION 1..14: note = SLN9025 HCDR3
GYYSCGVSTTGYNY

SEQ ID NO: 103 - protein, 21 aa, synthetic construct
  REGION 1..21: note = SLN9026 HCDR3
SNTFYNCGALNLGIGAGLVAY

SEQ ID NO: 104 - protein, 18 aa, synthetic construct
  REGION 1..18: note = SLN9077 HCDR3
RFGPTFYTGNLGSNLYNY

SEQ ID NO: 105 - protein, 20 aa, synthetic construct
  REGION 1..20: note = 1902-2-1-mm-F7 HCDR3
LLMSDYYSCRVRFHQSDFRD

SEQ ID NO: 106 - protein, 20 aa, synthetic construct
  REGION 1..20: note = 1902-2-1-hh-B12 HCDR3
DRWSMKYYSDYALFPGGYNY

SEQ ID NO: 107 - protein, 22 aa, synthetic construct
  REGION 1..22: note = 1902-2-1-hm-D12 HCDR3
RDCALRYCTRSYCTREAHFFKY

SEQ ID NO: 108 - protein, 128 aa, synthetic construct
  REGION 1..128: note = SLN9056
EVQLVESGGGLVQPGGSLRLSCAASGHAYGGNYMGWFRQAPGKGLEGVAVLYTGGGSTYYADSVKGRFTISEDNSKNTVYLQMNSLRAEDTAVYYCALALGSARWYTSSLDARAYNIWGQGTLVTVSS

SEQ ID NO: 109 - protein, 127 aa, synthetic construct
  REGION 1..127: note = SLN9057
EVQLVESGGGLVQPGGSLRLSCAASGYTGSSSYMGWFRQAPGKGLEGVSQINSRSGSTYYADSVKGRFTISRDNSKNTLYLQMNSLRAEDTAVYYCAERFGPTFYAINLGSNLYNYWGQGTLVTVSS

SEQ ID NO: 110 - protein, 125 aa, synthetic construct
  REGION 1..125: note = SLN0042
DVQLVESGGGSVQAGGSLRLSCAASGYTRSFTCMGWFRQAPGKEREGVATINPGTGSTYYADSVKGRFSISQDNAKNTVYLQMNTLKPEDTAMYYCAATQLFGCGSLAKSLFGYWGQGTLVTVSS

SEQ ID NO: 111 - protein, 129 aa, synthetic construct
  REGION 1..129: note = SLN0043
DVQLVESGGGSVQAGGSLRLSCAASGVTWSRDWMAWFRQAPGKEREGVAAFVPSIGSTFYVDSVKGRFTISQDNAKNTLFLQMNSLKPEDTAMYYCATDPLWGRKYGGSWSDPSEYNYWGQGTQVTVSS

SEQ ID NO: 112 - protein, 123 aa, synthetic construct
  REGION 1..123: note = SLN0044
DVQLVESGGGSVQAGGSLRLSCAASIYTYSTACMAWFRQAPGKEREGVAAIARYGDTTYTDSVKGRFTISRDVAKNILYLQMSSLKPEDTAMYYCASKSPNSGCDEYAHYETFGQGTQVTVSS

SEQ ID NO: 113 - protein, 125 aa, synthetic construct
  REGION 1..125: note = SLN0045
EVQLVESGGGSVQAGGSLRLSCTASGYTYSRACMAWFRQAAGKQREWVSWIIADGSTGYADSVKGRFTISRDNAKNTLYLDMTSLKPEDTAMYYCAAAYNGGDRCYTLIGLYNHWGQGTQVTVSS

SEQ ID NO: 114 - protein, 124 aa, synthetic construct
  REGION 1..124: note = SLN0049
EVQLVESGGGSVQAGGSLRLSCAASRATNSRYSMGWFRQAPGKEREGVAAIDSIGRTSYADSVKGRFTISQDGAKNTLYLQMNSLKPEDTAMYYCAATAWRDWATLREYEYGYWGQGTQVTVSS

SEQ ID NO: 115 - protein, 128 aa, synthetic construct
  REGION 1..128: note = SLN0056
EVQLVESGGGSVQAGGSLRLSCRASGHAYGGNYMGWFRQAPGKEREGVAVLYTGGGSTYYADSVKGRFTISEDNAKNTVYLQMDSLKLEDTAMYYCALALGSARWYTSSLDARAYNIWGQGTQVTVSS

SEQ ID NO: 116 - protein, 124 aa, synthetic construct
  REGION 1..124: note = SLN0057
HVQLVESGGGSVQAGGSLRLSCAASGHTYSIYYMAWFRQAPGKEREGVAVIYRGGSTTYADSAKGRFTVSQDNGKNTVYLQMNSLKAEDTAMYYCAAAHVLHVSSLLPGGYPYWGQGTLVTVSS

SEQ ID NO: 117 - protein, 123 aa, synthetic construct
  REGION 1..123: note = SLN0059
HVQLVESGGGSVQSGGSLRLSCAASGITISNYYMTWFRQAPGKEREGVAGLTRADVTLYADSVKGRFTISRDNAKNTLYLQMNSLKPEDTAMYYCAAADRFRVGLREADFSAWGQGTQVTVSS

SEQ ID NO: 118 - protein, 125 aa, synthetic construct
  REGION 1..125: note = SLN0062
QVQLVESGGGSVQSGGSLRLSCAASGYTFSRNCMGWFRQTPGKERERVATVYPGGGSTYYADSVKGRFTFSQDNAKNAIYLQMNTLKPEDTGMYYCAASRRIWSCGSGAGSYDYWGQGTQVTVSS

SEQ ID NO: 119 - protein, 128 aa, synthetic construct
  REGION 1..128: note = SLN0064
HVQLVESGGGSVQAGGSLRLSCAASGYSVSWFCMGWFRQAPGLEREGVAVIDVDSIARYGDSVKGRFTISQGNSKDTVYLQMNGLKPEDTAMYYCAAKTERQCRWNWMDWRTYDYPHWGQGTQVTVSS

SEQ ID NO: 120 - protein, 123 aa, synthetic construct
  REGION 1..123: note = SLN0065
EVQLVESGGGSVQAGGSLRLSCLYTDSLFWMGWFRQTPGNEREGVAVTGRDGSTIYADSVQGRFTISRDNAKNTLYLQMNSLKPDDTAMYYCAADQTRYSSLRLLAPNRSASWGQGTQVTVSS

SEQ ID NO: 121 - protein, 127 aa, synthetic construct
  REGION 1..127: note = SLN0071
EVQLVESGGGSVQAGGSLRLSCAGSEYTYSRCCMGWFRQAPGKEREGVVSINVLGGTSYADSVKGRFTISRDNAENTLYLQMNSLKPEDTAMYYCAAGLASAPWRPCGTTTEEYKYWGQGTQVTVSS

SEQ ID NO: 122 - protein, 127 aa, synthetic construct
  REGION 1..127: note = SLN0072
EVQLVESGGGSVQAGGSLRLSCVASGYTGSSSYMGWFRQVPGKEREGVAQINSRSGSTYYADSVKGRFTISQDNAKNTVYLQMTSLKPEDTGMYYCAERFGPTFYPVPLGSNLYNYWGQGTQVTVSS

SEQ ID NO: 123 - protein, 125 aa, synthetic construct
  REGION 1..125: note = SLN0046
DVQLVESGGGSVQAGGSLRLSCTASGYTYSRACMAWFRQAAGKQREWVSWIIADGSTGYADSVKGRFTISRDNAKNTLYLDMTSLKPEDTAMYYCAAAYNGGDRCYTLIGLYNHWGQGTQVTVSS

SEQ ID NO: 124 - protein, 127 aa, synthetic construct
  REGION 1..127: note = SLN0058
HVQLVESGGGSVQAGGSLRLSCTASGYMYTSDCMGWFRQAPGKEREGVATIATAGGSTGYADSVKGRFTISQDNAKNTMYLQMNNLKPEDTAMYYCAAWGGAWWYPWCEFVFSNGYWGQGTLVTVSS

SEQ ID NO: 125 - protein, 126 aa, synthetic construct
  REGION 1..126: note = SLN9008
DVQLVESGGGSVQAGGSLRLSCQASGYTGGGHCMAWFRQAPGKDREGIAAIYTGTGSTYYADESVKGRFIISQDNARSTVYLQMDSLKPEDTGMYYCAATLLYGCGAWSPRLFGYWGQGTQVTVSS

SEQ ID NO: 126 - protein, 128 aa, synthetic construct
  REGION 1..128: note = SLN9013
QVQLVESGGGSVQAGGSLRLSCAASGYTYSRGCMGWFRQAPGKEREGVASFLHGAASADYADSVKGRFTISQDNAKNTMYLQMNNLKPEDTAMYYCAANPVVASCHYRLRSKYAYNYWGEGTQVTVSS

SEQ ID NO: 127 - protein, 128 aa, synthetic construct
  REGION 1..128: note = SLN9015
DVQLVESGGGSVQAGGSLRLSCRASGHAYSGNYMGWFRQAPGKEREGVAVLYTGGGSTYYADSVKGRFTISEDNAKNTVYLQMDSLKLEDTAMYYCALVLGSARWYTSSLDARAYNIWGQGTQVTVSS

SEQ ID NO: 128 - protein, 123 aa, synthetic construct
  REGION 1..123: note = SLN9025
HVQLVESGGGSVQAGGSLNVSCAASGYTFNQHCMGWFRQAPGKQREGFLYTYVSFNVTHYADSVKGRFTISHDSAENAMYLQMNSLGPEDSGMYYCAAGYYSCGVSTTGYNYWGQGTQVTVSS

SEQ ID NO: 129 - protein, 130 aa, synthetic construct
  REGION 1..130: note = SLN9026
HVQLVESGGGSVQAGGSLRLSCAASGDTYSSKCMAWFRQVPGKEREGVAAIDTSRGRAYLTDSVKGRFTISQDNAKNTVYLQMNSLKPEDTAMYYCAASNTFYNCGALNLGIGAGLVAYWGQGTQVTVSS

SEQ ID NO: 130 - protein, 129 aa, synthetic construct
  REGION 1..129: note = 1902-2-1-mm-F7
HVQLVESGGGSVQTGGSLRLSCAASGYTISRACMGWFRQAPGKEREMVARVYSDGSQSYYPDSVKGRFTISQDNAKNTVYLQMDRLEPEDTAIYYCAALLMSDYYSCRVRFHQSDFRDWGQGTQVTVSS

SEQ ID NO: 131 - protein, 128 aa, synthetic construct
  REGION 1..128: note = 1902-2-1-hh-B12
EVQLVESGGGSVQAGGSLRLSCVASGYTFGKYCMGWIRQVPGKEREGVAAIDSDGTTRYADSVQGRFTISQDNAKNTLVLEMNSLKLEDSAMYYCAADRWSMKYYSDYALFPGGYNYWGQGTLVTVSS

SEQ ID NO: 132 - protein, 130 aa, synthetic construct
  REGION 1..130: note = 1902-2-1-hm-D12
DVQLVESGGGSVQAGESLRLSCGASGYTYRNYCMAWFRLAPGKEREGVANMNSYDWTDYDDSVKGRFTISQDNAANTWYLQMNSLKPEDTAMYYCALRDCALRYCTRSYCTREAHFFKYWGQGTQVTVSS

SEQ ID NO: 133 - protein, 128 aa, synthetic construct
  REGION 1..128: note = SLN0097
EVQLVESGGGLVQPGGSLRLSCAASGHAYGGNYMGWFRQAPGKGLEGVSVLYTGGGSTYYADSVKGRFTISRDNSKNTLYLQMNSLRAEDTAVYYCALALGSARWYTSSLDARAYNIWGQGTLVTVSS

SEQ ID NO: 134 - protein, 128 aa, synthetic construct
  REGION 1..128: note = SLN9079
EVQLVESGGGLVQPGGSLRLSCAASGHAYGGNYMGWFRQAPGKGLEGVAVLYTGGGSTYYADSVKGRFTISEDNSKNTLYLQMNSLRAEDTAVYYCALALGSARWYTSSLDARAYNIWGQGTLVTVSS

SEQ ID NO: 135 - protein, 127 aa, synthetic construct
  REGION 1..127: note = SLN9060
EVQLVESGGGLVQPGGSLRLSCAASGYTGSSSYMGWFRQAPGKGLEGVSQIQSRSGSTYYADSVKGRFTISRDNSKNTLYLQMNSLRAEDTAVYYCAERFGPTFYPVPLGSNLYNYWGQGTLVTVSS

SEQ ID NO: 136 - protein, 127 aa, synthetic construct
  REGION 1..127: note = SLN9061
EVQLVESGGGLVQPGGSLRLSCAASGYTGSSSYMGWFRQAPGKGLEGVSQINARSGSTYYADSVKGRFTISRDNSKNTLYLQMNSLRAEDTAVYYCAERFGPTFYPVPLGSNLYNYWGQGTLVTVSS

SEQ ID NO: 137 - protein, 127 aa, synthetic construct
  REGION 1..127: note = SLN9062
EVQLVESGGGLVQPGGSLRLSCAASGYTGSSSYMGWFRQAPGKGLEGVCQINSRSGSTYYADSVKGRFTCSRDNSKNTLYLQMNSLRAEDTAVYYCAERFGPTFYPVPLGSNLYNYWGQGTLVTVSS

SEQ ID NO: 138 - protein, 127 aa, synthetic construct
  REGION 1..127: note = SLN9077
EVQLVESGGGLVQPGGSLRLSCAASGYTGSSSYMGWFRQAPGKGLEGVSQINSRSGSTYYADSVKGRFTISRDNSKNTLYLQMNSLRAEDTAVYYCAERFGPTFYTGNLGSNLYNYWGQGTLVTVSS

SEQ ID NO: 139 - protein, 127 aa, synthetic construct
  REGION 1..127: note = SLN9058
EVQLVESGGGLVQPGGSLRLSCAASGYTGSSSYMGWFRQAPGKGREGVSQINSRSGSTYYADSVKGRFTISRDNSKNTLYLQMNSLRAEDTAVYYCAERFGPTFYPVPLGSNLYNYWGQGTLVTVSS

SEQ ID NO: 140 - protein, 128 aa, synthetic construct
  REGION 1..128: note = SLN0104
EVQLVESGGGLVQPGGSLRLSCAASGHAYGGNYMGWFRQAPGKGLEGVAVLYTGGGSTYYADSVKGRFTISRDNAKNSLYLQMNSLRAEDTAVYYCALALGSARWYTSSLDARAYNIWGQGTLVTVSS

SEQ ID NO: 141 - protein, 128 aa, synthetic construct
  REGION 1..128: note = SLN0100
EVQLVESGGGLVQPGGSLRLSCAASGHAYGGNYMGWFRQAPGKGLEGVSVLYTGGGSTYYADSVKGRFTISRDNAKNSLYLQMNSLRAEDTAVYYCALALGSARWYTSSLDARAYNIWGQGTLVTVSS

SEQ ID NO: 142 - protein, 127 aa, synthetic construct
  REGION 1..127: note = SLN0105
EVQLVESGGGLVQPGGSLRLSCAASGYTGSSSYMGWFRQAPGKGLEGVSQINSRSGSTYYADSVKGRFTISRDNSKNTLYLQMNSLRAEDTAVYYCAERFGPTFYPVPLGSNLYNYWGQGTLVTVSS

SEQ ID NO: 143 - protein, 174 aa, synthetic construct
  REGION 1..174: note = GLP-1-9056
HGEGTFTSDVSSYLEEQAAKEFIAWLVKGGGGGGGSGGGGSGGGGSEVQLVESGGGLVQPGGSLRLSCAASGHAYGGNYMGWFRQAPGKGLEGVAVLYTGGGSTYYADSVKGRFTISEDNSKNTVYLQMNSLRAEDTAVYYCALALGSARWYTSSLDARAYNIWGQGTLVTVSS

SEQ ID NO: 144 - protein, 506 aa, synthetic construct
  REGION 1..506: note = 0072-PE38
EVQLVESGGGSVQAGGSLRLSCVASGYTGSSSYMGWFRQVPGKEREGVAQINSRSGSTYYADSVKGRFTISQDNAKNTVYLQMTSLKPEDTGMYYCAERFGPTFYPVPLGSNLYNYWGQGTQVTVSSEFPKPSTPPGSSGGAPPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 145 - protein, 385 aa, synthetic construct
  REGION 1..385: note = SLN0041(OVA)
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 146 - protein, 28 aa, synthetic construct
  REGION 1..28: note = GLP-1
HGEGTFTSDVSSYLEEQAAKEFIAWLVK

SEQ ID NO: 147 - protein, 219 aa, synthetic construct
  REGION 1..219: note = PE-38
PTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 148 - protein, 129 aa, synthetic construct
  REGION 1..129: note = SLN0066
HVQLVESGGGSVQAGGSLRLSCVASGIGHGFNNNCMGWFRQAPGKEREGVAAVYTGGGTPYYADSVKGRFTLSQDNAKNTLYLQMNGLDPEDTAMYYCVADIWRTYRCGAGDTTVFDYRGQGTLVTVSS

SEQ ID NO: 149 - protein, 5 aa, synthetic construct
  REGION 1..5: note = CDR1
YYCMG

SEQ ID NO: 150 - protein, 5 aa, synthetic construct
  REGION 1..5: note = CDR1
SYCMG

SEQ ID NO: 151 - protein, 5 aa, synthetic construct
  REGION 1..5: note = CDR1
HYCMG

SEQ ID NO: 152 - protein, 5 aa, synthetic construct
  REGION 1..5: note = CDR1
SYCMA

SEQ ID NO: 153 - protein, 5 aa, synthetic construct
  REGION 1..5: note = CDR1
TGCMA

SEQ ID NO: 154 - protein, 16 aa, synthetic construct
  REGION 1..16: note = CDR2
FDTSDGSKYYADSVKG

SEQ ID NO: 155 - protein, 15 aa, synthetic construct
  REGION 1..15: note = CDR2
INSDGRTSYADSVKG

SEQ ID NO: 156 - protein, 15 aa, synthetic construct
  REGION 1..15: note = CDR2
IRRDGVTRYADSVKG

SEQ ID NO: 157 - protein, 15 aa, synthetic construct
  REGION 1..15: note = CDR2
IDTDGSTSYTDSVKG

SEQ ID NO: 158 - protein, 16 aa, synthetic construct
  REGION 1..16: note = CDR2
IIRGERGEWYADSVKG

SEQ ID NO: 159 - protein, 15 aa, synthetic construct
  REGION 1..15: note = CDR2
ITSDGWTRYADSVKG

SEQ ID NO: 160 - protein, 18 aa, synthetic construct
  REGION 1..18: note = CDR3
RPFYVGGTCLPRLTDFSI

SEQ ID NO: 161 - protein, 22 aa, synthetic construct
  REGION 1..22: note = CDR3
VILPPTDGMEYSCNYPAAEYNY

SEQ ID NO: 162 - protein, 17 aa, synthetic construct
  REGION 1..17: note = CDR3
QRDCGISGLLWQSSVGY

SEQ ID NO: 163 - protein, 19 aa, synthetic construct
  REGION 1..19: note = CDR3
APWLLAYGDYCVTRPDFGE

SEQ ID NO: 164 - protein, 14 aa, synthetic construct
  REGION 1..14: note = CDR3
DYRYPCDGRSNYDY

SEQ ID NO: 165 - protein, 18 aa, synthetic construct
  REGION 1..18: note = CDR3
TPEGSCSTGVSTPSTFRV

SEQ ID NO: 166 - protein, 27 aa, synthetic construct
  REGION 1..27: note = FR1
HVQLVESGGGSVQAGGSLRLSCEAFEY

SEQ ID NO: 167 - protein, 27 aa, synthetic construct
  REGION 1..27: note = FR1
EVQLVESGGGLVQPGGSLRLSCAAFEY

SEQ ID NO: 168 - protein, 27 aa, synthetic construct
  REGION 1..27: note = FR1
EVQLVESGGGLVQPGGSLRLSCEAFEY

SEQ ID NO: 169 - protein, 30 aa, synthetic construct
  REGION 1..30: note = FR1
HVQLVESGGGSVQSGGSLRLSCAASAYIYS

SEQ ID NO: 170 - protein, 30 aa, synthetic construct
  REGION 1..30: note = FR1
EVQLVESGGGLVQPGGSLRLSCAASAYIYS

SEQ ID NO: 171 - protein, 30 aa, synthetic construct
  REGION 1..30: note = FR1
QVQLVESGGGSVQSGGSLRLSCVDSGYTYG

SEQ ID NO: 172 - protein, 30 aa, synthetic construct
  REGION 1..30: note = FR1
QVQLVESGGGVVQPGGSLRLSCAASGYTYG

SEQ ID NO: 173 - protein, 30 aa, synthetic construct
  REGION 1..30: note = FR1
QVQLVESGGGVVQPGGSLRLSCADSGYTYG

SEQ ID NO: 174 - protein, 30 aa, synthetic construct
  REGION 1..30: note = FR1
HVQLVESGGGSVQAGGSLRLSCAHPEYTSS

SEQ ID NO: 175 - protein, 30 aa, synthetic construct
  REGION 1..30: note = FR1
EVQLVESGGGLVQPGGSLRLSCAASEYTSS

SEQ ID NO: 176 - protein, 30 aa, synthetic construct
  REGION 1..30: note = FR1
EVQLVESGGGLVQPGGSLRLSCAHPEYTSS

SEQ ID NO: 177 - protein, 30 aa, synthetic construct
  REGION 1..30: note = FR1
QVQLVESGGGSVHAGGSLRLSCAVSGYTHS

SEQ ID NO: 178 - protein, 30 aa, synthetic construct
  REGION 1..30: note = FR1
DVQLVESGGGSVQVGGSLRLSCVDSGYTYT

SEQ ID NO: 179 - protein, 15 aa, synthetic construct
  REGION 1..15: note = FR2
WFRQAPGKEREGVAS

SEQ ID NO: 180 - protein, 15 aa, synthetic construct
  REGION 1..15: note = FR2
WFRQAPGKGLEGVAS

SEQ ID NO: 181 - protein, 15 aa, synthetic construct
  REGION 1..15: note = FR2
WFRQAPGKERERVSV

SEQ ID NO: 182 - protein, 15 aa, synthetic construct
  REGION 1..15: note = FR2
WFRQAPGKGLERVSV

SEQ ID NO: 183 - protein, 15 aa, synthetic construct
  REGION 1..15: note = FR2
WFRQAPGKEREGVAA

SEQ ID NO: 184 - protein, 15 aa, synthetic construct
  REGION 1..15: note = FR2
WFRQAPGKGLEGVAA

SEQ ID NO: 185 - protein, 15 aa, synthetic construct
  REGION 1..15: note = FR2
WIRQAPGKEREGVAA

SEQ ID NO: 186 - protein, 15 aa, synthetic construct
  REGION 1..15: note = FR2
WIRQAPGKGLEGVSA

SEQ ID NO: 187 - protein, 15 aa, synthetic construct
  REGION 1..15: note = FR2
WIRQAPGKGLEGVAA

SEQ ID NO: 188 - protein, 15 aa, synthetic construct
  REGION 1..15: note = FR2
WFRQAPGEEREGVAV

SEQ ID NO: 189 - protein, 32 aa, synthetic construct
  REGION 1..32: note = FR3
RFTISKDIAKNTLYLEMNILQAEDTAMYYCAA

SEQ ID NO: 190 - protein, 32 aa, synthetic construct
  REGION 1..32: note = FR3
RFTISRDNSKNTLYLQMNSLRAEDTAVYYCAA

SEQ ID NO: 191 - protein, 32 aa, synthetic construct
  REGION 1..32: note = FR3
RFTISKDNSKNTLYLQMNSLRAEDTAVYYCAA

SEQ ID NO: 192 - protein, 32 aa, synthetic construct
  REGION 1..32: note = FR3
RFTISQDNAKNTLYLQMNSLKPEDTAMYYCAA

SEQ ID NO: 193 - protein, 32 aa, synthetic construct; identical to SEQ ID NO: 26
  REGION 1..32: note = FR3

SEQ ID NO: 194 - protein, 32 aa, synthetic construct
  REGION 1..32: note = FR3
RFTISLDNAKSTLYLQMNSLKAEDTAMYYCAT

SEQ ID NO: 195 - protein, 32 aa, synthetic construct
  REGION 1..32: note = FR3
RFTISRDNSKNTLYLQMNSLRAEDTAVYYCAT

SEQ ID NO: 196 - protein, 32 aa, synthetic construct
  REGION 1..32: note = FR3
RFTISLDNSKNTLYLQMNSLRAEDTAVYYCAT

SEQ ID NO: 197 - protein, 32 aa, synthetic construct
  REGION 1..32: note = FR3
RFTISKDNAKNTLYLQMNNLKPEDTAMYYCAA

SEQ ID NO: 198 - protein, 32 aa, synthetic construct
  REGION 1..32: note = FR3
RFTISQDNAENTLYLQMNSLKSEDTAMYYCAA

SEQ ID NO: 199 - protein, 32 aa, synthetic construct
  REGION 1..32: note = FR3
RFTISKDNALNTLYLQMNSLKPEDTAMYYCAT

SEQ ID NO: 200 - protein, 11 aa, synthetic construct
  REGION 1..11: note = FR4
WGQGTQVTVSS

SEQ ID NO: 201 - protein, 11 aa, synthetic construct
  REGION 1..11: note = FR4
WGQGTLVTVSS

SEQ ID NO: 202 - skipped

SEQ ID NO: 203 - protein, 16 aa, synthetic construct
  REGION 1..16: note = 1-CDR2; 2-CDR2; 3-CDR2; 4-CDR2; 5-CDR2; 6-CDR2
  VAR_SEQ 1: note = X = F, I or missing
  VAR_SEQ 2: note = X = D or I
  VAR_SEQ 3: note = X = D, N, R or T
  VAR_SEQ 4: note = X = G, R, S or T
  VAR_SEQ 5: note = X = D or E
  VAR_SEQ 6: note = X = G or R
  VAR_SEQ 7: note = X = G, R, S, V or W
  VAR_SEQ 8: note = X = E, K or T
  VAR_SEQ 9: note = X = R, S, W or Y
  VAR_SEQ 11: note = X = A or T
XXXXXXXXXYXDSVKG

SEQ ID NO: 204 - protein, 30 aa, synthetic construct
  REGION 1..30: note = 1-FR1-M; 1-FR1-H1; 1-FR1-H2; 1-FR1-H3; 1-FR1-H4; 1-FR1-H5; 2-FR1-M; 2-FR1-H1; 2-FR1-H2; 2-FR1-H3; 3-FR1-M; 3-FR1-H1; 3-FR1-H2; 3-FR1-H3; 3-FR1-H4; 4-FR1-M; 4-FR1-H1; 4-FR1-H2; 4-FR1-H3; 4-FR1-H4; 4-FR1-H5; 5-FR1; 6-FR1
  VAR_SEQ 1: note = X = D, E, H or Q
  VAR_SEQ 11: note = X = L, S or V
  VAR_SEQ 13: note = X = H or Q
  VAR_SEQ 14: note = X = A, P, S or V
  VAR_SEQ 23: note = X = A, E or V
  VAR_SEQ 24: note = X = A, D, H or V
  VAR_SEQ 25: note = X = F, P or S
  VAR_SEQ 26: note = X = A, E or G
  VAR_SEQ 28: note = X = I, T or missing
  VAR_SEQ 29: note = X = H, S, Y or missing
  VAR_SEQ 30: note = X = G, S, T or missing
XVQLVESGGGXVXXGGSLRLSCXXXXYXXX

SEQ ID NO: 205 - protein, 15 aa, synthetic construct
  REGION 1..15: note = 1-FR2-M; 1-FR2-H5; 5-FR2; 1-FR2-H1; 1-FR2-H2; 1-FR2-H3; 1-FR2-H4; 2-FR2-M; 2-FR2-H3; 2-FR2-H1; 2-FR2-H2; 3-FR2-M; 3-FR2-H4; 3-FR2-H1; 3-FR2-H2; 3-FR2-H3; 4-FR2-M; 4-FR2-H5; 4-FR2-H1; 4-FR2-H2; 4-FR2-H3; 4-FR2-H4; 6-FR2
  VAR_SEQ 2: note = X = F or I
  VAR_SEQ 8: note = X = E or K
  VAR_SEQ 9: note = X = E or G
  VAR_SEQ 10: note = X = L or R
  VAR_SEQ 12: note = X = G or R
  VAR_SEQ 14: note = X = A or S
  VAR_SEQ 15: note = X = A, S or V
WXRQAPGXXXEXVXX

SEQ ID NO: 206 - protein, 32 aa, synthetic construct
  REGION 1..32: note = 1-FR3-M; 1-FR3-H1; 1-FR3-H2; 2-FR3-H1; 4-FR3-H1; 4-FR3-H2; 4-FR3-H3; 1-FR3-H3; 1-FR3-H4; 1-FR3-H5; 4-FR3-H4; 4-FR3-H5; 2-FR3-M; 2-FR3-H2; 2-FR3-H3; 3-FR3-M; 3-FR3-H1; 3-FR3-H2; 3-FR3-H3; 3-FR3-H4; 4-FR3-M; 5-FR3; 6-FR3
  VAR_SEQ 6: note = X = K, L, Q or R
  VAR_SEQ 8: note = X = I or N
  VAR_SEQ 9: note = X = A or S
  VAR_SEQ 10: note = X = E, K or L
  VAR_SEQ 11: note = X = N or S
  VAR_SEQ 16: note = X = E or Q
  VAR_SEQ 19: note = X = I, N or S
  VAR_SEQ 21: note = X = K, Q or R
  VAR_SEQ 22: note = X = A, P or S
  VAR_SEQ 27: note = X = M or V
  VAR_SEQ 32: note = X = A or T
RFTISXDXXXXTLYLXMNXLXXEDTAXYYCAX

SEQ ID NO: 207 - protein, 11 aa, synthetic construct
  REGION 1..11: note = 1-FR4-M; 2-FR4-M; 3-FR4-M; 4-FR4-M; 5-FR4; 6-FR4; 1-FR4-H1; 1-FR4-H2; 1-FR4-H3; 1-FR4-H4; 1-FR4-H5; 2-FR4-H1; 2-FR4-H2; 2-FR4-H3; 3-FR4-H1; 3-FR4-H2; 3-FR4-H3; 3-FR4-H4; 4-FR4-H1; 4-FR4-H2; 4-FR4-H3; 4-FR4-H4; 4-FR4-H5
  VAR_SEQ 6: note = X = L or Q
WGQGTXVTVSS

SEQ ID NO: 208 - protein, 124 aa, synthetic construct
  REGION 1..124: note = VHH
HVQLVESGGGSVQAGGSLRLSCEAFEYYYCMGWFRQAPGKEREGVASFDTSDGSKYYADSVKGRFTISKDIAKNTLYLEMNILQAEDTAMYYCAARPFYVGGTCLPRLTDFSIWGQGTQVTVSS

SEQ ID NO: 209 - protein, 124 aa, synthetic construct
  REGION 1..124: note = VHH
EVQLVESGGGLVQPGGSLRLSCAAFEYYYCMGWFRQAPGKGLEGVSSFDTSDGSKYYADSVKGRFTISRDNSKNTLYLQMNSLRAEDTAVYYCAARPFYVGGTCLPRLTDFSIWGQGTLVTVSS

SEQ ID NO: 210 - protein, 124 aa, synthetic construct
  REGION 1..124: note = VHH
EVQLVESGGGLVQPGGSLRLSCAAFEYYYCMGWFRQAPGKGLEGVASFDTSDGSKYYADSVKGRFTISRDNSKNTLYLQMNSLRAEDTAVYYCAARPFYVGGTCLPRLTDFSIWGQGTLVTVSS

SEQ ID NO: 211 - protein, 124 aa, synthetic construct
  REGION 1..124: note = VHH
EVQLVESGGGLVQPGGSLRLSCAAFEYYYCMGWFRQAPGKGLEGVASFDTSDGSKYYADSVKGRFTISKDNSKNTLYLQMNSLRAEDTAVYYCAARPFYVGGTCLPRLTDFSIWGQGTLVTVSS

SEQ ID NO: 212 - protein, 124 aa, synthetic construct
  REGION 1..124: note = VHH
EVQLVESGGGLVQPGGSLRLSCEAFEYYYCMGWFRQAPGKGLEGVASFDTSDGSKYYADSVKGRFTISKDNSKNTLYLQMNSLRAEDTAVYYCAARPFYVGGTCLPRLTDFSIWGQGTLVTVSS

SEQ ID NO: 213 - protein, 124 aa, synthetic construct
  REGION 1..124: note = VHH
EVQLVESGGGLVQPGGSLRLSCEAFEYYYCMGWFRQAPGKEREGVASFDTSDGSKYYADSVKGRFTISKDNSKNTLYLQMNSLRAEDTAVYYCAARPFYVGGTCLPRLTDFSIWGQGTLVTVSS

SEQ ID NO: 214 - protein, 130 aa, synthetic construct
  REGION 1..130: note = VHH
HVQLVESGGGSVQSGGSLRLSCAASAYIYSSYCMGWFRQAPGKERERVSVINSDGRTSYADSVKGRFTISQDNAKNTLYLQMNSLKPEDTAMYYCAAVILPPTDGMEYSCNYPAAEYNYWGQGTQVTVSS

SEQ ID NO: 215 - protein, 130 aa, synthetic construct
  REGION 1..130: note = VHH
EVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSS

SEQ ID NO: 216 - protein, 130 aa, synthetic construct
  REGION 1..130: note = VHH
EVQLVESGGGLVQPGGSLRLSCAASAYIYSSYCMGWFRQAPGKGLERVSVINSDGRTSYADSVKGRFTISQDNSKNTLYLQMNSLRAEDTAVYYCAAVILPPTDGMEYSCNYPAAEYNYWGQGTLVTVSS

SEQ ID NO: 217 - protein, 130 aa, synthetic construct
  REGION 1..130: note = VHH
EVQLVESGGGLVQPGGSLRLSCAASAYIYSSYCMGWFRQAPGKERERVSVINSDGRTSYADSVKGRFTISQDNSKNTLYLQMNSLRAEDTAVYYCAAVILPPTDGMEYSCNYPAAEYNYWGQGTLVTVSS

SEQ ID NO: 218 - protein, 125 aa, synthetic construct
  REGION 1..125: note = VHH
QVQLVESGGGSVQSGGSLRLSCVDSGYTYGHYCMGWFRQAPGKEREGVAAIRRDGVTRYADSVKGRFTISLDNAKSTLYLQMNSLKAEDTAMYYCATQRDCGISGLLWQSSVGYWGQGTQVTVSS

SEQ ID NO: 219 - protein, 125 aa, synthetic construct
  REGION 1..125: note = VHH
QVQLVESGGGVVQPGGSLRLSCAASGYTYGHYCMGWFRQAPGKGLEGVAAIRRDGVTRYADSVKGRFTISRDNSKNTLYLQMNSLRAEDTAVYYCATQRDCGISGLLWQSSVGYWGQGTLVTVSS

SEQ ID NO: 220 - protein, 125 aa, synthetic construct
  REGION 1..125: note = VHH
QVQLVESGGGVVQPGGSLRLSCAASGYTYGHYCMGWFRQAPGKGLEGVAAIRRDGVTRYADSVKGRFTISLDNSKNTLYLQMNSLRAEDTAVYYCATQRDCGISGLLWQSSVGYWGQGTLVTVSS

SEQ ID NO: 221 - protein, 125 aa, synthetic construct
  REGION 1..125: note = VHH
QVQLVESGGGVVQPGGSLRLSCADSGYTYGHYCMGWFRQAPGKGLEGVAAIRRDGVTRYADSVKGRFTISLDNSKNTLYLQMNSLRAEDTAVYYCATQRDCGISGLLWQSSVGYWGQGTLVTVSS

SEQ ID NO: 222 - protein, 125 aa, synthetic construct
  REGION 1..125: note = VHH
QVQLVESGGGVVQPGGSLRLSCADSGYTYGHYCMGWFRQAPGKEREGVAAIRRDGVTRYADSVKGRFTISLDNSKNTLYLQMNSLRAEDTAVYYCATQRDCGISGLLWQSSVGYWGQGTLVTVSS

SEQ ID NO: 223 - protein, 127 aa, synthetic construct
  REGION 1..127: note = VHH
HVQLVESGGGSVQAGGSLRLSCAHPEYTSSSYCMAWIRQAPGKEREGVAAIDTDGSTSYTDSVKGRFTISKDNAKNTLYLQMNNLKPEDTAMYYCAAAPWLLAYGDYCVTRPDFGEWGQGTQVTVSS

SEQ ID NO: 224 - protein, 127 aa, synthetic construct
  REGION 1..127: note = VHH
EVQLVESGGGLVQPGGSLRLSCAASEYTSSSYCMAWIRQAPGKGLEGVSAIDTDGSTSYTDSVKGRFTISRDNSKNTLYLQMNSLRAEDTAVYYCAAAPWLLAYGDYCVTRPDFGEWGQGTLVTVSS

SEQ ID NO: 225 - protein, 127 aa, synthetic construct
  REGION 1..127: note = VHH
EVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSS

SEQ ID NO: 226 - protein, 127 aa, synthetic construct
  REGION 1..127: note = VHH
EVQLVESGGGLVQPGGSLRLSCAHPEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSS

SEQ ID NO: 227 - protein, 127 aa, synthetic construct
  REGION 1..127: note = VHH
EVQLVESGGGLVQPGGSLRLSCAHPEYTSSSYCMAWIRQAPGKGLEGVAAIDTDGSTSYTDSVKGRFTISKDNSKNTLYLQMNSLRAEDTAVYYCAAAPWLLAYGDYCVTRPDFGEWGQGTLVTVSS

SEQ ID NO: 228 - protein, 127 aa, synthetic construct
  REGION 1..127: note = VHH
EVQLVESGGGLVQPGGSLRLSCAHPEYTSSSYCMAWIRQAPGKEREGVAAIDTDGSTSYTDSVKGRFTISKDNSKNTLYLQMNSLRAEDTAVYYCAAAPWLLAYGDYCVTRPDFGEWGQGTLVTVSS

SEQ ID NO: 229 - protein, 516 aa, synthetic construct
  REGION 1..516: note = VHH
EVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSSGGGGSGGGGSGGGGSEVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 230 - protein, 777 aa, synthetic construct
  REGION 1..777: note = VHH
EVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSSGGGGSGGGGSGGGGSEVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGKGGGGSGGGGSGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 231 - protein, 126 aa, synthetic construct
  REGION 1..126: note = VHH
EVQLVESGGGSVQVGGSLRLSCVDTSYTYTHYCMGWFRQAPGKEREGVAVMTTDGWTRYADSVKGRFIISKDNAKNTLYLQMNSLKLEDTAMYYCATNPGDSCTTGISTPPTFRTWGQGTQVTVSS

SEQ ID NO: 232 - protein, 126 aa, synthetic construct
  REGION 1..126: note = VHH
DVQLVESGGGSVQVGGSLRLSCVDTSYTYTHYCMGWFRQAPGKEREGVAVMTTDGWTRYADSVKGRFIISKDNAKNTLYLQMNSLKLEDTAMYYCATNPWDSCTTGISTPSTFRTWGQGTQVTVSS

SEQ ID NO: 233 - protein, 127 aa, synthetic construct
  REGION 1..127: note = VHH
EVQLVESGGGSVQAGGSLRLSCAASISIRSFNYMAWFRQAPGKEREGVAASYAGGVSTYYTDSVKGRFTISQDNAQKRVYLQMNSLKPEDTAVYYCAATQDVIWRTGPLESDEYRYWGQGTQVTVSS

SEQ ID NO: 234 - protein, 122 aa, synthetic construct
  REGION 1..122: note = VHH
EVQLVESGGGSVQAGGSLKLSCVVSAYTYSVACMGWVRQAPGKEREGVAAIDSYGRTSYPDSVKGRFTISRDNTKNTLYLQTNSLKPEDTGMYYCAADLTRTLVDGCNWTHWGQGTQVTVSS

SEQ ID NO: 235 - protein, 129 aa, synthetic construct
  REGION 1..129: note = VHH
KVQLVESGGGSVQAGGSLRLSCAASGYTYSIGCMGWFRQAPGKEREGVAAIDSDGSTSYADSVKGRFTISKHNAKNTLYLQMDSLKPEDTAMYYCAADVHPIGHPVAGPHLKSADFGYWGQGTQVTVSS

SEQ ID NO: 236 - protein, 124 aa, synthetic construct
  REGION 1..124: note = VHH
DVQLVESGGGSVQAGGSLRLSCAASTYTGSRINMAWFRQATGKEREGVATIMIGGPYTHYADSLKGRLTISQDSAKNTMYLQMNSLKPEDTAVYYCAADSRIVGSLVDSDYKYWGQGTQVTVSS

SEQ ID NO: 237 - protein, 126 aa, synthetic construct
  REGION 1..126: note = VHH
DVQLVESGGGSVQVGGSLKLSCVDSGYTYSHYCMAWFRQAPGKEREGVAVIASDGWTRYADSVKGRFTISKDSAKNTLYLQMNSLKPEDTAMYYCASDPTDLCTTGLSTPSKFLNWGQGTQVTVSS

SEQ ID NO: 238 - protein, 125 aa, synthetic construct
  REGION 1..125: note = VHH
EVQLVESGGGSVQSGGSLRLSCAASGDTYSRSCMGWFRQAPGKEREGVADIDSDGSRTYADSVKGRFTISQDNDKNTLYLQMNSLKPEDTAMYYCAADRVSIDGGCHESVHLNFWGQGTQVTVSS

SEQ ID NO: 239 - protein, 124 aa, synthetic construct
  REGION 1..124: note = VHH
QVQLVESGGGSVQAGGSLRLFCAASGYDYDTWFYMAWFRQAPGKEREGVAAIDTIGNTNYADSVKGRFTISKGNAKNSLYLQINSLKPEDTAIYFCAAGQSLPLALSSNEYEFWGQGTQVTVSS

SEQ ID NO: 240 - protein, 123 aa, synthetic construct
  REGION 1..123: note = VHH
QVQLVESGGGSVHAGGSLRLSCAVSGYTHSTGCMAWFRQAPGKEREGVASIIRGERGEWYADSVKGRFTISQDNAENTLYLQMNSLKSEDTAMYYCAADYRYPCDGRSNYDYWGQGTQVTVSS

SEQ ID NO: 241 - protein, 126 aa, synthetic construct
  REGION 1..126: note = VHH
DVQLVESGGGSVQVGGSLRLSCVDSGYTYTHYCMGWFRQAPGEEREGVAVITSDGWTRYADSVKGRFTISKDNALNTLYLQMNSLKPEDTAMYYCATTPEGSCSTGVSTPSTFRVWGQGTQVTVSS

SEQ ID NO: 242 - protein, 136 aa, synthetic construct
  REGION 1..136: note = VHH
DVQLVESGGGSVQSGGSLRLSCAASGRTWSNYCMGWFRQAPGKEREGVAVIDSDGSTSYVDSVKGRFTISEDNAKNTLYLQMNSLKPEDTAMYYCAAAGVIRYDDDRALSDLCGVEVTSAADFAYWGQGTQVTVSS

SEQ ID NO: 243 - protein, 134 aa, synthetic construct
  REGION 1..134: note = VHH
HVQLVESGGGSVQSGGSLRLSCVASGYTSGYTYSSYCIAWFRQAPGKEREGVAQIDSDGSTTYADSVKGRFTISQDNVKNTLYLQMNSLKPDDTAMYYCAAEPRPHTYYSCTYLNHGSLDFGYWGQGTQVTVSS

SEQ ID NO: 244 - protein, 129 aa, synthetic construct
  REGION 1..129: note = VHH
HVQLVESGGGSVQSGGSLRLSCAASGMTWSRYCMGWFRQASGKEREGVAAIGSDGDTSYADSVKGRFTISQDNAKKTLYLQMNSLKPEDTAMYYCAAAGVAGTFEGLTCPTVEYYYAYWGQGTQVTVSS

SEQ ID NO: 245 - protein, 125 aa, synthetic construct
  REGION 1..125: note = VHH
QVQLVESGGGSVQAGGSLRLSCAASGITWSRYCMAWFRQVPGKEREGVAAMNNAGSTSYIESVKARFTISEDNAKNTLYLQMNSLKPEDTAMYYCAVAGSTDGTCAYEAYLFANWGRGTQVTVSS

SEQ ID NO: 246 - protein, 512 aa, synthetic construct
  REGION 1..512: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCEAFEYYYCMGWFRQAPGKEREGVASFDTSDGSKYYADSVKGRFTISKDIAKNTLYLEMNILQAEDTAMYYCAARPFYVGGTCLPRLTDFSIWGQGTQVTVSSGGGGSGGGGSGGGGSKVQLVESGGGSVQAGGSLRLSCAASGYTYSIGCMGWFRQAPGKEREGVAAIDSDGSTSYADSVKGRFTISKHNAKNTLYLQMDSLKPEDTAMYYCAADVHPIGHPVAGPHLKSADFGYWGQGTQVTVSS

SEQ ID NO: 247 - protein, 513 aa, synthetic construct
  REGION 1..513: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCEAFEYYYCMGWFRQAPGKEREGVASFDTSDGSKYYADSVKGRFTISKDIAKNTLYLEMNILQAEDTAMYYCAARPFYVGGTCLPRLTDFSIWGQGTQVTVSSGGGGSGGGGSGGGGSHVQLVESGGGSVQSGGSLRLSCAASAYIYSSYCMGWFRQAPGKERERVSVINSDGRTSYADSVKGRFTISQDNAKNTLYLQMNSLKPEDTAMYYCAAVILPPTDGMEYSCNYPAAEYNYWGQGTQVTVSS

SEQ ID NO: 248 - protein, 508 aa, synthetic construct
  REGION 1..508: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCEAFEYYYCMGWFRQAPGKEREGVASFDTSDGSKYYADSVKGRFTISKDIAKNTLYLEMNILQAEDTAMYYCAARPFYVGGTCLPRLTDFSIWGQGTQVTVSSGGGGSGGGGSGGGGSQVQLVESGGGSVQSGGSLRLSCVDSGYTYGHYCMGWFRQAPGKEREGVAAIRRDGVTRYADSVKGRFTISLDNAKSTLYLQMNSLKAEDTAMYYCATQRDCGISGLLWQSSVGYWGQGTQVTVSS

SEQ ID NO: 249 - protein, 514 aa, synthetic construct
  REGION 1..514: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSDVQLVESGGGSVQVGGSLRLSCVDTSYTYTHYCMGWFRQAPGKEREGVAVMTTDGWTRYADSVKGRFIISKDNAKNTLYLQMNSLKLEDTAMYYCATNPWDSCTTGISTPSTFRTWGQGTQVTVSSGGGGSGGGGSGGGGSKVQLVESGGGSVQAGGSLRLSCAASGYTYSIGCMGWFRQAPGKEREGVAAIDSDGSTSYADSVKGRFTISKHNAKNTLYLQMDSLKPEDTAMYYCAADVHPIGHPVAGPHLKSADFGYWGQGTQVTVSS

SEQ ID NO: 250 - protein, 515 aa, synthetic construct
  REGION 1..515: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSDVQLVESGGGSVQVGGSLRLSCVDTSYTYTHYCMGWFRQAPGKEREGVAVMTTDGWTRYADSVKGRFIISKDNAKNTLYLQMNSLKLEDTAMYYCATNPWDSCTTGISTPSTFRTWGQGTQVTVSSGGGGSGGGGSGGGGSHVQLVESGGGSVQSGGSLRLSCAASAYIYSSYCMGWFRQAPGKERERVSVINSDGRTSYADSVKGRFTISQDNAKNTLYLQMNSLKPEDTAMYYCAAVILPPTDGMEYSCNYPAAEYNYWGQGTQVTVSS

SEQ ID NO: 251 - protein, 510 aa, synthetic construct
  REGION 1..510: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSDVQLVESGGGSVQVGGSLRLSCVDTSYTYTHYCMGWFRQAPGKEREGVAVMTTDGWTRYADSVKGRFIISKDNAKNTLYLQMNSLKLEDTAMYYCATNPWDSCTTGISTPSTFRTWGQGTQVTVSSGGGGSGGGGSGGGGSQVQLVESGGGSVQSGGSLRLSCVDSGYTYGHYCMGWFRQAPGKEREGVAAIRRDGVTRYADSVKGRFTISLDNAKSTLYLQMNSLKAEDTAMYYCATQRDCGISGLLWQSSVGYWGQGTQVTVSS

SEQ ID NO: 252 - protein, 515 aa, synthetic construct
  REGION 1..515: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCAHPEYTSSSYCMAWIRQAPGKEREGVAAIDTDGSTSYTDSVKGRFTISKDNAKNTLYLQMNNLKPEDTAMYYCAAAPWLLAYGDYCVTRPDFGEWGQGTQVTVSSGGGGSGGGGSGGGGSKVQLVESGGGSVQAGGSLRLSCAASGYTYSIGCMGWFRQAPGKEREGVAAIDSDGSTSYADSVKGRFTISKHNAKNTLYLQMDSLKPEDTAMYYCAADVHPIGHPVAGPHLKSADFGYWGQGTQVTVSS

SEQ ID NO: 253 - protein, 516 aa, synthetic construct
  REGION 1..516: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCAHPEYTSSSYCMAWIRQAPGKEREGVAAIDTDGSTSYTDSVKGRFTISKDNAKNTLYLQMNNLKPEDTAMYYCAAAPWLLAYGDYCVTRPDFGEWGQGTQVTVSSGGGGSGGGGSGGGGSHVQLVESGGGSVQSGGSLRLSCAASAYIYSSYCMGWFRQAPGKERERVSVINSDGRTSYADSVKGRFTISQDNAKNTLYLQMNSLKPEDTAMYYCAAVILPPTDGMEYSCNYPAAEYNYWGQGTQVTVSS

SEQ ID NO: 254 - protein, 511 aa, synthetic construct
  REGION 1..511: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCAHPEYTSSSYCMAWIRQAPGKEREGVAAIDTDGSTSYTDSVKGRFTISKDNAKNTLYLQMNNLKPEDTAMYYCAAAPWLLAYGDYCVTRPDFGEWGQGTQVTVSSGGGGSGGGGSGGGGSQVQLVESGGGSVQSGGSLRLSCVDSGYTYGHYCMGWFRQAPGKEREGVAAIRRDGVTRYADSVKGRFTISLDNAKSTLYLQMNSLKAEDTAMYYCATQRDCGISGLLWQSSVGYWGQGTQVTVSS

SEQ ID NO: 255 - protein, 510 aa, synthetic construct
  REGION 1..510: note = VHH
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGSSGGGGSHVQLVESGGGSVQAGGSLRLSCEAFEYYYCMGWFRQAPGKEREGVASFDTSDGSKYYADSVKGRFTISKDIAKNTLYLEMNILQAEDTAMYYCAARPFYVGGTCLPRLTDFSIWGQGTQVTVSSGGGGSGGGGSGGGGSHVQLVESGGGSVQAGGSLRLSCAHPEYTSSSYCMAWIRQAPGKEREGVAAIDTDGSTSYTDSVKGRFTISKDNAKNTLYLQMNNLKPEDTAMYYCAAAPWLLAYGDYCVTRPDFGEWGQGTQVTVSS

SEQ ID NO: 256 - protein, 15 aa, synthetic construct
  REGION 1..15: note = linker
GGGGSGGGGSGGGGS

SEQ ID NO: 257 - protein, 229 aa, synthetic construct; identical to SEQ ID NO: 20
  REGION 1..229: note = Fc

SEQ ID NO: 258 - protein, 246 aa, synthetic construct
  REGION 1..246: note = factorH
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 259 - DNA, 48 bp, synthetic construct
  misc_feature 1..48: note = Forward primer
  misc_feature 31: note = n = c or g
  misc_feature 33: note = n = g or t
  source 1..48: mol_type = other DNA
ggcggtggcagctcaggaggcggcggatccnangtgcagctggtggag

SEQ ID NO: 260 - DNA, 46 bp, synthetic construct
  misc_feature 1..46: note = Reverse primer
  misc_feature 40: note = n = a or g
  source 1..46: mol_type = other DNA
aatccagaggttgattgtcgacgtacgctatgaggagacngtgacc

SEQ ID NO: 261 - protein, 495 aa, synthetic construct; identical to SEQ ID NO: 21
  REGION 1..495: note = VEGF inhibiting VHHs

SEQ ID NO: 262 - protein, 782 aa, synthetic construct
  REGION 1..782: note = SLN12140 and VEGF inhibiting VHHs
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSSGGGGSGGGGSGGGGSEVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSS

SEQ ID NO: 263 - protein, 128 aa, synthetic construct; identical to SEQ ID NO: 108
  REGION 1..128: note = hTf binding VHH

SEQ ID NO: 264 - protein, 272 aa, synthetic construct
  REGION 1..272: note = properdin inhibiting VHHs
EVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSSGGGGSGGGGSGGGGSEVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSS

SEQ ID NO: 265 - protein, 415 aa, synthetic construct
  REGION 1..415: note = SLN12147 and hTf binding VHH
EVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSSGGGGSGGGGSGGGGSEVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSSGGGGSGGGGSGGGGSEVQLVESGGGLVQPGGSLRLSCAASGHAYGGNYMGWFRQAPGKGLEGVAVLYTGGGSTYYADSVKGRFTISEDNSKNTVYLQMNSLRAEDTAVYYCALALGSARWYTSSLDARAYNIWGQGTLVTVSS

SEQ ID NO: 266 - protein, 659 aa, synthetic construct
  REGION 1..659: note = SLN12140 and hTf binding VHH
EVQLVESGGGLVQPGGSLRLSCAASAYIYSSYCMGWFRQAPGKGLERVSVINSDGRTSYADSVKGRFTISRDNSKNTLYLQMNSLRAEDTAVYYCAAVILPPTDGMEYSCNYPAAEYNYWGQGTLVTVSSGGGGSGGGGSGGGGSEVQLVESGGGLVQPGGSLRLSCAASEYTSSSYCMAWIRQAPGKGLEGVAAIDTDGSTSYTDSVKGRFTISRDNSKNTLYLQMNSLRAEDTAVYYCAAAPWLLAYGDYCVTRPDFGEWGQGTLVTVSSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGHAYGGNYMGWFRQAPGKGLEGVAVLYTGGGSTYYADSVKGRFTISEDNSKNTVYLQMNSLRAEDTAVYYCALALGSARWYTSSLDARAYNIWGQGTLVTVSS

(19) United States
(12) Patent Application Publication
(10) Pub. No.: US 2025/0034239 A1
(43) Pub. Date: Jan. 30, 2025

(54) PROTEINS AND USE THEREOF

(71) Applicant: Linno Pharmaceuticals lnc., Shanghai (CN)

(72) Inventors: Zhaozhong HAN, Shanghai (CN); Hongya PAN, Shanghai (CN); Lingyu LI, Shanghai (CN); Mengfan PENG, Shanghai (CN); Jun LUO, Shanghai (CN); Haiyang LI, Shanghai (CN)

(21) Appl. No.: 18/912,612

(22) Filed: Oct. 11, 2024

Related U.S. Application Data
(63) Continuation-in-part of application No. 18/711,199, filed on May 17, 2024.
(63) Continuation-in-part of application No. 18/327,091, filed on Jun. 1, 2023, now Pat. No. 12,144,866.
Application No. 18/327,091 is a continuation of application No. PCT/CN2021/135059, filed on Dec. 2, 2021.
(63) Continuation-in-part of application No. 18/459,465, filed on Sep. 1, 2023, now Pat. No. 12,162,931.
Application No. 18/459,465 is a continuation of application No. PCT/CN2023/108521, filed on Jul. 21, 2023.

(30) Foreign Application Priority Data
Nov. 25, 2022 (WO) PCT/CN2022/134249

(51) Int. Cl. C07K 16/22; A61K 39/00

(52) U.S. Cl. CPC C07K 16/22; A61K 2039/505; C07K 2317/31; C07K 2317/565; C07K 2317/569; C07K 2317/76; C07K 2317/94

(57) ABSTRACT

The disclosure provides proteins specifically binding to Vascular Endothelial Growth Factor (VEGF), transferrin and/or properdin.

### CROSS-REFERENCE TO RELATED APPLICATIONS

The application is a continuation in part of U.S. Application No. U.S. application Ser. No. 18/711,199, filed on May 17, 2024, which is a U.S. National Stage filing under 35 U.S.C. § 371 of International Application No. PCT/CN2021/132760, filed Nov. 24, 2021; a continuation in part of U.S. application Ser. No. 18/327,091, filed Jun. 1, 2023, which is a continuation of application No. PCT/CN2021/135059, filed on Dec. 2, 2021; and a continuation in part of U.S. application Ser. No. 18/459,465, filed Sep. 1, 2023, which is a continuation of application No. PCT/CN2023/108521, filed on Jul. 21, 2023, which claims the benefit of priority to PCT Application No. PCT/CN2022/134249, filed Nov. 25, 2022, the contents of each of which applications are incorporated herein by reference in their entireties.

### SEQUENCE LISTING

The instant application contains a Sequence Listing submitted electronically as a .xml file named “086980-8002US01_ST26.xml”. The .xml copy, created on Sep. 13, 2024 is 375,342 bytes in size. The Sequence Listing is hereby incorporated by reference in its entirety.

### FIELD OF THE INVENTION

The present application relates to the field of biomedicine. The present application provides some proteins specifically binding to Vascular Endothelial Growth Factor (VEGF), transferrin and properdin, respectively. The present application also provides the use of these proteins.

### TECHNICAL BACKGROUND

Vascular Endothelial Growth Factor (VEGF) is a growth factor capable of promoting the division and proliferation of endothelial cells, promoting the formation of new blood vessels, and improving vascular permeability. It exercises such biological activities through binding to appropriate growth factor receptor(s) and activating intracellular signaling pathway(s). Within tumor mass, multiple types of cells including tumor cells, and tumor-infiltrating macrophages and mast cells can secrete high levels of VEGF that stimulate sprouting of surrounding blood vessels towards tumor mass, homing, proliferation and migration of endothelial progenitor cells, and collectively induce angiogenesis to support continuous growth of tumor mass. High levels of VEGF also improve vascular permeability, promote fibroblast invasion, fibrous deposition and tumor stroma formation that contributes to drug resistance and tumor metastasis. Thus, VEGF inhibition has been studied as one of the most promising options for tumor treatment. The VEGF family includes VEGF-A, VEGF-B, VEGF-C, VEGF-D and PIGF. VEGF-A is a homodimeric glycoprotein with a molecular weight of 45 KD. As the most specific and most critical angiogenesis factor among the family members, VEGF-A primarily binds VEGFR-2 to activate the downstream signaling pathways for exercising its biological activities.

VEGF-A inhibiting antibodies and tyrosine kinase inhibiting compounds have been exploited as therapeutics for VEGF-driven diseases like tumor or wet age-related macular degeneration (wAMD). There is a need to obtain the VEGFA-binding proteins with various bioactivities for treating VEGFA-associated diseases.

Transmembrane drug delivery systems could be applied for blood-brain barrier (BBB)-crossing drug delivery, oral drug delivery, intracellular drug delivery and the like. Currently, among the non-invasive blood-brain barrier drug delivery technologies such as receptor-mediated BBB-crossing drug delivery, transferrin receptor (TfR) has been the most widely studied target protein. Those TfR-targeting vehicles include antibody clones known as OX26 and 8D3 in a format as Fabs, scFv, or dual-variable-domain immunoglobulin, a peptide known as GYR22, and an engineered Fc region of human IgG with TfR-binding properties. Although most of such approaches are still at a preclinical stage, some studies have shown promising results in clinical trials, even with several limitations.

TfR antibody affinity and valency can alter intracellular trafficking of the TfR and delivered therapeutic entities; high-affinity bivalent TfR antibodies were shown to divert the antibody-TfR complex into lysosomes, resulting in degradation and reduced levels of TfR. Therefore, TfR antibodies used for transmembrane drug delivery need to be optimal regarding affinity to TfR and/or monovalent to achieve higher efficiencies of transcytosis into the brain. The efficiency of TfR-targeting delivery technologies may vary upon biological or biochemical features of the to-be-delivered entities, anti-TfR antibodies may also cause acute clinical signs and a decrease of circulate reticulocyte count. Ubiquitous expression of TfR throughout the body, suggests that a TfR-targeting strategy will probably result in enhanced uptake of a TfR-targeted drug into peripheral compartments, such as the bone marrow, liver and spleen.

With a few exceptions, oral administration is currently an option only for small drug molecules that show acceptable intestinal absorption. As a rapidly expanding class of drugs, biologics are presently predominantly given by injection. Significant research efforts over decades have explored technologies to enable oral delivery of biologics, but progress has been relatively less impressive. Drug delivery strategies in this area mostly utilize absorption or permeation enhancers such as SNAC or focus on biologics with a relatively smaller mass, such as glucagon-like peptide 1 (GLP-1) analogues. However, safety concerns, including those related to many surfactants used for formulation, have hindered the clinical translation of these approaches. The key challenge in the field of oral delivery of macromolecular biologics concerns the difficulty in overcoming the formidable intestinal epithelial barrier, rather than additional barriers such as the stomach acid and mucosal enzymes, which can be addressed via relatively established technologies. A key requirement for technologies enabling therapeutically-relevant oral delivery of biologics is safety. Rather than disrupting and increasing the permeability of the intestinal epithelium non-selectively (i.e., an effect that a classical permeation enhancer would display), it is desirable to engineer delivery systems that selectively permeate the intestinal mucosa.

The complement system, as a vital part of innate and adaptive immunity, plays an important role in the clearance of pathogens, cell debris and mutated cells. However, unregulated activation of this system has a significant or critical role in the pathogenesis of human diseases including eye diseases, periodontal diseases, cancer, autoimmune diseases, CNS/PNS diseases, kidney diseases and chronic hemolytic diseases. Complement inhibition has been successfully applied to clinical or experimental treatment of few human diseases such as paroxysmal nocturnal hemoglobinuria (PNH), atypical hemolytic uremic syndrome (aHUS), generalized myasthenia gravis (gMG); Neuromyelitis optica spectrum disorder (NMOSD), thrombotic microangiopathy (TMAs), age-related macular degeneration (AMD), IgA nephropathy (IgAN), and Alzheimer's disease (AD).

The complement system can be activated via three routes as classical (CP), lectin (LP) or alternative (AP) pathway, respectively. The AP represents a true safeguard system that is always active and also accounts for approximately 80-90% of terminal pathway activation by forming a powerful amplification loop for the three complement pathways. The C3 convertase, either in the fluid phase or on cell surfaces, has a short half-life of around 90 seconds under physiological conditions. Properdin, a glycoprotein with low levels in plasma and high levels at inflammatory sites where it is dumped by the activated neutrophils, is the only positive regulator of the complement system by binding to and stabilizing surface-bound C3 convertases (C3bBb) and C5 convertases (C3bBbC3b) by extending the half-life of the nascent convertases by 5 to 10 folds, leading to an accelerated and efficient amplification of C3b deposition on the surface of targets. Therapeutic inhibitors of properdin would block complement at an earlier stage by interfering with the unregulated amplification of the AP and leaving CP and LP activation to physiological functions and thus potentially ameliorate human diseases more effectively and safely where the AP participates in the pathogenesis, in particular in diseases where properdin levels are increased and where properdin has been shown to play an important role in the pathogenesis.

In the past ten years, targeting complement system has been gradually gained attentions for treatment of human diseases. By interfering with terminal pathway effector generation, eculizumab (Soliris, Alexion Pharm), one humanized monoclonal antibody against human complement C5 protein, was firstly approved by the FDA in 2007 for the treatment of PNH, and subsequently expanding the indications to aHUS, gMG and NMOSD. Encouraged by such successful clinical applications, C5 antagonists in variable formats such as modified peptide, aptamer, small molecular compound (SMC), siRNA or antisense oligonucleotide (ASO) have been actively developed in clinical or preclinical settings. Molecular targets have been extended to complement proteins that are dominant in complement activation via classical, lectin or alternative pathways, including C3, complement factor B, complement factor D, MASP-2 or MASP-3, CIs, and complement factor H or I, et. al. Particularly, OMS721 (Omeros), a human monoclonal antibody targeting mannose-binding lectin-associated serine protease-2 (MASP-2), significantly reduced the urinary albumin/creatinine ratio of patients in a phase 2 clinical trial for the treatment of IgA nephropathy. The efficacy was unprecedented in other therapies, which also earned it the FDA's breakthrough therapy designation. Furthermore, other orally bioavailable drugs are progressing through phase 2 with a focus on the amplification loop. LNP023 (Novartis) blocks CFB and is in clinical trials for a number of indications including PNH and renal disease. Another potential target for convertase formation is properdin, a fully-human anti-properdin Fab (CLG561) was developed by Novartis for use in AMD; it had been evaluated as monotherapy or in combination with the anti-C5 mAb LFG316 in a phase 2 trial for geographic atrophy (NCT02515942).

Inhibition or modulation of properdin is an important therapeutic strategy to mitigate symptoms and slow or prevent progression of disease associated with alternative pathway. It's a viable and promising therapeutic strategy to block alternative pathway without inhibiting the classical complement pathway by depleting, neutralizing, or inactivating properdin.

### SUMMARY OF THE INVENTION

In one aspect, the present disclosure provides an isolated antibody or an antigen-binding fragment thereof capable of binding VEGF-A (Vascular Endothelial Growth Factor A), and use thereof. In some embodiments, the isolated antibody or an antigen-binding fragment thereof capable of binding VEGF-A, having one or more properties selected from the group consisting of: 1) an ability of specifically binding to VEGF-A; 2) an ability of preventing VEGF-A from binding to its corresponding receptor(s); and 3) an ability of inhibiting VEGF-driven biological functions. In some embodiments, the VEGF-driven biological function comprises angiogenesis. In some embodiments, the VEGF-A comprises human VEGF-A.

In another aspect, the present disclosure provides the present disclosure provides an isolated antibody or an antigen-binding fragment thereof capable of binding transferrin. In some embodiments, the isolated antibody or an antigen-binding fragment thereof capable of binding transferrin can extend half-life in circulation of its associated entities. In some embodiments, the isolated antibody or an antigen-binding fragment thereof capable of binding transferrin enables a drug comprising the transferrin-binding protein to cross blood-brain barrier (BBB). In some embodiments, the isolated antibody or an antigen-binding fragment thereof is capable of specifically binding to transferrin and not disturbing the interaction between transferrin and transferrin receptor 1. The present disclosure also provides a method to translocate a molecule across cellular membrane by using the isolated antibody or an antigen-binding fragment thereof capable of binding transferrin. The present disclosure further provides a method for extending half-life in circulation of a therapeutic entity, wherein the therapeutic entity is linked with the isolated antibody or an antigen-binding fragment thereof capable of binding transferrin directly or indirectly.

In another aspect, the present disclosure provides an isolated antibody or an antigen-binding fragment thereof capable of binding properdin, which may have one or more of the following properties: 1) inhibits alternative pathway by binding properdin, 2) inhibits interaction between properdin and C3, 3) selectively inhibits alternative pathway rather than classical pathway or lectin pathway, and 4) has species-crossing properdin-binding and complement-inhibitory activity in AP-specific pathways in mammal. The isolated antibody or an antigen-binding fragment thereof capable of binding properdin also shows serum stability both in plasma and formulation buffer. By multiple subcutaneous dosing said isolated antibody or an antigen-binding fragment thereof capable of binding properdin, properdin was depleted from serum and AP activity was inhibited consistently. In some embodiments, said isolated antigen binding protein may bind to specifically epitopes domain of properdin. In some embodiments, said epitopes comprise TSR5, TSR6, and/or TSR0 of properdin.

In some embodiments, the present disclosure provides an isolated antibody or an antigen-binding fragment thereof comprising a heavy chain variable region comprising HCDR1, HCDR2 and/or HCDR3, wherein
the HCDR1 comprises an amino acid sequence selected from the group consisting of: SEQ ID NOs: 3, 6, 9, 12, 39, 40, 41, 42, 43, 44, 45, 46, 47, 48, 49, 50, 51, 52, 53, 54, 55, 56, 57, 58, 59, 149, 150, 151, 152 and 153;
the HCDR2 comprises an amino acid sequence selected from the group consisting of: SEQ ID NOs: 2, 5, 8, 11, 60, 61, 62, 63, 64, 65, 66, 67, 68, 69, 70, 71, 72, 73, 74, 75, 76, 77, 78, 79, 80, 81, 82, 154, 155, 156, 157, 158 and 159; and/or
the HCDR3 comprises an amino acid sequence selected from the group consisting of: SEQ ID NOs: 1, 4, 7, 10, 83, 84, 85, 86, 87, 88, 89, 90, 91, 92, 93, 94, 95, 96, 97, 98, 99, 100, 101, 102, 103, 104, 105, 106, 107, 160, 161, 162, 163, 164 and 165.

In some embodiments, the isolated antibody or antigen-binding fragment thereof comprises a heavy chain variable region comprising HCDR1, HCDR2 and HCDR3 as shown in Table 1.

In some embodiments, the isolated antibody or antigen-binding fragment thereof comprises a heavy chain variable region as shown in Table 1.

TABLE 1
CDR amino acid sequences and variable region amino acid
sequences of the antigen-binding proteins of present disclosure.

| Antigen-binding | HCDR1 | HCDR2 | HCDR3 | VH |
|---|---|---|---|---|
| proteins | (SEQ ID NO) | (SEQ ID NO) | (SEQ ID NO) | (SEQ ID NO) |
| SLN-6068 | 3 | 2 | 1 | 13 |
| SLN-6043 | 6 | 5 | 4 | 14 |
| SLN-6062 | 9 | 8 | 7 | 15 |
| SLN-6065 | 12 | 11 | 10 | 16 |
| SLN9056 | 39 | 60 | 83 | 108 |
| SLN9057 | 40 | 61 | 84 | 109 |
| SLN0042 | 41 | 62 | 85 | 110 |
| SLN0043 | 42 | 63 | 86 | 111 |
| SLN0044 | 43 | 64 | 87 | 112 |
| SLN0045 | 44 | 65 | 88 | 113 |
| SLN0049 | 45 | 66 | 89 | 114 |
| SLN0056 | 39 | 60 | 83 | 115 |
| SLN0057 | 46 | 67 | 90 | 116 |
| SLN0059 | 47 | 68 | 91 | 117 |
| SLN0062 | 48 | 69 | 92 | 118 |
| SLN0064 | 49 | 70 | 93 | 119 |
| SLN0065 | 50 | 71 | 94 | 120 |
| SLN0071 | 51 | 72 | 95 | 121 |
| SLN0072 | 40 | 61 | 96 | 122 |
| SLN0046 | 44 | 65 | 97 | 123 |
| SLN0058 | 52 | 73 | 98 | 124 |
| SLN9008 | 53 | 74 | 99 | 125 |
| SLN9013 | 54 | 75 | 100 | 126 |
| SLN9015 | 39 | 60 | 101 | 127 |
| SLN9025 | 55 | 76 | 102 | 128 |
| SLN9026 | 56 | 77 | 103 | 129 |
| 1902-2-1-mm-F7 | 57 | 80 | 105 | 130 |
| 1902-2-1-hh-B12 | 58 | 81 | 106 | 131 |
| 1902-2-1-hm-D12 | 59 | 82 | 107 | 132 |
| SLN0097 | 39 | 60 | 83 | 133 |
| SLN9079 | 39 | 60 | 83 | 134 |
| SLN9060 | 40 | 78 | 96 | 135 |
| SLN9061 | 40 | 79 | 96 | 136 |
| SLN9062 | 40 | 61 | 96 | 137 |
| SLN9077 | 40 | 61 | 104 | 138 |
| SLN9058 | 40 | 61 | 96 | 139 |
| SLN0104 | 39 | 60 | 83 | 140 |
| SLN0100 | 39 | 60 | 83 | 141 |
| SLN0105 | 40 | 61 | 96 | 142 |
| SLN0066 | | | | 148 |
| SLN7150 | 149 | 154 | 160 | 208 |
| SLN12066 | 149 | 154 | 160 | 209 |
| SLN12067 | 149 | 154 | 160 | 210 |
| SLN12068 | 149 | 154 | 160 | 211 |
| SLN12069 | 149 | 154 | 160 | 212 |
| SLN12070 | 149 | 154 | 160 | 213 |
| SLN7160 | 150 | 155 | 161 | 214 |
| SLN12075 | 150 | 155 | 161 | 215 |
| SLN12076 | 150 | 155 | 161 | 216 |
| SLN12077 | 150 | 155 | 161 | 217 |
| SLN7162 | 151 | 156 | 162 | 218 |
| SLN12078 | 151 | 156 | 162 | 219 |
| SLN12079 | 151 | 156 | 162 | 220 |
| SLN12080 | 151 | 156 | 162 | 221 |
| SLN12081 | 151 | 156 | 162 | 222 |
| SLN7151 | 151 | | | 231 |
| SLN7152 | 151 | | | 232 |
| SLN12039 | 151 | 159 | 165 | 241 |
| SLN12036 | 152 | 157 | 163 | 223 |
| SLN12082 | 152 | 157 | 163 | 224 |
| SLN12083 | 152 | 157 | 163 | 225 |
| SLN12084 | 152 | 157 | 163 | 226 |
| SLN12085 | 152 | 157 | 163 | 227 |
| SLN12086 | 152 | 157 | 163 | 228 |
| SLN12030 | 153 | 158 | 164 | 240 |
| SLN7153 | | | | 233 |
| SLN7154 | | | | 234 |
| SLN7155 | | | | 235 |
| SLN7156 | | | | 236 |
| SLN7159 | | | | 237 |
| SLN7161 | | | | 238 |
| SLN12027 | | | | 239 |
| SLN12041 | | | | 242 |
| SLN12042 | | | | 243 |
| SLN12044 | | | | 244 |
| SLN12045 | | | | 245 |
| SLN9056 | | | | 263 |

In some embodiments, the antibody comprises single domain antibody, camelid antibody, single chain antibody, monoclonal antibody, chimeric antibody, multi-specific antibody, humanized antibody and fully human antibody. In some embodiments, the antigen-binding fragment comprises Fab, Fab′, F(ab)₂, F(ab′)₂, sdAb, Fv, scFv fragment and bi-paratopic antigen-binding protein. In some embodiments, the isolated antibody or antigen-binding fragment thereof is a VHH.

In another aspect, the present application provides a bi-paratopic antigen-binding protein, wherein the bi-paratopic antigen-binding protein comprises a first antigen-binding domain, and a second antigen-binding domain, wherein the first antigen-binding domain and/or the second antigen binding domain comprises the isolated antibody or antigen-binding fragment thereof disclosed herein. In some embodiments, the first antigen-binding domain and the second antigen-binding domain target the same antigen. In some embodiments, the first antigen-binding domain and the second antigen-binding domain target different antigens. In some embodiments, the bi-paratopic antigen-binding protein comprises an amino acid sequence as shown in Table 2.

In another aspect, the present application provides a polypeptide, the polypeptide comprises the isolated antibody or an antigen-binding fragment thereof disclosed herein or the bi-paratopic antigen-binding protein disclosed herein. In some embodiments, the polypeptide further comprises one or more other functional domain(s), a therapeutic entity, a functionally active protein. In some embodiments, the functional domain of the polypeptide comprises a complement-inhibiting entity. In some embodiments, the functional domain of the polypeptide comprises a Fc region. In some embodiments, the functional domain of the polypeptide comprises a VHH region. In some embodiments, the therapeutic entity is an engineered cytotoxic pseudomonas exotoxin A (PE38), or a glucagon-like peptide-1 (GLP-1) or its variant. In some embodiments, the functionally active protein is a factor H or a VEGF inhibiting protein. In some embodiments, the polypeptide comprises an amino acid sequence as shown in Table 2.

TABLE 2

| Proteins/ | SEQ ID | Proteins/ | SEQ ID | Proteins/ | SEQ ID |
|---|---|---|---|---|---|
| Polypeptides | NO | Polypeptides | NO | Polypeptides | NO |
| SLN6071 | 17 | SLN7207 | 230 | SLN12059 | 253 |
| SLN6075 | 18 | SLN12021 | 246 | SLN12060 | 254 |
| SLN6079 | 19 | SLN12022 | 247 | SLN12061 | 255 |
| SLN6073 | 21 | SLN12023 | 248 | SLN6073 | 261 |
| SLN6074 | 23 | SLN12024 | 249 | SLN8284 | 262 |
| GLP-1-9056 | 143 | SLN12025 | 250 | SLN12147 | 264 |
| 0072-PE38 | 144 | SLN12026 | 251 | SLN12149 | 265 |
| SLN12140 | 229 | SLN12058 | 252 | SLN12150 | 266 |

In another aspect, the present application provides one or more isolated nucleic acid molecules, encoding the isolated antibody or antigen-binding fragment thereof disclosed herein, the bi-paratopic antigen-binding protein disclosed herein, or the polypeptides disclosed herein.

In another aspect, the present application provides a vector, the vector comprises the nucleic acid molecules encoding the isolated antibody or antigen-binding fragment thereof disclosed herein, the bi-paratopic antigen-binding protein disclosed herein, or the polypeptides disclosed herein.

In another aspect, the present application provides a cell, the cell comprises the nucleic acid molecules or the vector of the present application.

In another aspect, the present application provides an immunoconjugate, comprising the isolated antibody or antigen-binding fragment thereof disclosed herein, the bi-paratopic antigen-binding protein disclosed herein, or the polypeptides disclosed herein.

In another aspect, the present application provides a pharmaceutical composition, the pharmaceutical composition comprises the isolated antibody or antigen-binding fragment thereof disclosed herein, the bi-paratopic antigen-binding protein disclosed herein, or the polypeptides disclosed herein.

In another aspect, the present disclosure provides a method of preparing the isolated antibody or antigen-binding fragment thereof disclosed herein, the bi-paratopic antigen-binding protein disclosed herein, or the polypeptides disclosed herein, comprising culturing the cell under conditions that allow the isolated antibody or antigen-binding fragment thereof disclosed herein, the bi-paratopic antigen-binding protein disclosed herein, or the polypeptides disclosed herein to be expressed.

In another aspect, the present application provides a method of preventing and/or treating a disease, comprising administering to a patient in need thereof an effective amount of the isolated antibody or antigen-binding fragment thereof disclosed herein, the bi-paratopic antigen-binding protein disclosed herein, the polypeptides disclosed herein, the immunoconjugate disclosed herein, the isolated nucleic acid molecule disclosed herein, the vector disclosed herein, the cell disclosed herein, and/or the pharmaceutical composition disclosed herein.

In another aspect, the present application provides a use of the isolated antibody or antigen-binding fragment thereof disclosed herein, the bi-paratopic antigen-binding protein disclosed herein, or the polypeptides disclosed herein, and/or the pharmaceutical composition of the present application, in preparation of a drug, and said drug is used for preventing and/or treating a disease.

In another aspect, the present disclosure provides a method for delivering a therapeutic drug or diagnostic agent to cells or organs in a subject, comprising using the isolated antibody or antigen-binding fragment thereof disclosed herein, the bi-paratopic antigen-binding protein disclosed herein, or the polypeptides disclosed herein.

Additional aspects and advantages of the present disclosure will become readily apparent to those skilled in this art from the following detailed description, wherein only illustrative embodiments of the present disclosure are shown and described. As to be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE DRAWINGS

The novel features of the invention are set forth with particularity in the appended claims. A better understanding of the features and advantages of the present invention will be obtained by reference to the following detailed descriptions that set forth illustrative embodiments, in which the principles of the invention are employed, and the accompanying drawings (also “figure”, “figures”, “FIG.” and “FIGs.” herein), of which:

FIG. 1 illustrates the purity of VHH-Fcs analysis on non-reducing SDS-PAGE.

FIGS. 2A-2B illustrate VHH-Fc binding to hVEGFA121 and Receptor Blocking Activity.

FIG. 3 illustrates the epitope binning results.

FIG. 4 illustrates the purity of Bi/Tri-specific VHH-Fc analysis on SDS-PAGE.

FIGS. 5A-5B illustrate the Bi/Tri-specific VHH-Fc Binding to hVEGFA121 and Receptor Blocking Activity.

FIGS. 6A-6H illustrate the humanized VHH-Fc binding to hVEGFA121 and Receptor Blocking Activity.

FIGS. 7A-7D illustrate the humanized Bi-specific VHH-Fc binding to hVEGFA121 or mVEGF120 and Receptor Blocking Activity.

FIGS. 8A-8C illustrate RBA assays of hVEGFA (165 and 121), mVEGFA (120) on human VEGFR2.

FIGS. 9A-9B illustrate the inhibition of HUVEC cell proliferation.

FIGS. 10A-10E illustrate the dual functional recombinant proteins inhibiting VEGF-driven angiogenesis and factor H-regulated complement activation.

FIG. 11 illustrates representative SDS-PAGE of the transferrin binding protein-OVA-his proteins expressed in HEK293 cells.

FIGS. 12A-12B illustrate representative transferrin-mediated binding of the selected transferrin binding protein-OVA-His proteins to human transferrin at pH7.4 or pH6.0.

FIGS. 13A-13D illustrate transferrin-mediated binding of the selected transferrin binding protein-OVA-His proteins to the transferrin receptor-expressing cells.

FIG. 14 illustrates epitope binning of representative transferrin binding protein of the present disclosure.

FIGS. 15A-15D illustrate representative ELISA data show binding of the humanized transferrin binding protein variants to the native human transferrin at pH7.4 or pH6.0.

FIG. 16A illustrates circulation half-life of OVA fused to the transferrin binding protein of the present disclosure clones with different epitopes. FIG. 16B illustrates circulation half-life of GLP-1-VHH in mice. FIG. 16C illustrates VHH-enabled circulation half-life extension is affinity-dependent.

FIGS. 17A-17C illustrate the transferrin binding protein enabled translocation of the fused OVA across monolayer of Caco-2 cells or through intestinal epithelium via transcytosis.

FIGS. 18A-18H illustrate that the transferrin binding protein enabled the associated OVA to cross blood-brain barrier in mice, and dependency of such capabilities is associated with epitope utilization and affinity.

FIGS. 19A-19B illustrate the intracellular delivery of the transferrin binding protein-PE38 on different cells.

FIG. 20 illustrates the biodistribution of the transferrin binding protein variants after systemic administration.

FIG. 21 Illustrates production of human, mouse and cyno properdin analysis on non-reducing and reducing SDS-PAGE.

FIG. 22 Illustrates production of the recombinant VHH-Fc analysis on non-reducing and reducing SDS-PAGE.

FIGS. 23A-23C Illustrate ELISA binding of the recombinant VHH-Fc to human (FIG. 23A), cyno (FIG. 23B) or mouse (FIG. 23C) properdin.

FIGS. 24A-24B Illustrate complement-inhibitory activities of the recombinant VHH-Fc within human (FIG. 24A) or mouse (FIG. 24B) serum.

FIG. 25 Illustrates epitope binning of the properdin (full length)-binding VHH's.

FIG. 26 Illustrates production of bi-paratopic VHH's analysis on non-reducing and reducing SDS-PAGE.

FIGS. 27A-27D Illustrate binding of the bi-paratopic VHH's to human (FIG. 27A and FIG. 27C) or mouse (FIG. 27B and FIG. 27D) properdin.

FIGS. 28A-28D Illustrate complement-inhibitory activities of the bi-paratopic VHH's within human (FIG. 28A and FIG. 28C) or mouse (FIG. 28B and FIG. 28D) serum.

FIGS. 29A-29H Illustrate humanization of the selected properdin-binding VHH's. Human properdin binding (FIG. 29A and FIG. 29C), mouse properdin binding (FIG. 29B and FIG. 29D), human AP activity (FIG. 29E and FIG. 29F), mouse AP activity (FIG. 29G and FIG. 29H).

FIG. 30 Illustrates epitope mapping of the properdin (truncated variants)-binding VHH's.

FIGS. 31A-31B Illustrate impact of properdin inhibitors on interactions between C3 and properdin. Properdin binding assay (FIG. 31A), Competition assay (FIG. 31B).

FIG. 32 Illustrates production of the VHH-CFH analysis on non-reducing and reducing SDS-PAGE.

FIGS. 33A-33D Illustrate properdin-binding activities of SLN12140 and SLN7207 in human (FIG. 33A and FIG. 33C) and mouse (FIG. 33B and FIG. 33D) serum.

FIGS. 34A-34D Illustrate AP inhibiting activities of SLN12140 and SLN7207 in human (FIG. 34A and FIG. 34C) and mouse (FIG. 34B and FIG. 34D) serum.

FIGS. 35A-35C Illustrate pathway-selectivity of SLN12140 in complement inactivation. AP (FIG. 35A), CP (FIG. 35B), and LP (FIG. 35C).

FIGS. 36A-36D Illustrate cross-species reactivity of SLN12140 upon AP activity assay. AP activity in human serum (FIG. 36A), AP activity in cyno serum (FIG. 36B), AP activity in mouse serum (FIG. 36C) and AP activity in rat serum (FIG. 36D).

FIG. 37 Illustrates serum stability of SLN12140 both in plasma and formulation buffer.

FIG. 38 Illustrates subcutaneous and intravenous single dose pharmacokinetic studies in mice.

FIGS. 39A-39C Illustrate dose-dependent subcutaneous PK of SLN12140 at a dosage of 3 mpk (FIG. 39A), 10 mpk (FIG. 39B) or 30 mpk (FIG. 39C), respectively.

FIG. 40 Illustrates multiple subcutaneous dosing SLN12140 once a week for 3 weeks sustainably reduce the target concentration to a low level and consistently inhibit AP activity.

FIG. 41 Illustrates multiple subcutaneous dosing SLN12140 once a week for 7 weeks in hCD89 Tg mice sustainably reduce the target concentration to a low level and stable pharmacokinetic characteristics of SLN12140.

FIG. 42 Illustrates representative SDS-PAGE gels of the fusion proteins expressed in HEK293 cells. Purified fusion proteins SLN6073, SLN8284, SLN12140 under non-reducing conditions (lane 1,3,5) and reducing conditions (lane 2, 4, 6).

FIGS. 43A-43C Illustrate representative binding of SLN8284, SLN12140, SLN6073 to different species' properdin in vitro. FIG. 43A) Human properdin binding of SLN8284, SLN6073, SLN12140. FIG. 43B) Cynomolgus macaques properdin binding of SLN8284, SLN6073, SLN12140. FIG. 43C) Mouse properdin binding of SLN8284, SLN6073, SLN12140.

FIGS. 44A-44B Illustrate inhibition of the alternative complement pathway in rabbit erythrocytes by various concentrations of fusion proteins SLN8284, SLN6073, SLN12140. FIG. 44A) Inhibition of the alternative complement pathway of SLN8284, SLN6073, SLN12140 in human serum. FIG. 44B) Inhibition of the alternative complement pathway of SLN8284, SLN6073, SLN12140 in mouse serum.

FIGS. 45A-45B Illustrate in vitro binding of a VEGF by SLN8284, SLN6073, SLN12140 as detected by ELISA. FIG. 45A) Human VEGF121 binding of SLN8284, SLN6073, SLN12140. FIG. 45B) Mouse VEGF120 binding of SLN8284, SLN6073, SLN12140.

FIGS. 46A-46B Illustrate effect of blocking the interaction between VEGFA and VEGFR2 by SLN8284, SLN6073, SLN12140. FIG. 46A) Blocking the interaction between hVEGFA and hVEGFR2 by SLN8284, SLN6073, SLN12140. FIG. 46B) Blocking the interaction between mVEGFA and mVEGFR2 by SLN8284, SLN6073, SLN12140.

FIG. 47 Illustrates inhibition of VEGF-induced proliferation of human umbilical vein endothelial cells (HUVECs) by SLN8284, SLN6073, SLN12140.

FIG. 48 Illustrates representative SDS-PAGE gels of the fusion proteins expressed in HEK293 cells. Purified fusion proteins SLN12147, SLN12149, SLN12150 under non-reducing conditions (lane 1,3,5) and reducing conditions (lane 2, 4, 6).

FIGS. 49A-49C Illustrate Elisa Binding results of SLN12140, SLN12147, SLN12149 and SLN12150 to different species' properdin in vitro. FIG. 49A) Human properdin binding. FIG. 49B) Cynomolgus macaques properdin binding. FIG. 49C) Mouse properdin binding.

FIGS. 50A-50D Illustrate ELISA binding results of SLN12140, SLN12147, SLN12149, SLN12150 and SLN9056 to human transferrin (hTf) in vitro. FIG. 50A) Binding results of SLN12147, SLN12149 and SLN9056 with hTf at pH7.4;

FIG. 50B) Binding results of SLN12147, SLN12149 and SLN9056 with hTf at pH6.0; FIG. 50C) Binding results of SLN12140, SLN12150 with hTf at pH7.4; FIG. 50D) Binding results of SLN12140, SLN12150 with hTf at pH6.0.

FIGS. 51A-51C Illustrate inhibiting different species AP activities with SLN12140, SLN12147, SLN12149 and SLN12150 in ELISA. FIG. 51A) Human AP activity with fusion proteins. FIG. 51B) Cynomolgus AP activity with fusion proteins. FIG. 51C) Mouse AP activity with fusion proteins.

FIGS. 52A-52D Illustrate SLN12140 and SLN12150 PK results in Mouse. FIG. 52A) Drugs in Mouse serum. FIG. 52B) Drugs in Mouse Brain tissue. FIG. 52C) Drugs in Mouse CSF. FIG. 52D) Drugs ID % in Mouse Brain tissue.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

The term “antibody” is used in the broadest sense, and may include but not limited to monoclonal antibodies (including full-length monoclonal antibodies containing two light chains and two heavy chains), polyclonal antibodies, multi-specific antibodies (e.g., bispecific antibodies), murine antibodies, human antibodies (fully human antibodies), humanized antibodies, chimeric antibodies, single chain antibodies (e.g., scFv), antibody derivatives, and antibody fragments that bind to an antigen (e.g., Fab′, VHH, and (Fab)₂ fragments). The term “antibody” may also include all recombinant forms of antibodies, such as antibodies expressed in prokaryotic cells, unglycosylated antibodies, and any antigen-binding antibody fragments and derivatives thereof described herein. The “antibody” may generally comprise a protein in which at least two heavy chains (HC) and two light chains (LC) are linked to each other by disulfide bonds, or an antigen-binding fragment thereof. Each heavy chain may be composed of a heavy chain variable region (VH) and a heavy chain constant region. The VH region can be further distinguished as hypervariable regions, termed complementarity determining region (CDR), interspersed with more conserved regions termed framework region (FR). Each VH may be composed of three CDRs and four FRs regions, which may be arranged from amino-terminus to carboxy-terminus in the following order: FR1, CDR1, FR2, CDR2, FR3, CDR3 and FR4. The variable regions of the heavy chains contain binding domains that interact with an antigen (e.g., properdin). In the art, the CDR of an antibody may be defined by a variety of methods, for example, the Kabat definition rules based on sequence variability (see, Kabat et al., Sequences of Proteins of Immunological Interest, Fifth Edition, National Institutes of Health, Besse Star, Maryland (1991)), the Chothia definition rules based on the location of the structural loop regions (see, Al-Lazikani et al., J Mol Biol 273: 927-48, 1997), and the IMGT definition rules based on the concepts in IMGT-ONTOLOGY and IMGT Scientific chart rules. In the present application, the CDRs may be defined by the Kabat definition rules.

The term “antigen binding domain” herein generally refers to a domain capable of binding to a target. For example, the binding may require some complementarity in binding sequence. For example, the binding may require special structure. For example, in the present application, the antigen binding domain of antigen binding protein may specifically bind to antigen (e.g., properdin, VEGF family, transferrin). For example, the antigen binding domain may belong to an antibody or an antigen binding fragment, and make them bind to the target with greater affinity, avidity, easiness, and/or duration than it binds to other targets. For example, the antigen binding domain may have a measurable and reproducible interaction, such as the binding between an antigen and an antibody, whereby the existence of a target may be determined in the presence of a heterogeneous population of molecules (including biomolecules). “Binding sequence” refers to a specific amino sequence on the target (e.g., antigen), which is complementary to the antigen binding protein.

The term “antigen binding fragment” herein generally refers to one or more fragments of an antibody that specifically bind to a target. The antigen binding function of an antibody can be achieved by a full-length fragment of the antibody. The antigen binding function of an antibody can also be achieved by: a heavy chain comprising a fragment of Fv, scFv, dsFv, Fab′ or F(ab′)₂, or a light chain comprising a fragment of Fv, scFv, dsFv, Fab′ or F(ab′)₂. The term “Fab” generally refers to a fragment comprising a heavy-chain variable domain and a light-chain variable domain, and also comprising a light-chain constant domain and a heavy-chain first constant domain (CH1). The term “Fab′” generally refers to a fragment that is different from Fab by the addition of a few residues (comprising one or more cysteines from the hinge region of an antibody) to a carboxyl terminus of the heavy-chain CH1. The term “F(ab′)₂” generally refers to a dimer of Fab′, comprising an antibody fragment in which two Fab fragments are linked by a disulfide bridge on the hinge region. The term “Fv” generally refers to the smallest antibody fragment that comprises a complete antigen recognition and binding site. In some cases, this fragment may consist of a dimer in which one heavy-chain variable region and one light-chain variable region are tightly non-covalently bound. The term “dsFv” generally refers to a disulfide-stabilized Fv fragment, with a disulfide bond between a single light-chain variable region and a single heavy-chain variable region. The term “dAb fragment” generally refers to an antibody fragment consisting of a VH domain. The term “scFv” generally refers to a molecule produced by covalently linking and pairing one heavy-chain variable domain with one light-chain variable domain of an antibody by means of a flexible peptide linker. The term “Fd” generally refers to a fragment consisting of the VH and CH domains. For example, the term “antigen binding fragment” may include one class of antibody VHHs, which lacks the antibody light chain and has only the heavy chain variable region.

The term “antigen binding protein” herein generally refers to a polypeptide molecule capable of specifically recognizing and/or neutralizing a particular antigen. For example, in the present application, the term “antigen binding protein” may include an “antibody” or an “antigen binding fragment”, as long as they exhibit the desired antigen-binding activity. For example, the said isolated antigen binding protein may comprise a single domain protein, for example, the said isolated antigen binding protein may include any molecule comprising an antigen-binding portion thereof. For example, the said isolated antigen binding protein may comprise a VHH-Fc protein or a Fc-VHH-VHH protein.

The term “camelid antibody” generally refers to an antibody derived from a camelid species. For example, in a camel, dromedary, llama, alpaca or guanaco. Camelid antibody lacks a light chain, and thus includes only heavy chains with complete and diverse antigen binding capabilities.

The term “VHH” also known as VHH domains, VHH antibody fragments, and VHH antibodies, generally refers to the antigen binding immunoglobulin (variable) domain of “heavy chain antibodies”. For example, having the structure of FR1-CDR1-FR2-CDR2-FR3-CDR3-FR4 and specifically binding to an epitope without requiring the presence of a second immunoglobulin variable domain.

The term “chimeric antibodies” generally refers to an antibody in which the variable region is derived from one species and the constant region is derived from another species. Generally, the variable region is derived from an antibody (“parent antibody”) in an experimental animal such as a rodent, and the constant region is derived from a human antibody, such that the possibility of causing an adverse immune response in an individual human by the resulting chimeric antibody is reduced as compared with the parental (e.g., mouse-derived) antibody.

The term “derivative” “variant” or “analogue” are used interchangeably, and generally refers to a polypeptide or polynucleotide of the present application, including any substitution, variation, modification, substitution, deletion and/or addition of one (or more) amino acid residues from/to the sequence, so long as the resulting polypeptide or polynucleotide substantially retains at least one of its endogenous functions. For example, the derivative may have at least 30%, at least 35%, at least 40%, at least 45%, at least 50%, at least 55%, at least 60%, at least 65%, at least 70%, at least 75%, at least 80%, at least 85%, at least 90%, at least 95% or at least 99% similarity to its corresponding sequence.

The term “epitopes” generally refers to a domain or an amino acid sequence specifically bind to antigen binding protein. For example, the term “epitopes” may include chemically active surface molecular groups (e.g., a sugar side chain, a phosphoryl group, or a sulfonyl group). For example, the term “epitope” may have specific tertiary structural features, and/or specific charge features.

The term “fully human antibodies” generally refers to an antibody that contains only the sequence of human immunoglobulin proteins. The fully human antibody can greatly reduce the side immune effects caused by heterologous antibodies on the human body. Methods for obtaining the fully human antibody in the art may include the phage display technology, the transgenic mouse technology, the ribosome display technology, the RNA-polypeptide technology, etc.

The term “bi-paratopic antigen-binding protein” generally refers to an antigen-binding molecule comprising a first antigen-binding domain and a second antigen-binding domain. For example, the two antigen-binding domain binds to two different epitopes. For example, non-overlapping epitopes of the respective antigen. For example, the first antigen-binding domain and the second antigen-binding domain may target the same antigen. For example, the first antigen-binding domain and the second antigen-binding domain target different epitopes of the same antigen. The part of an antigen-binding protein that recognize the epitope is called a paratope.

The term “fusion protein” generally refers to a protein composed of two or more polypeptides. The two or more polypeptide components can be bound directly or indirectly through a peptide linker/spacer. For example, said polypeptides are not normally bound in their natural state, they are held together by peptide bonds through their respective amino and carboxyl termini to form a contiguous polypeptide. For example, the term “fusion protein” comprises an antigen binding protein which is prepared by the method described in present application, and a functionally active protein. For example, the said functionally active protein may be factor H. For example, the said functionally active protein may be VEGF inhibiting protein. For example, the said functionally active protein may be transferrin inhibiting protein. For example, the fusion protein may include a prophylactic or therapeutic drug fused to a heterologous protein, polypeptide, or peptide. Wherein, the heterologous protein, polypeptide or peptide may or may not be different types or therapeutic drugs. For example, the fusion protein may comprise two different proteins, polypeptides or peptides with immunomodulatory activity. For example, the fusion protein may retain or improve the activity compared to the activity of the original polypeptide or protein. Typically, the fusion protein can be produced by in vitro recombinant techniques well known in the art. For example, the fusion protein may comprise the antigen binding protein. For example, the fusion protein may comprise biologic molecules. For example, the fusion protein may compose of properdin inhibiting proteins and VEGF inhibiting proteins. For example, the fusion protein may compose of properdin inhibiting proteins and transferrin inhibiting proteins.

The term “humanized antibodies” generally refers to an antibody in which some or all of the amino acids outside the CDR of a non-human antibody (e.g., a mouse antibody) have been replaced by corresponding amino acids derived from human immunoglobulins. In the CDR, small additions, deletions, insertions, substitutions, or modifications to the amino acids may also be allowed, as long as they still retain the capability of the antibody to bind to a specific antigen. The humanized antibody may optionally comprise at least a portion of a constant region of a human immunoglobulin. The “humanized antibody” reserves the antigen specificity similar to that of the original antibody. The “humanized” form of a non-human (e.g., a mouse) antibody may minimally comprise a chimeric antibody derived from a non-human immunoglobulin sequence. In some cases, CDR residues in a human immunoglobulin (receptor antibody) may be replaced with CDR residues from a non-human species (donor antibody) (e.g., a mouse, a rat, a rabbit, or a non-human primate) with the desired properties, affinity, and/or capability. In some cases, FR residues of the human immunoglobulin may be replaced with corresponding non-human residues. In addition, the humanized antibody may comprise an amino acid modification that is not present in the receptor antibody or in the donor antibody. These modifications may be made to further improve the properties such as binding affinity of the antibody.

The term “immunoconjugate” generally refers to a conjugate formed by conjugating (e.g., covalently linking via a linking molecule) the additional therapeutic agent to the isolated antigen binding protein, which conjugate can deliver the additional therapeutic agent to a target cell via specific binding of the isolated antigen binding protein to an antigen on the target cell.

The term “monoclonal antibodies” generally refers to an antibody obtained from a group of substantially homogeneous antibodies, that is, a cluster in which several antibodies are the same, except for a few natural mutants that may exist. The monoclonal antibody is generally highly specific for a single antigen site. Moreover, unlike conventional polyclonal antibody preparations (which generally comprise different antibodies directed against different determinants), each monoclonal antibody is directed against a single determinant on the antigen. In addition to their specificity, the advantage of monoclonal antibodies lies in that they may be synthesized by hybridoma culture, without being contaminated by other immunoglobulins. The modifier “monoclonal” indicates the characteristics of an antibody obtained from a substantially homogeneous antibody population, and is not construed as requiring the production of the antibody by any specific method. For example, the monoclonal antibodies may be prepared in hybridoma cells, or may be prepared by recombinant DNA methods.

The term “polypeptide”, “polypeptides”, “peptide” and “protein” are used interchangeably, and generally refer to a polymer of amino acids of any length. The polymer may be linear or branched, it may comprise modified amino acids, and it may be interrupted by non-amino acids. These terms also encompass amino acid polymers that have been modified. These modifications may comprise: disulfide bond formation, glycosylation, lipidation, acetylation, phosphorylation, or any other manipulation (e.g., binding to a labeling component). The term “amino acid” includes natural and/or unnatural or synthetic amino acids, including glycine, D and L optical isomers, amino acid analogs and peptidomimetics.

The term “associated entity” generally refers to any monomeric or multimeric protein, protein fragment, nucleotide sequence, small molecular compound, a delivery vehicle, or a modified transgene vector that was specifically linked to an antigen binding protein. The associated entity includes but is not limited to antibodies and binding parts thereof, such as immunologically functional fragments. For example, the antigen binding protein may be transferrin-binding protein. In the present application, the term “therapeutic entity” generally refers to any monomeric or multimeric protein or protein fragment that have the function of curing or preventing diseases.

The term “ortholog” generally refers to an amino acid sequence that shares a certain percentage of sequence identity and functional similarity with the reference amino acid sequence. For example, orthologs may comprise structurally similar sequences in different species due to evolution from a common ancestor. Ortholog may be identified using any method known in the art, preferably by using the BLAST tool to compare a reference sequence to a separate second sequence or sequence fragment or sequence database. For example, the ortholog may have at least 30%, at least 35%, at least 40%, at least 45%, at least 50%, at least 55%, at least 60%, at least 65%, at least 70%, at least 75%, at least 80%, at least 85%, at least 90%, at least 95% or at least 99% similarity to its corresponding sequence.

The term “isolated” antigen binding protein generally refers to an antigen binding protein that has been identified, isolated, and/or recovered from (e.g., native or recombinant) components of the environment in which it is produced. Contaminant components of the environment in which it is produced are generally substances that interfere with its investigational, diagnostic or therapeutic use, and may include enzymes, hormones, and other proteinaceous or non-proteinaceous solutes. An isolated antigen binding protein or an antibody is generally prepared by at least one purification step. The isolated antigen binding protein of the present application generally specifically binds to properdin.

The term “isolated nucleic acid molecule” generally refers to a genome, an mRNA, a cDNA, or a synthetic-origin DNA or RNA or a certain combination thereof. It is not associated with the all or some of polynucleotides found in nature, or is linked to polynucleotides to which it is not linked in nature.

The term “vector” generally refers to a nucleic acid molecule capable of self-replication in a suitable host. It transfers an inserted nucleic acid molecule into and/or between host cells. The vector may include a vector mainly for inserting DNA or RNA into cells, a vector mainly for replicating DNA or RNA, and a vector mainly for expressing DNA or RNA transcription and/or translation. The vector also includes a vector having a variety of the functions defined above. The vector may be a polynucleotide that may be transcribed and translated into a polypeptide when introduced into a suitable host cell. Generally, the vector may produce a desired expression product by culturing a suitable host cell containing the vector.

The term “properdin”, “factor P”, and “Pillemer molecular” are used interchangeably, and are positive regulator of the alternative complement activation. For example, the term “properdin” may be oligomerization of a rod-like monomer into cyclic dimers, trimers, and tetramers. For example, the term “properdin” may be human properdin, which has about 469 amino acid soluble glycoprotein found in plasma and seven thrombospondin type I repeats (TSR) with the N-terminal domain. For example, the term “properdin” may be mouse properdin, which has about 457 amino acid soluble glycoprotein found in plasma and seven TSRs with the N-terminal domain. Said TSRs can be divided according to the common sense of people in the field. For example, the term “properdin” may comprise full length, truncated, and variant properdin.

The term “VEGF”, or “vascular endothelial growth factor” are used interchangeably, and generally refers to a family of signaling proteins that can stimulate for example angiogenesis, vasculogenesis and/or lymphangiogenesis. Members of the VEGF family include VEGF-A, VEGF-B, VEGF-C, VEGF-D, and PIGF (Placental Growth Factor). In the present application, the term “VEGF” may comprise its functional active fragment, ortholog, analogue, and variant.

The term “transferrin” generally refers to a glycoprotein which can bind to and transport multivalent ions. For example, the transferrin may be a single-chain glycoprotein. For example, the transferrin may have a molecular weight of about 77,000D. For example, the transferrin may have a polysaccharide. For example, the transferrin may have two ion binding sites. For example, the ion binding sites may have different affinity with iron ions. For example, the multivalent ion may be an iron ion, a chromium ion, a manganese ion, a cadmium ion or a nickel ion thereof. For example, each molecule of transferrin may bind two trivalent iron atoms. For example, transferrin could be iron-containing holo-transferrin, or iron-free apo-transferrin. For example, the transferrin may be a mice transferrin. For example, the amino sequence of mice transferrin may be as set forth in GenBank: EDL21066.1, AAL34533.1, or AAL34533.1. For example, the transferrin may be a human transferrin. For example, the amino sequence of human transferrin may be as set forth in GenBank: AAH59367.1, AAH59367.1, or AAB22049.1. In the present application, the term “transferrin” may comprise its functional active fragment, ortholog, and variant.

The term “transferrin receptor” generally refers to a carrier protein of transferrin. For example, the transferrin may be a transmembrane glycoprotein. For example, the transferrin receptor may mediate endocytosis of transferrin associated to two iron ions. For example, the transferrin receptor may maintain iron homeostasis in cells. For example, the transferrin receptor may be transferrin receptor 1 (TfR1) or transferrin receptor 2 (TfR2). For example, TfR1 and TfR2 may show homologies around 45-66% in the extracellular domain but present with different expression patterns in the body. For example, the TfR1 may have higher affinity to transferrin than that of TfR2. For example, TfR2 may have a 25-fold lower affinity with transferrin compared to that of TfR1. For example, the TfR2 may mainly express in tissues involved in regulating iron metabolism, such as the liver and small intestines, while the TfR1 is generally found on the surface of most body cells. The term “transferrin receptor 1” generally refers to a 97-kDa type2 membrane protein expressed as a homodimer in the cell membrane. TfR1-mediated transferrin internalization is classically described as the canonical iron import pathway. For example, the transferrin receptor may be a mouse transferrin receptor. For example, the amino sequence of mouse transferrin receptor may be as set forth in GenBank: AAH54522.1, CAA40624.1, or NP_001344227.1. For example, the transferrin receptor may be a human transferrin receptor. For example, the amino sequence of human transferrin receptor may be as set forth in GenBank: AAA61153.1, AAF04564.1, or AAB19499.1. In the present application, the term “transferrin receptor” may comprise its functional active fragment, ortholog, and variant.

The term “transferrin-binding protein” generally refers to a protein including an antigen-binding portion, and optionally it is allowed the antigen-binding portion to adopt a scaffold or skeleton portion in a conformation that promotes the binding of the antigen binding protein to the antigen. For example, the antigen binding protein may include, but not limit to, an antibody, an antigen binding fragment (Fab, Fab′, F(ab)2, a Fv fragment, F(ab′)2, scFv, di-scFv and/or dAb), an immunoconjugate, a multiple specific antibody (e.g., a bispecific antibody), an antibody fragment, an antibody derivative, an antibody analogue or a fusion protein, as long as they show the desired antigen binding activity. For example, the antigen binding protein may be capable of specifically binding to transferrin. For example, the antigen binding protein may not disturb the interaction between transferrin and transferrin receptor 1. For example, the antigen binding protein may not affect Tf/TfR1 binding. For example, the antigen binding protein may maintain the normal physiological function of iron transport.

The term “cell” generally refers to an individual cell, a cell line or a cell culture, which may contain or already contain a plasmid or vector comprising a nucleic acid molecule of the present application, or which is capable of expressing the antibody or antigen binding fragment thereof in the present application. The cell may include a progeny of a single host cell. Due to natural, accidental or deliberate mutations, progeny cells and original parent cells may not be necessarily identical in terms of morphology or genome, as long as they are capable of expressing the antibody or antigen-binding fragment thereof in the present application. The cells may be obtained by transfecting cells in vitro using the vector of the present application. The cells may be prokaryotic cells (e.g., Escherichia coli), or eukaryotic cells (e.g., yeast cells; e.g., COS cells, Chinese hamster ovary (CHO) cells, HeLa cells, HEK293 cells, COS-1 cells, NS0 cells, or myeloma cells). In some cases, the cells may be mammalian cells. For example, the mammalian cells may be CHO-K1 cells.

The term “cellular membrane” generally refers to an interface that separates the different media and components inside and outside the cell in the cell structure. Plasma membranes are generally believed to be represented as the basic unit of phospholipid bilayer molecules, that is, phospholipid bilayers, on which various types of membrane proteins, as well as sugars and glycolipids bound to membrane proteins, are inlaid. Through the pores and certain properties of transmembrane proteins, it can achieve selective and controllable material transport. For example, the cellular membrane may belong to a polarized. For example, the cellular membrane may belong to a unpolarized cell. In the present application, the term “polarized cell” generally refers to a cell with stable asymmetry in structure and function, such as a cell at resting potential, ready to transmit nerve impulse signals. The term “unpolarized cell” generally refers to a cell with a change in resting potential and transmits nerve impulse signals. For example, the polarized cell and/or unpolarized cell may belong to the blood brain barrier of central nervous system. For example, the polarized cell and/or unpolarized cell may belong to the intestinal epithelium. For example, the polarized cell and/or unpolarized cell may belong to a solid tissue.

The term “blood-brain barrier (BBB)”, generally refers to the physiological barrier between the peripheral circulation and the brain and spinal cord. It is formed by the tight junctions in the plasma membrane of brain capillary endothelial cells and constitutes a tight barrier that restricts the transport of molecules to the brain, even very small molecules such as urea (60 Daltons). For example, the brain capillary endothelial cells may have weaker pinocytosis. For example, the blood-brain barrier may include the BBB in the brain, the blood-spinal cord barrier in the spinal cord, and the blood-retinal barrier in the retina. For example, the BBB may also include the blood-CSF barrier (choroid plexus), where the barrier is composed of ependymal cells instead of capillary endothelial cells.

The term “half-life” generally refers to the time required to reduce the 50% levels in the serum concentration of an amino acid sequence, compound, or polypeptide due to degradation of the sequence or compound by natural mechanisms and/or clearance or sequestration of the sequence or compound in the body. The half-life can be evaluated by methods known to those skilled in the art. The in vivo half-life of the amino acid sequence, compound or polypeptide of the present invention can be determined in any known manner, such as by pharmacokinetic analysis. Suitable techniques are obvious to the person skilled in the art, for example, as described in paragraph o) on page 57 of WO 08/020079. Also as mentioned in paragraph o) on page 57 of WO 08/020079, parameters such as t1/2-α, t1/2-β and area under the curve (AUC) can be used to express the half-life. In this respect, it should be noted that the term “half-life” in the present application specifically refers to t1/2-β or terminal half-life (where t1/2-α and/or AUC can be ignored). For example, refer to the following experimental parts and standard manuals, such as Kenneth, A, etc.: Chemical Stability of Pharmaceuticals: A Handbook for Pharmacists and Peters, etc., Pharmacokinete analysis: A Practical Approach (pharmacokinetics) Analysis: Practice Method) (1996). Also refer to “Pharmacokinetics”, M Gibaldi &#x26; DPerron, Published by Marcel Dekker, 2^{nd} Edition, (1982).

T the terms “affinity” generally refers to the strength of the sum of non-covalent interactions between a single binding site of a molecule (e.g., polypeptide or antibody) and its binding partner (e.g., target or antigen). Unless otherwise specified, when used herein, “binding affinity” refers to the relationship between the members of a binding pair (e.g., in a polypeptide-polynucleotide-complex, or between a polypeptide and its target, or between an antibody and an antibody). The intrinsic binding affinity of 1:1 interaction between antigens. The affinity of a molecule X to its partner Y can usually be represented by the dissociation constant (Kd). Affinity can be measured by common methods known in the art, such as surface plasmon resonance, and also includes those methods reported herein. The higher affinity of molecule X to the binding partner Y can be seen in lower Kd and/or EC50 values.

The term “patient” generally refers to a human or non-human animal, including but not limited to a cat, dog, horse, pig, cow, sheep, rabbit, mouse, rat, or monkey.

The term “pharmaceutically acceptable adjuvant” generally comprises pharmaceutically acceptable carriers, excipients, or stabilizers, which are nontoxic for the cells or mammals that are exposed to them at the dose and concentration used. Generally, the physiologically acceptable carrier is a PH buffered aqueous solution. Examples of the physiologically acceptable carrier may comprise: buffers, such as phosphate, citrate, and other organic acids; antioxidants, including ascorbic acid; low-molecular-weight (less than about 10 residues) polypeptides, and proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrin; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™.

The term “pharmaceutical combination” and “combination product” is used interchangeably, and generally refers to a product resulting from the admixture or combination of more than one active ingredients, and includes both fixed and non-fixed combinations of active ingredients. The term “fixed combination” means that the active ingredients, and one or more combination partners are both administered to a patient simultaneously in the form of a single entity or dose. The term “non-fixed combination” means that the active ingredients and one or more combination partners are administered to a patient simultaneously, jointly or sequentially (without a specific time limit) as separate entities, wherein such administration provides two compounds at therapeutically effective levels in the patient's body. For example, one active ingredient of pharmaceutical combination may be an antigen binding protein prepared by the method described in present application.

The term “pharmaceutical composition” generally refers to a composition suitable for administration to a patient. For example, the term “pharmaceutical composition” contains one or more antigen binding proteins, which is generally prepared by the method described in present application. A pharmaceutical composition may also contain one or more suitable (pharmaceutically effective) carriers, stabilizers, excipients, diluents, solubilizers, surfactants, emulsifiers, preservatives and/or adjuvants. For example, an acceptable component of a composition is nontoxic to the patient at the dose and concentration used. The pharmaceutical composition in present application includes, but is not limited to liquid, frozen and lyophilized compositions.

The term “treatment” generally refers to the administration of an internal or external therapeutic agent to a patient who has one or more disease symptoms, and furthermore, the therapeutic agent is known to show a therapeutic effect against these symptoms. Generally, therapeutic agent is administered to the patient at an amount (therapeutically effective amount) for effectively alleviating one or more disease symptoms. The desired therapeutic effect comprises reducing the rate of disease progression, ameliorating or alleviating the disease state, and regressing or improving the prognosis.

The term “optional” or “optionally” means that the event or situation described subsequently may occur but does not have to occur.

The term “comprise” generally refers to the meaning of including, inclusive, containing, or encompassing. In some cases, it also means “is/are” and “consist of”.

The term “about” generally refers to a variation within a range of 0.5%-10% above or below a specified value, for example, a variation within a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, and 10% above or below a specified value.

### DETAILS DESCRIPTION OF THE INVENTION

### Proteins Capable of Binding VEGF

In one aspect, the present application provides an isolated antigen-binding protein, wherein the isolated antigen-binding protein can have one or more properties selected from the group consisting of: 1) an ability of specifically binding to VEGFA (Vascular Endothelial Growth Factor A); 2) an ability of preventing VEGF from binding its corresponding receptor(s); and 3) an ability of inhibiting VEGF-driven biological functions.

In some embodiments, the VEGFA comprises a human VEGFA. In some embodiments, the VEGF-driven biological function comprises angiogenesis.

In the present application, the VEGFA can include a variant of the VEGFA. For example, the variant can be: 1) a protein or polypeptide formed by substitution, deletion, or addition of one or more amino acids in the amino acid sequence of the VEGFA protein; and 2) a protein or polypeptide with at least about 85% (e.g., at least about 85%, about 90%, about 91%, about 92%, about 93%, about 94%, about 95%, about 96%, about 97%, about 98%, about 99% or higher) of sequence homology with the VEGFA protein.

In some embodiments, the isolated antigen-binding protein comprises at least one CDR of the variable region of the heavy chain of an antibody, for example, a single domain antibody, wherein the variable region comprises any one of the amino acid sequences as set forth in SEQ ID NO: 13-16. In some embodiment, the sequence of the CDRs can be defined by any known numbering strategy, for example, Kabat, Chothia, IMGT, or combination thereof. In the present application, the CDRs amino acid sequence can be specified upon Chothia and Kabat schemes.

In the present application, the isolated antigen-binding protein can comprise a HCDR3, the HCDR3 can comprise an amino acid sequence as set forth in any one of SEQ ID NO: 1, SEQ ID NO: 4, SEQ ID NO: 7, and SEQ ID NO: 10.

In the present application, the isolated antigen-binding protein can comprise a HCDR2, the HCDR2 can comprise an amino acid sequence as set forth in any one of SEQ ID NO: 2, SEQ ID NO: 5, SEQ ID NO: 8, and SEQ ID NO: 11.

In the present application, the isolated antigen-binding protein can comprise a HCDR1, the HCDR1 can comprise an amino acid sequence as set forth in any one of SEQ ID NO: 3, SEQ ID NO: 6, SEQ ID NO: 9, and SEQ ID NO: 12.

In the present application, the isolated antigen-binding protein can comprise a HCDR1, a HCDR2, and a HCDR3, the HCDR1 can comprise an amino acid sequence as set forth in any one of SEQ ID NO: 3, SEQ ID NO: 6, SEQ ID NO: 9, and SEQ ID NO: 12, the HCDR2 can comprise an amino acid sequence as set forth in any one of SEQ ID NO: 2, SEQ ID NO: 5, SEQ ID NO: 8, and SEQ ID NO: 11, the HCDR3 can comprise an amino acid sequence as set forth in any one of SEQ ID NO: 1, SEQ ID NO: 4, SEQ ID NO: 7, and SEQ ID NO: 10.

In the present application, the isolated antigen-binding protein can comprise a HCDR1, a HCDR2, and a HCDR3, wherein the HCDR1, the HCDR2, and the HCDR3 can comprise the amino acid sequences selected any one of the groups consisting of:
the HCDR1 comprises an amino acid sequence as set forth in SEQ ID NO: 3, the HCDR2 comprises an amino acid sequence as set forth in SEQ ID NO: 2, and the HCDR3 comprises an amino acid sequence as set forth in SEQ ID NO: 1;
   the HCDR1 comprises an amino acid sequence as set forth in SEQ ID NO: 6, the HCDR2 comprises an amino acid sequence as set forth in SEQ ID NO: 5, and the HCDR3 comprises an amino acid sequence as set forth in SEQ ID NO: 4;
   the HCDR1 comprises an amino acid sequence as set forth in SEQ ID NO: 9, the HCDR2 comprises an amino acid sequence as set forth in SEQ ID NO: 8, and the HCDR3 comprises an amino acid sequence as set forth in SEQ ID NO: 7; and
   the HCDR1 comprises an amino acid sequence as set forth in SEQ ID NO: 12, the HCDR2 comprises an amino acid sequence as set forth in SEQ ID NO: 11, and the HCDR3 comprises an amino acid sequence as set forth in SEQ ID NO: 10.

In the present application, the isolated antigen-binding protein can comprise a H-FR1, wherein a C-terminus of the H-FR1 is directly or indirectly linked to an N-terminus of the HCDR1, and the H-FR1 comprises an amino acid sequence as set forth in SEQ ID NO: 35. In some embodiments, the H-FR1 comprises an amino acid sequence as set forth in SEQ ID NO: 24 or SEQ ID NO: 28.

In the present application, the isolated antigen-binding protein can comprise a H-FR2, wherein the H-FR2 is located between the HCDR1 and the HCDR2, and the H-FR2 comprises an amino acid sequence as set forth in SEQ ID NO: 36. In some embodiments, the H-FR2 can comprises an amino acid sequence as set forth in SEQ ID NO: 25, SEQ ID NO: 29, SEQ ID NO: 31, or SEQ ID NO: 33.

In the present application, the isolated antigen-binding protein can comprise a H-FR3, wherein the H-FR3 is located between the HCDR2 and the HCDR3, and the H-FR3 comprises an amino acid sequence as set forth in SEQ ID NO: 37. In some embodiments, the H-FR3 can comprise an amino acid sequence as set forth in SEQ ID NO: 26, SEQ ID NO: 30, SEQ ID NO: 32, or SEQ ID NO: 34.

In the present application, the isolated antigen-binding protein can comprise a H-FR4, wherein an N-terminus of the H-FR4 is directly or indirectly linked to a C-terminus of the HCDR3, and the H-FR4 comprises an amino acid sequence as set forth in SEQ ID NO: 27.

In the present application, the isolated antigen-binding protein can comprise a H-FR1, a H-FR2, a H-FR3, and a H-FR4, wherein the H-FR1 can comprise an amino acid sequence as set forth in any one of SEQ ID NO: 24 and SEQ ID NO: 28, the H-FR2 can comprise an amino acid sequence as set forth in any one of SEQ ID NO: 25, SEQ ID NO: 29, SEQ ID NO: 31 and SEQ ID NO: 33, the H-FR3 can comprise an amino acid sequence as set forth in any one of SEQ ID NO: 26, SEQ ID NO: 30, SEQ ID NO: 32, and SEQ ID NO: 34, and the H-FR4 can comprise an amino acid sequence as set forth in SEQ ID NO: 27.

In the present application, the isolated antigen-binding protein can comprise a H-FR1, a H-FR2, a H-FR3, and a H-FR4, wherein the H-FR1, the H-FR2, the H-FR3 and the H-FR4 can comprise the amino acid sequences selected any one from the group consisting of:
the H-FR1 comprises an amino acid sequence as set forth in SEQ ID NO: 24, the H-FR2 comprises an amino acid sequence as set forth in SEQ ID NO: 25, the H-FR3 comprises an amino acid sequence as set forth in SEQ ID NO: 26, and the H-FR4 comprises an amino acid sequence as set forth in SEQ ID NO: 27;
   the H-FR1 comprises an amino acid sequence as set forth in SEQ ID NO: 24, the H-FR2 comprises an amino acid sequence as set forth in SEQ ID NO: 31, the H-FR3 comprises an amino acid sequence as set forth in SEQ ID NO: 32, and the H-FR4 comprises an amino acid sequence as set forth in SEQ ID NO: 27;
   the H-FR1 comprises an amino acid sequence as set forth in SEQ ID NO: 24, the H-FR2 comprises an amino acid sequence as set forth in SEQ ID NO: 33, the H-FR3 comprises an amino acid sequence as set forth in SEQ ID NO: 34, and the H-FR4 comprises an amino acid sequence as set forth in SEQ ID NO: 27; and
   the H-FR1 comprises an amino acid sequence as set forth in SEQ ID NO: 28, the H-FR2 comprises an amino acid sequence as set forth in SEQ ID NO: 29, the H-FR3 comprises an amino acid sequence as set forth in SEQ ID NO: 30, and the H-FR4 comprises an amino acid sequence as set forth in SEQ ID NO: 27.

In the present application, the isolated antigen-binding protein can comprise a heavy chain variable region (VH), and the VH can comprise an amino acid sequence as set forth in any one of SEQ ID NO: 13-16.

In the present application, the isolated antigen-binding protein can comprise an antibody or its antigen-binding fragment.

In some embodiments, the antibody can be selected from the group consisting of a single monoclonal antibody, single strand antibody, chimeric antibody, multi-specific antibody, humanized antibody and fully human antibody.

In some embodiments, the antigen-binding fragment can be selected from the group consisting of Fab, Fab′, F(ab)₂, F(ab′)₂, sdAb, Fv and ScFv fragment or derived bi-paratopic antigen-binding protein.

In the present application, the isolated antigen-binding protein can comprise a single domain antibody.

In the present application, the single domain antibody can comprise a HCDR3, the HCDR3 can comprise an amino acid sequence as set forth in any one of SEQ ID NO: 1, SEQ ID NO: 4, SEQ ID NO: 7, and SEQ ID NO: 10.

In the present application, the single domain antibody can comprise a HCDR2, the HCDR2 can comprise an amino acid sequence as set forth in any one of SEQ ID NO: 2, SEQ ID NO: 5, SEQ ID NO: 8 and SEQ ID NO: 11.

In the present application, the single domain antibody can comprise a HCDR1, the HCDR1 can comprise an amino acid sequence as set forth in any one of SEQ ID NO: 3, SEQ ID NO: 6, SEQ ID NO: 9, and SEQ ID NO: 12.

In the present application, the single domain antibody can comprise a HCDR1, a HCDR2, and a HCDR3, the HCDR1 can comprise an amino acid sequence as set forth in any one of SEQ ID NO: 3, SEQ ID NO: 6, SEQ ID NO: 9, and SEQ ID NO: 12, the HCDR2 can comprise an amino acid sequence as set forth in any one of SEQ ID NO: 2, SEQ ID NO: 5, SEQ ID NO: 8, and SEQ ID NO: 11, the HCDR3 can comprise an amino acid sequence as set forth in any one of SEQ ID NO: 1, SEQ ID NO: 4, SEQ ID NO: 7, and SEQ ID NO: 10.

In the present application, the single domain antibody can comprise a HCDR1, a HCDR2, and a HCDR3, wherein the HCDR1, the HCDR2, and the HCDR3 can comprise the amino acid sequences selected any one of the groups consisting of:
the HCDR1 can comprise an amino acid sequence as set forth in SEQ ID NO: 3, the HCDR2 can comprise an amino acid sequence as set forth in SEQ ID NO: 2, and the HCDR3 can comprise an amino acid sequence as set forth in SEQ ID NO: 1;
   the HCDR1 can comprise an amino acid sequence as set forth in SEQ ID NO: 6, the HCDR2 can comprise an amino acid sequence as set forth in SEQ ID NO: 5, and the HCDR3 can comprise an amino acid sequence as set forth in SEQ ID NO: 4;
   the HCDR1 can comprise an amino acid sequence as set forth in SEQ ID NO: 9, the HCDR2 can comprise an amino acid sequence as set forth in SEQ ID NO: 8, and the HCDR3 can comprise an amino acid sequence as set forth in SEQ ID NO: 7; and
   the HCDR1 can comprise an amino acid sequence as set forth in SEQ ID NO: 12, the HCDR2 can comprise an amino acid sequence as set forth in SEQ ID NO: 11, and the HCDR3 can comprise an amino acid sequence as set forth in SEQ ID NO: 10.

In the present application, the single domain antibody can comprise a H-FR1, wherein a C-terminus of the H-FR1 is directly or indirectly linked to an N-terminus of the HCDR1, and the H-FR1 can comprise an amino acid sequence as set forth in SEQ ID NO: 35. In some embodiments, the H-FR1 can comprise an amino acid sequence as set forth in SEQ ID NO: 24 or SEQ ID NO: 28.

In the present application, the single domain antibody can comprise a H-FR2, wherein the H-FR2 is located between the HCDR1 and said HCDR2, and the H-FR2 can comprise an amino acid sequence as set forth in SEQ ID NO: 36. In some embodiments, the H-FR2 can comprise an amino acid sequence as set forth in SEQ ID NO: 25, SEQ ID NO: 29, SEQ ID NO: 31, or SEQ ID NO: 33.

In the present application, the single domain antibody can comprise a H-FR3, wherein the H-FR3 is located between the HCDR2 and the HCDR3, and the H-FR3 can comprise an amino acid sequence as set forth in SEQ ID NO: 37. In some embodiments, the H-FR3 can comprise an amino acid sequence as set forth in SEQ ID NO: 26, SEQ ID NO: 30, SEQ ID NO: 32, or SEQ ID NO: 34.

In the present application, the single domain antibody can comprise a H-FR4, wherein an N-terminus of the H-FR4 is directly or indirectly linked to a C-terminus of the HCDR3, and the H-FR4 can comprise an amino acid sequence as set forth in SEQ ID NO: 27.

In the present application, the single domain antibody can comprise a H-FR1, a H-FR2, a H-FR3, and a H-FR4, wherein the H-FR1 can comprise an amino acid sequence as set forth in any one of SEQ ID NO: 24 and SEQ ID NO: 28, the H-FR2 can comprise an amino acid sequence as set forth in any one of SEQ ID NO: 25, SEQ ID NO: 29, SEQ ID NO: 31, and SEQ ID NO: 33, the H-FR3 can comprise an amino acid sequence as set forth in any one of SEQ ID NO: 26, SEQ ID NO: 30, SEQ ID NO: 32, and SEQ ID NO: 34, and the H-FR4 can comprise an amino acid sequence as set forth in SEQ ID NO: 27.

In the present application, the single domain antibody can comprise a H-FR1, a H-FR2, a H-FR3, and a H-FR4, wherein the H-FR1, the H-FR2, the H-FR3 and the H-FR4 can comprise the amino acid sequences selected any one from the group consisting of:
the H-FR1 can comprise an amino acid sequence as set forth in SEQ ID NO: 24, the H-FR2 can comprise an amino acid sequence as set forth in SEQ ID NO: 25, the H-FR3 can comprise an amino acid sequence as set forth in SEQ ID NO: 26, and the H-FR4 can comprise an amino acid sequence as set forth in SEQ ID NO: 27;
   the H-FR1 can comprise an amino acid sequence as set forth in SEQ ID NO: 24, the H-FR2 can comprise an amino acid sequence as set forth in SEQ ID NO: 31, the H-FR3 can comprise an amino acid sequence as set forth in SEQ ID NO: 32, and the H-FR4 can comprise an amino acid sequence as set forth in SEQ ID NO: 27;
   the H-FR1 can comprise an amino acid sequence as set forth in SEQ ID NO: 24, the H-FR2 can comprise an amino acid sequence as set forth in SEQ ID NO: 33, the H-FR3 can comprise an amino acid sequence as set forth in SEQ ID NO: 34, and the H-FR4 can comprise an amino acid sequence as set forth in SEQ ID NO: 27; and
   the H-FR1 can comprise an amino acid sequence as set forth in SEQ ID NO: 28, the H-FR2 can comprise an amino acid sequence as set forth in SEQ ID NO: 29, the H-FR3 can comprise an amino acid sequence as set forth in SEQ ID NO: 30, and the H-FR4 can comprise an amino acid sequence as set forth in SEQ ID NO: 27.

In the present application, the single domain antibody can comprise a heavy chain variable region (VHH), and the VHH can comprise an amino acid sequence as set forth in any one of SEQ ID NO: 13-16.

In another aspect, the present application provides a bi-paratopic antigen-binding protein, wherein the bi-paratopic antigen-binding protein can comprise a first antigen-binding domain, and a second binding domain. In some embodiments, the first antigen-binding protein and the second antigen-binding domain can be linked via a linker. In some embodiments, the first antigen-binding protein and the second antigen-binding protein can be linked without a linker.

In the present application, the first antigen-binding domain of the bi-paratopic antigen-binding protein can comprise the isolated antigen-binding protein of the present application. In the present application, the second antigen-binding domain of the bi-paratopic antigen-binding protein can comprise the isolated antigen-binding protein of the present application. In some embodiments, the first antigen-binding domain and the second antigen-binding domain of the bi-paratopic antigen-binding protein can comprise the isolated antigen-binding protein of the present application.

In the present application, the first antigen-binding domain and the second antigen-binding domain of the bi-paratopic antigen-binding protein can target the same antigen.

In the present application, the first antigen-binding domain and the second antigen-binding domain of the bi-paratopic antigen-binding protein can bind the different epitopes.

In the present application, the first antigen-binding domain and the second antigen-binding domain of the bi-paratopic antigen-binding protein can have the different amino acid sequences.

In some embodiments, the bi-paratopic antigen-binding protein can comprise CDRs amino acid sequences selected from the group consisting of: 1) First antigen binding domain: the HCDR1 comprising the amino acid sequence as set forth in SEQ ID NO: 3, the HCDR2 comprising the amino acid sequence as set forth in SEQ ID NO: 2, the HCDR3 comprising the amino acid sequence as set forth in SEQ ID NO: 1; Second antigen binding domain: the HCDR1 comprising the amino acid sequence as set forth in SEQ ID NO: 6, the HCDR2 comprising the amino acid sequence as set forth in SEQ ID NO: 5, the HCDR3 comprising the amino acid sequence as set forth in SEQ ID NO: 4; 2) First antigen binding domain: the HCDR1 comprising the amino acid sequence as set forth in SEQ ID NO: 3, the HCDR2 comprising the amino acid sequence as set forth in SEQ ID NO: 2, the HCDR3 comprising the amino acid sequence as set forth in SEQ ID NO: 1; Second antigen binding domain: the HCDR1 comprising the amino acid sequence as set forth in SEQ ID NO: 9, the HCDR2 comprising the amino acid sequence as set forth in SEQ ID NO: 8, the HCDR3 comprising the amino acid sequence as set forth in SEQ ID NO: 7; and 3) First antigen binding domain: the HCDR1 comprising the amino acid sequence as set forth in SEQ ID NO: 3, the HCDR2 comprising the amino acid sequence as set forth in SEQ ID NO: 2, the HCDR3 comprising the amino acid sequence as set forth in SEQ ID NO: 1; Second antigen binding domain: the HCDR1 comprising the amino acid sequence as set forth in SEQ ID NO: 12, the HCDR2 comprising the amino acid sequence as set forth in SEQ ID NO: 11, the HCDR3 comprising the amino acid sequence as set forth in SEQ ID NO: 10.

In some embodiments, the bi-paratopic antigen-binding protein can comprise VHH sequences selected from any one of the groups consisting of: the first antigen-binding protein can comprise a VHH amino acid sequence as set forth in SEQ ID NO: 13; the second antigen-binding protein can comprise a VHH amino acid sequence as set forth in SEQ ID NO: 14; the first antigen-binding protein can comprise a VHH amino acid sequence as set forth in SEQ ID NO: 13; the second antigen-binding protein can comprise a VHH amino acid sequence as set forth in SEQ ID NO: 15; and the first antigen-binding protein can comprise a VHH amino acid sequence as set forth in SEQ ID NO: 13; the second antigen-binding protein can comprise a VHH amino acid sequence as set forth in SEQ ID NO: 16.

In the present application, the bi-paratopic antigen-binding protein can comprise an amino acid sequence as set forth in any one of SEQ ID NO: 17-19.

In another aspect, the present application provides a polypeptide, wherein the polypeptide can comprise the isolated binding protein, and other functional domain.

In another aspect, the present application provides a polypeptide, wherein the polypeptide can comprise the bi-paratopic antigen-binding protein, and other functional domain.

In some embodiments, the functional domain can comprise a Fc region. In some embodiments, the Fc region can comprise a human Fc region. In some embodiments, the Fc region can comprise a human IgG Fc region or its variants, for example, the Fc region can comprise at least about 85% (e.g., at least about 85%, about 90%, about 91%, about 92%, about 93%, about 94%, about 95%, about 96%, about 97%, about 98%, about 99% or higher) of sequence homology with the wild-type human IgG Fc region. In some embodiments, the Fc region can comprise an amino acid sequence as set forth in SEQ ID NO: 20 or with at least about 85% of sequence homology.

In the present application, the functional domain can comprise a complement-inhibiting entity. For example, the complement-inhibiting entity can comprise a complement H (CFH) fragment. For example, the complement H (CFH) fragment can comprise a human complement H (CFH) fragment. For example, the complement H (CFH) fragment can comprise an amino acid sequence as set forth in SEQ ID NO: 22.

In some embodiments, the polypeptide can comprise an amino acid sequence as set forth in SEQ ID NO: 21 or SEQ ID NO: 23.

In some embodiments, the antigen-binding protein SLN6068 comprises an amino acid sequence of SEQ ID NO: 13. In some embodiments, the antigen-binding protein SLN6043 comprises an amino acid sequence of SEQ ID NO: 14. In some embodiments, the antigen-binding protein SLN6062 comprises an amino acid sequence of SEQ ID NO: 15. In some embodiments, the antigen-binding protein SLN6065 comprises an amino acid sequence of SEQ ID NO: 16. In some embodiments, the antigen-binding protein SLN6071 comprises an amino acid sequence of SEQ ID NO: 17. In some embodiments, the antigen-binding protein SLN6075 comprises an amino acid sequence of SEQ ID NO: 18. In some embodiments, the antigen-binding protein SLN6079 comprises an amino acid sequence of SEQ ID NO: 19. In some embodiments, the antigen-binding protein SLN6073 comprises an amino acid sequence of SEQ ID NO: 21. In some embodiments, the antigen-binding protein SLN6074 comprises an amino acid sequence of SEQ ID NO: 23.

In the present application, the antigen-binding protein can be isolated or purified.

In another aspect, the present application provides one or more isolated nucleic acid molecules, wherein the nucleic acid molecules encode the isolated antigen-binding protein, the bi-paratopic antigen-binding protein or the polypeptide. In the present application, the nucleic acid encoding the isolated antigen-binding protein, the bi-paratopic antigen-binding protein or the polypeptide can be prepared by various methods known in the art, including, but not limited to, overlapping PCR by using restrictive fragment operation or using synthetic oligonucleotide. See, e.g., Sambrook et al., Molecular Cloning, A Laboratory Manual, Cold Spring Harbor Laboratory Press, Cold Spring Harbor, N.Y., 1989; and Ausube et al. Current Protocols in Molecular Biology, Greene Publishing and Wiley-Interscience, New York N.Y., 1993.

In another aspect, the present application provides one or more vectors including the one or more nucleic acid molecules of the present application. Each vector can include one or more nucleic acid molecules. Moreover, the vector can further include other gene(s), e.g., a marker gene that allows the selection of the vector in an appropriate host cell and under appropriate conditions. Moreover, the vector can further include an expression control element that allows the coding region to be properly expressed in an appropriate host. Such control element is well known by persons skilled in the art, e.g., it can include promoter, ribosome binding site, enhancer and other control elements regulating the transcription of gene or translation of mRNA, and the like. The one or more nucleic acid molecules of the present application can be operatively linked to the expression control element. The vector can include, e.g., plasmid, cosmid, virus, phage, or other vectors commonly used in, e.g., genetic engineering. For example, the vector is an expression vector.

In another aspect, the present application provides a cell, and the cell can include the one or more nucleic acid molecules of the present application and/or the one or more vectors of the present application. For example, each cell can include one nucleic acid molecule or one vector of the present application. For example, each cell or each kind of cell can include a plurality of (e.g., two or more, e.g., two kinds or more kinds) nucleic acid molecules or vectors of the present application. For example, the vector of the present application can be introduced into cells, e.g., eukaryotic cells, such as cells from plants, fungi or yeast cells, etc. The vectors of the present application can be introduced into cells by methods known in the art, such as electroporation, lipofectine transfection, lipofectamin transfection, and the like.

In another aspect, the present application provides a method of preparing the isolated antigen-binding protein, the bi-paratopic antigen-binding protein or the polypeptide. The method can include culturing the host cell of the present application under conditions that allow the antibody or its antigen-binding fragment to be expressed. For example, the method can include using an appropriate medium, an appropriate temperature, and culturing time, that are understood by persons of ordinary skills in the art.

In another aspect, the present application provides a pharmaceutical composition comprising the isolated antigen-binding protein, the bi-paratopic antigen-binding protein or the polypeptide, and optionally pharmaceutically acceptable adjuvants.

The pharmaceutically acceptable adjuvants can include buffers, antioxidants, preservatives, low molecular weight polypeptides, proteins, hydrophilic polymers, amino acids, carbohydrates, chelating agents, counterions, metal complexes and/or nonionic surfactants, etc.

In the present application, the pharmaceutical composition can be formulated for oral administration, intravenous administration, intramuscular administration, in situ administration at the tumor site, inhalation, rectal administration, vaginal administration, transdermal administration or administration via subcutaneous depot.

In another aspect, the present application provides use of the isolated antigen-binding protein, the bi-paratopic antigen-binding protein, the polypeptide the nucleic acid molecules, the vectors, the cell, and/or the pharmaceutical composition in preparation of a drug, wherein the drug is used for preventing or treating a disease.

In another aspect, the present application provides the isolated antigen-binding protein, the bi-paratopic antigen-binding protein, the polypeptide the nucleic acid molecules, the vectors, the cell, and/or the pharmaceutical composition, for use in preventing or treating a disease.

In another aspect, the present application provides a method of preventing or treating a disease in a subject in need thereof, including administering to the subject the isolated antigen-binding protein, the bi-paratopic antigen-binding protein, the polypeptide the nucleic acid molecules, the vectors, the cell, and/or the pharmaceutical composition.

In the present application, the disease can comprise a VEGFA-associated disease. For example, the disease can comprise a tumor. For example, the disease can comprise an age-related macular degeneration. For example, the disease can comprise a VEGFA-driven pathogenic process.

### Proteins Capable of Binding Transferrin

### Transferrin-Binding Protein

In an aspect, the present application the present application provides a transferrin-binding protein which is capable of specifically binding to transferrin and not disturbing the interaction between transferrin and transferrin receptor 1.

For example, the transferrin-binding protein may be capable of extending half-life in circulation of its associated entities.

For example, the associated entity may be any monomeric or multimeric protein, protein fragment, nucleotide sequence, small molecular compound, a delivery vehicle, or a modified transgene vector that was specifically linked to an antigen binding protein. For example, the associated entity may include but not limit to antibodies and binding parts thereof, such as immunologically functional fragments. For example, the antigen binding protein may be transferrin-binding protein.

For example, the transferrin-binding protein may enable a fusion protein comprising the transferrin-binding protein to cross blood-brain barrier (BBB).

For example, the BBB may be a physiological barrier between the peripheral circulation and the brain and spinal cord. For example, the BBB may comprise the tight junctions in the plasma membrane of brain capillary endothelial cells and constitutes a tight barrier that restricts the transport of molecules to the brain, even very small molecules such as urea (60 Daltons). For example, the brain capillary endothelial cells may have weaker pinocytosis. For example, the blood-brain barrier may comprise the BBB in the brain, the blood-spinal cord barrier in the spinal cord, and the blood-retinal barrier in the retina. For example, the BBB may also comprise the blood-CSF barrier (choroid plexus), where the barrier is composed of ependymal cells instead of capillary endothelial cells.

For example, the fusion protein may enter the brain tissue by crossing the BBB.

For example, the transferrin-binding protein may be used for oral delivery of a drug. For example, the dosing form of the oral delivery of a drug may be a capsule, a granule, or a tablet. For example, the transferrin-binding protein may enable intracellular delivery of its associated entities to transferrin receptor-expressing cells. For example, the transferrin-binding protein may be capable of specifically binding to transferrin and not disturbing the interaction between transferrin and transferrin receptor 1.

For example, the transferrin may be a human transferrin. For example, the transferrin may be iron-containing holo-transferrin. For example, the iron-containing holo-transferrin may comprise the transferrin and Fe³⁺. For example, the transferrin may bind to Fe³⁺. For example, the transferrin may bind to two Fe³⁺. For example, the iron-containing holo-transferrin may bind to the transferrin receptor. For example, the iron-containing holo-transferrin may bind to the transferrin receptor 1. For example, the iron-containing holo-transferrin may be transported to the endosome by the transferrin receptor. For example, the iron-containing holo-transferrin may be transported through endocytosis. For example, the iron-containing holo-transferrin may be transported through transcytosis. For example, the iron-containing holo-transferrin may separate from Fe³⁺. For example, the transferrin may be iron-free apo-transferrin.

For example, the iron-free apo-transferrin may be come from the iron-containing holo-transferrin separated from Fe³⁺. For example, the iron-free apo-transferrin may be capable of binding to Fe³⁺. For example, the transferrin-binding protein may prosses a higher affinity to iron-containing holo-transferrin than that to iron-free apo-transferrin.

For example, the affinity may be measured by surface plasmon resonance-based assays. For example, the affinity may be measured by enzyme-linked immunosorbent assays (ELISA). For example, the affinity may be measured by competition assays (RIA). For example, the affinity may be measured by flow cytometry (FACS). For example, the affinity may be expressed as the binding equilibrium constant (KD). For example, the affinity of transferrin-binding protein to iron-containing holo-transferrin may be 2-fold, 4-fold, 6-fold, 8-fold, 10-fold, 20-fold, 40-fold, 60-fold, 80-fold, 100-fold higher than that to iron-free apo-transferrin.

For example, the transferrin-binding protein may comprise monoclonal antibody. For example, the transferrin-binding protein may comprise single chain antibody fragment. For example, the transferrin-binding protein may comprise single domain antibody fragment. For example, the transferrin-binding protein may comprise engineered protein. For example, the transferrin-binding protein may comprise a peptide. For example, the antibody or antibody fragment of the transferrin-binding protein may comprise animal-derived sequence. For example, the antibody or antibody fragment of the transferrin-binding protein may comprise humanized sequence. For example, the antibody or antibody fragment of the transferrin-binding protein may comprise fully human sequence. For example, the antibody or antibody fragment of the transferrin-binding protein may comprise a chimeric sequence. For example, the antibody or antibody fragment of the transferrin-binding protein may comprise a synthetic sequence. For example, the antibody or antibody fragment of the transferrin-binding protein may be a single domain antibody fragment VHH.

In the present application, the transferrin-binding protein may include at least one CDR in a heavy chain variable region VH, wherein the VH includes an amino acid sequence as set forth in any of SEQ ID NOs: 108-142.

For example, the VH may include an amino acid sequence as set forth in any one of SEQ ID NOs: 108-142.

For example, the transferrin-binding protein may include the HCDR3 of the VH with an amino acid sequence as set forth in any one of SEQ ID NOs: 83-107.

For example, the transferrin-binding protein may include the HCDR2 of the VH with an amino acid sequence as set forth in any one of SEQ ID NOs: 60-82.

For example, the transferrin-binding protein may include the HCDR1 of the VH with an amino acid sequence as set forth in any one of SEQ ID NOs: 39-59.

For example, the transferrin-binding protein may comprise a HCDR1, a HCDR2, and a HCDR3, the HCDR1 may comprise an amino acid sequence as set forth in any one of SEQ ID NOs: 39-59, the HCDR2 may comprise an amino acid sequence as set forth in any one of SEQ ID NOs: 60-82, and the HCDR3 may comprise an amino acid sequence as set forth in any one of SEQ ID NOs: 83-107.

For example, the transferrin-binding protein may comprise a Fc region. For example, the Fc region may comprise a human Fc region. For example, the Fc region may comprise a human IgG Fc region. For example, the Fc region may comprise a human IgG1 Fc region. For example, the Fc region may comprise a human IgG4 Fc region.

For example, the transferrin-binding protein may comprise an antibody or its antigen binding fragment. The antibody may be selected from the group consisting of monoclonal antibody, single strand antibody, chimeric antibody, polyspecific antibody, humanized antibody and fully human antibody. The antigen binding fragments is selected from the group consisting of Fab, Fab′, F(ab)₂, F(ab′)₂, sdAb, Fv, dAb and ScFv fragment.

For example, the transferrin-binding protein may be a VHH.

For example, the VHH may comprise a CDR3, and the CDR3 may comprise an amino acid sequence as set forth in any one of SEQ ID NOs: 83-107.

For example, the VHH may comprise a CDR2, and the CDR2 may comprise an amino acid sequence as set forth in any one of SEQ ID NOs: 60-82.

For example, the VHH may comprise a CDR1, and the CDR1 may comprise an amino acid sequence as set forth in any one of SEQ ID NOs: 39-59.

For example, the VHH may comprise a CDR1, a CDR2, and a CDR3, the CDR1 may comprise an amino acid sequence as set forth in any one of SEQ ID NOs: 39-59, the CDR2 may comprise an amino acid sequence as set forth in any one of SEQ ID NOs: 60-82, and the CDR3 may comprise an amino acid sequence as set forth in any one of SEQ ID NOs: 83-107.

For example, the VHH may comprise a CDR1, a CDR2, and a CDR3, the CDR1, the CDR2, and the CDR3 may comprise an amino acid sequence as set forth in SEQ ID NO: 39, SEQ ID NO: 60, and SEQ ID NO: 83, respectively; or the CDR1, the CDR2, and the CDR3 may comprise an amino acid sequence as set forth in S SEQ ID NO: 39, SEQ ID NO: 60, and SEQ ID NO: 101, respectively; or the CDR1, the CDR2, and the CDR3 may comprise an amino acid sequence as set forth in SEQ ID NO: 40, SEQ ID NO: 61, and SEQ ID NO: 84, respectively; or the CDR1, the CDR2, and the CDR3 may comprise an amino acid sequence as set forth in SEQ ID NO: 40, SEQ ID NO: 61, and SEQ ID NO: 96, respectively; or the CDR1, the CDR2, and the CDR3 may comprise an amino acid sequence as set forth in SEQ ID NO: 40, SEQ ID NO: 78, and SEQ ID NO: 96, respectively; or the CDR1, the CDR2, and the CDR3 may comprise an amino acid sequence as set forth in SEQ ID NO: 40, SEQ ID NO: 79, and SEQ ID NO: 96, respectively; or the CDR1, the CDR2, and the CDR3 may comprise an amino acid sequence as set forth in SEQ ID NO: 40, SEQ ID NO: 61, and SEQ ID NO: 104, respectively; or the CDR1, the CDR2, and the CDR3 may comprise an amino acid sequence as set forth in SEQ ID NO: 41, SEQ ID NO: 62, and SEQ ID NO: 85, respectively; or the CDR1, the CDR2, and the CDR3 may comprise an amino acid sequence as set forth in SEQ ID NO: 42, SEQ ID NO: 63, and SEQ ID NO: 86, respectively; or the CDR1, the CDR2, and the CDR3 may comprise an amino acid sequence as set forth in SEQ ID NO: 43, SEQ ID NO: 64, and SEQ ID NO: 87, respectively; or the CDR1, the CDR2, and the CDR3 may comprise an amino acid sequence as set forth in SEQ ID NO: 44, SEQ ID NO: 65, and SEQ ID NO: 88, respectively; or the CDR1, the CDR2, and the CDR3 may comprise an amino acid sequence as set forth in SEQ ID NO: 44, SEQ ID NO: 65, and SEQ ID NO: 97, respectively; or the CDR1, the CDR2, and the CDR3 may comprise an amino acid sequence as set forth in SEQ ID NO: 45, SEQ ID NO: 66, and SEQ ID NO: 89, respectively; or the CDR1, the CDR2, and the CDR3 may comprise an amino acid sequence as set forth in SEQ ID NO: 46, SEQ ID NO: 67, and SEQ ID NO: 90, respectively; or the CDR1, the CDR2, and the CDR3 may comprise an amino acid sequence as set forth in SEQ ID NO: 47, SEQ ID NO: 68, and SEQ ID NO: 91, respectively; or the CDR1, the CDR2, and the CDR3 may comprise an amino acid sequence as set forth in SEQ ID NO: 48, SEQ ID NO: 69, and SEQ ID NO: 92, respectively; or the CDR1, the CDR2, and the CDR3 may comprise an amino acid sequence as set forth in SEQ ID NO: 49, SEQ ID NO: 70, and SEQ ID NO: 93, respectively; or the CDR1, the CDR2, and the CDR3 may comprise an amino acid sequence as set forth in SEQ ID NO: 50, SEQ ID NO: 71, and SEQ ID NO: 94, respectively; or the CDR1, the CDR2, and the CDR3 may comprise an amino acid sequence as set forth in SEQ ID NO: 51, SEQ ID NO: 72, and SEQ ID NO: 95, respectively; or the CDR1, the CDR2, and the CDR3 may comprise an amino acid sequence as set forth in SEQ ID NO: 52, SEQ ID NO: 73, and SEQ ID NO: 98, respectively; or the CDR1, the CDR2, and the CDR3 may comprise an amino acid sequence as set forth in SEQ ID NO: 53, SEQ ID NO: 74, and SEQ ID NO: 99, respectively; or the CDR1, the CDR2, and the CDR3 may comprise an amino acid sequence as set forth in SEQ ID NO: 54, SEQ ID NO: 75, and SEQ ID NO: 100, respectively; or the CDR1, the CDR2, and the CDR3 may comprise an amino acid sequence as set forth in SEQ ID NO: 55, SEQ ID NO: 76, and SEQ ID NO: 102, respectively; or the CDR1, the CDR2, and the CDR3 may comprise an amino acid sequence as set forth in SEQ ID NO: 56, SEQ ID NO: 39, and SEQ ID NO: 103, respectively; or the CDR1, the CDR2, and the CDR3 may comprise an amino acid sequence as set forth in SEQ ID NO: 57, SEQ ID NO: 80, and SEQ ID NO: 105, respectively; or the CDR1, the CDR2, and the CDR3 may comprise an amino acid sequence as set forth in SEQ ID NO: 58, SEQ ID NO: 81, and SEQ ID NO: 106, respectively; or the CDR1, the CDR2, and the CDR3 may comprise an amino acid sequence as set forth in SEQ ID NO: 59, SEQ ID NO: 82, and SEQ ID NO: 107, respectively.

For example, the VHH may comprise an amino acid sequence as set forth in any one of SEQ ID NOs: 108-142. For example, the SLN9056 comprises an amino acid sequence as set forth in SEQ ID NO: 108. For example, the SLN9057 comprises an amino acid sequence as set forth in SEQ ID NO: 109. For example, the SLN0042 comprises an amino acid sequence as set forth in SEQ ID NO: 110. For example, the SLN0043 comprises an amino acid sequence as set forth in SEQ ID NO: 111. For example, the SLN0044 comprises an amino acid sequence as set forth in SEQ ID NO: 112. For example, the SLN0045 comprises an amino acid sequence as set forth in SEQ ID NO: 113. For example, the SLN0049 comprises an amino acid sequence as set forth in SEQ ID NO: 114. For example, the SLN0056 comprises an amino acid sequence as set forth in SEQ ID NO: 115. For example, the SLN0057 comprises an amino acid sequence as set forth in SEQ ID NO: 116. For example, the SLN0059 comprises an amino acid sequence as set forth in SEQ ID NO: 117. For example, the SLN0062 comprises an amino acid sequence as set forth in SEQ ID NO: 118. For example, the SLN0064 comprises an amino acid sequence as set forth in SEQ ID NO: 119. For example, the SLN0065 comprises an amino acid sequence as set forth in SEQ ID NO: 120. For example, the SLN0071 comprises an amino acid sequence as set forth in SEQ ID NO: 121. For example, the SLN0072 comprises an amino acid sequence as set forth in SEQ ID NO: 122. For example, the SLN0046 comprises an amino acid sequence as set forth in SEQ ID NO: 123. For example, the SLN0058 comprises an amino acid sequence as set forth in SEQ ID NO: 124. For example, the SLN9008 comprises an amino acid sequence as set forth in SEQ ID NO: 125. For example, the SLN9013 comprises an amino acid sequence as set forth in SEQ ID NO: 126. For example, the SLN9015 comprises an amino acid sequence as set forth in SEQ ID NO: 127. For example, the SLN9025 comprises an amino acid sequence as set forth in SEQ ID NO: 128. For example, the SLN9026 comprises an amino acid sequence as set forth in SEQ ID NO: 129. For example, the SLN9056 comprises an amino acid sequence as set forth in SEQ ID NO: 108.

The antigen binding protein of the present application may compete with the reference antibody to bind to the Tf, wherein the reference antibody may include a heavy chain variable region and a light chain variable region, the heavy chain variable region of the reference antibody may include HCDR1, HCDR2 and HCDR3, the HCDR1 may include an amino acid sequence as set forth in any one of SEQ ID NOs: 39-59, the HCDR2 may include an amino acid sequence as set forth in any one of SEQ ID NOs: 60-82, and the HCDR3 may include an amino acid sequence as set forth in any one of SEQ ID NOs: 83-107.

### Polypeptide

In another aspect, the present application provides a polypeptide which comprising the transferrin-binding protein.

For example, the polypeptide may comprise a therapeutic entity.

For example, the therapeutic entity may be an engineered cytotoxic pseudomonas exotoxin A (PE38). For example, the PE38 may comprises an amino acid sequence as set forth in SEQ ID NO: 147.

For example, the therapeutic entity may be a glucagon-like peptide-1 (GLP-1) or its variant. For example, the GLP-1 may comprises an amino acid sequence as set forth in SEQ ID NO: 146.

For example, the variant may be a polypeptide that has significant sequence identity with the parent polypeptide and retain the biological activity of the parent polypeptide. For example, the amino acid sequence of variant may have at least about 50%, 75%, 80%, 90%, 95%, 96%, 97%, 98%, 98.2%, 98.4%, 98.6%, 98.8%, 99%, 99.1%, 99.2%, 99.3%, 99.4%, 99.5%, 99.6%, 99.7%, 99.8%, 99.9% or more identity with the amino acid sequence of the parent polypeptide. For example, the variant may also be a protein or polypeptide with one or more amino acids substituted, deleted or added to the amino acid sequence of the protein and/or the polypeptide. For example, the variant may include a protein or polypeptide that has amino acid changes by at least one, such as 1-30, 1-20 or 1-10, and also such as 1, 2, 3, 4 or 5 amino acid substitution, deletion and/or insertion. For example, the substitution may be conservative.

For example, the therapeutic entity and the transferrin-binding protein may be directly linked. For example, the therapeutic entity and the transferrin-binding protein may be linked through gene fusion or recombinant DNA technologies. For example, the therapeutic entity and the transferrin-binding protein may be indirectly linked. For example, the therapeutic entity and the transferrin-binding protein may be linked through a spacer. For example, the therapeutic entity and the transferrin-binding protein may be linked through a linker. For example, the linker may be a peptide with amino acid sequence. For example, the linker may be derived from artificial synthesis.

### Isolated Nucleic Acid Molecules, Vector, Cell

In another aspect, the present application provides one or more isolated nucleic acid molecules encoding the isolated transferrin-binding protein of the present application or the polypeptide.

The nucleic acid molecule of the present application may be isolated. For example, it may be produced or synthesized by the following methods: (i) amplified in vitro, for example produced by polymerase chain reaction (PCR) amplification, (ii) produced by cloning and recombination, (iii) purified, for example by enzyme digestion and gel electrophoresis fractionation, or (iv) synthesized, for example chemically synthesized. In certain embodiments, the isolated nucleic acid is a nucleic acid molecule prepared by a recombinant DNA technology.

In the present application, the nucleic acid encoding the antibody or an antigen-binding fragment thereof may be prepared by a variety of methods known in the art, including but not limited to restriction fragment operation or overlap extension PCR employing synthetic oligonucleotides.

In another aspect, the present application provides a vector which may include the nucleic acid molecule of the present application.

In another aspect, the present application provides one or more vectors including one or more nucleic acid molecules of the present application. Each vector may contain one or more of the nucleic acid molecules. Furthermore, the vector may also contain other genes, e.g., a marker gene that allows selection of the vector in an appropriate host cell and under appropriate conditions. Furthermore, the vector may also contain an expression control element that allows a coding region to be correctly expressed in an appropriate host. Such a control element is well known to those skilled in the art, and may include, for example, a promoter, a ribosome binding site, an enhancer, and other control elements that regulate gene transcription or mRNA translation. The one or more nucleic acid molecules of the present application may be operably linked to the expression control element. The vector may include, for example, a plasmid, a cosmid, a virus, a phage, or other vectors commonly used in, for example, genetic engineering. For example, the vector is an expression vector.

In another aspect, the present application provides a cell which may include the nucleic acid molecule of the present application or the vector of the present application.

In another aspect, the present application provides a host cell, which may include one or more nucleic acid molecules of the present application and/or one or more vectors of the present application.

In certain embodiments, each kind of or each host cell may include one or one kind of nucleic acid molecule or vector of the present application. In certain embodiments, each kind of or each host cell may include multiple (e.g., 2 or more) or multiple kinds of (e.g., 2 or more kinds of) nucleic acid molecules or vectors of the present application. For example, the vector of the present application may be introduced into the host cell, e.g., a eukaryotic cell, such as a cell from a plant, a fungal cell or a yeast cell, etc. The vector of the present application may be introduced into the host cell by a method known in the art, such as electroporation, lipofectine transfection, lipofectamin transfection, and the like.

### Pharmaceutical Composition

In another aspect, the present application provides a pharmaceutical composition which may include the transferrin-binding protein of the present application, the nucleic acid molecule of the present application, the vector of the present application and/or the cell of the present application, and optionally a pharmaceutically-acceptable adjuvant.

For example, the pharmaceutical composition of the present application may be directly used for therapeutic or diagnosis, and thus may be used for preventing and treating diseases Furthermore, other therapeutic entities may be used at the same time.

The pharmaceutical composition of the present application may contain a safe and effective amount (e.g., 0.001-99 wt %, 0.01-90 wt %, or 0.1-80 wt %) of the transferrin-binding protein of the present application and a pharmaceutically-acceptable adjuvant (which may include a carrier or excipient). Such a carrier may include, but are not limited to, saline, a buffer, glucose, water, glycerol, ethanol, and a combination thereof. A pharmaceutical preparation should be matched with the mode of administration. The pharmaceutical composition of the present application may be made into an injection form, for example, it may be prepared by a conventional method with physiological saline or an aqueous solution containing glucose and other adjuvants. The pharmaceutical composition, such as an injection or a solution, should be manufactured under aseptic conditions. The administration amount of the active ingredient is a therapeutically effective amount. Moreover, the transferrin-binding protein of the present application may also be used together with other therapeutic entities.

The transferrin-binding protein or pharmaceutical composition described herein may be formulated, administered and administered in a manner consistent with good medical practice. Considerations in this case include a specific condition being treated, a specific mammal being treated, the clinical symptoms of an individual patient, the etiology of a condition, a drug delivery site, an administration method, and other factors known to medical practitioners. A therapeutic entity needs not, but is optionally formulated together with and/or administered simultaneously with one or more entities currently used for preventing or treating the condition under consideration. The effective amount of such other entities depends on the amount of the therapeutic agent present in the preparation, the type of condition or treatment, and other factors discussed above. Generally, these entities may be used at any dose determined empirically/clinically as appropriate and by any route determined empirically/clinically as appropriate. Compared with single therapy, the dose of the antibody administered in the combined therapy may be reduced. It is easy to monitor the progress of this therapy by conventional techniques.

### The Method

In another aspect, the present application the present application provides one method to translocate a molecule across cellular membrane by using a transferrin-binding protein.

For example, the method may be used for drug delivery across cellular membrane. For example, the cellular membrane may belong to a polarized cell. For example, the polarized cell may be a functional cell. For example, the polarized cell may be a nerve cell. For example, the cellular membrane may belong to a unpolarized cell. For example, the unpolarized cell may be a nerve cell. For example, the unpolarized cell may transmit nerve signals. For example, the polarized and/or unpolarized cell may express transferrin receptor on its cellular membrane. For example, the polarized and/or unpolarized cell may express human transferrin receptor on its cellular membrane. For example, the polarized and/or unpolarized cell may express transferrin receptor 1. For example, the polarized and/or unpolarized cell may express human transferrin receptor 1. For example, the unpolarized cell may belong to blood brain barrier. For example, the unpolarized cell may belong to intestinal epithelium. For example, the unpolarized cell may belong to multiple layers of cells in a solid tissue.

In the present application, the method of drug delivery across cellular membrane may comprise crossing blood brain barrier, crossing intestinal epithelium, crossing multiple layers of cells in a solid tissue, intracellular delivery of drug, and/or recycling of an endocytosed drug back to circulation. For example, the drug delivery across cellular membrane may belong to an unpolarized cell. For example, the drug delivery across cellular membrane may comprise crossing blood brain barrier of central nervous system-targeted systemically dosed drug. For example, the drug delivery across cellular membrane may comprise crossing intestinal epithelium of orally administered drug. For example, the drug delivery across cellular membrane may comprise penetration of drug through multiple layers of cells in a solid tissue. For example, the drug delivery across cellular membrane may belong to a polarized cell. For example, the drug delivery across cellular membrane may comprise intracellular delivery of drug. For example, the drug delivery across cellular membrane may comprise recycling of an endocytosed drug back to circulation.

For example, the drug may comprise a small molecular compound. For example, the small molecular compound may be a chemically synthesized drug. For example, the molecular weight of the small molecular compound may be less than 1000 Daltons. For example, the small molecular compound may have a therapeutic effect. For example, the small molecular compound may have anti-tumor activity.

For example, the drug may comprise a synthetic peptide. For example, the synthetic peptide may comprise α-amino acid. For example, the α-amino acid may be linked through peptide chain. For example, the synthetic peptide may be synthesized through solid phase peptide synthesis. For example, the synthetic peptide may be synthesized through liquid phase peptide synthesis.

For example, the drug may comprise a recombinant protein. For example, the recombinant protein may be a semi-synthetic or synthetic origin polypeptide. For example, the recombinant protein may be expressed by using recombinant DNA technology to connect DNA molecules from different sources. For example, the recombinant protein may be no longer fully or partially associated with the protein in natural state. For example, the recombinant protein may be linked to a peptide other than the peptide linked in its natural state. For example, the recombinant protein may not exist in the natural state.

For example, the drug may comprise an antibody. For example, the drug may comprise an antibody fragment. For example, the antigen fragment may be a Fab, a Fab′, a F(ab)₂, a Fv fragment, a F(ab′)₂, a scFv, a di-scFv and/or dAb.

For example, the drug may comprise an enzyme. For example, the enzyme may be a catalytically active protein.

For example, the drug may comprise a piece of nucleotide acid sequence. For example, the nucleotide acid sequence may be a DNA sequence. For example, the nucleotide sequence may be an RNA sequence. For example, the nucleotide sequence may be genomic DNA, cDNA, synthetic DNA, proviral DNA, viral DNA, mRNA, synthetic RNA or a combination thereof. For example, the nucleotide sequence may encode peptide or protein. For example, the synthetic RNA may modulate mRNA stability or translation. For example, the synthetic RNA could be small interfering RNA (siRNA) or antisense oligoes (ASO).

For example, the drug may comprise a liposome. For example, the liposome may be a vesicle composed one or more layers of concentrically arranged lipid bilayers. For example, the liposome may comprise a water phase. For example, the water phase may comprise nucleic acid.

For example, the drug may comprise a nano-sized particles such as lipid nanoparticle. For example, the lipid nanoparticle may contain multiple lipid molecules physically bound to each other by intermolecular forces. For example, the intermolecular forces may be covalent or non-covalent. For example, the lipid nanoparticle may comprise one or more lipids. For example, the lipids may be cationic lipids, non-cationic lipids and PEG-lipids.

For example, the drug may comprise a drug vehicle. For example, the drug vehicle may be a standard composition suitable for human administration. For example, the drug vehicle may be a typical adjuvant used in animal vaccination.

For example, the drug may comprise a modified virus. For example, the modified virus may be a genetically engineered virus. For example, the modified virus may inhibit the growth of cancerous or hyperproliferative cells in vivo or in vitro. For example, the modified virus may induce death on cancerous or hyperproliferative cells in vivo or in vitro. For example, the modified virus may be an oncolytic virus.

For example, the drug may comprise a gene-therapy vector. For example, the gene-therapy vector may be capable of delivering the target gene into the target cell. For example, the target gene may be released in the target cell. For example, the target gene may be integrated into the nucleus. For example, the gene-therapy vector may exert the therapeutic function of the target gene. For example, the gene-therapy vector may be a plasmid vector, a phage vector, a viral vector or a non-viral vector thereof. For example, the gene-therapy vector may be a cloning vector or an expression vector thereof. For example, the gene-therapy vector may be a temperature sensitive vector, a fusion expression vector or a non-fusion expression vector thereof.

For example, the drug may be associated to the transferrin-binding protein. For example, the dug may be associated to the transferrin-binding protein through genetic fusion. For example, the drug may be associated to the transferrin-binding protein through chemical conjugation.

For example, the method may be used for extending half-life in circulation of a therapeutic entity. For example, the therapeutic entity may be linked with the transferrin-binding protein directly or indirectly. For example, the method may be used for delivering a therapeutic drug to transferrin receptor-expressing cells or organs, comprising or using a transferrin-binding entity. For example, the method may be used for delivering a targeted drug to cross blood-brain barrier, intestinal epithelium and/or cell membranes that express transferrin receptor, comprising using a transferrin-binding entity. For example, the method may be used for oral delivery of a therapeutic or diagnostic entity, the therapeutic or diagnostic entity is linked with the antigen binding protein. For example, the method may be used for intracellular delivery of a therapeutic entity, the therapeutic entity is linked with the antigen binding protein directly or indirectly.

### Proteins Capable of Binding Properdin

In one aspect, the present application provides an isolated antigen binding protein, which may specifically bind to properdin in an ELISA binding assay with an isolated antigen binding protein concentration of about 100 ng/ml or less (e.g., said concentration is no greater than about 50 ng/ml, no greater than about 55 ng/ml, no greater than about 60 ng/ml, no greater than about 65 ng/ml, no greater than about 70 ng/ml, no greater than about 75 ng/ml, no greater than about 80 ng/ml, no greater than about 85 ng/ml, no greater than about 90 ng/ml, or no greater than about 95 ng/ml or less). For example, said properdin may comprise human properdin, cyno properdin, and mouse properdin.

In the present application, said isolated antigen binding protein may inhibit alternative pathway by binding protein to induce hemolysis. For example, percent of hemolysis in alternative pathway experiments can be determined by co-incubation of complement-preserved serum and erythrocytes. For example, said complement-preserved serum may derived from human or mouse. For example, said percent of hemolysis may be about 60% or less (e.g., said percent of hemolysis is no greater than about 55%, no greater than about 50%, no greater than about 45%, no greater than about 40%, no greater than about 35%, no greater than about 30%, no greater than about 25%, no greater than about 20%, no greater than about 15%, no greater than about 10%, or no greater than about 5% or less) at the isolated antigen binding protein concentration of 500 nM.

In the present application, said isolated antigen binding protein may specifically bind to TSR5, TSR6, and/or TSR0 domain of properdin. For example, the binding epitopes can be determined by combination between truncated variants of human properdin-biotin and thrombospondin repeats (TSRs).

In the present application, said isolated antigen binding protein may inhibit interaction between properdin and C3. For example, the inhibition activity of said isolated antigen binding protein can be determined by competitive binding assay. The isolated binding protein can competitively bind to properdin, so that inhibit the combination of properdin and C3. For example, said isolated antigen binding protein shows inhibition activity with properdin binding to C3 in dose-dependent manner.

In the present application, said isolated antigen binding protein may selectively inhibit alternative pathway rather than classical pathway or lectin pathway. For example, the pathway selectivity can be determined by percent of hemolysis. For example, said isolated antigen binding protein can inhibit alternative pathway with IC50 of about 50 nM or less (e.g., said IC50 is no greater than about 45 nM, no greater than about 40 nM, no greater than about 35 nM, no greater than about 30 nM, no greater than about 25 nM, no greater than about 20 nM, no greater than about 15 nM, no greater than about 10 nM, or no greater than about 5 nM or less). For example, said isolated antigen binding protein exhibits no inhibitory activity in the classical pathway, while control shows inhibitory ability, with IC50 of 57 nM. For example, said isolated antigen binding protein exhibits no inhibitory activity in the lectin pathway, while control shows inhibitory ability, with IC50 of 45 nM.

In the present application, said isolated antigen binding protein may have species-crossing properdin-binding and complement-inhibitory activity in AP-specific pathways in mammal. For example, the species-crossing complement inhibitory activity can be determined by detecting hemolysis in different species. For example, said species can be human, cyno, mouse and rat.

In one aspect, the present application provides an isolated antigen binding protein, which may comprise at least one CDR in a heavy-chain variable region VH. The VH may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 240, and SEQ ID NO: 241.

In the present application, the CDR of the isolated antigen binding protein may be divided in any form, and any form of divided CDR may fall within the scope of the present application, as long as the VH is identical to an amino acid sequence shown in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 240, and SEQ ID NO: 241.

The CDRs of an antibody, also known as complementarity determining regions, are part of the variable region. The amino acid residues of this region may be in contact with an antigen or an antigenic epitope. The CDRs can be determined by a variety of coding systems, such as CCG, Kabat, Chothia, IMGT, AbM, consensus Kabat/Chothia, and the like. These coding systems are known in the art and the person skilled in the art can determine the CDR regions using different coding systems depending on the sequence and structure of the antibody. Using different coding systems, the CDR regions may differ. In the present application, the CDR encompasses CDR sequences divided according to any CDR division manner; and variants thereof are also contemplated. The said variants comprise an amino acid sequence of the CDR substituted, deleted and/or added with one or more amino acids (e.g., 1-30, 1-20 or 1-10; further e.g., 1, 2, 3, 4, 5, 6, 7, 8 or 9 amino acid substitutions, deletions and/or insertions). Homologs are also encompassed, comprising an amino acid sequence having at least about 85% (e.g., having at least about 85%, about 90%, about 91%, about 92%, about 93%, about 94%, about 95%, about 96%, about 97%, about 98%, about 99% or more) sequence homology to an amino acid sequence of the CDR. In some embodiments, the isolated antigen binding protein described herein is defined by the Kabat coding system.

In the present application, said isolated antigen binding protein may bind to properdin. For example, human properdin, cyno properdin, mouse properdin, rat properdin and the like.

In the present application, said isolated antigen binding protein may comprise a heavy chain variable region VH, which may comprise at least one, two or three of CDR3, CDR2 and CDR1.

In the present application, said CDR3 of said isolated antigen binding protein may comprise an amino acid sequence as set forth in any one of SEQ ID NO: 160, SEQ ID NO: 161, SEQ ID NO: 162, SEQ ID NO: 163, SEQ ID NO: 164, and SEQ ID NO: 165. For example, the CDR3 sequence of said isolated antigen binding protein may be defined according to the Kabat coding system.

In the present application, said CDR2 of said isolated antigen binding protein may comprise an amino acid sequence as set forth in SEQ ID NO: 203: X₁X₂X₃X₄X₅X₆X₇X₈X₉YX₁₁DSVKG, in which X₁ is F or I or absent, X₂ is D or I, X₃ is D or N or R or T, X₄ is G or R or S or T, X₅ is D or E, X₆ is G or R, X₇ is G or R or S or V or W, X₈ is E or K or T, X₉ is R or S or W or Y, and X₁₁ is A or T. For example, the CDR2 sequence of said isolated antigen binding protein may be defined according to the Kabat coding system.

In the present application, said CDR2 of said isolated antigen binding protein may comprise an amino acid sequence as set forth in any one of SEQ ID NO: 154, SEQ ID NO: 155, SEQ ID NO: 156, SEQ ID NO: 157, SEQ ID NO: 158, and SEQ ID NO: 159.

In the present application, said CDR1 of said isolated antigen binding protein may comprise an amino acid sequence as set forth SEQ ID NO: 202: X₁X₂CMX₅, in which X₁ is H or S or T or Y, X₂ is G or Y, and X₅ is A or G. For example, the CDR1 sequence of said isolated antigen binding protein may be defined according to the Kabat coding system.

In the present application, said CDR1 of said isolated antigen binding protein may comprise an amino acid sequence as set forth in any one of SEQ ID NO: 149, SEQ ID NO: 150, SEQ ID NO: 151, SEQ ID NO: 152, and SEQ ID NO: 153.

For example, said CDR3 of said isolated antigen binding protein may comprise an amino acid sequence as set forth in any one of SEQ ID NO: 160, SEQ ID NO: 161, SEQ ID NO: 162, SEQ ID NO: 163, SEQ ID NO: 164, and SEQ ID NO: 165, said CDR2 of said isolated antigen binding protein may comprise an amino acid sequence as set forth in any one of SEQ ID NO: 154, SEQ ID NO: 155, SEQ ID NO: 156, SEQ ID NO: 157, SEQ ID NO: 158, and SEQ ID NO: 159, and said CDR1 of said isolated antigen binding protein may comprise an amino acid sequence as set forth in any one of SEQ ID NO: 149, SEQ ID NO: 150, SEQ ID NO: 151, SEQ ID NO: 152, and SEQ ID NO: 153.

For example, said CDR3 of said isolated antigen binding protein may comprise an amino acid sequence as set forth in SEQ ID NO: 160, said CDR2 of said isolated antigen binding protein may comprise an amino acid sequence as set forth in SEQ ID NO: 154, and said CDR1 of said isolated antigen binding protein may comprise an amino acid sequence as set forth in SEQ ID NO: 149. For example, said CDR3 of said isolated antigen binding protein may comprise an amino acid sequence as set forth in SEQ ID NO: 161, said CDR2 of said isolated antigen binding protein may comprise an amino acid sequence as set forth in SEQ ID NO: 155, and said CDR1 of said isolated antigen binding protein may comprise an amino acid sequence as set forth in SEQ ID NO: 150. For example, said CDR3 of said isolated antigen binding protein may comprise an amino acid sequence as set forth in SEQ ID NO: 162, said CDR2 of said isolated antigen binding protein may comprise an amino acid sequence as set forth in SEQ ID NO: 156, and said CDR1 of said isolated antigen binding protein may comprise an amino acid sequence as set forth in SEQ ID NO: 151. For example, said CDR3 of said isolated antigen binding protein may comprise an amino acid sequence as set forth in SEQ ID NO: 163, said CDR2 of said isolated antigen binding protein may comprise an amino acid sequence as set forth in SEQ ID NO: 157, and said CDR1 of said isolated antigen binding protein may comprise an amino acid sequence as set forth in SEQ ID NO: 152. For example, said CDR3 of said isolated antigen binding protein may comprise an amino acid sequence as set forth in SEQ ID NO: 164, said CDR2 of said isolated antigen binding protein may comprise an amino acid sequence as set forth in SEQ ID NO: 158, and said CDR1 of said isolated antigen binding protein may comprise an amino acid sequence as set forth in SEQ ID NO: 153. For example, said CDR3 of said isolated antigen binding protein may comprise an amino acid sequence as set forth in SEQ ID NO: 165, said CDR2 of said isolated antigen binding protein may comprise an amino acid sequence as set forth in SEQ ID NO: 159, and said CDR1 of said isolated antigen binding protein may comprise an amino acid sequence as set forth in SEQ ID NO: 151. For example, said isolated antigen binding protein may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 240, and SEQ ID NO: 241, or an antibody having the same CDR (e.g., CDR1, CDR2 or CDR3).

In the present application, said isolated antigen binding protein may further comprise framework regions FR1, FR2, FR3, and FR4.

In the present application, said FR1 of said isolated antigen binding protein may comprise an amino acid sequence as set forth SEQ ID NO: 204: X₁VQLVESGGGX₁₁V X₁₃X₁₄GGSLRLSCX₂₃X₂₄X₂₅X₂₆YX₂₈X₂₉X₃₀, in which X₁ is D or E or H or Q, X₁ is L or S or V, X₁₃ is H or Q, X₁₄ is A or P or S or V, X₂₃ is A or E or V, X₂₄ is A or D or H or V, X₂₅ is F or P or S, X₂₆ is A or E or G, X₂₈ is I or T or absent, X₂₉ is H or S or Y or absent, and X₃₀ is G or S or T or absent. For example, said FR1 sequence of said isolated antigen binding protein may be defined according to the Kabat coding system.

In the present application, said FR1 of said isolated antigen binding protein may comprise an amino acid sequence as set forth in any one of SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, and SEQ ID NO: 178.

In the present application, said FR2 of said isolated antigen binding protein may comprise an amino acid sequence as set forth SEQ ID NO: 205: WX₂RQAPG X₈X₉X₁₀EX₁₂VX₁₄X₁₅, in which X₂ is F or I, X₈ is E or K, X₉ is E or G, X₁₀ is L or R, X₁₂ is G or R, X₁₄ is A or S, and X₁₅ is A or S or V. For example, said FR2 sequence of said isolated antigen binding protein may be defined according to the Kabat coding system.

In the present application, said FR2 of said isolated antigen binding protein may comprise an amino acid sequence as set forth in any one of SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, and SEQ ID NO: 188.

In the present application, said FR3 of said isolated antigen binding protein may comprise an amino acid sequence as set forth SEQ ID NO: 206: RFTISX₆DX₈X₉X₁₀X₁₁TLYLX₁₆MNX₁₉LX₂₁X₂₂EDTAX₂₇YYCAX₃₂, in which X₆ is K or L or Q or R, X₈ is I or N, X₉ is A or S, X₁₀ is E or K or T, X₁₁ is N or S, X₁ is E or Q, X₁₉ is I or N or S, X₂₁ is K or Q or R, X₂₂ is A or P or S, X₂₇ is M or V, and X₃₂ is A or T. For example, the FR3 sequence of said isolated antigen binding protein may be defined according to the Kabat coding system.

In the present application, said FR3 of said isolated antigen binding protein may comprise an amino acid sequence as set forth in any one of SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, and SEQ ID NO: 199.

In the present application, said FR4 of said isolated antigen binding protein may comprise an amino acid sequence as set forth SEQ ID NO: 207: WGQGTX₆VTVSS, in which X₆ is L or Q. For example, said FR4 sequence of said isolated antigen binding protein may be defined according to the Kabat coding system.

In the present application, said FR4 of said isolated antigen binding protein may comprise an amino acid sequence as set forth in any one of SEQ ID NO: 200 and SEQ ID NO: 201.

For example, said FR1 of said isolated antigen binding protein may comprise an amino acid sequence as set forth in any one of SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, and SEQ ID NO: 178; said FR2 of said isolated antigen binding protein may comprise an amino acid sequence as set forth in any one of SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, and SEQ ID NO: 188; said FR3 of said isolated antigen binding protein may comprise an amino acid sequence as set forth in any one of SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, and SEQ ID NO: 199, and said FR4 of said isolated antigen binding protein may comprise an amino acid sequence as set forth in any one of SEQ ID NO: 200 and SEQ ID NO: 201.

For example, said FR1 of said isolated antigen binding protein may comprise an amino acid sequence as set forth in SEQ ID NO: 166; said FR2 of said isolated antigen binding protein may comprise an amino acid sequence as set forth in SEQ ID NO: 179; said FR3 of said isolated antigen binding protein may comprise an amino acid sequence as set forth in SEQ ID NO: 189, and said FR4 of said isolated antigen binding protein may comprise an amino acid sequence as set forth in SEQ ID NO: 200. For example, said isolated antigen binding protein may comprise an amino acid sequence as set forth in SEQ ID NO: 208, or an antibody having the same FR (e.g., FR1, FR2, FR3, or FR4).

For example, said FR1 of said isolated antigen binding protein may comprise an amino acid sequence as set forth in SEQ ID NO: 167; said FR2 of said isolated antigen binding protein may comprise an amino acid sequence as set forth in SEQ ID NO: 180; said FR3 of said isolated antigen binding protein may comprise an amino acid sequence as set forth in SEQ ID NO: 190, and said FR4 of said isolated antigen binding protein may comprise an amino acid sequence as set forth in SEQ ID NO: 201. For example, said isolated antigen binding protein may comprise an amino acid sequence as set forth in SEQ ID NO: 209, or an antibody having the same FR (e.g., FR1, FR2, FR3, or FR4).

For example, said FR1 of said isolated antigen binding protein may comprise an amino acid sequence as set forth in SEQ ID NO: 167; said FR2 of said isolated antigen binding protein may comprise an amino acid sequence as set forth in SEQ ID NO: 180; said FR3 of said isolated antigen binding protein may comprise an amino acid sequence as set forth in SEQ ID NO: 190, and said FR4 of said isolated antigen binding protein may comprise an amino acid sequence as set forth in SEQ ID NO: 201. For example, said isolated antigen binding protein may comprise an amino acid sequence as set forth in SEQ ID NO: 210, or an antibody having the same FR (e.g., FR1, FR2, FR3, or FR4).

For example, said FR1 of said isolated antigen binding protein may comprise an amino acid sequence as set forth in SEQ ID NO: 167; said FR2 of said isolated antigen binding protein may comprise an amino acid sequence as set forth in SEQ ID NO: 180; said FR3 of said isolated antigen binding protein may comprise an amino acid sequence as set forth in SEQ ID NO: 191, and said FR4 of said isolated antigen binding protein may comprise an amino acid sequence as set forth in SEQ ID NO: 201. For example, said isolated antigen binding protein may comprise an amino acid sequence as set forth in SEQ ID NO: 211, or an antibody having the same FR (e.g., FR1, FR2, FR3, or FR4).

For example, said FR1 of said isolated antigen binding protein may comprise an amino acid sequence as set forth in SEQ ID NO: 168; said FR2 of said isolated antigen binding protein may comprise an amino acid sequence as set forth in SEQ ID NO: 180; said FR3 of said isolated antigen binding protein may comprise an amino acid sequence as set forth in SEQ ID NO: 191, and said FR4 of said isolated antigen binding protein may comprise an amino acid sequence as set forth in SEQ ID NO: 201. For example, said isolated antigen binding protein may comprise an amino acid sequence as set forth in SEQ ID NO: 212, or an antibody having the same FR (e.g., FR1, FR2, FR3, or FR4).

For example, said FR1 of said isolated antigen binding protein may comprise an amino acid sequence as set forth in SEQ ID NO: 168; said FR2 of said isolated antigen binding protein may comprise an amino acid sequence as set forth in SEQ ID NO: 179; said FR3 of said isolated antigen binding protein may comprise an amino acid sequence as set forth in SEQ ID NO: 191, and said FR4 of said isolated antigen binding protein may comprise an amino acid sequence as set forth in SEQ ID NO: 201. For example, said isolated antigen binding protein may comprise an amino acid sequence as set forth in SEQ ID NO: 203 or an antibody having the same FR (e.g., FR1, FR2, FR3, or FR4).

For example, said FR1 of said isolated antigen binding protein may comprise an amino acid sequence as set forth in SEQ ID NO: 169; said FR2 of said isolated antigen binding protein may comprise an amino acid sequence as set forth in SEQ ID NO: 181; said FR3 of said isolated antigen binding protein may comprise an amino acid sequence as set forth in SEQ ID NO: 192, and said FR4 of said isolated antigen binding protein may comprise an amino acid sequence as set forth in SEQ ID NO: 200. For example, said isolated antigen binding protein may comprise an amino acid sequence as set forth in SEQ ID NO: 214, or an antibody having the same FR (e.g., FR1, FR2, FR3, or FR4).

For example, said FR1 of said isolated antigen binding protein may comprise an amino acid sequence as set forth in SEQ ID NO: 170; said FR2 of said isolated antigen binding protein may comprise an amino acid sequence as set forth in SEQ ID NO: 182; said FR3 of said isolated antigen binding protein may comprise an amino acid sequence as set forth in SEQ ID NO: 190, and said FR4 of said isolated antigen binding protein may comprise an amino acid sequence as set forth in SEQ ID NO: 201. For example, said isolated antigen binding protein may comprise an amino acid sequence as set forth in SEQ ID NO: 215, or an antibody having the same FR (e.g., FR1, FR2, FR3, or FR4).

For example, said FR1 of said isolated antigen binding protein may comprise an amino acid sequence as set forth in SEQ ID NO: 170; said FR2 of said isolated antigen binding protein may comprise an amino acid sequence as set forth in SEQ ID NO: 182; said FR3 of said isolated antigen binding protein may comprise an amino acid sequence as set forth in SEQ ID NO: 193, and said FR4 of said isolated antigen binding protein may comprise an amino acid sequence as set forth in SEQ ID NO: 201. For example, said isolated antigen binding protein may comprise an amino acid sequence as set forth in SEQ ID NO: 216, or an antibody having the same FR (e.g., FR1, FR2, FR3, or FR4).

For example, said FR1 of said isolated antigen binding protein may comprise an amino acid sequence as set forth in SEQ ID NO: 170; said FR2 of said isolated antigen binding protein may comprise an amino acid sequence as set forth in SEQ ID NO: 181; said FR3 of said isolated antigen binding protein may comprise an amino acid sequence as set forth in SEQ ID NO: 193, and said FR4 of said isolated antigen binding protein may comprise an amino acid sequence as set forth in SEQ ID NO: 201. For example, said isolated antigen binding protein may comprise an amino acid sequence as set forth in SEQ ID NO: 217, or an antibody having the same FR (e.g., FR1, FR2, FR3, or FR4).

For example, said FR1 of said isolated antigen binding protein may comprise an amino acid sequence as set forth in SEQ ID NO: 171; said FR2 of said isolated antigen binding protein may comprise an amino acid sequence as set forth in SEQ ID NO: 183; said FR3 of said isolated antigen binding protein may comprise an amino acid sequence as set forth in SEQ ID NO: 194, and said FR4 of said isolated antigen binding protein may comprise an amino acid sequence as set forth in SEQ ID NO: 200. For example, said isolated antigen binding protein may comprise an amino acid sequence as set forth in SEQ ID NO: 218, or an antibody having the same FR (e.g., FR1, FR2, FR3, or FR4).

For example, said FR1 of said isolated antigen binding protein may comprise an amino acid sequence as set forth in SEQ ID NO: 172; said FR2 of said isolated antigen binding protein may comprise an amino acid sequence as set forth in SEQ ID NO: 184; said FR3 of said isolated antigen binding protein may comprise an amino acid sequence as set forth in SEQ ID NO: 195, and said FR4 of said isolated antigen binding protein may comprise an amino acid sequence as set forth in SEQ ID NO: 201. For example, said isolated antigen binding protein may comprise an amino acid sequence as set forth in SEQ ID NO: 219, or an antibody having the same FR (e.g., FR1, FR2, FR3, or FR4).

For example, said FR1 of said isolated antigen binding protein may comprise an amino acid sequence as set forth in SEQ ID NO: 172; said FR2 of said isolated antigen binding protein may comprise an amino acid sequence as set forth in SEQ ID NO: 184; said FR3 of said isolated antigen binding protein may comprise an amino acid sequence as set forth in SEQ ID NO: 196, and said FR4 of said isolated antigen binding protein may comprise an amino acid sequence as set forth in SEQ ID NO: 201. For example, said isolated antigen binding protein may comprise an amino acid sequence as set forth in SEQ ID NO: 220, or an antibody having the same FR (e.g., FR1, FR2, FR3, or FR4).

For example, said FR1 of said isolated antigen binding protein may comprise an amino acid sequence as set forth in SEQ ID NO: 173; said FR2 of said isolated antigen binding protein may comprise an amino acid sequence as set forth in SEQ ID NO: 184; said FR3 of said isolated antigen binding protein may comprise an amino acid sequence as set forth in SEQ ID NO: 196, and said FR4 of said isolated antigen binding protein may comprise an amino acid sequence as set forth in SEQ ID NO: 201. For example, said isolated antigen binding protein may comprise an amino acid sequence as set forth in SEQ ID NO: 221, or an antibody having the same FR (e.g., FR1, FR2, FR3, or FR4).

For example, said FR1 of said isolated antigen binding protein may comprise an amino acid sequence as set forth in SEQ ID NO: 173; said FR2 of said isolated antigen binding protein may comprise an amino acid sequence as set forth in SEQ ID NO: 183; said FR3 of said isolated antigen binding protein may comprise an amino acid sequence as set forth in SEQ ID NO: 196, and said FR4 of said isolated antigen binding protein may comprise an amino acid sequence as set forth in SEQ ID NO: 201. For example, said isolated antigen binding protein may comprise an amino acid sequence as set forth in SEQ ID NO: 222, or an antibody having the same FR (e.g., FR1, FR2, FR3, or FR4).

For example, said FR1 of said isolated antigen binding protein may comprise an amino acid sequence as set forth in SEQ ID NO: 174; said FR2 of said isolated antigen binding protein may comprise an amino acid sequence as set forth in SEQ ID NO: 185; said FR3 of said isolated antigen binding protein may comprise an amino acid sequence as set forth in SEQ ID NO: 197, and said FR4 of said isolated antigen binding protein may comprise an amino acid sequence as set forth in SEQ ID NO: 200. For example, said isolated antigen binding protein may comprise an amino acid sequence as set forth in SEQ ID NO: 223, or an antibody having the same FR (e.g., FR1, FR2, FR3, or FR4).

For example, said FR1 of said isolated antigen binding protein may comprise an amino acid sequence as set forth in SEQ ID NO: 175; said FR2 of said isolated antigen binding protein may comprise an amino acid sequence as set forth in SEQ ID NO: 186; said FR3 of said isolated antigen binding protein may comprise an amino acid sequence as set forth in SEQ ID NO: 190, and said FR4 of said isolated antigen binding protein may comprise an amino acid sequence as set forth in SEQ ID NO: 201. For example, said isolated antigen binding protein may comprise an amino acid sequence as set forth in SEQ ID NO: 224, or an antibody having the same FR (e.g., FR1, FR2, FR3, or FR4).

For example, said FR1 of said isolated antigen binding protein may comprise an amino acid sequence as set forth in SEQ ID NO: 175; said FR2 of said isolated antigen binding protein may comprise an amino acid sequence as set forth in SEQ ID NO: 187; said FR3 of said isolated antigen binding protein may comprise an amino acid sequence as set forth in SEQ ID NO: 190, and said FR4 of said isolated antigen binding protein may comprise an amino acid sequence as set forth in SEQ ID NO: 201. For example, said isolated antigen binding protein may comprise an amino acid sequence as set forth in SEQ ID NO: 225, or an antibody having the same FR (e.g., FR1, FR2, FR3, or FR4).

For example, said FR1 of said isolated antigen binding protein may comprise an amino acid sequence as set forth in SEQ ID NO: 170; said FR2 of said isolated antigen binding protein may comprise an amino acid sequence as set forth in SEQ ID NO: 187; said FR3 of said isolated antigen binding protein may comprise an amino acid sequence as set forth in SEQ ID NO: 190, and said FR4 of said isolated antigen binding protein may comprise an amino acid sequence as set forth in SEQ ID NO: 201. For example, said isolated antigen binding protein may comprise an amino acid sequence as set forth in SEQ ID NO: 226, or an antibody having the same FR (e.g., FR1, FR2, FR3, or FR4).

For example, said FR1 of said isolated antigen binding protein may comprise an amino acid sequence as set forth in SEQ ID NO: 170; said FR2 of said isolated antigen binding protein may comprise an amino acid sequence as set forth in SEQ ID NO: 187; said FR3 of said isolated antigen binding protein may comprise an amino acid sequence as set forth in SEQ ID NO: 193, and said FR4 of said isolated antigen binding protein may comprise an amino acid sequence as set forth in SEQ ID NO: 201. For example, said isolated antigen binding protein may comprise an amino acid sequence as set forth in SEQ ID NO: 227, or an antibody having the same FR (e.g., FR1, FR2, FR3, or FR4).

For example, said FR1 of said isolated antigen binding protein may comprise an amino acid sequence as set forth in SEQ ID NO: 176; said FR2 of said isolated antigen binding protein may comprise an amino acid sequence as set forth in SEQ ID NO: 185; said FR3 of said isolated antigen binding protein may comprise an amino acid sequence as set forth in SEQ ID NO: 191, and said FR4 of said isolated antigen binding protein may comprise an amino acid sequence as set forth in SEQ ID NO: 201. For example, said isolated antigen binding protein may comprise an amino acid sequence as set forth in SEQ ID NO: 228, or an antibody having the same FR (e.g., FR1, FR2, FR3, or FR4).

For example, said FR1 of said isolated antigen binding protein may comprise an amino acid sequence as set forth in SEQ ID NO: 177; said FR2 of said isolated antigen binding protein may comprise an amino acid sequence as set forth in SEQ ID NO: 179; said FR3 of said isolated antigen binding protein may comprise an amino acid sequence as set forth in SEQ ID NO: 198, and said FR4 of said isolated antigen binding protein may comprise an amino acid sequence as set forth in SEQ ID NO: 200. For example, said isolated antigen binding protein may comprise an amino acid sequence as set forth in SEQ ID NO: 240, or an antibody having the same FR (e.g., FR1, FR2, FR3, or FR4).

For example, said FR1 of said isolated antigen binding protein may comprise an amino acid sequence as set forth in SEQ ID NO: 178; said FR2 of said isolated antigen binding protein may comprise an amino acid sequence as set forth in SEQ ID NO: 188; said FR3 of said isolated antigen binding protein may comprise an amino acid sequence as set forth in SEQ ID NO: 199, and said FR4 of said isolated antigen binding protein may comprise an amino acid sequence as set forth in SEQ ID NO: 200. For example, said isolated antigen binding protein may comprise an amino acid sequence as set forth in SEQ ID NO: 241, or an antibody having the same FR (e.g., FR1, FR2, FR3, or FR4).

In the present application, said heavy-chain variable region may comprise VHH. The VHH may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 240, and SEQ ID NO: 241.

In the present application, said isolated antigen binding protein may comprise a heavy-chain constant region.

For example, the Fc region of said isolated antigen binding protein may be a human Fc region. For example, said human Fc region may be modified to achieve the desired property (e.g., an amino acid mutation). For example, said human Fc region may comprise an amino acid sequence as set forth in SEQ ID NO: 257.

In the present application, said isolated antigen binding protein may be directly or indirectly linked to a second antigen binding domain.

For example, said isolated antigen binding protein may be linked by its N-terminus or C-terminus to the N-terminus or C-terminus of said second antigen binding domain. For example, said isolated antigen binding protein may be linked by its N-terminus or C-terminus to the N-terminus or C-terminus of said second antigen binding domain with a linker. For example, said linker of said isolated antigen binding protein may be a simple covalent bond (e.g., a peptide bond), a synthetic polymer (e.g., a polyethylene glycol (PEG) polymer), or any kind of bond created from a chemical reaction. For example, said linker of said isolated antigen binding protein may be a poly-glycine linker. For example, said linker of said isolated antigen binding protein may comprises an amino acid sequence as set forth in SEQ ID NO: 256: GGGGSGGGGSGGGGS.

In the present application, said second antigen binding domain of said isolated antigen binding protein may bind to a different target from said isolated antigen binding protein.

In the present application, said second antigen binding domain of said isolated antigen binding protein may bind to the same target as said isolated antigen binding protein.

For example, said second antigen binding domain of said isolated antigen binding protein may bind to properdin. For example, said second antigen binding domain of said isolated antigen binding protein may bind to different epitopes of properdin from the isolated antigen binding protein. For example, said second antigen binding domain of said isolated antigen binding protein may bind to the same epitopes of properdin with the isolated antigen binding protein. For example, said second antigen binding domain of said isolated antigen binding protein may bind to TSR5, TSR6, and/or TSR0 domain of properdin. For example, said second antigen binding domain of said isolated antigen binding protein may comprises an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 240, and SEQ ID NO: 241.

For example, said isolated antigen binding protein may comprise an amino acid sequence as set forth in SEQ ID NO: 229.

For example, said isolated antigen binding protein may comprise an amino acid sequence as set forth in SEQ ID NO: 264.

For example, said isolated antigen binding protein may comprise an antibody or an antigen binding fragment thereof. For example, said isolated antigen binding protein may comprise Fab, Fab′, F(ab)₂, Fv fragments, F(ab′)₂, scFv, di-scFv, VHH and/or dAb. For example, said isolated antigen binding protein may be selected from the group consisting of: monoclonal antibodies, single chain antibodies, chimeric antibodies, humanized antibodies, and fully human antibodies.

For example, said isolated antigen binding protein may be a camelid antibody.

In the present application, said isolated antigen binding protein may have a competitive target binding capability with reference antibodies, wherein said reference antibodies may comprise a heavy chain variable region VH, which may comprise at least one, two or three of CDR3, CDR2 and CDR1.

In the present application, the CDR3 of said reference antibodies may comprise an amino acid sequence as set forth in any one of SEQ ID NO: 260, SEQ ID NO: 261, and SEQ ID NO: 262. For example, the CDR3 sequence of said reference antibodies may be defined according to the Kabat coding system.

In the present application, the CDR2 of said reference antibodies may comprise an amino acid sequence as set forth in SEQ ID NO: 203: X₁X₂X₃X₄X₅X₆X₇X₈X₉YX₁₁DSVKG, in which X₁ is F or I or absent, X₂ is D or I, X₃ is D or N or R or T, X₄ is G or R or S or T, X₅ is D or E, X₆ is G or R, X₇ is G or R or S or V or W, X₈ is E or K or T, X₉ is R or S or W or Y, and X_{II} is A or T. For example, the CDR2 sequence of said reference antibodies may be defined according to the Kabat coding system.

In the present application, the CDR2 of said reference antibodies may comprise an amino acid sequence as set forth in any one of SEQ ID NO: 154, SEQ ID NO: 155, and SEQ ID NO: 156.

In the present application, the CDR1 of said reference antibodies may comprise an amino acid sequence as set forth SEQ ID NO: 202: X₁X₂CMX₅, in which X₁ is H or S or T or Y, X₂ is G or Y, and X₅ is A or G. For example, the CDR1 sequence of said reference antibodies may be defined according to the Kabat coding system.

In the present application, the CDR1 of said reference antibodies may comprise an amino acid sequence as set forth in any one of SEQ ID NO: 149, SEQ ID NO: 150, and SEQ ID NO: 151.

For example, the CDR3 of said reference antibodies may comprise an amino acid sequence as set forth in any one of SEQ ID NO: 160, SEQ ID NO: 161, and SEQ ID NO: 162, the CDR2 of said reference antibodies may comprise an amino acid sequence as set forth in any one of SEQ ID NO: 154, SEQ ID NO: 155, and SEQ ID NO: 156, and the CDR1 of said reference antibodies may comprise an amino acid sequence as set forth in any one of SEQ ID NO: 149, SEQ ID NO: 150, and SEQ ID NO: 151.

For example, the CDR3 of said reference antibodies may comprise an amino acid sequence as set forth in SEQ ID NO: 160, the CDR2 of said reference antibodies may comprise an amino acid sequence as set forth in SEQ ID NO: 154, and the CDR1 of said reference antibodies may comprise an amino acid sequence as set forth in SEQ ID NO: 149. For example, the CDR3 of said reference antibodies may comprise an amino acid sequence as set forth in SEQ ID NO: 161, the CDR2 of said reference antibodies may comprise an amino acid sequence as set forth in SEQ ID NO: 155, and the CDR1 of said reference antibodies may comprise an amino acid sequence as set forth in SEQ ID NO: 150. For example, the CDR3 of said reference antibodies may comprise an amino acid sequence as set forth in SEQ ID NO: 162, the CDR2 of said reference antibodies may comprise an amino acid sequence as set forth in SEQ ID NO: 156, and the CDR1 of said reference antibodies may comprise an amino acid sequence as set forth in SEQ ID NO: 151. For example, said isolated antigen binding protein may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, and SEQ ID NO: 222, or an antibody having the same CDR (e.g., CDR1, CDR2 or CDR3).

For example, the isolated antigen binding protein may comprise its functional active fragment, ortholog, and variant, which keep the similar biologic activity. For example, the sequence similarity may have at least 30%, at least 35%, at least 40%, at least 45%, at least 50%, at least 55%, at least 60%, at least 65%, at least 70%, at least 75%, at least 80%, at least 85%, at least 90%, at least 95% or at least 99% similarity to its corresponding sequence.

### Fusion Protein

In another aspect, the present application provides a fusion protein that may comprise the isolated antigen binding protein of the present application.

In the present application, said fusion protein may comprise a functionally active protein.

In the present application, said functionally active protein of said fusion protein may be directly or indirectly linked to said isolated antigen binding protein.

For example, said functionally active protein may be linked by its N-terminus or C-terminus to the N-terminus or C-terminus of said isolated antigen binding protein. For example, said functionally active protein may be linked by its N-terminus or C-terminus to the N-terminus or C-terminus of said isolated antigen binding protein with a linker. For example, said linker may be a simple covalent bond (e.g., a peptide bond), a synthetic polymer (e.g., a polyethylene glycol (PEG) polymer), or any kind of bond created from a chemical reaction. For example, said linker may be a poly-glycine linker. For example, said linker may comprises an amino acid sequence as set forth in SEQ ID NO: 256: GGGGSGGGGSGGGGS.

For example, said functionally active protein may be factor H. For example, said factor H may comprise an amino acid sequence as set forth in SEQ ID NO: 258. For example, said factor H of said fusion protein may comprise its functional active fragment, ortholog, and variant. For example, the sequence similarity may have at least 30%, at least 35%, at least 40%, at least 45%, at least 50%, at least 55%, at least 60%, at least 65%, at least 70%, at least 75%, at least 80%, at least 85%, at least 90%, at least 95% or at least 99% similarity to its corresponding sequence.

For example, said fusion protein may comprise an amino acid sequence as set forth in SEQ ID NO: 230.

For example, said functionally active protein of said fusion protein may be VEGF inhibiting protein. For example, said VEGF inhibiting protein. of said fusion protein may comprise an amino acid sequence as set forth in SEQ ID NO: 261. For example, said VEGF inhibiting protein of said fusion protein may comprise its functional active fragment, ortholog, and variant. In some embodiments, the sequence similarity may have at least 30%, at least 35%, at least 40%, at least 45%, at least 50%, at least 55%, at least 60%, at least 65%, at least 70%, at least 75%, at least 80%, at least 85%, at least 90%, at least 95% or at least 99% similarity to its corresponding sequence.

For example, said fusion protein may comprise an amino acid sequence as set forth in SEQ ID NO: 262.

For example, said functionally active protein of said fusion protein may be transferrin inhibiting protein. For example, said factor H of said fusion protein may comprise an amino acid sequence as set forth in SEQ ID NO: 263. For example, said transferrin inhibiting protein of said fusion protein may comprise its functional active fragment, ortholog, and variant. In some embodiments, the sequence similarity may have at least 30%, at least 35%, at least 40%, at least 45%, at least 50%, at least 55%, at least 60%, at least 65%, at least 70%, at least 75%, at least 80%, at least 85%, at least 90%, at least 95% or at least 99% similarity to its corresponding sequence.

For example, said fusion protein may comprise an amino acid sequence as set forth in SEQ ID NO: 265. For example, said fusion protein may comprise an amino acid sequence as set forth in SEQ ID NO: 266.

For example, the fusion protein may include a prophylactic or therapeutic drug fused to a heterologous protein, polypeptide, or peptide. Wherein, the heterologous protein, polypeptide or peptide may or may not be different types or therapeutic drugs.

For example, the fusion protein may comprise two or more different proteins, polypeptides or peptides with immunomodulatory activity. For example, the fusion protein may retain or improve the activity compared to the activity of the original polypeptide or protein. Typically, the fusion protein can be produced by in vitro recombinant techniques well known in the art. For example, the fusion protein may comprise the antigen binding protein.

### Polypeptides and Immunoconjugates

In another aspect, the present application provides one or more polypeptides that may comprise the isolated antigen binding protein of the present application.

In another aspect, the present application provides one or more immunoconjugates that may comprise the isolated antigen binding protein of the present application. In certain embodiments, the immunoconjugate may further comprise a pharmaceutically acceptable therapeutic agent.

### Nucleic Acid, Vector and Cell

In another aspect, the present application further provides an isolated nucleic acid molecule or isolated nucleic acid molecules. The nucleic acid molecule(s) may encode the antigen binding protein of the present application. For example, each of the nucleic acid molecule(s) may encode the complete antigen binding protein, or a portion thereof (e.g., one or more of CDR1-3, FR1-4, VH, VHH or heavy chain).

The nucleic acid molecule(s) of the present application may be isolated. For example, it may be produced or synthesized by the following methods: (i) in vitro amplification, for example by polymerase chain reaction (PCR) amplification, (ii) clonal recombination, (iii) purification, for example, by fractionation through restriction digestion and gel electrophoresis, or (iv) synthesis, for example, by chemical synthesis. In some embodiments, the isolated nucleic acid(s) is/are a nucleic acid molecule(s) prepared by the recombinant DNA technology.

In the present application, the nucleic acid(s) encoding the antibody and the antigen-binding fragment thereof may be prepared by a variety of methods known in the art. These methods include, but are not limited to, the overlap extension PCR using restriction fragment operations or using synthetic oligonucleotides. For specific operations, see Sambrook et al., Molecular Cloning, A Laboratory Manual, Cold Spring Harbor Laboratory Press, Cold Spring Harbor, N.Y., 1989; and Ausube et al., Current Protocols in Molecular Biology, Greene Publishing and Wiley-Interscience, New York N.Y., 1993.

In another aspect, the present application provides a vector or vectors, each of which comprises the nucleic acid molecule(s) of the present application. Each vector may comprise one or more said nucleic acid molecule(s). In addition, the vector may also comprise other genes, for example, a marker gene that is allowed to select this vector in a suitable host cell and under a suitable condition. In addition, the vector may also comprise an expression control element that allows a coding region to be expressed correctly in a suitable host. Such a control element is well known to those skilled in the art, which, for example, may include a promoter, a ribosome binding site, an enhancer, and other control elements that regulate gene transcription or mRNA translation. In some embodiments, the expression control sequence is a regulatable element. A specific structure of the expression control sequence may vary depending on the function of the species or cell type, but generally includes a 5′ non-transcribed sequence and 5′ and 3′ non-translated sequences, for example, a TATA box, a capped sequence, a CAAT sequence, etc., which are involved in transcription and translation initiation, respectively. For example, the 5′ non-transcribed expression control sequence may include a promoter region, and the promoter region may include a promoter sequence for functionally linked to the nucleic acid for transcriptional control. The expression control sequence may further comprise an enhancer sequence or an upstream activator sequence. In the present application, suitable promoters may comprise, for example, promoters for SP6, T3, and T7 polymerases, human U6 RNA promoters, CMV promoters, and their artificial hybrid promoters (such as CMV), wherein a portion of a promoter may be fused with a portion of a promoter of an additional cellular protein (such as human GAPDH and glyceraldehyde-3-phosphate dehydrogenase) gene, and the promoter may or may not contain additional introns. The nucleic acid molecule(s) of the present application may be operably linked to the expression control element. The vector may comprise, for example, a plasmid, a cosmid, a virus, a bacteriophage, or other vectors commonly used in, for example, genetic engineering. For example, the vector is an expression vector.

In another aspect, the present application provides a host cell, which may comprise the nucleic acid molecule(s) of the present application and/or the vector or vectors of the present application. In some embodiments, each type of or each host cell may comprise one or one type of the nucleic acid molecule or vector of the present application. In some embodiments, each type of or each cell may comprise a plurality of (e.g., 2 or more) or a plurality of types of (e.g., 2 or more types of) vectors of the present application. For example, the vector of the present application may be introduced into the host cell, for example, a eukaryotic cell, such as a plant-originated cell, a fungal cell, or a yeast cell, etc. The vector of the present application may be introduced into the host cell by methods known in the art, such as electroporation, lipofectine transfection, lipofectamin transfection, etc.

### Preparation Method

In another aspect, the present application provides a preparation method for the isolated antigen binding protein. The method may comprise culturing the host cell of the present application under such a condition that the isolated antigen binding protein is expressed. For example, an appropriate medium, an appropriate temperature, a culture time and the like may be used, and these methods are understood by those of ordinary skills in the art.

Any method suitable for producing a monoclonal antibody may be used to produce the isolated antigen binding protein (e.g., the anti-properdin antibody) of the present application. For example, animals may be immunized with linked or naturally occurring properdin or fragments thereof. Suitable immunization methods may be used, including adjuvants, immunostimulants, and repeated booster immunizations, and one or more routes may be used.

Any suitable form of properdin may be used as an immunogen (antigen) to produce a non-human antibody specific to properdin and to screen the biological activity of the antibody. An eliciting immunogen may be a human properdin, a recombinant mouse, or peptides containing single/multiple epitopes. The immunogen may be used alone, or in combination with one or more immunogenicity enhancers known in the art. The immunogen may be purified from a natural source, or produced in a genetically modified cell. An DNA encoding the immunogen may be genomic or non-genomic (e.g. cDNA) in source. A suitable genetic vector may be used to express the DNA encoding the immunogen, and the vector comprises, but is not limited to, an adenovirus vector, an adeno-associated virus vector, a baculovirus vector, a material, and a non-viral vector.

An exemplary method for discovering the isolated antigen binding protein of the present application is described in Example 3.1.

Immunization may be performed using recombinant mouse properdin in healthy camels. An essential constant domain sequence may be optimized by screening antibodies with the biological assays described in the Examples below, so as to produce the desired biological activity.

An exemplary method for humanizing the isolated antigen binding protein of the present application is described in Example 3.2.

The sequence of the DNA molecule of the isolated antigen binding protein or the fragment thereof in the present application may be obtained by conventional techniques, such as methods using PCR amplification or genomic library screening and the like.

Once relevant sequences are obtained, they may be obtained on a large scale by recombination. This is generally done by cloning them into vectors, then transferring then into cells, and then isolating the relevant sequences from the proliferated host cell by means of a conventional method.

In addition, the relevant sequences may also be synthesized by using an artificial synthesis method, especially when a fragment is short. Generally, a fragment with a very long sequence may be obtained by first synthesizing multiple small fragments, and then linking these small fragments. Then, the nucleic acid molecules may be introduced into various existing DNA molecules (or such as vectors) and cells known in the art.

The present application also relates to vectors comprising the aforementioned appropriate nucleic acid molecules and appropriate promoters or control sequences. These vectors may be used for transforming appropriate host cells to enable them to express proteins. The host cells may be prokaryotic cells, such as bacterial cells; or lower eukaryotic cells, such as yeast cells; or higher eukaryotic cells, such as mammalian cells. For example, the animal cells may comprise (but are not limited to): CHO-S, CHO-K1, and HEK-293 cells.

The step of transforming the host cells with recombinant DNAs in the present application may be performed using techniques well known in the art. An obtained transformant may be cultured by a conventional method, and it expresses the polypeptide encoded by the nucleic acid molecule(s) of the present application. According to the host cells used, they are cultured in a conventional medium under suitable conditions. Generally, the host cells are cultured and transformed under conditions suitable for the expression of the isolated antigen binding protein of the present application. Then, the isolated antigen binding protein of the present application is purified and obtained using conventional immunoglobulin purification steps, such as protein A-Sepharose, hydroxyapatite chromatography, gel electrophoresis, dialysis, ion exchange chromatography, hydrophobic chromatography, molecular sieve chromatography, or affinity chromatography, and other conventional separation and purification means well known to those skilled in the art.

The resulting monoclonal antibody may be identified by a conventional means. For instance, the binding specificity of the monoclonal antibody may be determined by immunoprecipitation or in vitro binding assays, such as fluorescence activated cell sorting (FACS), radioimmunoassay (RIA), or enzyme-linked immunosorbent assay (ELISA).

### Pharmaceutical Composition and Pharmaceutical Combination

In another aspect, the present application provides a pharmaceutical composition. The pharmaceutical composition may comprise the isolated antigen binding protein, the polypeptide, the immunoconjugate, the isolated nucleic acid molecule, the vector, the cell, and/or a pharmaceutically acceptable adjuvant and/or excipient described herein. In the present application, the pharmaceutically acceptable adjuvant may include a buffer, an antioxidant, a preservative, a low molecular weight polypeptide, a protein, a hydrophilic polymer, an amino acid, a sugar, a chelating agent, a counter ion, a metal complex, and/or a non-ionic surfactant. Except insofar as any conventional media or agent is incompatible with the cells described herein, its use in the pharmaceutical compositions of the present application is contemplated. In the present application, the pharmaceutically acceptable excipient may include an additive other than the main drug in the pharmaceutical preparation, and may also be referred to as an auxiliary material. For example, the excipients may include binders, fillers, disintegrants, lubricants in tablets. For example, the excipients may include wine, vinegar, medicinal juices, etc. in a traditional Chinese medicine pill. For example, the excipient may comprise a base portion of a semisolid formulation ointment, cream. For example, the excipients may include preservatives, antioxidants, flavoring agents, fragrances, cosolvents, emulsifiers, solubilizers, tonicity adjusting agents, colorants in liquid formulations. A pharmaceutical preparation should match the mode of administration. The pharmaceutical composition of the present application may be prepared into an injection form, for example, by means of a conventional method using normal saline or an aqueous solution containing glucose and other adjuvants. The pharmaceutical composition such as an injection and a solution should be manufactured under an aseptic condition. The dosage of an active ingredient is a therapeutically effective amount. In addition, the isolated antigen binding protein of the present application may also be used together with other therapeutic agents.

In another aspect, the present application provides a pharmaceutical combination comprising the isolated antigen binding protein and one or more active ingredients.

The isolated antigen binding protein, pharmaceutical composition or pharmaceutical combination described herein may be formulated, dosed, and administered in line with good medical practices. The considerations in this case comprise the specific disorder being treated, the specific mammal being treated, the clinical condition of a single patient, the cause of the disorder, the site of agent delivery, the method of administration, the schedule of administration, and other factors known to a medical practitioner. A therapeutic agent (e.g., an anti-properdin antibody) does not need to be but is optionally formulated and/or administered together with one or more agents that are currently used for preventing or treating the disorder in question. The effective amount of such other agents depends on the amount of the therapeutic agent (e.g., an anti-properdin antibody) existing in the preparation, the type of disorder or treatment, and other factors discussed above. Generally, these agents may be used at any dose that is empirically/clinically determined to be appropriate and via any route that is empirically/clinically determined to be appropriate. Compared with a single therapy, the dose of the antibody administered in a combination therapy may be reduced. The progress of such a therapy may be easily monitored by conventional techniques.

### Kit, Use and Method

In another aspect, the present application provides a method for detecting or determining properdin, which method may comprise using said isolated antigen binding protein or said polypeptide.

In the present application, the methods may include in vitro methods, ex vivo methods, methods of non-diagnostic or non-therapeutic interest. For example, the method may include a method for detecting the presence and/or amount of properdin for non-diagnostic purposes, which may include the steps of:
1) contacting a sample with an antigen binding protein of the present application; and
2) detecting the presence and/or amount of the antigen binding protein bound by the sample to determine the presence and/or level of expression of properdin in the sample obtained from the subject.

For example, said isolated antigen binding protein of said method may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 240, SEQ ID NO: 241, SEQ ID NO: 264, SEQ ID NO: 265 and SEQ ID NO: 266.

For example, said isolated antigen binding protein of said method may comprise an amino acid sequence as set forth in any one of SEQ ID NO: 240, and SEQ ID NO: 241. For example, said isolated antigen binding protein of said method may comprise an amino acid sequence as set forth in SEQ ID NO: 240. For example, said isolated antigen binding protein of said method may comprise an amino acid sequence as set forth in SEQ ID NO: 241.

In another aspect, the present application provides a kit for properdin that may include use of the isolated antigen binding protein or the polypeptide. In the present application, the kit may further comprise instructions that document a method for detecting the presence and/or amount of properdin. For example, the methods may include in vitro methods, ex vivo methods, methods of non-diagnostic or non-therapeutic interest.

For example, said kit may be an ELISA kit comprising said isolated antigen binding protein or the polypeptide. For example, said ELISA kit may detect properdin by direct ELISA, indirect ELISA, Sandwich ELISA or competitive ELISA.

For example, said isolated antigen binding protein or said polypeptide may be used as capture antibodies.

For example, said isolated antigen binding protein or said polypeptide may be used as detecting antibodies. For example, said detecting antibodies may link to HRP (horse radish peroxidase). For example, said detecting antibodies may link to ALP (alkaline phosphatase).

For example, said capture antibodies may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 240, SEQ ID NO: 241, SEQ ID NO: 264, SEQ ID NO: 265 and SEQ ID NO: 266. For example, said capture antibodies may comprise an amino acid sequence as set forth in any one of SEQ ID NO: 240, and SEQ ID NO: 241.

For example, said capture antibodies may comprise an amino acid sequence as set forth in SEQ ID NO: 240. For example, said capture antibodies may comprise an amino acid sequence as set forth in SEQ ID NO: 241.

For example, said detecting antibodies may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 240, SEQ ID NO: 241, SEQ ID NO: 264, SEQ ID NO: 265 and SEQ ID NO: 266.

In another aspect, the application provides a use of the isolated antigen binding protein or the polypeptide in the preparation of a kit for use in a method of detecting the presence and/or amount of properdin. For example, the methods may include in vitro methods, ex vivo methods, methods of non-diagnostic or non-therapeutic interest.

For example, said isolated antigen binding protein of said use may comprise an amino acid sequence as set forth in any one of SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 240, SEQ ID NO: 241, SEQ ID NO: 264, SEQ ID NO: 265 and SEQ ID NO: 266. For example, said isolated antigen binding protein of said use may comprise an amino acid sequence as set forth in any one of SEQ ID NO: 240, and SEQ ID NO: 241. For example, said isolated antigen binding protein of said use may comprise an amino acid sequence as set forth in SEQ ID NO: 240. For example, said isolated antigen binding protein of said use may comprise an amino acid sequence as set forth in SEQ ID NO: 241.

In another aspect, the present application provides a method of inhibiting alternative complement pathway comprising administering to a subject in need thereof an effective amount of the isolated antigen binding protein, the polypeptide, the immunoconjugate, the isolated nucleic acid molecule, the vector, the cell and/or the pharmaceutical composition, and/or a pharmaceutically acceptable therapeutic agent. In the present application, the method of modulating an immune response may include in vitro methods, ex vivo methods, methods of non-diagnostic or non-therapeutic interest.

In another aspect, the present application provides a method of inhibiting alternative complement pathway comprising administering to a subject in need thereof an effective amount of the pharmaceutical composition, pharmaceutical combination, and/or a pharmaceutically acceptable therapeutic agent. In the present application, the method of modulating an immune response may include in vitro methods, ex vivo methods, methods of non-diagnostic or non-therapeutic interest.

In another aspect, the present application provides a method of inhibiting properdin binding to C3 comprising administering to a subject in need thereof an effective amount of the isolated antigen binding protein, the polypeptide, the immunoconjugate, the isolated nucleic acid molecule, the vector, and/or the cell. The method may be an ex vivo or in vitro method.

In another aspect, the present application provides an isolated antigen binding protein, said polypeptide, said immunoconjugate, said isolated nucleic acid molecule, said vector, said pharmaceutical composition for preventing and/or treating diseases. For example, said diseases may be caused by properdin. For example, said diseases may be mediated by alternative pathway. For example, said diseases may comprise autoimmune thrombotic thrombocytopenic purpura (TTP), hemolytic uremic syndrome (HUS), atypical hemolytic uremic syndrome (aHUS), paroxysmal nocturnal hemoglobinuria (PNH), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMO), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

In another aspect, the kit, the pharmaceutical composition and/or the pharmaceutical combination is used for the prevention and/or treatment of diseases in the present application. For example, said diseases may be caused by properdin. For example, said diseases may be mediated by alternative pathway. For example, said diseases may comprise autoimmune thrombotic thrombocytopenic purpura (TTP), hemolytic uremic syndrome (HUS), atypical hemolytic uremic syndrome (aHUS), paroxysmal nocturnal hemoglobinuria (PNH), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMO), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

In another aspect, the present application provides a use of the isolated antigen binding protein, the polypeptide, the immunoconjugate, the isolated nucleic acid molecule, the vector, the cell and/or the pharmaceutical composition for the preparation of a medicament for the prevention and/or treatment of diseases in the present application. For example, said diseases may be caused by properdin. For example, said diseases may be mediated by alternative pathway. For example, said diseases may comprise autoimmune thrombotic thrombocytopenic purpura (TTP), hemolytic uremic syndrome (HUS), atypical hemolytic uremic syndrome (aHUS), paroxysmal nocturnal hemoglobinuria (PNH), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMO), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

In another aspect, the present application provides the use of a pharmaceutical combination for the manufacture of a medicament for the prevention and/or treatment of diseases in the present application. For example, said diseases may be caused by properdin. For example, said diseases may be mediated by alternative pathway. For example, said diseases may comprise autoimmune thrombotic thrombocytopenic purpura (TTP), hemolytic uremic syndrome (HUS), atypical hemolytic uremic syndrome (aHUS), paroxysmal nocturnal hemoglobinuria (PNH), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMO), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

In another aspect, the present application provides a method of preventing and/or treating a disease or disorder comprising administering the isolated antigen binding protein, the isolated nucleic acid molecule, the vector, the cell, the pharmaceutical composition to a subject in need thereof. For example, said diseases may be caused by properdin. For example, said diseases may be mediated by alternative pathway. For example, said diseases may comprise autoimmune thrombotic thrombocytopenic purpura (TTP), hemolytic uremic syndrome (HUS), atypical hemolytic uremic syndrome (aHUS), paroxysmal nocturnal hemoglobinuria (PNH), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMO), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

In another aspect, the present application provides a method of preventing and/or treating a disease or disorder comprising administering the pharmaceutical combination to a subject in need thereof. For example, said diseases may be caused by properdin. For example, said diseases may be mediated by alternative pathway. For example, said diseases may comprise autoimmune thrombotic thrombocytopenic purpura (TTP), hemolytic uremic syndrome (HUS), atypical hemolytic uremic syndrome (aHUS), paroxysmal nocturnal hemoglobinuria (PNH), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMO), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

The pharmaceutical compositions, pharmaceutical combinations, and methods described herein can be used in conjunction with other types of therapies, such as chemotherapy, surgery, radiation, gene therapy, and the like.

In the present application, the subject and/or patients may include a human or non-human animal. For example, the non-human animal may be selected from the group consisting of: monkey, chicken, goose, cat, dog, mouse and rat. Furthermore, non-human animals may also include any animal species other than humans, such as livestock animals, or rodents, or primates, or domestic animals, or poultry animals. The human may be caucasian, african, asian, amphibian, or other ethnicity, or a hybrid of various ethnicities. As another example, the person may be an elderly person, an adult, a teenager, a child, or an infant.

An effective amount in humans can be presumed from an effective amount in experimental animals. For example, Freiich et al describe the dose correlation between animals and humans (based on milligrams per square meter of body surface) (Freirich et al, Cancer Chemother. Rep. 50, 219 (1966)). The body surface area can be approximately determined from the height and weight of the patient. See, e.g., Scientific Tables, Geigy Pharmaceuticals, Ardsley, N.Y., 537 (1970).

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

### Example 1.1: Discovery of VEGF-Binding VHHs

### 1) VHH Immune Library Construction

Immunization was performed using recombinant human VEGFA165 (UniProt identifier P15692-4) in two healthy camels. On day 90 after finishing 6 rounds of immunization, Camel serum was collected and used to measure titers of the antigen-specific antibodies.

Briefly, ELISA analysis was performed to measure the antibody titer in camel serum. Specially, human VEGFA165 was diluted to a final concentration as of 5 μg/ml in Phosphate-buffered saline (PBS), 100 μl of the solution was used for coating in each well of a 96-well ELISA microplate and incubate at room temperature (RT) for 1 hour (hr). Each well was washed three times with 300 μl washing buffer (0.05% Tween-20 in PBS, or PBST) to remove unbound human VEGFA165, and then 200 μl of blocking buffer (2% BSA in PBS) were added to each well and incubated at room temperature for 2 hr at RT. A test serum sample was serially 2-fold diluted to make 15 test samples from 1/100 to 1/1638400. After blocking, each well was washed with 300 μl washing buffer for three times and then each of the serially diluted test sample was added to each well and incubate at RT for 1 hr. After three times washing, 100 μl of 1:10000 diluted secondary antibody conjugated to horseradish peroxidase (HRP-Goat anti-Llama IgG(H+L), Novex cat #A16060) was added to each well and incubate at RM for 1 hr. After three times of washing again, 100 μl of TMB solution were added to each well and allowed to react at RT for 10 mins. After adding 100 μl of stop solution (2 mol/L (M) HCl), the microplates were measured at OD450 nm.

To construct a phage-displayed VHH library, 200 ml of blood was collected from each of the immunized camels and PBMCs were recovered for VHH immune library construction by following a standardized protocol. The final phage-displayed VHH library had 3.4×10⁹ independent clones, with 91% of them encoding VHH-gp3 fusion proteins.

### 2) Phage Panning Method

For Round 1 panning, 300 μl (6.0×10¹³ CFU) phages was incubated with 10 μg of biotinylated recombinant human VEGFA121-Avi-His tag or mouse VEGFA120-Avi-His tag protein within 1 ml of blocking buffer (1% BSA in PBS) at RT for 1 hr to make phages/target mixture. At the same time, 100 μL of Streptavidin-coated Dynabeads M-280 (Invitrogen, 11206D) were washed with 1 mL of blocking buffer for five times in an Eppendorf microtube. Thereafter, the phage/target mixture was incubated with the Mag-beads prepared as above on a rotator at RT for 30 min. To recover the phages binding to the Mag-beads, the reaction tube was placed on a magnetic rack for 30s. After the supernatant was removed, the beads were washed with 1 mL of PBST for 10 times, followed by one-time washing with PBS. The phages were eluted by adding 1 ml of trypsin (10 μg/ml in PBS) at 37° C. for 30 min.

To amplify the eluted phages, the eluted phages were added into a 15-ml tube containing 4 ml of E. Coli TG1 culture at exponential phase (A600≈0.6) and incubated without shaking at 37° C. for 30 min. After the infection, the bacterial culture was centrifuged at 4,000 rpm for 20 min at 4° C. and resuspended in 500 μl of 2×YT medium for spreading onto 2×YT-GA plates containing 2% Glucose and 100 μg/ml Ampicillin in 2×YT, and incubated overnight at 37° C. To amplify phages, collect the bacteria on the second day and inoculate with 100 ml of 2×YT-GA at 37° C. with shaking at 250 rpm to reach a cell density at an A600 at 0.6 (approximately 1-2h). The phages were rescued by adding helper phage M13K07 at a phage: bacteria ratio of 1000. After Incubating the culture at 37° C. without shaking for 30 min, the culture was continued for 30 min with shaking at 180 rpm. Medium was changed to 2×YT-AK (Amp: 100 μg/ml, Kan: 50 μg/ml) before the culture was placed back to incubator for shaking at 250 rpm at 30° C. for 4 hrs.

Following amplification, the bacterial pellets was removed by centrifugation for 20 min at 6000 rpm at 4° C., and the phage were precipitated from the supernatant by adding ¼ volume of PEG solution (20% Polyethylene glycol 6000, 2.5M NaCl) and staying on ice overnight. After centrifugation at 10,000 rpm for 30 min, the pellets were resuspended in 5 ml of PBS, insoluble debris were removed by centrifugation at 8000 rpm for 10 min at 4° C. PEG precipitation was repeated once as described above. The final phage pellets were resuspended in 1 ml of PBS, and tittered to be used as input for the next round of selection. Round 2 and 3 were performed as described for Round 1, the variation was the decreased amount of the antigen or antigens derived from different species to have cross-reactive phage clones. Panning summary was listed in Table 3.

TABLE 1
Panning summary

| Panning round | Library | input (CFU) | output (CFU) | Ratio(I/O) |
|---|---|---|---|---|
| 1^{st} (10 ug) | 1904A-H-1st | 3 × 10¹² | 1 × 10⁷ | 3 × 10⁵ |
| | 1904A-M-1st | 3 × 10¹² | 1 × 10⁷ | 3 × 10⁵ |
| 2^{nd} (1.5 ug) | 1904A-H-H-2nd | 7 × 10¹² | 3 × 10⁷ | 2.3 × 10⁵ |
| | 1904A-H-M-2nd | 7 × 10¹² | 2 × 10⁷ | 3.5 × 10⁵ |
| | 1904A-M-H-2nd | 7 × 10¹² | 13 × 10⁷ | 0.5 × 10⁵ |
| | 1904A-M-M-2nd | 7 × 10¹² | 2.5 × 10⁷ | 2.8 × 10⁵ |
| 3^{rd} (0.5 ug) | 1904A-H-H-H-3rd | 8 × 10¹² | 5 × 10⁸ | 1.6 × 10⁴ |
| | 1904A-H-M-M-3rd | 3 × 10¹² | 3 × 10⁸ | 1 × 10⁴ |
| | 1904A-M-H-H-3rd | 8 × 10¹² | 2 × 10⁸ | 4 × 10⁴ |
| | 1904A-M-M-M-3rd | 10 × 10¹² | 13 × 10⁸ | 0.76 × 10⁴ |

3) Primary Screening of Panning Outputs with Phage ELISA

Individual bacterial colonies were picked and inoculated into 200 μl 2×YT-GA medium, cultured at 37° C. with shaking (250 rpm) for 4˜5 hr. Then 10 N1 of culture was transferred into a new deep 96-well plate containing 200 μl of 2×YT-GA and incubated as above till OD600 reached around 0.5. M13K07 helper phages were added to a final concentration of 1×10¹⁰ cfu, and plates were incubated at 37° C. for 30 min without shaking. After the bacteria were harvested by centrifugation at 4000 rpm for 20 min and resuspended in 350 μl of 2×YT-AK medium, the bacterial culture was kept incubation at 30° C. overnight with shaking (800 rpm). After the overnight culture was spun at 4000 rpm at 4° C. for 30 min, the supernatants were collected for phage ELISA.

For phage ELISA, immunoplates were coated with 100 μl of 1 μg/ml streptavidin per well and incubate at 4° C. overnight. After washing with PBST for 3 times and blocking with 200 μl of 1% BSA/PBS at RT for 1 hr, the recombinant hVEGFA121-biotin or mVEGFA120-biotin (0.1 μg/ml) was added at 100 μl per well and incubated at RT for 1 hr. Plates were washed with PBST for 3 times and 50 μl phage supernatant and 50 μl 1% BSA/PBST were mixed and added into each well, incubated at RT for 1 hr. Plates were washed with PBST for 3 times and 100 μl of goat anti-M13-HRP (SinoBio, 11973-MM05T-H, diluted at 1/5000 in 1% BSA/PBST) were added into each well and incubated at RT for 1 hr. Plates were then washed as before and 100 μl of TMB substrate solution were added and incubated at RT for 15 min. 100 l/well of stop solution were added to stop the reaction before the plates were scanned with a microplate reader at 450 nm. Target-binding phages were subjected to DNA sequencing to determine the encoded VHH identities, and phages with different amino acid sequences of VHH were considered as unique clones. In total, 139 unique clones with different CDR sequences were identified as positive in target-binding assays with phage ELISA. After secondary ELISA assays by using periplasmic extractions (PPE) from the E. coli culture, part of them were selected for production of recombinant VHH-Fc proteins. Summary of screening results was listed in Table 4.

TABLE 2
summary of primary screening

| | | H VEGFA121- | | | | | |
|---|---|---|---|---|---|---|---|
| | | binder | H/M VEGFA- | total | ratio | unique | Accumulative |
| Screening | Library | (Only) | binder | binder | (binder/96) | sequence | number |
| 5.18-1^{st} | 1904A-H-H-H-3rd | 84 | 8 | 92 | 96% | 24 | 53 |
| | 1904A-M-H-H-3rd | 16 | 3 | 19 | 20% | 23 | |
| | 1904A-H-M-M-3rd | 7 | 10 | 17 | 18% | 6 | |
| | 1904A-M-M-M-3rd | 7 | 5 | 12 | 13% | 0 | |
| 5.26-2^{nd} | HMM-3rd-Plate1 | 0 | 16 | 16 | 17% | 3 | 32 |
| | HMM-3rd-Plate2 | 0 | 16 | 16 | 17% | 5 | |
| | HMM-3rd-Plate3 | 0 | 12 | 12 | 13% | 6 | |
| | HMM-3rd-Plate4 | 1 | 19 | 20 | 21% | 6 | |
| | HMM-3rd-Plate5 | 0 | 12 | 12 | 13% | 4 | |
| | HMM-3rd-Plate6 | 0 | 18 | 18 | 19% | 8 | |
| 6.03-3^{rd} | HMM-3rd-Plate7 | | | 16 | 17% | 4 | 20 |
| | HMM-3rd-Plate8 | | | 12 | 13% | 2 | |
| | HMM-3rd-Plate9 | | | 14 | 15% | 1 | |
| | HMM-3rd-Plate10 | | | 13 | 14% | 6 | |
| | HMM-3rd-Plate11 | | | 19 | 20% | 4 | |
| | HMM-3rd-Plate12 | | | 16 | 17% | 3 | |
| 6.18-4^{th} | HH-2nd-Plate13 | 10 | 4 | 14 | 15% | 8 | 20 |
| | HH-2nd-Plate14 | 5 | 1 | 6 | 6% | 3 | |
| | HH-2nd-Plate15 | 9 | 3 | 12 | 13% | 5 | |
| | HH-2nd-Plate16 | 8 | 2 | 10 | 10% | 4 | |
| 7.8-5^{th} | HH-2nd-Plate17 | 7 | 2 | 9 | 9% | 3 | 14 |
| | HH-2nd-Plate18 | 7 | 1 | 8 | 8% | 0 | |
| | HH-2nd-Plate19 | 10 | 3 | 13 | 14% | 1 | |
| | HH-2nd-Plate20 | 2 | 0 | 2 | 2% | 1 | |
| | HH-2nd-Plate21 | 2 | 0 | 2 | 2% | 1 | |
| | HH-2nd-Plate22 | 2 | 1 | 3 | 3% | 3 | |
| | HH-2nd-Plate23 | 7 | 1 | 8 | 8% | 1 | |
| | HH-2nd-Plate24 | 8 | 2 | 10 | 10% | 4 | |

| | | |
|---|---|---|
| Total | 139 | 139 |

### 4) Production and Purification of Recombinant VHH-Fc Proteins

VHH domain of selected clones were amplified by using degenerated primers (Fw: 5′ (C/g)A(g/T)gTgCAgCTggTggAgTCTgg, Rv: 5′ TgAggAgAC(A/g)gTgACC(A/T)g) to create constructs for expressing recombinant VHH-hIgG4 Fc fusion proteins within HEK293 cells. After the DNA sequences were verified with DNA sequencing, the recombinant plasmids were prepared by following standard protocols and filtered through a 0.22-μm filter before use for transfection of HEK293 cells.

To express the recombinant VHH-Fc proteins, 100 ml of Expi293F™ Cells in OPM-CD05 Medium (OPM, cat #81075-001) were cultured to reach a cell density of approximately 3˜5×10⁶ viable cells/mL with viability more than 95%. Plasmids were diluted with OPM-CD05 Medium to a concentration of 1.5 ag/mi in a total volume of 5 ml. Transfection reagent PEI (Polysciences, cat #24765-1) was diluted with OPM-CD05 Medium to a same volume of 5 ml to have a DNA:PEI ratio as 1:6 (m/m) when the diluted DNA and PEI were mixed together. After being incubated at RT for 15 minutes, the DNA/PEI complex were added onto the prepared Expi293F™ cells by swirling gently. Then the cells cultures were placed in a 37° C. incubator with ≥80% relative humidity and 5% CO₂ on an orbital shake. At 24 hr post the transfection, 5% prepton (1 mg/ml) and 2% glucose (330 g/L) were added to the culture slowly. After days of culturing, the cell culture supernatant was collected by sequential centrifugations at 1200 rpm for 10 min and 3900 rpm for 20 min before being used for Protein A purification.

VHH-Fcs were purified with Protein A Focurose 4FF (BIOON, HZ1011-2). Briefly, 1.5 ml of Protein A slurry were loaded onto a 20-ml column (G-bios, C006197-0025). After the columns were equilibrated with PBS of 10-fold of CV (column volume), the cell culture supernatant prepared as above were loaded and flow throw the Protein-A columns by gravity for 2 times. After the columns were washed with PBS for 10 times of CV, 2 ml of 0.1 M Glycine-HCl buffer (pH3.0) were used to elute the VHH-Fc proteins. The eluted proteins were neutralized with 100 ul of 1 M (pH 8.5) Tris-HCL buffer. The Protein A affinity column was regenerated and preserved by washing with PBS, ddH2O and 20% ethanol sequentially. For the eluted protein, it was desalted through an Amicon UltraCel 30K centrifugal device (Milipore, UFC903016). Briefly, eluted protein was diluted in 10 ml PBS and concentrated to 1.5 ml by centrifugation for 3 times. The final protein solution was formulated in PBS to less than 1 ml and filtrated with 0.22-μm filters.

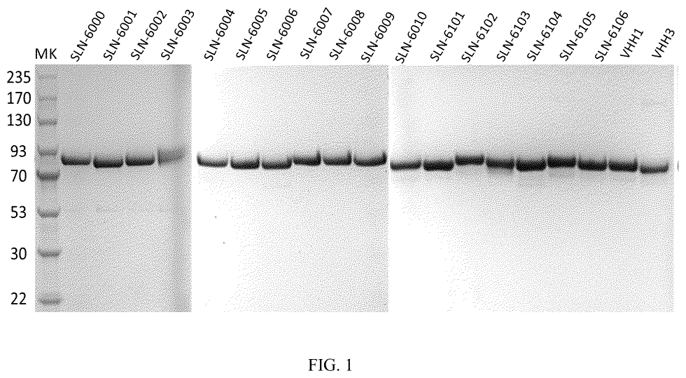

Purity of VHH-Fcs were analyzed with SDS-PAGE. Briefly, 2 ag protein in 4×LDS Sample buffer (Genscript, M00676-10) was loaded and analyzed with SurePAGE gel (Genscript, M00653) in Tris-MOPS SDS buffer (Genscript, M00138) at a constant voltage of 160-V for 50 min. Proteins were visualized with Coomassie stain (TIANGEN, cat #PA101) following the manufacturer's instructions. The results were shown as in FIG. 1.

### 5) Characterization of VHH-Fcs

For binding ELISA, immunoplates were coated with 100 μl/well 1 μg/ml streptavidin and incubate at 4° C. overnight. Wells were washed with PBST for 3 times and blocked with 200 μl of 1% BSA/PBS at RT for 1 hr. Washed with PBST for 3 times and add hVEGFA121-biotin or mVEGFA120-biotin (0.1 μg/ml) 100 al/well and incubated at RT for 1 hr. Plates were washed with PBST for 3 times, 100 al/well 5-fold serially diluted VHH-Fcs from 2 μg/ml was added. and incubate at RT for 1 hr. Plates were washed with PBST for 3 times and add 100 al goat anti-human Fc-HRP (Sigma, A0170) diluted 1/5000 in 1% BSA/PBST to each well and incubate at RT for 1 hr. Plates were then washed as before and add 100 al TMB substrate and incubate at RT for 15 min. 100 al per well stop solution was added to stop the reaction, and the plates were read with microplate reader at 450 nm.

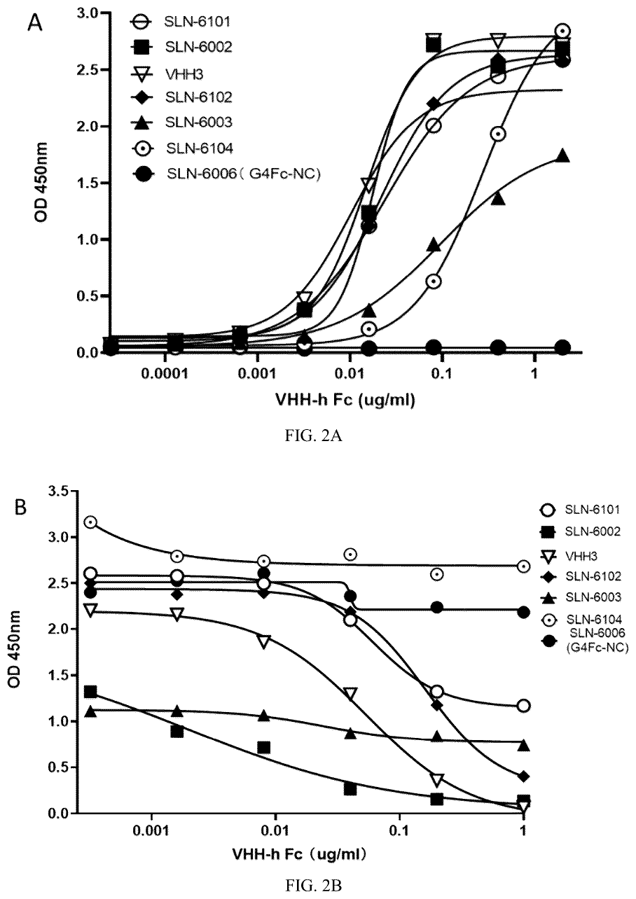

For receptor blocking assay, refer to the method above. The difference was the serially diluted VHH-Fc was pre-mixed with hVEGFA-R2-Mouse Fc (0.1 μg/ml) before being added into each well, and the secondary antibody was Goat anti-mouse IgG-Fc HRP (Abcam, ab98717). VHH-Fc binding to hVEGFA121 and receptor blocking activity was show in FIGS. 2A-2B. C8, F6, C2, E7 and B3 were the top 5 strongest binders to hVEGFA121.

| Name | No. |
|---|---|
| C8 | SLN6001 |
| F6 | SLN6005 |
| C2 | SLN6002 |
| E7 | SLN6004 |
| B3 | SLN6101 |
| H7 | SLN6102 |
| A10 | SLN6103 |
| D2 | SLN6003 |
| G4Fc-NC | SLN6006 |

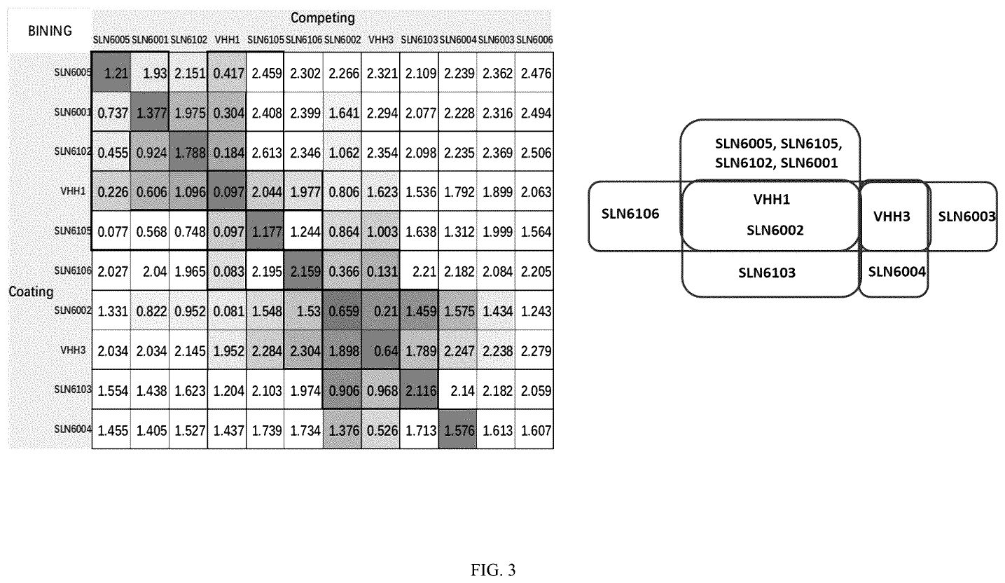

For epitope binning assay, immunoplates were coated with 100 μl/well of 5 μg/ml VHH Fc fusion protein and incubate at 4° C. overnight. Wells were washed with PBST for 3 times and blocked with 200 μl of 1% BSA/PBS at RT for 1 hr. 60 μl hVEGFA121-biotin (2 μg/ml) and 60 μl VHH-Fc fusion protein (5 μg/ml) were pre-mixed and transfer 100 μl to each well that had been coated with VHH-Fc and blocked with BSA, and continued incubation at RT for 1 hr. Plates were washed with PBST for 3 times and add 100 μl SA-HRP (Sigma, S5512) diluted 1/5000 in 1% BSA/PBST to each well and incubate at RT for 1 hr. Plates were then washed as before and add 100 μl TMB substrate and incubate at RT for 15 min. 100 μl/well of stop solution was added to stop the reaction, and the plates were read with microplate reader at 450 nm. Results was show in FIG. 3. VHHs with competitive target binding capabilities were grouped to a same bin. The results indicated F6, C8, H7 and VHH1 belong to Bin #1, C2, VHH3 and A10 belong to Bin #2, D2 might have different epitope with most of other VHH-Fc.

### 6) Bi/Tri-Specific VHH-Fc Production and Characterization

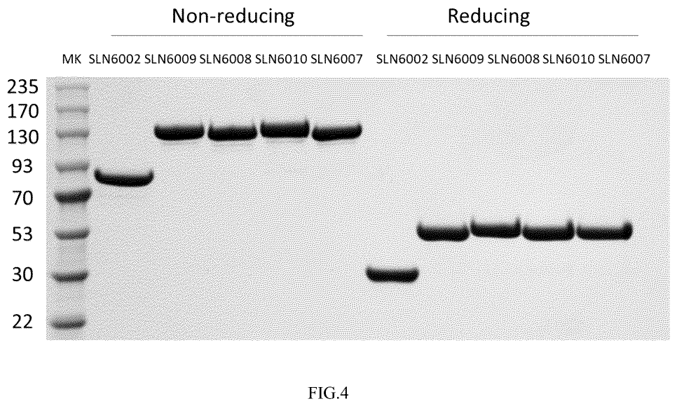

VHHs from different bins were combined with G4S linker to make Bi/Tri-paratopic VHH-Fc fusion proteins. pSLN7000 vector was used as above. Plasmid construction and protein purification can refer to the above. Production results was listed in Table 5. SDS-PAGE analysis and characterization result show in FIG. 4 and FIGS. 5A-5B respectively. In both of the target-binding and receptor-blocking assays, the bi-paratopic VHHs demonstrated much better activities than those of single VHHs. among them, C2-D2, C2-F6 and C2-C8 had superior activities and thus selected as basis for further optimization. Inclusion of three non-competitive VHHs in one construct (tri-paratopics) did not improve such activities further, therefore the bi-paratopic VHHs were selected as therapeutic proteins.

TABLE 5
Summary of Bi/Tri-paratopic VHH-Fcs

| | | Conc. | Vol. | Total yield |
|---|---|---|---|---|
| Pro. Name | No. | (mg/ml) | (ml) | (mg) |
| C2 + C8 | SLN6001 | 0.80 | 1.5 | 1.2047 |
| C2 + D2 | SLN6004 | 0.75 | 1.5 | 1.124 |
| C2 + E7 | SLN6003 | 0.58 | 1.5 | 0.8752 |
| C2 + F6 | SLN6002 | 0.45 | 1.5 | 0.6712 |
| D2 + C2 + C8 | SLN6051 | 0.88 | 0.5 | 0.44 |
| F6 + C2 + D2 | SLN6052 | 0.78 | 1.5 | 1.17 |
| E7 + C2 + D2 | SLN6053 | 0.75 | 0.4 | 0.3 |
| D2 + C2 + F6 | SLN6054 | 0.74 | 1.5 | 1.11 |
| C2 + D2 + F6 | SLN6055 | 0.9 | 1.5 | 1.36 |
| C2 + C8 + D2 | SLN6056 | 0.59 | 1.5 | 0.89 |
| C2 + D2 + E7 | SLN6057 | 0.59 | 1.5 | 0.88 |
| C2 + F6 + D2 | SLN6058 | 1.2 | 1.5 | 1.8 |
| C2 + F6 + E7 | SLN6059 | 0.58 | 1.5 | 0.88 |
| F6 + C2 + E7 | SLN6060 | 0.47 | 0.5 | 0.23 |
| Aflibercept | SLN6066 | 8.02 | 1.2 | 9.63 |

### 7) Humanization of VHH-Fc Leads and Characterization

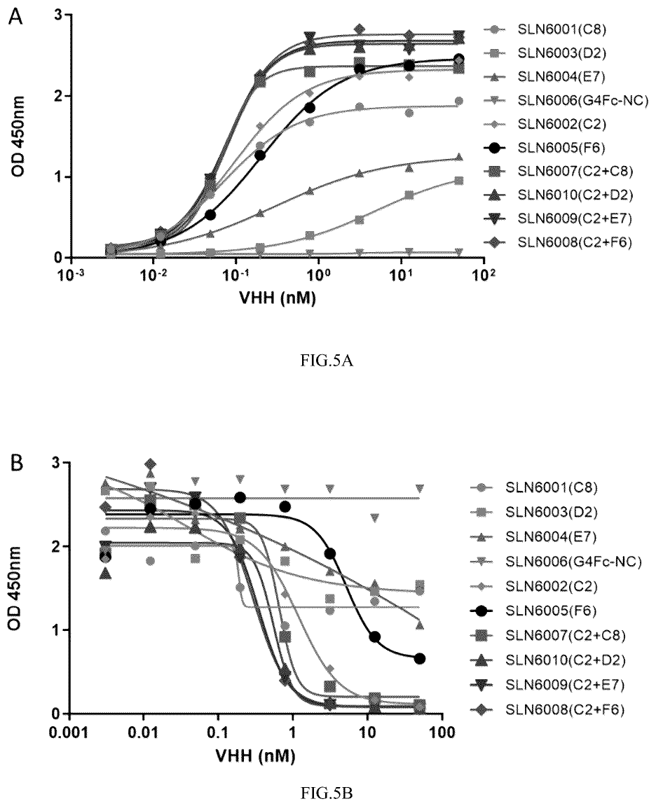

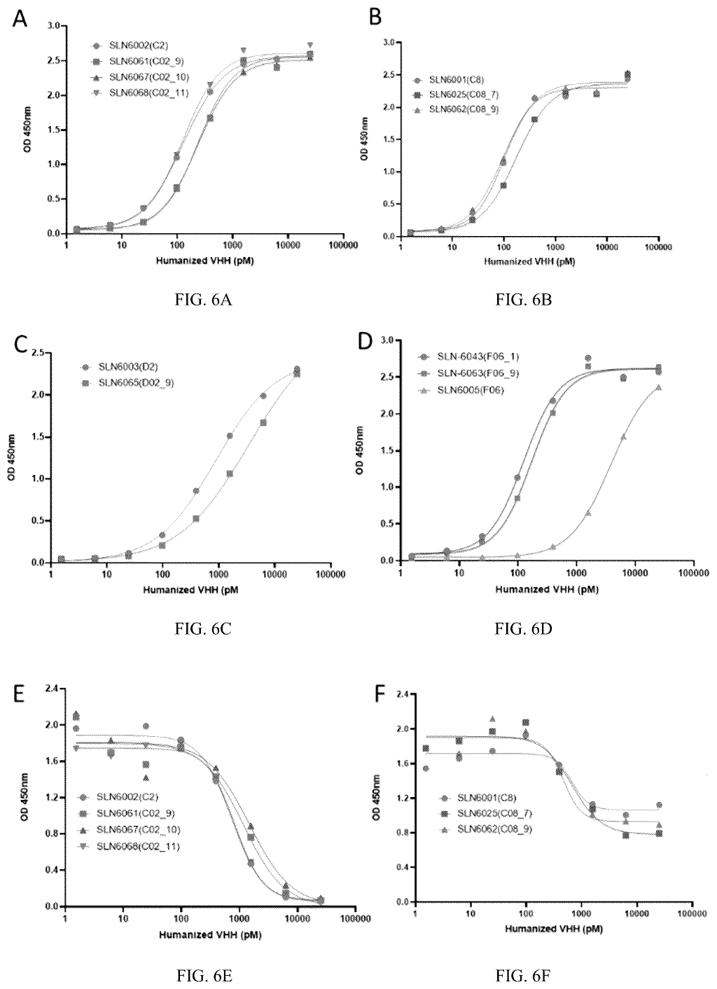

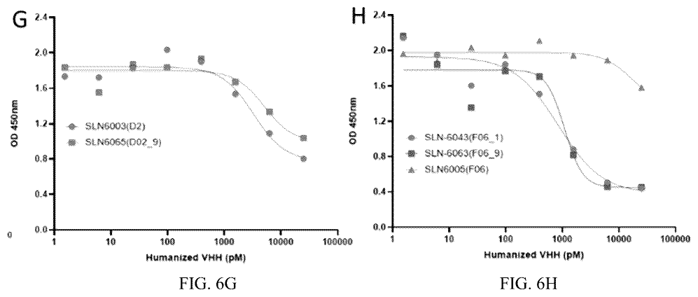

Four VHHs (C2, C8, F6, D2) were selected for humanization. Proceed with plasmid construction and protein production as above, the humanized VHH-Fc was characterized with target-binding (ELISA) and RBA. The result shown in FIGS. 6A-6H indicated that C2-11 (SLN-6068, SEQ ID NO: 13), C8-9 (SLN6062, SEQ ID NO: 15), F6-1 (SLN-6043, SEQ ID NO: 14) and D2-9 (SLN6065, SEQ ID NO: 16) sustained target-binding and receptor-blocking activities as those of the parental VHH's, and thus selected as humanized sequences for further development.

8) Bi-Paratopic VHH-Fcs with Humanized Sequences

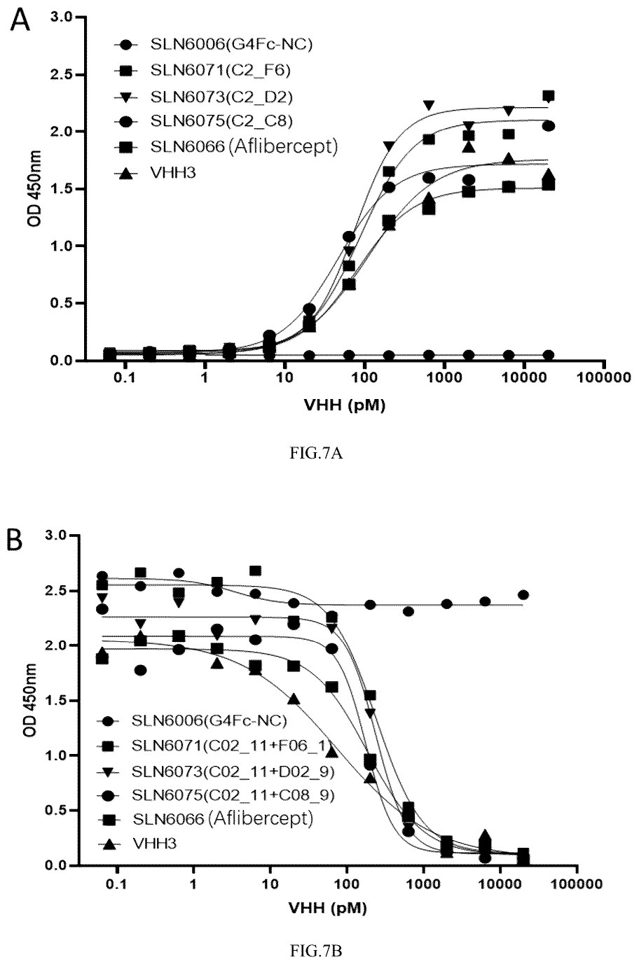

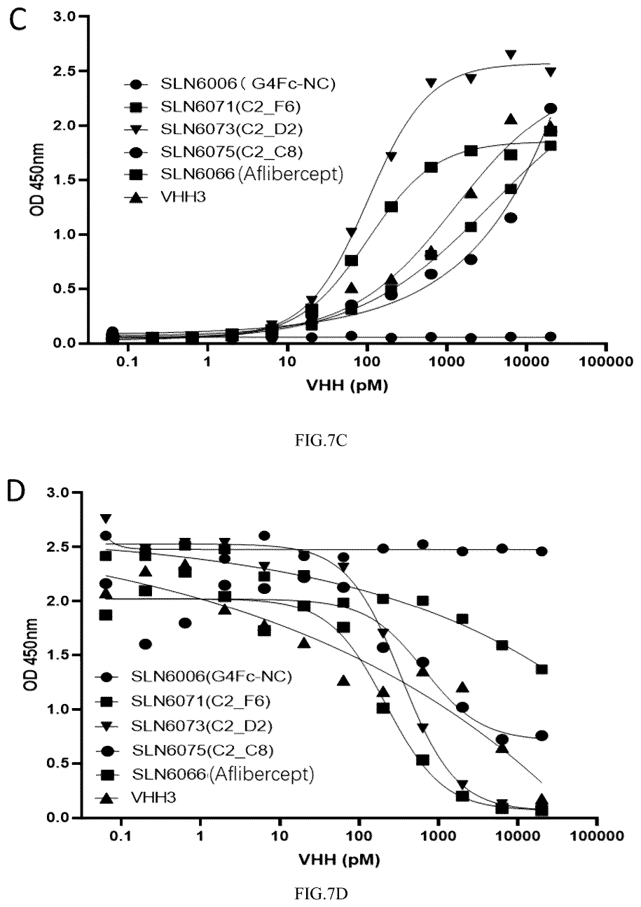

C2-11, F6-1, D2-9 and C8-9 were used for creation of bi-paratopic VHHs with humanized sequences through a G4S linker, by following procedures as described above. Three humanized bi-paratopic VHH-Fc fusion proteins were generated, named as SLN6071 (SEQ ID NO: 17), SLN6079 (SEQ ID NO: 19) and SLN6075 (SEQ ID NO: 18), the Fc region comprises an amino acid sequence of SEQ ID NO: 20. Target-binding ELISA and RBA shown in FIGS. 7A-7D indicated that such humanized bi-paratopic VHHs had biological activities comparable to that of Aflibercept, a VEGF antagonist approved by FDA for treatment of VEGF-driven angiogenesis.

### Example 1.2 RBA Assays of hVEGF or mVEGF on Human VEGFR2

Materials: The humanized bi-paratopic VHH-Fc fusion protein (SLN6071-6076, aflibercept (SLN6066, SEQ ID NO: 38), all made in house); bevacizumab (R & D, MAB9947-SP25 ug), hVEGFR2-mFc (made in house); hVEGFA (SLN4007, made in house), mVEGFA (SLN4011, made in house); Streptavidin (Sigma, CAT #85878); Goat-Anti-hFc-HRP (Sigma, CAT #A0170); Goat-Anti-mFc-HRP (Abcam, CAT #ab98717); TMB and stop solution (abeam, CAT #ab210902 and ab210900); Coating Buffer: 1×PBS; Washing buffer: 1×PBS+0.05% Tween20; Blocking buffer: 1×PBS+0.05% Tween20+1% BSA.

### Procedure:

1) Coat plates with 100 ul/well of 1 ug/ml streptavidin and incubate at 4° C. overnight. 2) Wash the plates with PBST 3 times. 3) Block the plates with 1% BSA in PBST at RT for 1 hr. 4) Add hVEGFA-biotin (0.07 ug/ml) or mVEGFA120-biotin (0.07 ug/ml) 100 ul/well and incubate at RT for 1 hr. 5) Wash the plates with PBST 3 times. 6) Prepare serial dilutions of the test articles (the humanized bi-paratopic VHH-VHH-Fcs, aflibercept, bevacizumab) starting from 25000 pM, before adding 100 ul of the serially diluted articles to the microplate wells for incubation at RT for 1 hr. 7) Wash the plate wells with PBST for 3 times before adding 100 ul of hVEGFR2 (0.14 ug/ml) and incubating at RT for 1 hr. 8) Wash the plate wells with PBST for 3 times before incubating with goat anti-human Fc-HRP (1:5000) at RT for 1 hr. 9) Wash the plate wells with PBST 3 times before adding 100 ul/well of TMB for incubation at RT for 15 min. 10). Quench with 100 ul/well stop solution before scanning the plates with microplate reader at 450 nm.

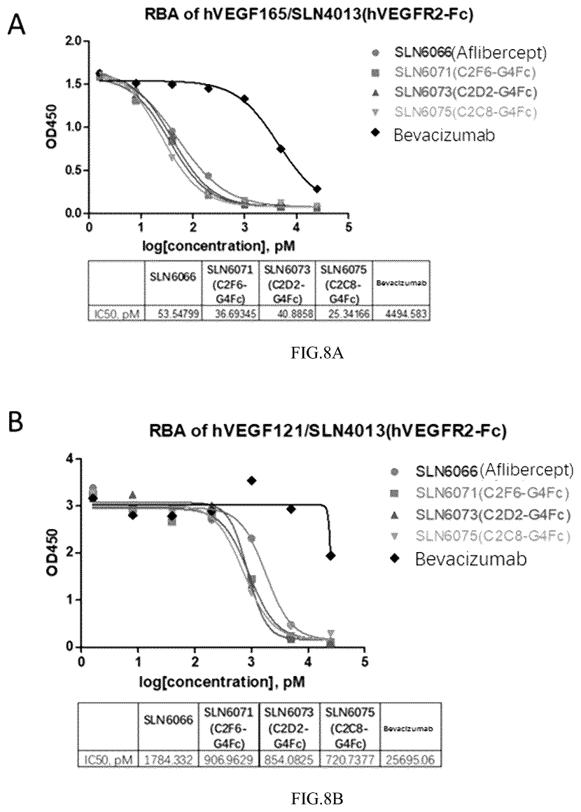

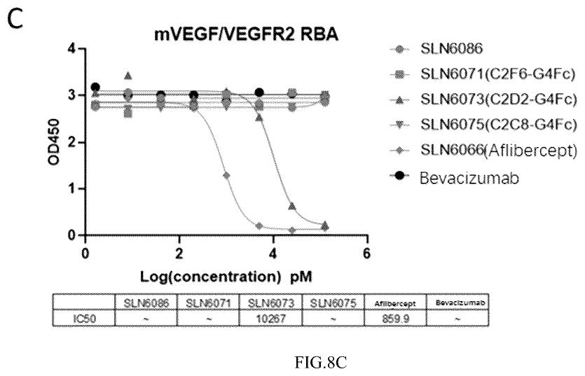

The results are shown in FIGS. 8A-8C indicating the RBA assays of hVEGFA(165 and 121), mVEGFA (120) on human VEGFR2: (A) RBA assays of hVEGFA(165) on human VEGFR2 showed IC50 of Aflibercept, SLN6071, SLN6073, SLN6075, Bevacizumab are 53.5 pM, 36.7 pM, 40.9 pM, 25.3 pM, 4494.6 pM, respectively. (B) RBA assays of hVEGFA(121) on human VEGFR2 showed IC50 of Eylea, SLN6071, SLN6073, SLN6075, Bevacizumab are 1784.3 pM, 907 pM, 854.1 pM, 720.7 pM, 25695.1 pM, respectively. (C) RBA assays of mVEGFA(120) on human VEGFR2 showed SLN6073 and Aflibercept can block VEGFA/VEGFR2 binding. IC50 of Aflibercept and SLN6073 are 859.9 pM, 10267 pM, respectively.

### Example 1.3 Inhibition of HUVEC Proliferation

Materials: HUVEC cells; EMC (Sclencell, 1001); Cell counting kit-8 (Donjndo)

Procedure: 1). Seed 3×10³ HUVEC cell per well in 96-well plates in complete medium (5% FBS, 1% EGFS, 1% P/S), and culture overnight; 2) Wash the well with 100 μL PBS, and starve the cell with the basal medium for 2 hrs; 3) Serially dilute the test articles (VEGF inhibitors) from 20000 nm/mL to 0.00128 nm/mL with basal medium (containing 1.0% FBS), mix and incubate with same volume of 100 ng/mL VEGF for 4 hrs; 4) Add 100 μL of the mixture as above onto the starved HUVEC cells, and continue culturing for 72 hrs; 5) Add 10 μL CCK-8 and incubate for 3 hrs; 6) Scan the plate at 450 nm; 7) calculate cell viability (%)=[(As−Ab)/(Ac−Ab)]×100; Inhibition rate (%)=[(Ac−As)/(Ac−Ab)]×100; As=OD450 of the experimental well, Ab=OD450 of the blank control, Ac=control well absorbance.

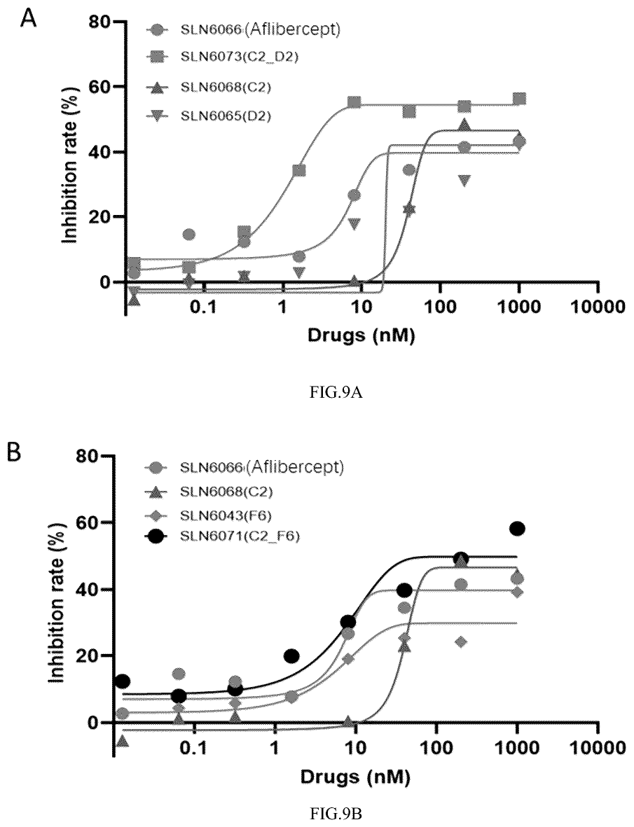

The results are shown in FIGS. 9A-9B indicating the inhibition of HUVEC cell proliferation. SLN6073 showed about three-fold stronger inhibition ability than Aflibercept. Bi-specific VHH-Fcs SLN6073, SLN6071 are better than parent mono-VHH-Fcs SLN6008, SLN6005, SLN6068 and SLN6043.

### Example 1.4 Dual Functional Recombinant Proteins Inhibiting VEGF-Driven Angiogenesis and Factor H-Regulated Complement Activation

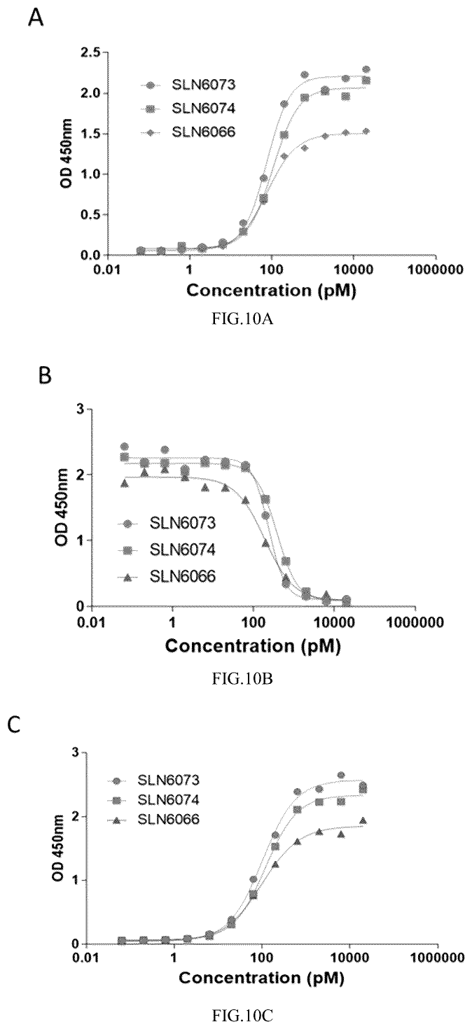

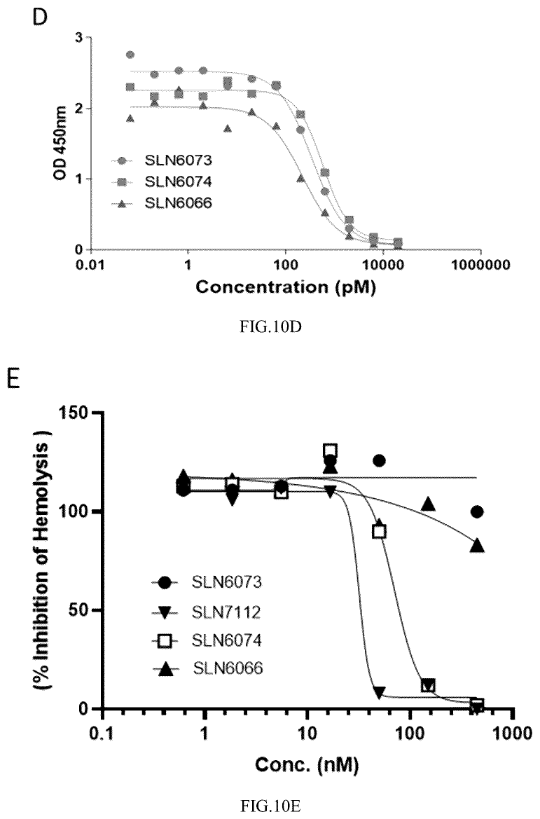

Truncated CFH (domain 1-4 and 19-20) was fused to the C-terminus of SLN6073 (VHH-Fc) to form SLN6074 (VHH-Fc-CFH) to make a dual functional recombinant protein inhibiting VEGF-driven angiogenesis and factor H-regulated complement activation. The results are shown in FIGS. 10A-10E. Left panel of (A) and (C) shows binding activity of Bi-specific VHH-Fc-CFH protein (SLN6074), parent VHH-Fc-CFH protein (SLN6073) and SLN6066 (Aflibercept) to hVEGFA121-Biotin and mVEGFA120-Biotin. Proteins were 5-fold serially diluted from 2 μg/ml and the binding to coated hVEGFA121-Biotin was detected by goat anti-human Fc-HRP. Right panel of (B) and (D) shows receptor blocking activity of Bi-specific VHH-Fc-CFH protein (SLN6074), parent VHH-Fc-CFH protein (SLN6073) and SLN6066 (Aflibercept). 50 μl hVEGFA121-biotin (2 μg/ml) and 50 μl 5-fold serially diluted proteins (5 μg/ml) were mixed and its binding to coated 5 μg/ml fusion proteins was detected by SA-HRP. (E) shows the inhibition of hemolysis. SLN6074 (VHH-Fc-CFH) demonstrated favorable binding to human and mouse VEGFA and also comparable activities to that of VHH-Fc regarding blocking the VEGFR-VEGF interaction, as assessed with the purified recombinant proteins by following procedures as described above. Complement inhibition activity was determined by following a standard procedure for measuring alternative pathway functions on red blood cells (hemolysis assay). SLN6074 (VHH-Fc-CFH) has comparable hemolysis inhibition to SLN7112 (Fc-CFH). Such dual functional protein provides a potential therapeutical strategy for AMD or tumor.

### Example 2

### Example 2.1 Establishment of VHH Immune Library

Human transferrin (human transferrin, Sigma T3309) was used to immunize two healthy and age-appropriate camels for 4 times. After the antibody tilter was qualified, 200 ml of peripheral blood was taken from each, lymphocytes were separated, and total RNA was extracted. Reverse transcription was applied to create cDNA for construction of a phage-displayed camel immune VHH antibody library. To check the quality of the VHH antibody library, 24 independent phage clones were subjected to DNA sequencing. The data indicated that 22 out of 24 clones contain standard VHH sequences fused to gp3 protein that was used for phage display, and each of the 22 clones had a unique VHH sequence with a CDR3 of 9 or more amino acids in length. This library was used for subsequent antibody discovery.

### Example 2.2 VHH Discovery

### 2.1-Panning

The phage library was taken out from −80° C. freezer, incubated at 37° C. for 10 min, and centrifuged at 5000 rpm for 5 min. 10¹²˜10¹³ CFU phages (input) were transferred to 1 mL of PBS solution with 1% BSA, 10 μg of biotinylated human or mouse transferrin protein in a microtube. The mixture was incubated at RT for 1 h before 100 μL suspension of streptavidin-coated magnetic Dyna-beads were added. After incubation at RT for 30 min on a rotating tumbler, the tube was placed on a magnetic rack for 30s to remove the solution mixture. The beads were washed with 1 mL of PBST (0.05% Tween 20 in PBS, pH7.4) for 10 times before a final wash with PBS.

Phages were eluted off the beads with 1 mL of 10 μg/mL trypsin in PBS by incubating at 37° C. for 30 min and separated from the beads on a magnetic rack. The supernatant (output) were transferred to a tube containing 4 mL TG1 (A600≈0.6) to infect the bacteria. After incubation at 37° C. for 30 min, the infected bacteria were collected by centrifugation at 4,500 rpm for 10 min at 4°. The pellet was resuspended in 500 μL and spread onto 25×25 cm 2×YT/Amp/Glu plate to amplify the phage-infected bacteria. After being incubated overnight at 37° C., the bacteria were scraped from the plate by adding 30 mL of 2×YT medium, and collected by centrifugation at 4,500 rpm for 10 min. The pellet was resuspended by adding 2 mL 2×YT medium containing 40% glycerol to have stocks at −80° C., or directly prepare phages (as below in phage preparation or packaging) for the next round of panning as needed.

Input and output phages were also titrated to determine phage enrichment efficiency of the panning process. Briefly, serial dilutions in PBS (10-fold, generally 10⁻¹˜10⁻⁹) of the input/output phages were prepared, 10 μL of the serially diluted phages was mixed with 90 μL of log-phase TG1 in a new tube before incubation at 37° C. for 30 min. Thereafter, 10 μL of the mixture were spotted onto pre-warmed 2×YT/Amp plate. The lid was left open to evaporate solution before the bacteria plates were cultured at 37° C. O/N. Colonies were counted and titer was calculated for input and output phages respectively, ratio of the output/input from each round of panning were used to determine efficiency of the panning process.

### 2.2 Phage Preparation or Packaging

The phage-infected TG1 bacteria were inoculated with 2×YT/Amp/Glu medium at 37° C. with shaking (250 rpm) to reach an A600 of 0.6 (approximately 1-2 h). Helper phage M13K07 were added at a phage: bacteria ratio of 1000. The culture was incubated at 37° C. without shaking for 30 min, followed by 30 min with shaking at 180 rpm.

The bacterial cultures were centrifuged in sterilized centrifuge bottles at 4,500 rpm for 10 min to recover the bacterial pellets. The bacterial pellets were resuspended in 100 mL 2×YT/Amp/Kan (Amp: 100 μg/mL, Kan: 50 μg/mL) and transferred into a 250 mL flask for incubation with shaking (250 rpm) for 4 h at 37° C.

The culture was centrifuged at 4,500 rpm for 10 min to collect supernatant. Phages were precipitated from the supernatant by adding ¼ volume of PEG solution (20% Polyethylene glycol 6000, 2.5 M NaCl) and incubation on ice overnight. After being collected by centrifugation at 4,500 rpm for 30 min at 4° C., the phage pellets were resuspended in 5 mL of PBS and incubated with shaking at 37° C. for 30 min. Insoluble debris was removed by centrifugation at 4,500 rpm for 10 min, and the soluble phages in supernatant were transferred to a new tube to repeat PEG precipitation once. The final phage solution in PBS was used for the next round of panning or mixed with glycerol for storage at −80° C.

### 2.3 Phage-Based ELISA Screening

Single colonies from panning outputs were individually picked from agar plates and inoculated with 2×YT/GA medium (2% Glucose, 100 μg/mL Ampicillin) in 96-well deep well plates at 37° C. overnight, with a shaking speed at 320 rpm. 10 μL of the overnight culture were transferred to 400 μL of 2YT/GA medium in a new deep-well plate. The culture grew at 37° C. with shaking (800 rpm) to an A600 of 0.6 (approximately 1 h), before helper phage M13K07 (4.2×10¹³ CFU/mL) was added at a phage: bacteria ratio of 1000 (100 μL in 100 mL). The culture were kept incubation at 37° C. without shaking for 30 min, followed by 30 min with shaking at 180 rpm. The bacterial cultures were centrifuged at 4,000 rpm for 30 min to discard the supernatant. The bacterial pellets were resuspended in 400 μL 2×YT/AK (Amp: 100 μg/mL, Kan: 50 μg/mL), and cultured with shaking (320 rpm) at 30° C. overnight. The bacterial cultures were centrifuged at 4,000 rpm for 30 min to collect the supernatant. The supernatant was used as phage solution for ELISA screening.

To conduct ELISA screening, 96-well microtiter plates were coated with 100 μL per well of Streptavidin at the concentration of 1 μg/mL (carbonate buffered saline or CBS at pH9.4) overnight at 4° C. Each well was blocked with 200 μL of blocking buffer (PBS pH7.4/0.05% Tween20/1% BSA) at RT for 1h. the wells were washed 3 times with PBST (PBS/0.05% Tween20, pH7.4) before 100 μL of biotinylated human or mouse transferrin protein (1 μg/mL in blocking buffer) were added and incubated at RT for 1 h. The wells were washed 3 times with PBST before 100 μL of the phage solution (above) was added and incubated at RT for 1 h. The wells were washed 3 times with PBST, 100 μL of mouse anti-M13 IgG-HRP (prepared in blocking buffer) was added to each well and incubated for 1h RT. The wells were washed 3 times with PBST, 100 μL of TMB solution was added and incubated at RT for 15 min in dark. The plate was scanned at 450 nm after the reaction was terminated with 100 μL of stop solution. The positive clones with specific binding to transferrin were selected for DNA sequencing, unique VHH's were identified upon the deduced amino acid sequences.

### 2.4 Production of Recombinant VHH-OVA-his Proteins

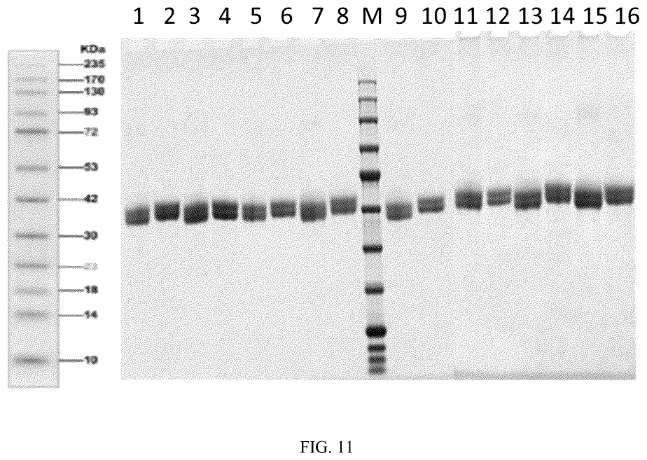

Unique VHH clones were selected for subcloning to create recombinant plasmids to produce VHH-OVA-His proteins (OVA comprises an amino acid sequence as set forth in SEQ ID NO: 145). After the plasmid sequence was verified by sequencing, small scale production of recombinant proteins was performed using transient transfection of HEK293 cells with the recombinant plasmid using lipofectamine or PEI as transfection reagent. Cultures were grown in shaking flasks media at scales ranging from 30 ml to 100 ml for 5-7 days. Cells were removed by centrifugation and culture supernatants were used for protein purification by Ni-NTA sepharose. The purified proteins were analyzed with 4-12% gradient SDS-PAGE gel under non-reducing or reducing conditions (FIG. 11).

### 2.5 Transferrin-Binding Assays

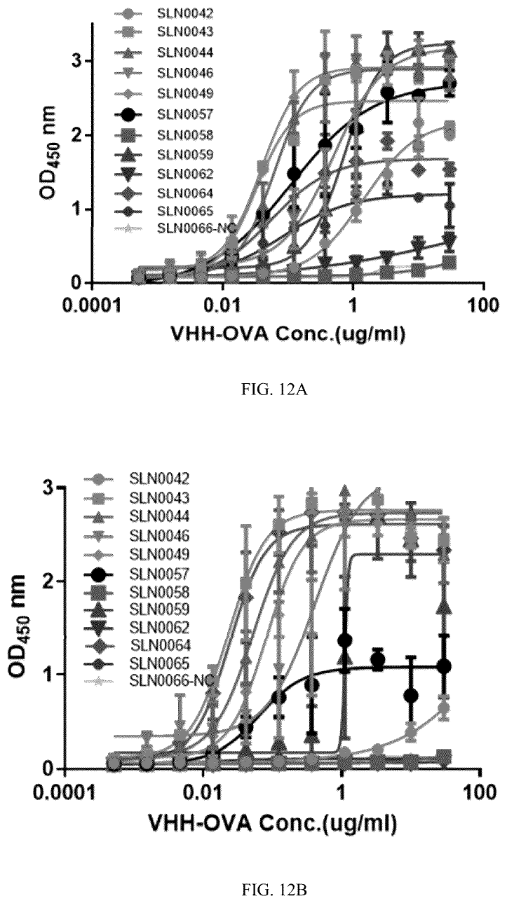

To test the recombinant proteins regarding transferrin binding capability and specificity, 96-well microplates were coated with streptavidin (1 μg/ml in CBS, 100 μl/well) and incubated at 4° C. overnight. After 3 times of washing with PBST, the plates were blocked with 1% BSA in PBST at RT for 1 hr. After 3 times of washing with PBST, biotinylated human or mouse transferrin protein (1 ug/ml in PBS, 100 ul/well) was added and incubate at RT for 1 h. After 3 times of washing with PBST, the purified VHH-OVA-His proteins were added and incubated at RT for 1 h (serially diluted in PBS either pH7.4 or pH6.0, starting from 10 μg/ml, 100 ul/well). Rabbit anti-chicken egg albumin (OVA) and a secondary anti-rabbit IgG-HRP were incubated sequentially, before the plates were washed with PBST 3 times and incubated with substrate solution and stop solution as described above for phage ELISA. Representative data were shown in FIG. 12A (pH7.4, holo-transferrin) or FIG. 12B (pH6.0, apo-transferrin) to illustrate binding activities of the recombinant VHH-OVA-His to the native human transferrin (Sigma).

2.6 VHH-Binding Did not Disturb Transferrin/TfR1 Interaction on HEK293F-hTFR1 cells

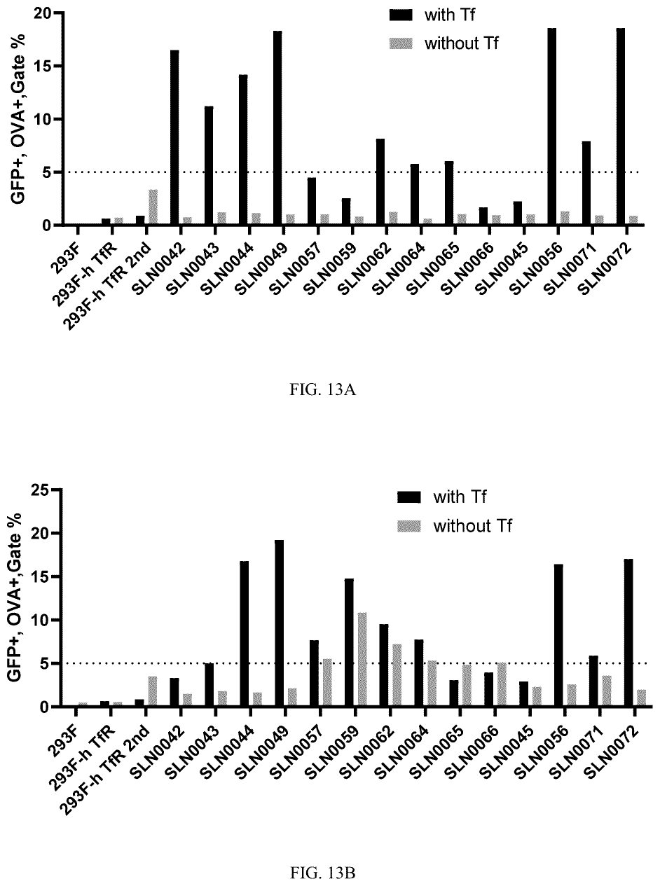

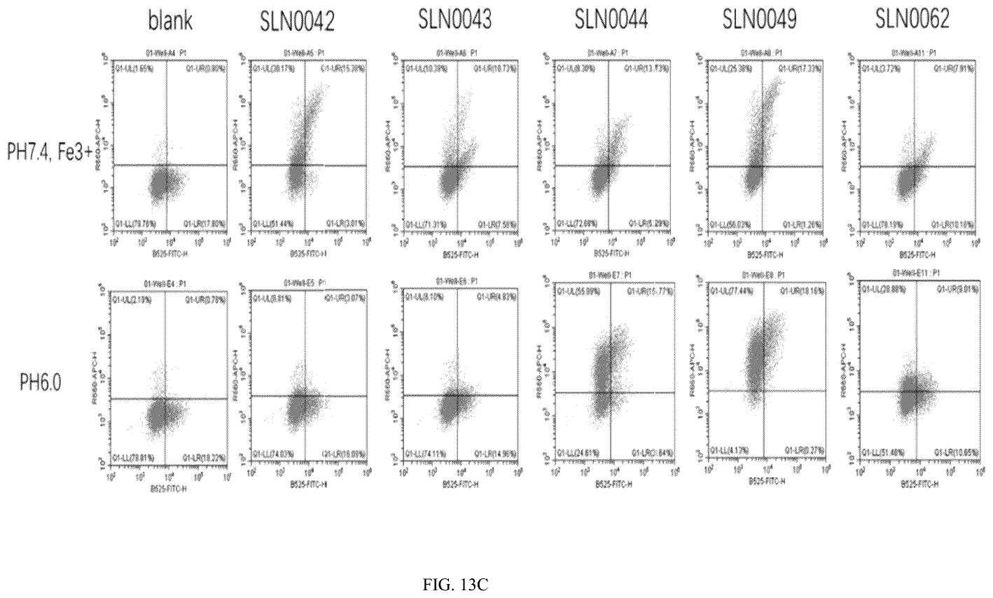

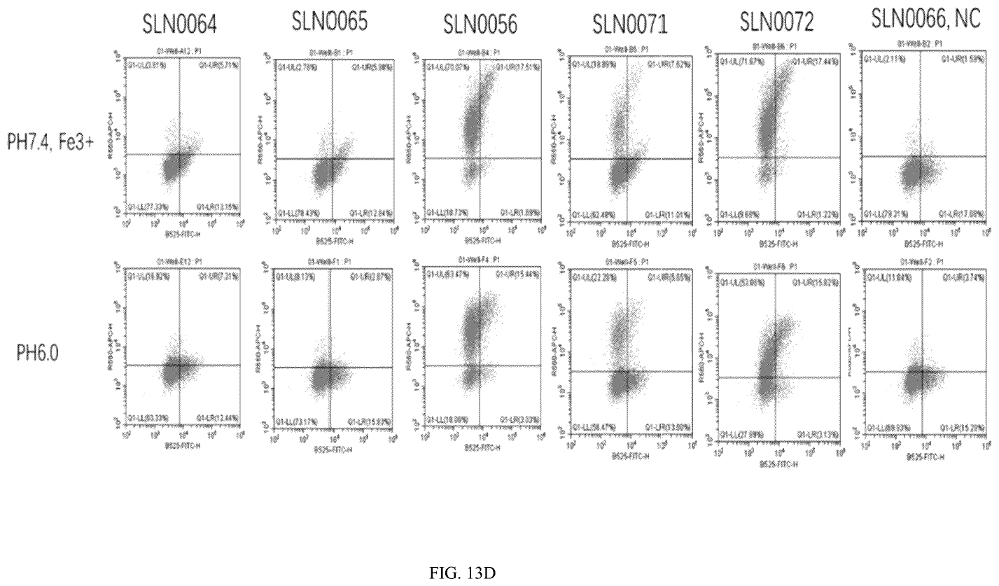

HEK293F-hTfR1 cells were prepared by transfecting hTfR1-expressing plasmid, with a GFP tag, into HEK293F cells. Stable pool was used for this assay. Briefly, the engineered HEK293F cells expressing hTfR1 were cultured with OPM-293 CD05 medium at 37° C., with 8% CO₂ and saturated humidity in cell shaker. After the HEK293F-hTfR1 cells were harvested, counted and blocked with blocking buffer at 37° C. for 30 min, 1×10⁶ of cells were used per reaction and mixed with VHH-OVA-His or a control protein solutions (final concentration at 10 μg/mL), with or without transferrin (final 5 μg/mL), with or without ironic citrate solution (final 0.2 mg/mL), at pH7.4 or pH6.0. The cells were washed twice by centrifugation at 300 g for 5 min at 4° C., before rabbit anti-OVA antibodies and a secondary anti-rabbit IgG-AF647 (all prepared in blocking buffer). The cells were scanned with CytoFLEX (Beckman). Data were analyzed using CytoExpert 2.4 software to determine 1) differential binding of VHH-OVA-His proteins to cells (transferrin-binding does not interfere transferrin/TfR1 interaction on the cell surface); 2) dependency on transferrin (specificity to transferrin); 3) dependency on TfR1 (specificity to TfR1-mediated cell binding); 4) dependency on pH value and Fe3+ (specificity to iron-associated holo-transferrin or iron-free apo-transferrin). The data (below and FIGS. 13A-13D) indicated that the selected transferrin-binding VHH's such as SLN0042, SLN0043, SLN0044, SLN0049, SLN0062, SLN0064, SLN0065, SLN0056, SLN0071 and SLN0072 did not interfere transferrin/TfR1 interaction and could be associated to TfR1 on cell surface only through binding to transferrin. The data also confirmed results from ELISA that showed that some selected VHH's had differential affinity to holo- and apo-transferrin.

TABLE 6
Summary of VHH/Tf binding profiles on different PH
conditions on hTfR1 overexpressed cell surface.

| | | | Non-Specific |
|---|---|---|---|
| VHH Variants | pH 7.4 | pH 6.0 | binding (pH 6.0) |
| SLN0042 | +++ | − | − |
| SLN0043 | ++ | − | − |
| SLN0044 | ++ | +++ | − |
| SLN0049 | +++ | +++ | − |
| SLN0057 | − | + | + |
| SLN0059 | − | + | + |
| SLN0062 | + | + | + |
| SLN0064 | + | + | + |
| SLN0065 | + | + | − |
| SLN0066 | − | − | − |
| SLN0045 | − | − | − |
| SLN0056 | +++ | +++ | − |
| SLN0071 | + | + | − |
| SLN0072 | +++ | +++ | − |

Note:
+++ ≥ 15%;
15% > ++ ≥ 10%;
10% > + ≥ 5%;
− < 5% Binding

### 2.7 Epitope Binning

Epitope binning was used to distinguish different “bin” with different proteins. Because the antibodies in different “bins” bind to different epitopes and show different functional characteristics.

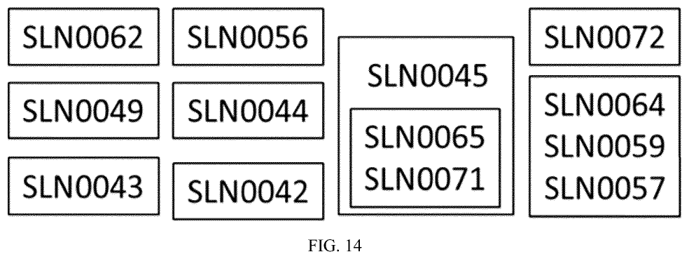

To conduct epitope binning, 96-well microplates were coated with 5 μg/mL of each VHH-OVA-His recombinant protein respectively (in CBS buffer, 100 μL/well) and incubated at 4° C. overnight. After washing and blocking as described above for ELISA, cross check board reactions were conducted by adding mixture of biotinylated human transferrin (final 1 μg/mL) and VHH-OVA-His protein (final 5 μg/mL) and incubated at RT for 1 h. The biotinylated transferrin binding to the VHH-OVA-His that were used for coating was detected with streptavidin-HRP and corresponding method. The data shown in FIG. 14 indicated that the selected VHH's belong to multiple bins, particularly different from reference antibodies (VHH1, VHH2, VHH3).

### 2.8 Transcytosis of VHH-OVA-his Proteins Across Monolayer of Caco-2 Cells

Caco-2 cells were seeded in 96-well trans-well plate at 2×10⁴ per well and cultured for 12 days to allow formation of cellular monolayer in inner chamber (medium was changed in every other days during the first week and every day during the second week). On day 13, the monolayer cells were washed with prewarmed PBS and then incubated in MEM medium with 0.1% BSA without FBS at 37° C. for 30 min to remove the endogenous Tf. Recombinant VHH-OVA-His proteins (10 μg/mL) with or without native human transferrin (5 μg/mL), both prepared in MEM medium (pH 7.4) with 0.1% BSA were added into the inner chamber (above the monolayer of Caco-2 cells) and incubated for 2 h or 24 h at 37° C. Medium from the outer chamber were collected at the indicated time points for quantitative measurement with ELISA method, by referring standard curve developed by using the corresponding VHH-OVA-His proteins as control. Integrity of the Caco-2 cell monolayer was tested with Lucifer yellow (LY) rejection assay according to the manufacturer's instructions. Wells with more than 0.1% (basal level of passive crossing) of lucifer yellow signal from outer chamber signals compared to that of inner chamber would be considered as imperfect cellular monolayer for the measurement and thus discarded.

Quantitative ELISA measurement was conducted as procedures described above by using specific antibodies. Briefly, microplates were coated with rabbit anti-OVA polyclonal antibodies (5 μg/mL in CBS buffer), test articles or control (VHH-OVA-His protein) for standard curve were added, and detected with biotinylated anti-OVA antibodies and subsequent streptavidin-HRP conjugates. Standard curve was prepared by using recombinant VHH-OVA-His proteins serially diluted from 1000 ng/mL to 0 with a dilution factor of 3. The assay was qualified with inter-assay CV's less than 20% and recovery rate more than 90%.

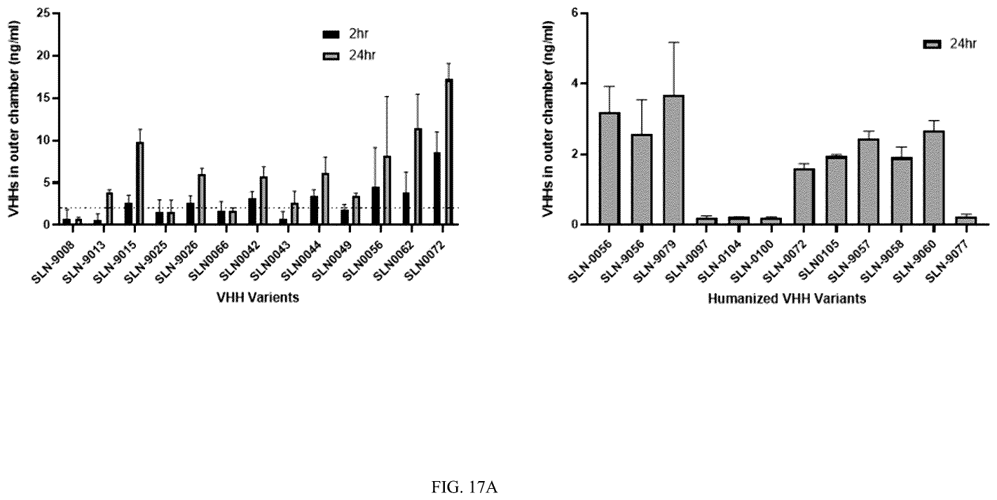

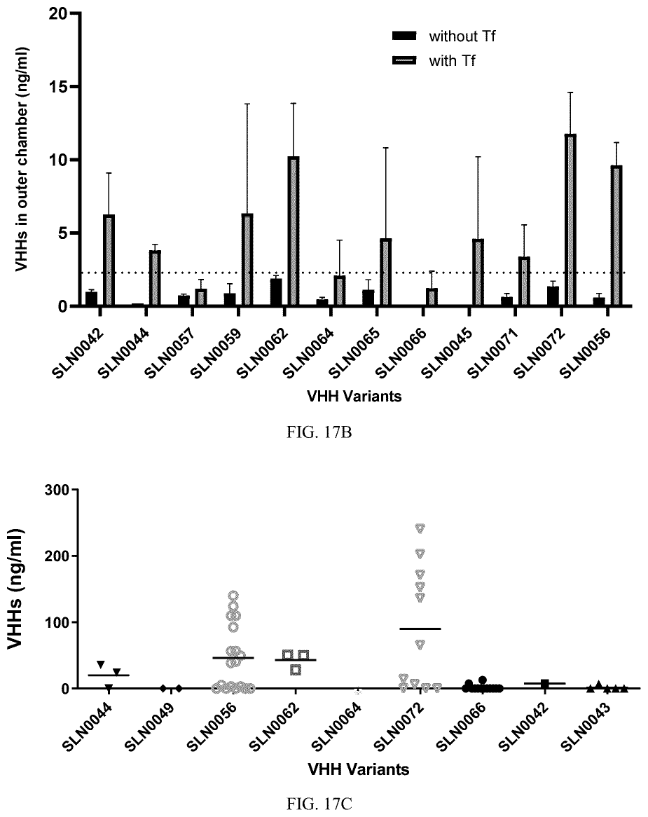

Data shown in FIGS. 17A-17C suggested that VHHs possess variable capabilities to cross the monolayer of Caco-2 cells, partially reflecting its affinity to human transferrin or epitope on transferrin for binding. The results also suggested essential roles of transferrin in the VHH-enabled transmembrane absorption of biological molecules through intestinal epithelium (FIG. 17B).

### Humanization of the Selected VHH's

VHH's with the expected properties in enabling transmembrane translocation of the fused OVA protein, as well as with less risk factors for development, were selected for humanization. CDR residues within the selected VHH's were determined and annotated with Kabat numbering system. N-glycosylation site, and deamination site within the CDRs, N-glycosylation sites or cysteine within or close to CDR will be regarded as risk factors for future development. VHH humanization was conducted by following standard procedures of CDR grafting and structural refinement. Upon the humanization design, recombinant DNA constructs were created to produce recombinant VHH-OVA-His as described above for ELISA- and FACS-based binding assays. The humanized sequences with an affinity equal to or better than that of the original VHH to transferrin, while having acceptable expression and stability levels were selected for further development.

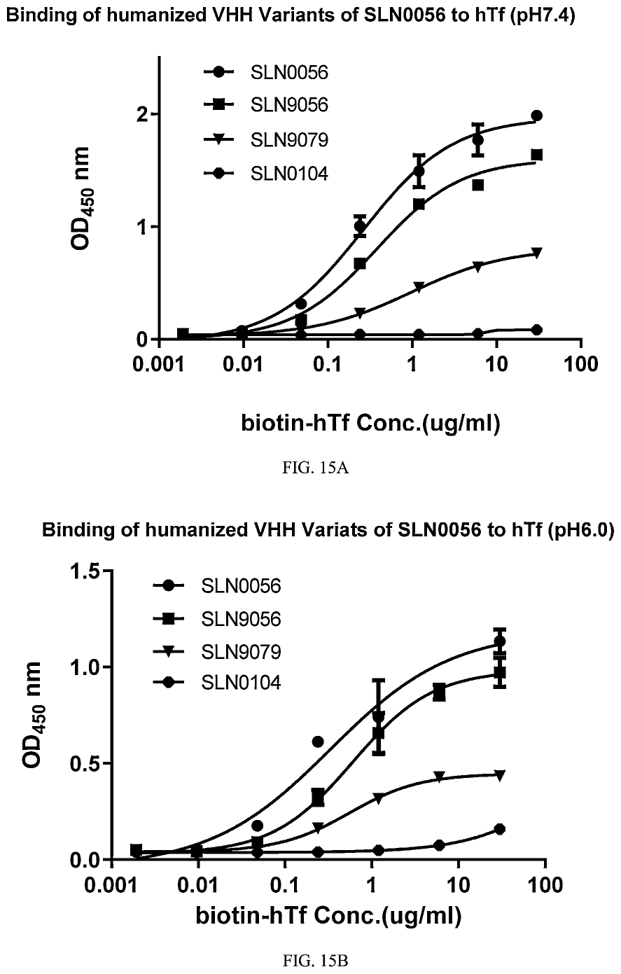

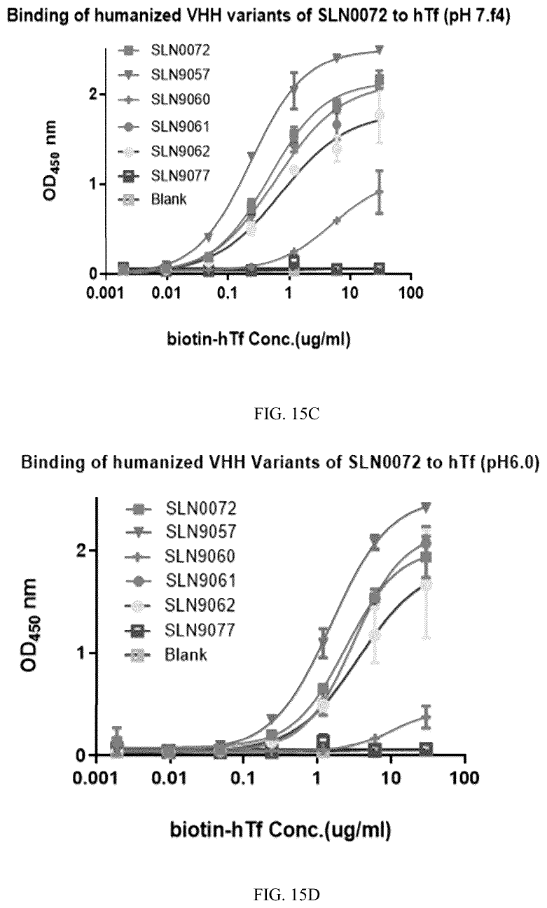

FIGS. 15A-15D indicated that the humanized VHH variants possess similar profile as the original VHH regarding its binding to transferrin.

### Example 2.3 Using Tf-Binding VHH for Half-Life Extension of its Associated Entities

Fusion proteins were produced with recombinant DNA technology by fusing the indicated protein at C- or N-terminus of VHH, with or without linkers. The proteins were purified to homogeneity, formulated in appropriate buffer, and quantitated with Bradford methods for use in in vivo tests. All animal procedures were reviewed and approved by institutional review board (IRB).

Mice of different genetic strains including C57BL/6, Balb/c and CR6 were grouped upon genetic background, gender and body weight. The test articles such as proteins fused to the transferrin-binding protein, as well as a negative control (a recombinant protein produced in a same way but containing a VHH that does not bind to transferrin) were administered through IV or SC injection at a dosage of 3 mg/kg, with or without co-dosing of human transferrin (5 mg/kg). For the groups in which transferrin was co-dosed, daily dosing of human transferrin at 5 mg/kg were continued for 10 days.

To determine serum half-life of the test articles in mice, 40 μL of whole blood was collected from the subcutaneous vein of the outer corner of the eye at multiple time points. Serum was recovered by centrifugation at 10000 rpm for 10 min after the collected whole blood was placed at room temperature for 1 hour, concentrations of VHH-fused proteins within serum were measured by quantitative ELISA. For OVA-VHH-His or GLP-1-VHH-His proteins, rabbit anti-OVA polyclonal antibodies (Sigma C6534-2 mL) or a monoclonal anti-GLP-1 antibody, respectively, was paired with a biotinylated rabbit anti-VHH antibody (GeneScript, A02015-200) for specific and quantitative measurement, referring a standard curve developed with the purified recombinant protein by using a protocol as qualified with inter-assay CV's less than 20% and recovery rate more than 90%. PK parameters were calculated by PKsovler software.

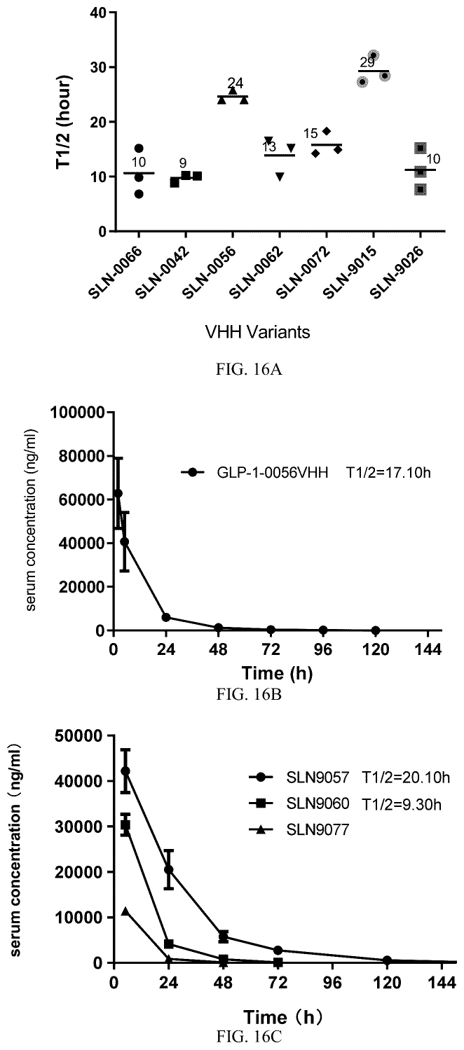

Results shown as in FIGS. 16A-16C indicated the transferrin-binding VHH enabled extension of the serum half-life of its associated OVA as well as GLP-1, and such effect was dependent on affinity of the VHH to transferrin and epitope utilized for binding.

### Example 2.4 Transferrin-Binding VHH Enabled Oral Delivery of the Associated Entities

As in half-life studies, grouped mice were put under anesthesia by using 250 mg/kg tribromoethanol, then the abdominal hair of mice was shaved and disinfected with the iodine and alcohol. The skin of the middle abdomen was cut at a length of about 0.5-1 cm by surgical scissors to expose the duodenum for injection of the test articles or a control (the purified VHH-OVA-His protein) with or without mixture of transferrin and Fe³⁺. Wound was sutured with iodine disinfection. Alternatively, the test articles or control were administered through intragastric injection by inserting gavage needle into the mouse stomach. After the oral dosing, blood samples were collected at multiple time point to recover serum as described above. Concentrations of VHH-fused proteins were quantitated by following procedure described in Example 3.

Data shown in FIGS. 17A-17C indicated that the transferrin-binding VHH enabled the orally dosed VHH-OVA fusion proteins to cross intestinal epithelium and be absorbed to blood stream, and therefore demonstrated potentials in delivering therapeutic biologics through oral dosing route.

### Example 2.5 Transferrin-Binding VHHs Enabled the Associated Entities to Cross Blood-Brain Barrier

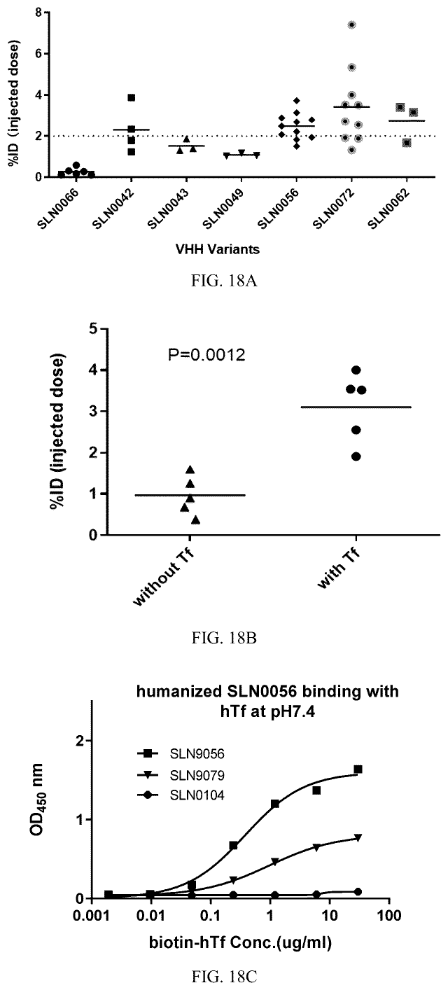

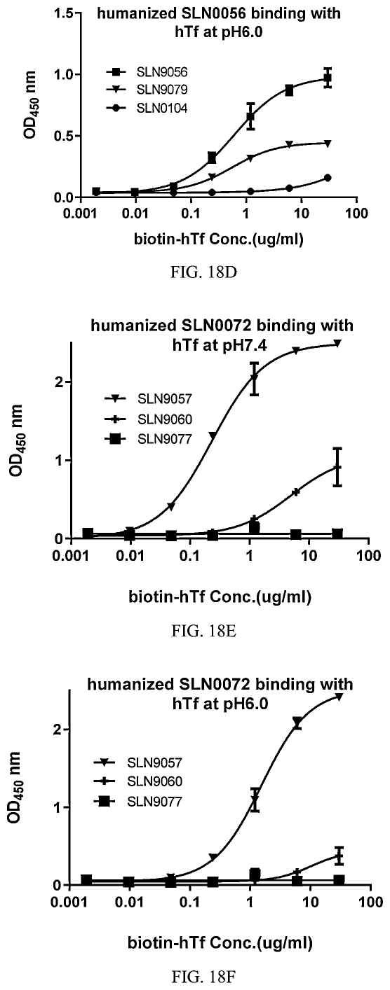

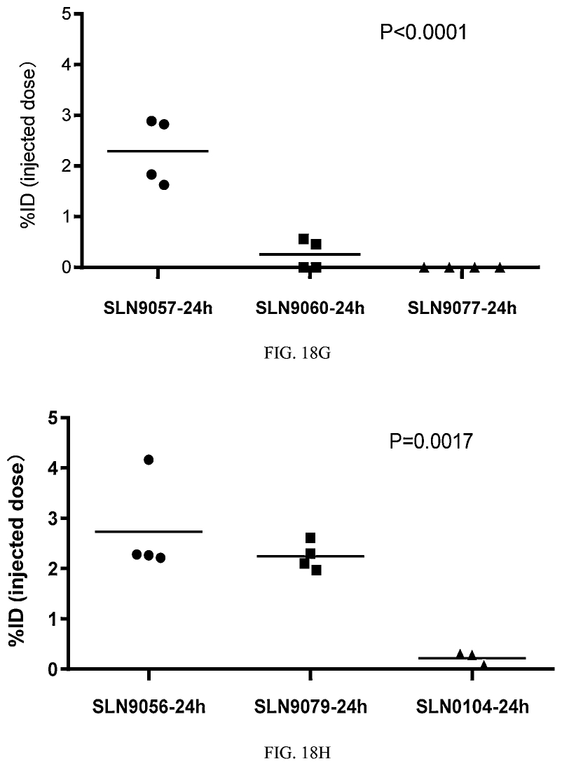

As in the half-life studies, mice were dosed with the purified recombinant proteins (e.g. VHH-OVA-His) at 10 mg/kg by IV injection, with or without 10 mg/kg of human transferrin by IP injection. At multiple time points post dosing, brains were collected and frozen in liquid nitrogen after perfusion with 20 mL of PBS. Proteins were extracted by following standard procedures for ELISA-based quantitative measurement. One quantitative ELISA format was using rabbit-anti-OVA polyclonal antibody (Sigma C6534-2 mL) for coating and biotinylated rabbit anti-VHH antibodies (GeneScript, A02015-200) for detection. Data shown in FIGS. 18A-18H indicated that, compared to the negative control (SLN0066, one VHH that does not bind to transferrin, SEQ ID NO: 148), the test articles (VHH's that demonstrated binding capabilities to transferrin) significantly elevated the levels of the test articles within brain tissue (regarding mean % ID or percentage of injection dosage, ˜1.03% to ˜3.5% at 24h after administration for the selected VHH's). the data also clearly indicated the VHH-enabled crossing BBB was associated to transferrin (groups co-dosed with transferrin had ˜3.2-fold higher % ID than that of the group treated with VHH-OVA-His only).

By using the humanized VHH variants that share same CDR's (and thus supposedly bind to the same epitope on transferrin) but with different affinities, impact of the VHH's affinity to transferrin on the capabilities to mediate the BBB-crossing was also determined. The data in FIGS. 18C-18H further supported the conclusion on dependency of transferrin binding of the VHH-enabled BBB-crossing capabilities, but also clearly suggested a relationship between VHH's affinity to transferrin and its capability to mediate the BBB-crossing. Higher BBB-crossing was observed with the VHH variants that possess the higher affinity to transferrin.

### Example 2.6 Transferrin-Binding VHH's Enabled Intracellular Delivery of its Associated Entities

Levels of transferrin receptors are also elevated on the membrane surface of multiple types of cells, for example tumor cells. Therefore, the transferrin-binding VHH's would be favorable for targeted drug delivery especially intracellular delivery to tumor cells. To test such hypothesis, recombinant VHH-PE38-His proteins were produced in E. coli and purified to homogeneity. Efficacy of intracellular delivery was tested on monolayer culture of Hepa-G2 cells (constitutively expressing TfR) and MDCK cells (with or without heterotopic TfR1 expression from transfected recombinant plasmid). Briefly, cells were seeded at a desired density into 96-well microplates within 100 μL of culture medium and cultured at 37° C. overnight. After cells were washed with fresh pre-warmed medium, serially diluted VHH-PE38-His with or without human transferrin prepared in culture medium were added to the cell culture. At multiple time points post the treatment, live cells were quantitated with CellTiter-Glo® Reagent as instructed by the manufacturer's manual. Luminescence signals were recorded at an integration time of 0.25−1 second per well and data were analyzed with GraphPad.

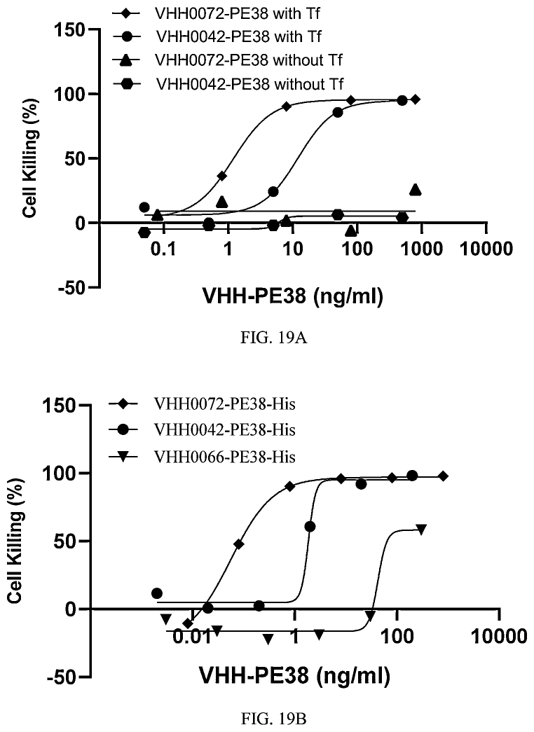

Data shown in FIGS. 19A-19B indicated that the transferrin-binding VHH's enabled intracellular delivery of the associated entities into cells that express transferrin receptor(s) on surface, and such capabilities were dependent on transferrin-binding activity of the VHH's. VHH's with different epitope utilization (belonging to different bins or having different CDR sequences) demonstrated differential efficacy in intracellular delivery as well.

### Example 2.7 the Transferrin-Binding VHH's Enabled Organ or Tissue-Targeted Delivery of the Associated Entities

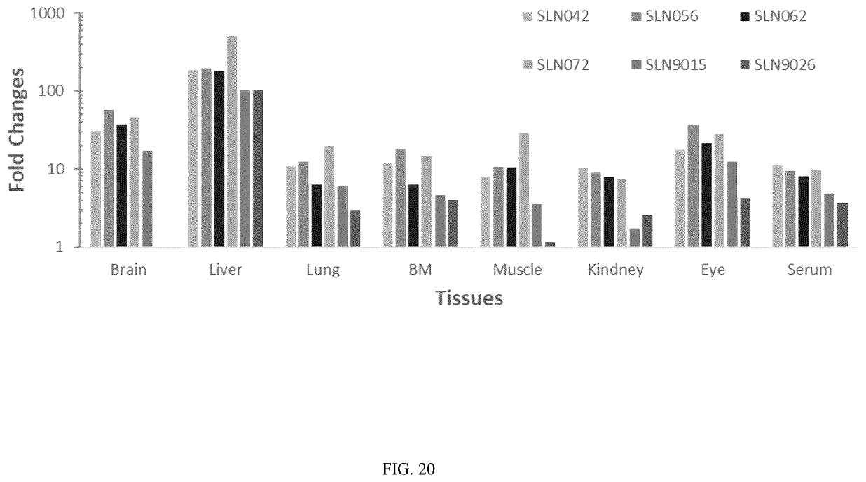

Biodistribution of the iv administered recombinant proteins (e.g VHH-OVA-His) was also investigated in mice with different genetic background. At multiple time points post iv injection, samples were collected from liver, lung, bone marrow, heart, muscle and blood et al. after blood vessels were perfused with 30 mL of PBS. Samples were weighted and homogenized to extract soluble proteins for quantitative ELISA-based measurement as described above. Referring to a standard curve developed with the purified recombinant proteins, the data were normalized upon tissue weights or volumes. Biodistribution of the VHH-OVA-His proteins was analyzed upon fold-changes compared to the control VHH that does not bind to transferrin. Data shown in FIG. 20 indicated the selected transferrin-binding VHH's enabled targeted delivery to liver, brain, bone marrow, lung, muscle, kidney, eyes and other organs or tissues which express transferrin receptor(s). The transferrin-binding VHH's in this invention may have potential applications for drug delivery targeting different tissues, especially for brain, liver and bone marrow.

### Example 3

### Example 3.1 Discovery of Properdin-Binding Antibody Fragments

### 1.1 Human, Mouse and Cyno Properdin Generation

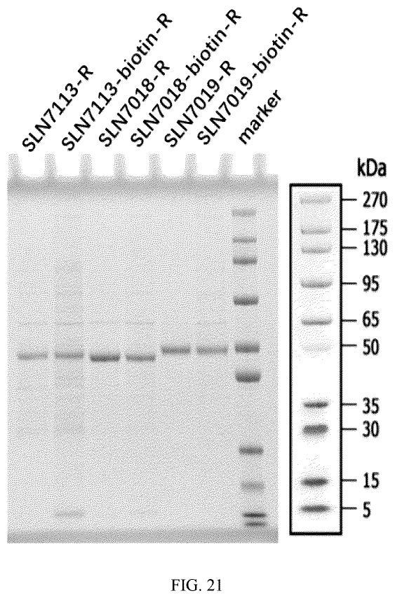

The sequence of human, mouse and cyno properdin from Uniprot database synthesized by GENEWIZ were subcloned to expression vector. Protein expression was performed using transient expression of HEK293 cells transfected using PEI (Polysciences, cat #24765-1). Cultures were grown in shaking flasks media at scales ranging from 100-200 ml for 5-7 days. Cells were removed by centrifugation and culture supernatants were used for protein purification by Ni sepharose with elution using a pH 7.4 PBS buffer. Purified proteins were analyzed with 4-12% SDS-PAGE under non-reduced and reduced conditions (FIG. 21).

### 1.2 VHH Immune Library Constructions

Immunization was performed using recombinant mouse properdin in two healthy camels. On day 60 after finishing 4 rounds of immunization, phage-displayed VHH library was constructed of PMBCs from immunized camels by following a standardized protocol. The final phage-displayed VHH library had 2.1x₁₀⁹ independent clones, with 92% of them encoding VHH-gp3 fusion proteins.

### 1.3 Phage Panning Method

As previously prepared, an immune VHH library was used for VHH selection and was subjected to four rounds of panning in 1.5 ml Eppendorf tubes. About 10¹² CFU phages were incubated with 10 μg of biotinylated properdin within 1 ml of blocking buffer (1% BSA in PBS) at RT for 1 h to make phages/target mixture. At the same time, 100 μl of streptavidin-coated Dynabeads M-280 (Invitrogen, 11206D) were washed with 1 ml of blocking buffer for five times in an Eppendorf microtube. Thereafter, the phage/target mixture was incubated with the Mag-beads prepared as above on a rotator at RT for 30 min. To recover the phages binding to the Mag-beads, the reaction tube was placed on a magnetic rack for 30 s. After the supernatant was removed, the beads were washed with PBS containing 0.5% Tween20 (0.5% PBST) for 10 times, followed by three-times by PBS. The phages were eluted with 1 ml of trypsin (10 μg/ml in PBS) at 37° C. for 30 min. After each round of selection, 100 μl of eluted phages were used to infect mid-log phase E. coli TG1 (OD₆₀₀=0.6) grown at 37° C. for phage titration. Enrichment value of properdin-specific VHHs was also assessed to monitor the progress of the selection process. Remaining eluted phages were used to infect E. coli TG1 (OD₆₀₀=0.6) for subsequent amplification. The bacteria were subsequently superinfected with M13KO7 helper phage at a ratio of 20:1 (phage: bacteria) to rescue phage particles. A mixture of kanamycin (50 μg/mL) and ampicillin (100 μg/mL) was added to the culture, and bacteria were further grown 4 h with shaking at 220 rpm at 30° C. The cultures were centrifuged at 4,000 g for 20 min, and the supernatants were added to 20% (w/v) polyethylene glycol 6000/2.5M NaCl (PEG/NaCl) to precipitate the phages. The samples were incubated on ice overnight and then centrifuged for 20 min at 8,000 g at 4° C. The pellets were resuspended in PBS, PEG precipitation was repeated once as described above. The final phage pellets were resuspended in 1 ml of PBS, and 10¹² phages were used in subsequent rounds of panning. The general panning procedure was repeated for another three rounds. The variation was the antigens derived from different species to have cross-reactive and affinity matched phage clones. Panning summary was listed in Table 7.

TABLE 7
Panning summary

| | Input | Output | Output/ |
|---|---|---|---|
| Campaign | (CFU) | (CFU) | Input |
| 1^{st} round with biotin-mouse FP | 4E12 | 4E7 | 1E−5 |
| 2^{nd} round with biotin-cyno FP | 1.8E12 | 6E8 | 3.3E−4 |
| 3^{rd} round panning with biotin- | 6E11 | 1.6E9 | 2.6E−3 |
| mouse FP and cyno FP | | | |
| 4^{th} round panning with biotin- | 4E12 | 7E9 | 1.75E−3 |
| cyno FP and mouse FP | | | |

### 1.4 Phage Screening Method

For phage-based Elisa screening, individual bacterial colonies were picked and inoculated into 200 μl 2×YT-GA medium, cultured at 37° C. with shaking (250 rpm) for 4-5 h. Then 10 μl of culture was transferred into a new deep 96-well plate containing 200 μl of 2×YT-GA medium and incubated as above till OD₆₀₀ reached around 0.6. VHH expression was induced with 1 mM IPTG (Sangon Biotech) for 16 h at 30° C. with shaking (250 rpm). After the overnight culture was spun at 4,000 rpm at 4° C. for 30 min, the supernatants were collected for phage ELISA.

Following four rounds of panning, output from 2^{nd}, 3^{rd} and 4^{th} round was screened by phage enzyme-linked immunosorbent assay (ELISA). Wells of MaxiSorp 96-well plates were coated with 1 μg/ml streptavidin in coating buffer overnight at 4° C. An equivalent concentration of BSA was used as a control for nonspecific binding. After washing with PBST, the remaining protein-binding sites in the wells were blocked for 1 h at 37° C. with 1% BSA. The blocking reagent was discarded and washed by 0.05% PBST for three times. The 5 μg/ml biotin-human/mouse/cyno properdin were added in the wells for 1 h at 37° C. The supernatant was discarded, and washed for three times with 0.05% PBST. 100 μl supernatants prepared above were added to appropriate wells and incubated while shaking for 1 h at 37° C. The supernatant was discarded, and nonspecific phages were eliminated by washing three times with 0.05% PBST. Detection of the interaction between antigen and the phage-VHH was performed using a 5000-fold diluted solution of anti-Myc-HRP (Abcam, ab62928). After incubation for 1 h at 37° C., plates were then washed as before and 100 μl of TMB substrate solution were added and incubated at RT for 15 min. 100 μl/well of stop solution were added to stop the reaction before the plates were scanned with a microplate reader at 450 nm. ELISA-positive clones were defined as those that exhibited at least three times stronger ELISA signals on antigen coated plates in comparison to signals on BSA-coated plates. In parallel, the genetic diversity of the ELISA positive clones was determined using DNA sequencing, and phages with different amino acid sequences of VHH were considered as unique clones. In total, 76 unique clones with different CDR sequences were identified as positive in target-binding assays with phage ELISA and 37 selected expression and purification in HEK293 cells.

TABLE 8
Screening summary

| Plate | | | Functional | Unique | New |
|---|---|---|---|---|---|
| ID | Panning output | Sequenced | seq | seq | seq |
| P1P2 | 2^{nd} round output | 74 | 57 | 13 | 13 |
| P3P4 | 4^{th} round output | 44 | 6 | 5 | 2 |
| P5 | 4^{th} round output | 18 | 16 | 7 | 0 |
| P6 | 4^{th} round output | 24 | 18 | 7 | 0 |
| P7 | 2^{nd} round output | 64 | 64 | 9 | 4 |
| P8 | 2^{nd} round output | 29 | 28 | 7 | 2 |
| P9 | 4^{th} round output | 54 | 52 | 6 | 0 |
| P10 | 4^{th} round output | 37 | 37 | 7 | 0 |
| P11 | 1^{st} round output | 60 | 54 | 7 | 23 |
| P12 | 2^{nd} round output | 57 | 50 | 7 | 19 |
| P13 | 1^{st} round output | 37 | 30 | 6 | 10 |
| P14 | 2^{nd} round output | 38 | 34 | 10 | 3 |
| Total | | 536 | 446 | 91 | 76 |

The properdin-binding VHH sequences were as follows:
>SLN7150 (SEQ ID NO: 208); SLN12066 (SEQ ID NO: 209); SLN12067 (SEQ ID NO: 210);
>SLN12068 (SEQ ID NO: 211); SLN12069 (SEQ ID NO: 212); SLN12070 (SEQ ID NO: 213);
>SLN7160 (SEQ ID NO: 214); SLN12075 (SEQ ID NO: 215); SLN12076 (SEQ ID NO: 216);
>SLN12077 (SEQ ID NO: 217); SLN7162 (SEQ ID NO: 218); SLN12078 (SEQ ID NO: 219);
>SLN12079 (SEQ ID NO: 220); SLN12080 (SEQ ID NO: 221); SLN12081 (SEQ ID NO: 222);
>SLN12036 (SEQ ID NO: 223); SLN12082 (SEQ ID NO: 224); SLN12083 (SEQ ID NO: 225);
>SLN12084 (SEQ ID NO: 226); SLN12085 (SEQ ID NO: 227); SLN12086 (SEQ ID NO: 228);
>SLN7151 (SEQ ID NO: 231); SLN7152 (SEQ ID NO: 232); SLN7153 (SEQ ID NO: 233);
>SLN7154 (SEQ ID NO: 234); SLN7155 (SEQ ID NO: 235); SLN7156 (SEQ ID NO: 236);
>SLN7159 (SEQ ID NO: 237); SLN7161 (SEQ ID NO: 238); SLN12027 (SEQ ID NO: 239);
>SLN12030 (SEQ ID NO: 240); SLN12039 (SEQ ID NO: 241); SLN12041 (SEQ ID NO: 242);
>SLN12042 (SEQ ID NO: 243); SLN12044 (SEQ ID NO: 244); SLN12045 (SEQ ID NO: 245).

### Example 3.2 Identification of Properdin-Binding Antibodies with Inhibitory Effects on Complement Activation Through Alternative Pathway

### 2.1 Expression and Purification of VHHs-FC

Unique VHH clones were selected for subcloning to create recombinant plasmids to produce VHH-FC proteins, degenerated primers (Forward: SEQ ID NO: 259, Reverse: SEQ ID NO: 260) were used. After the DNA sequences were verified with DNA sequencing, the recombinant plasmids were prepared and fusion protein expressed and purified by following standard protocols.

To express the recombinant VHH-Fc proteins, 100 ml of Expi293F™ Cells in OPM-CD05 Medium (OPM, cat #81075-001) were cultured to reach a cell density of approximately 3×10⁶ viable cells/ml with viability more than 95%. Plasmids were diluted with OPM-CD05 Medium to a concentration of 1.5 μg/ml in a total volume of 5 ml. Transfection reagent PEI was diluted with OPM-CD05 Medium to a same volume of 5 ml to have a DNA:PEI ratio as 1:4 (m/m) when the diluted DNA and PEI were mixed together. After being incubated at RT for 15 minutes, the DNA/PEI complex were added onto the prepared Expi293F™ cells by swirling gently. Then the cells cultures were placed in a 37° C. incubator with >80% relative humidity and 5% CO₂ on an orbital shake. At 24 h post the transfection, 5% peptone (1 mg/ml) and 2% glucose (330 g/l) were added to the culture slowly. After days of culturing, the cell culture supernatant was collected by sequential centrifugations at 1,200 rpm for 10 min and 3,900 rpm for 20 min before being used for Protein A purification.

VHH-Fcs were purified with Protein A (BIOON, HZ1011-2). 1 ml of Protein A slurry were loaded onto a 20-ml column (G-bios, C006197-0025). After the columns were equilibrated with PBS of 10-fold of CV (column volume), the cell culture supernatant prepared as above were loaded and flow throw the Protein-A columns by gravity for 2 times. After the columns were washed with PBS for 10 times of CV, 10 ml of 0.1 M Glycine-HCl buffer (pH 3.0) were used to elute the VHH-Fc proteins. The eluted proteins were neutralized with 100 μl of 1 M (pH 8.5) Tris-HCL buffer the pH was adjusted to 7.4. The Protein A affinity column was regenerated and preserved by washing with PBS, ddH₂O and 20% ethanol sequentially. For the eluted protein, it was desalted through an Amicon UltraCel 30K centrifugal device (Milipore, UFC903016). Briefly, eluted protein was diluted in 10 ml PBS and concentrated to 1.5 ml by centrifugation for 3 times at least. The final protein solution was formulated in PBS to less than 1 ml and filtrated with 0.22-μm filters.

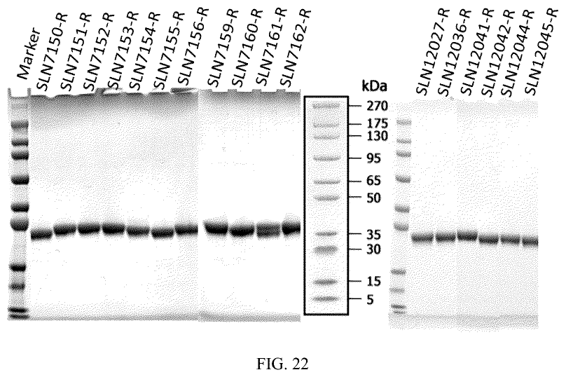

Purity of VHH-Fcs were analyzed with SDS-PAGE. Briefly, 2 ag protein in 4×LDS Sample buffer was loaded and analyzed with SurePAGE gel in Tris-MOPS SDS buffer (Genscript, M00138) at a constant voltage of 160-V for 50 min. Proteins were visualized with Coomassie stain (TIANGEN, cat #PA101) following the manufacturer's instructions. The purified proteins were analyzed with 4-12% gradient SDS-PAGE gel under non-reducing or reducing conditions (FIG. 22).

### 2.2 Elisa Binding

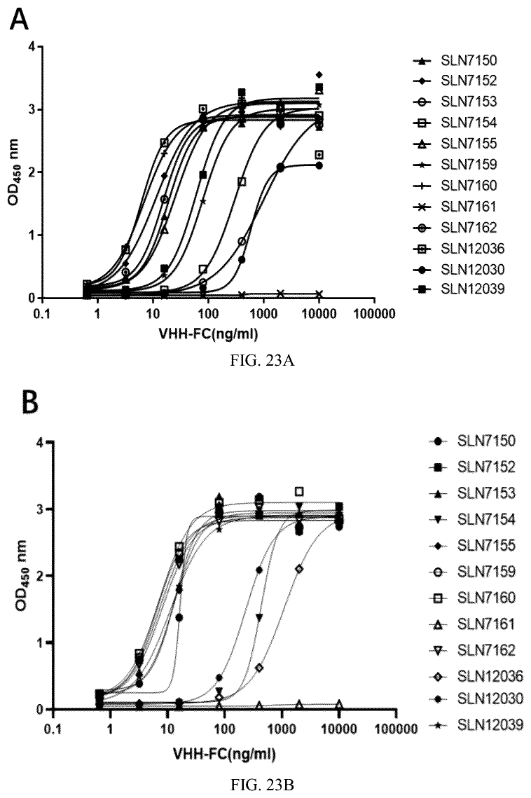

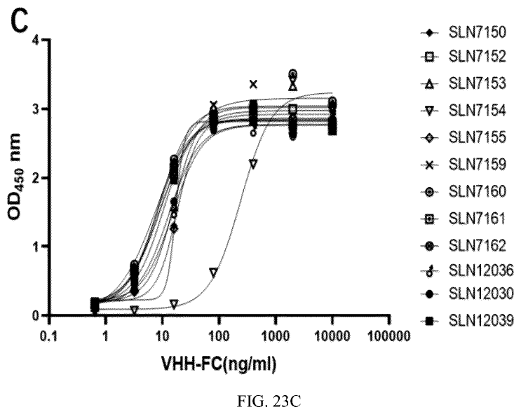

For binding ELISA, 96-well immunoplates were coated with 100 al/well 1 μg/ml streptavidin and incubate at 4° C. overnight. Wells were washed with PBST for 3 times and blocked with 200 al of 1% BSA/PBS at RT for 1 h. Washed with PBST for 3 times and add human properdin-biotin, mouse properdin-biotin or cyno properdin-biotin (5 μg/ml) 100 al/well and incubated at RT for 1 h. Plates were washed with PBST for 3 times, 100 μl/well 5-fold serially diluted VHH-Fcs from 10 μg/ml was added. and incubate at RT for 1 h. Plates were washed with PBST for 3 times and add 100 μl goat anti-human Fc-HRP (Sigma, A0170) diluted 1/5000 in 1% BSA/PBST to each well and incubate at RT for 1 h. Plates were then washed as before and add 100 μl TMB substrate and incubate at RT for 15 min. 100 μl per well stop solution was added to stop the reaction, and the plates were read with microplate reader at 450 nm. 37 recombinant VHH-Fc clones were showed human properdin binding activity (FIG. 23A), cyno properdin binding activity (FIG. 23B) and mouse properdin binding activity (FIG. 23C), all of the clones showed cross binding and affinity matched activity.

### 2.3 AP Activity

For human alternative pathway experiments, all test samples were diluted by PBS and added in duplicate (50 μl/well) to a U-bottom 96-well microtiter plate. At the same time, human complement-preserved serum (Quidel, A113) was diluted to 20% vol/vol in GVBS-EGTA (1×AP buffer: 0.1% gelatin, 145 mM NaCl, 2.5 mM sodium barbital, pH7.4 with 10 mM Mg/EGTA), incubate on ice for 30 minutes and added (50 μl/well) to the rows of the same 96-well plate such that the final concentration of human serum in each well was 10%. Then prepare the rabbit erythrocytes (4×10⁸/ml) were washed three times with 1 ml of 1×AP buffer and resuspended to a final concentration of 5×10⁷/ml (6 ml) in 1×AP buffer. After that, 50 μl aliquots of rabbit erythrocytes (2.5×10⁶ cells) were added to the plate as described above, mixed well, and incubated at 37° C. for 30 min. Each plate contained two wells of 50 μl of identically prepared rabbit erythrocytes, incubated with 50 μl PBS+50 μl 1×AP buffer alone (negative control) as a control for spontaneous hemolysis, two wells containing 100 μl ddH₂O serving as a control for 100% lysis and two wells containing 10 mM EDTA (Thermo 15575-038) as a serum blank control. After incubating, the plate was then centrifuged at 600 rpm for 2 min and 100 μl of the supernatant transferred to a new flat bottom 96-well plate. Hemoglobin release was determined at OD 405 nm using a microplate reader, and the percent hemolysis was determined using the following formula:

$$Percent hemolysis ( \% ) : 100 \times ( OD sample - OD of EDTA ) / \\ ( OD 100 \% lysis - OD negative control )$$

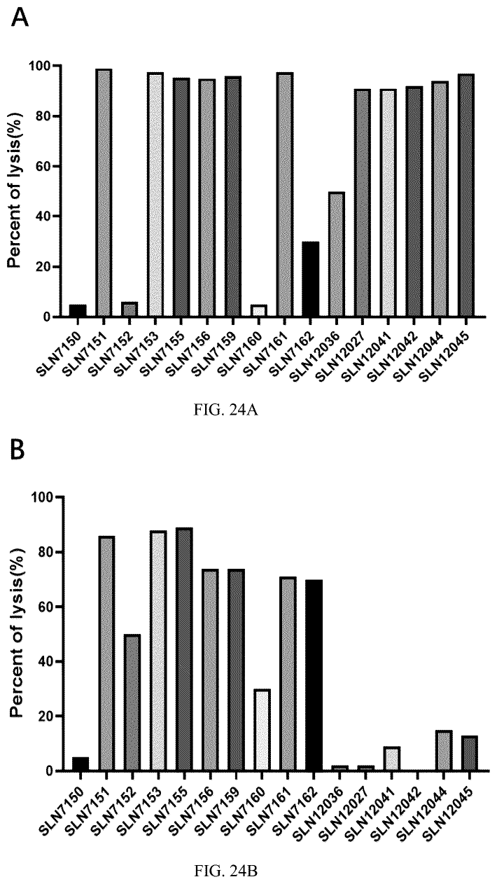

For mouse alternative pathway experiments, the process is basically the same as the above process, the difference is that the final concentration of mouse serum is 30% and that of human serum is 10%, and the incubation time is replaced by 30 min for 1 h in mouse alternative pathway assay (FIGS. 24A-24B)). Some clones showed complement inhibitory activity in human (FIG. 24A) and mouse (FIG. 24B) serum at 500 nM.

### Example 3.3 Bi-Paratopic Engineering of Properdin-Binding Antibody Fragments

3.1 Epitope Binning Assay with Full Length of Human/Mouse Properdin

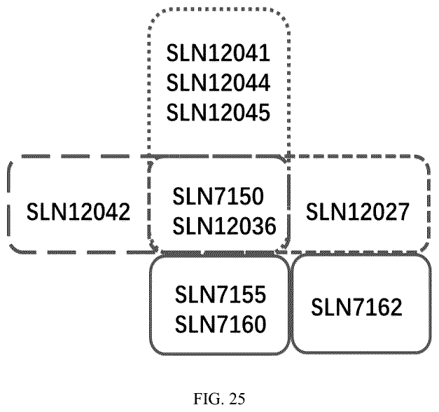

96 well immunoplates were coated with 100 μl/well of 5 μg/ml VHH Fc fusion protein and incubate at 4° C. overnight. Wells were washed with PBST for 3 times and blocked with 200 μl of 1% BSA/PBS at RT for 1 h. 60 μl human properdin-biotin or mouse properdin-biotin (0.5 μg/ml) and 60 μl VHH-Fc fusion protein (20 μg/ml) were pre-mixed and transfer 100 μl to each well that had been coated with VHH-Fe and blocked with BSA, and continued incubation at RT for 1 h. Plates were washed with PBST for 3 times and add 100 μl SA-HRP (Sigma, S5512) diluted 1/5000 in 1% BSA/PBST to each well and incubate at RT for 1 h. Plates were then washed as before and add 100 μl TMB substrate and incubate at RT for 15 min. 100 μl/well of stop solution was added to stop the reaction, and the plates were read with microplate reader at 450 nm. VHHs with competitive target binding capabilities were grouped to a same Bin. The results indicated SLN7150, SLN12036 and SLN12042 belong to Bin #1, SLN7150, SLN12036 and SLN12027 belong to Bin #2, SLN7150, SLN12036, SLN12041, SLN12044 and SLN12045 belong to Bin #3, SLN7160 and SLN7155 belong to Bin #4, SLN7162 might have different epitope with most of other VHH-Fcs belong to Bin #5 (FIG. 25).

### 3.2 Production of Bi-Paratopic VHH's

VHHs from different bins were combined with G4S linker to make bi-paratopic Fc-VHH-VHH fusion proteins as listed in Table 9. Plasmid construction and protein purification procedures can refer to the above. SDS-PAGE analysis and characterization result showed in FIG. 26.

TABLE 3
List of bi-paratopic VHHs

| Protein No. | Feature |
|---|---|
| SLN12021 | Fc-SLN7150-(G₄S)₃-SLN7155 |
| SLN12022 | Fc-SLN7150-(G₄S)₃-SLN7160 |
| SLN12023 | Fc-SLN7150-(G₄S)₃-SLN7162 |
| SLN12024 | Fc-SLN7152-(G₄S)₃-SLN7155 |
| SLN12025 | Fc-SLN7152-(G₄S)₃-SLN7160 |
| SLN12026 | Fc-SLN7152-(G₄S)₃-SLN7162 |
| SLN12058 | Fc-SLN12036-(G₄S)₃-SLN7155 |
| SLN12059 | Fc-SLN12036-(G₄S)₃-SLN7160 |
| SLN12060 | Fc-SLN12036-(G₄S)₃-SLN7162 |
| SLN12061 | Fc-SLN7150-(G₄S)₃-SLN12036 |

### 3.3 Characteristics of Bi-Paratopic VHH's

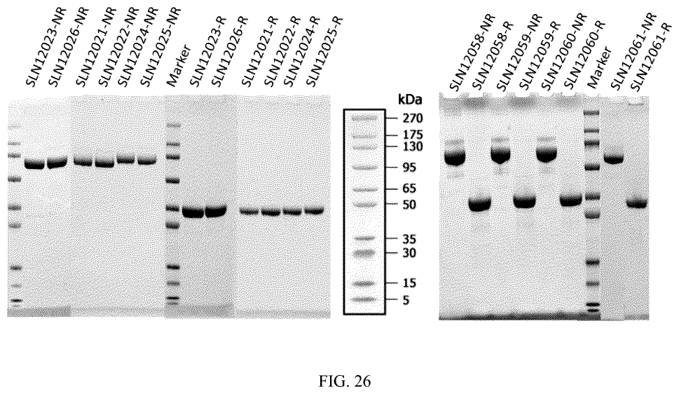

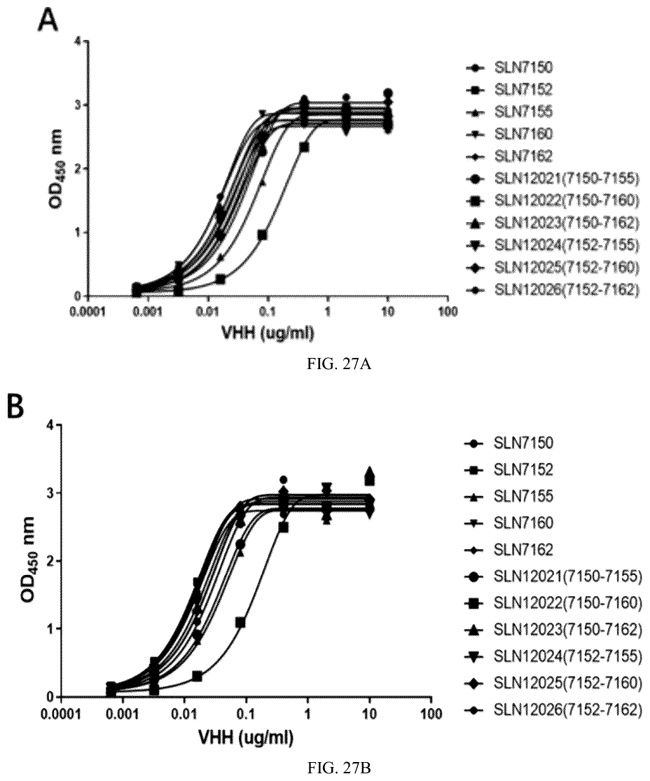

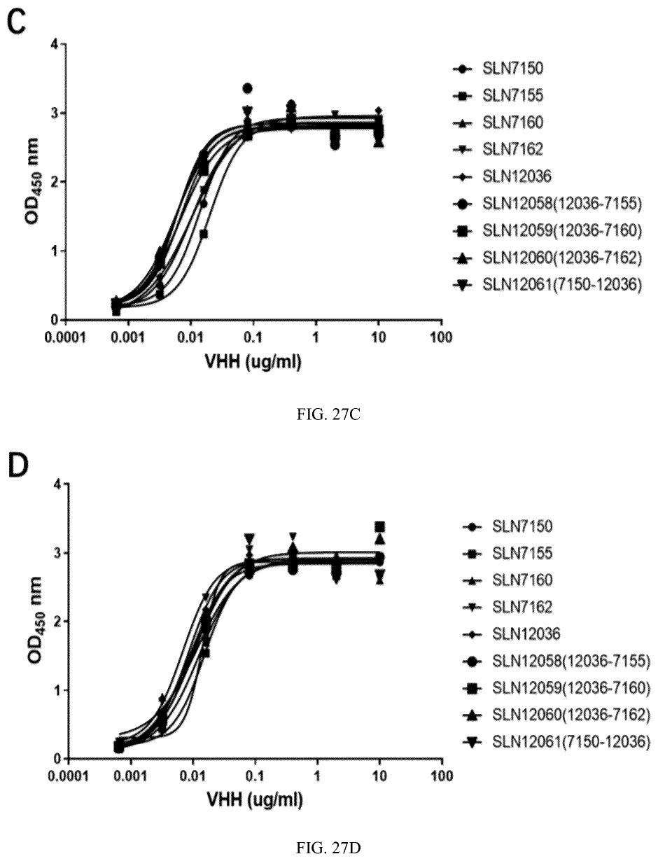

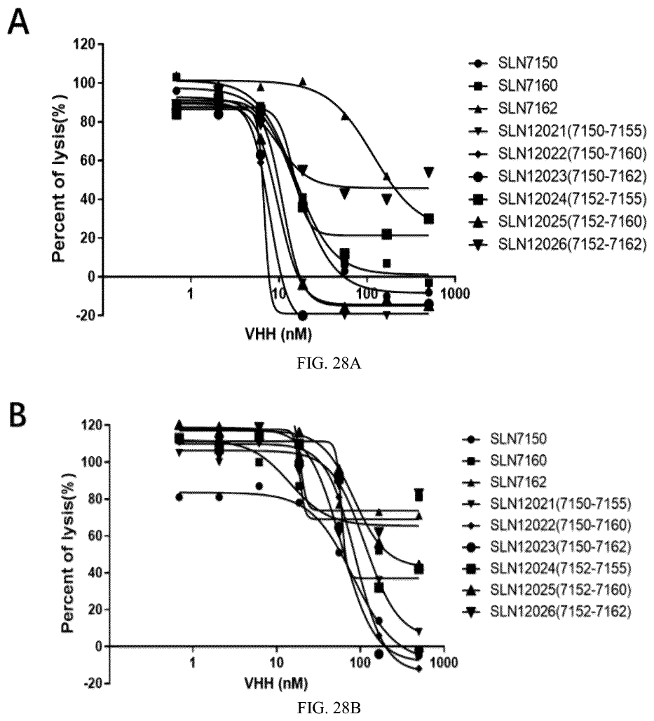

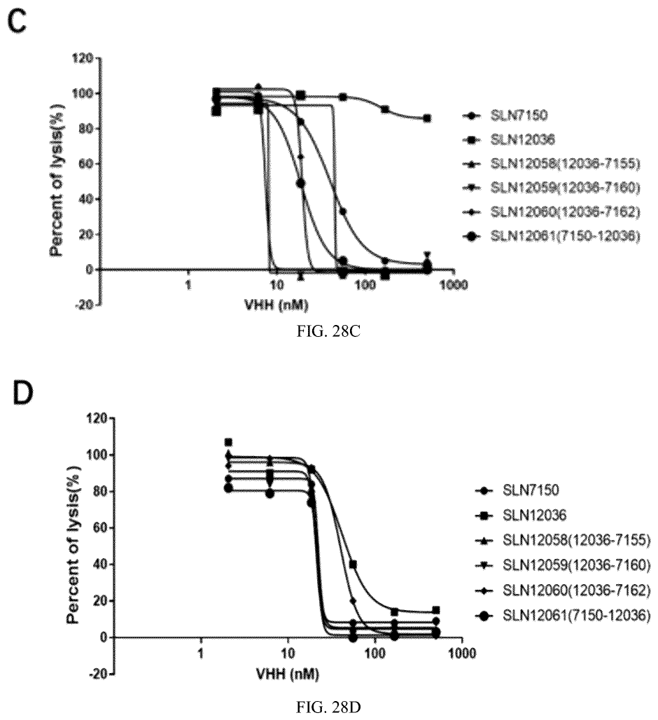

In the target-binding assays, as shown in FIGS. 27A-27D, the 10 bi-paratopic VHHs demonstrated negligible effect than those of single VHHs whether in human (FIG. 27A & FIG. 27C) or mice (FIG. 27B & FIG. 27D). In contrast, in the alternative pathway activity assays, as is shown in FIGS. 28A-28D), bi-paratopic VHH's shows better complement inhibitory activity than monovalent, especially in human serum.

### Example 3.4 Humanization of the Properdin-Binding VHH's

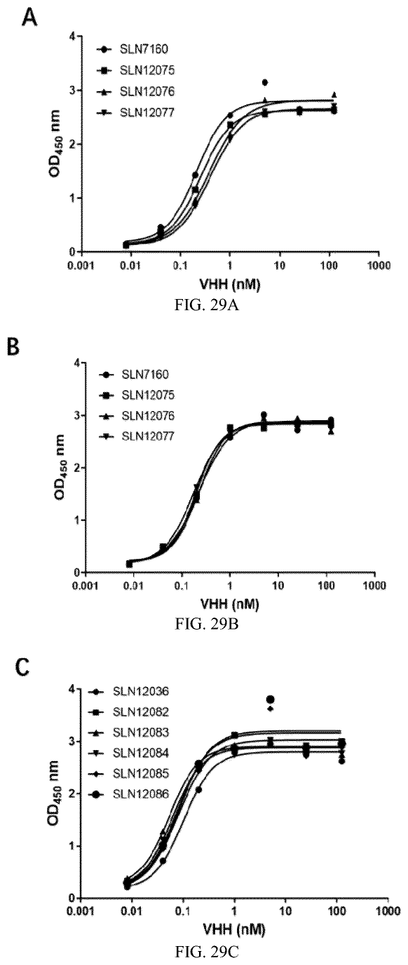

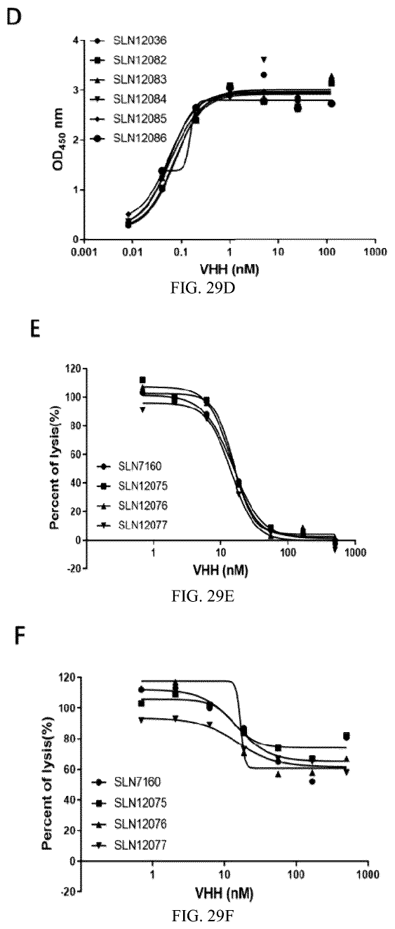

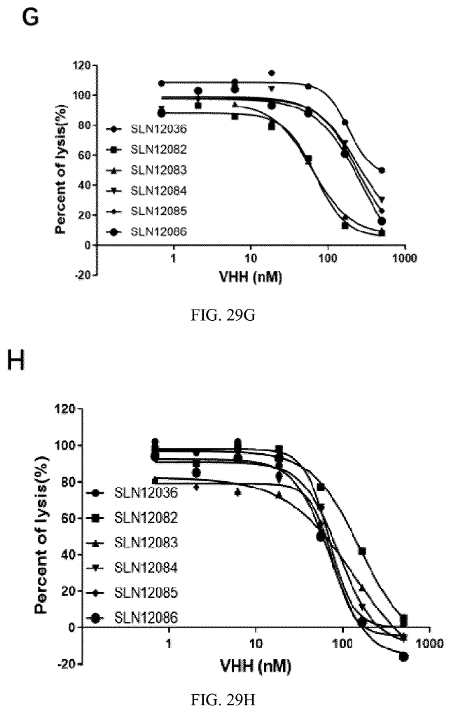

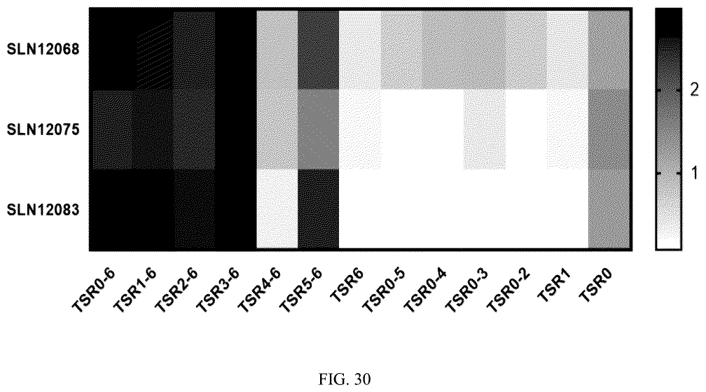

VHH humanization was conducted by standard procedures of CDR grafting and structural refinement. Upon the humanization design, recombinant DNA constructs were created to produce recombinant constructs as described above. The humanized sequences with an affinity equal to or better than that of the original VHH to properdin having acceptable expression and stability levels were selected for further development. FIGS. 29A-29H) indicated that the humanized VHH variant SLN7160 possess similar profile as the parental VHH regarding its binding to human (FIG. 29A) and mouse properdin (FIG. 29B). VHH variant SLN12036 possess similar profile as the parental VHH regarding its binding to human (FIG. 29C) and mouse properdin (FIG. 29D). Meanwhile, SLN7160 possess negligible effect of complement inhibition activity in human (FIG. 29E) and mouse serum (FIG. 29F), and it was noteworthy that humanized SLN12083 showed better human alternative pathway activity than the original VHH SLN12036 (FIG. 29G & FIG. 29H).

### Example 3.5 Epitope Mapping Assay with Truncated Human Properdin

96-well immunoplates were coated with 100 μl/well 1 μg/ml streptavidin and incubate at 4° C. overnight. Wells were washed with PBST for 3 times and blocked with 200 μl of 1% BSA/PBS at RT for 1 h. Washed with PBST for 3 times and add truncated variants of human properdin-biotin (50 μg/ml) 100 μl/well and incubated at RT for 1 h. Plates were washed with PBST for 3 times, 100 μl/well 10 μg/ml VHH-Fcs were added and incubate at RT for 1 h. Plates were washed with PBST for 3 times and add 100 μl goat anti-human Fc-HRP (Sigma, A0170) diluted 1/5000 in 1% BSA/PBST to each well and incubate at RT for 1 h. Plates were then washed as before and add 100 μl TMB substrate and incubate at RT for 15 min. 100 μl per well stop solution was added to stop the reaction, and the plates were read with microplate reader at 450 nm. Result was shown in FIG. 30, SLN12068, SLN12075 and SLN12083 binding with different thrombospondin repeats (TSRs) respectively by TSR0, TSR1, TSR2, TSR3, TSR4, TSR5, TSR6, TSR 0-6, TSR1-6, TSR2-6, TSR3-6, TSR4-6 TSR 5-6 and TSR6, and the biologically active TSR5-6 and TSR 0 domains play the important role in the binding of properdin with SLN12068, SLN12075 and SLN12083.

### Example 3.6 Impact of Properdin Inhibitors on Interactions Between C3 and Properdin

### 6.1 C3 Binding Assay

Maxisorp 96-well plates were coated with human C3 (Sigma, C2910-.1MG) 2 μg/ml, 100 μl/well in PBS, pH 7.4, and left overnight at 4° C. After washing 3 times with PBST, the wells were blocked with 2% BSA in PBS for 1 h at 37° C. Serial three-fold dilution (100 μl/well) of biotin human Properdin (starting at 90 μg/ml) were added to wells and incubated for 1 h at 37° C. The wells were washed three times with PBST and HRP-labeled streptavidin (1/5000 dilution) (sigma, s5512) was added to the wells. The plate was incubated for 1 h at 37° C. Plates were then washed as before and add 100 μl TMB substrate and incubate at RT for 15 min. 100 μl per well stop solution was added to stop the reaction, and the plates were read with microplate reader at 450 nm.

### 6.2 Competitive Binding Assay

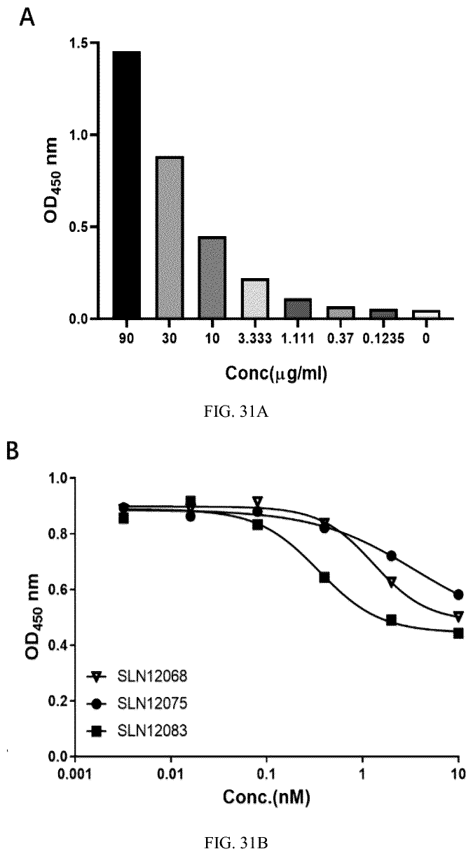

Maxisorp 96-well plates were coated with C3 (see binding assays), blocked with 1% BSA in PBST, and washed three times with PBST. Serially five-fold dilution FP inhibitors (starting at 50 nM), 100 μl/well, and a constant amount of properdin (20 μg/ml) 100 μl/well (in PBS) was added to each well, and incubated at 37° C. for 1 h. Wells were washed again three times with PBST, incubated with HRP-labeled Streptavidin (1/5000 dilution) (sigma, s5512) was added to the wells. The plate was incubated for 1 h at 37° C. Plates were then washed as before and add 100 μl TMB substrate and incubate at RT for 15 min. 100 μl per well stop solution was added to stop the reaction, and the plates were read with microplate reader at 450 nm. As is shown in FIG. 31A, properdin showed binding to C3 in dose-dependent manner. In addition, SLN12068, SLN12075 and SLN12083 also showed inhibition activity with properdin binding to C3. (FIG. 31B)

### Example 3.7 Fusion Protein of VHH to Engineered Factor H as a Dual-Inhibitor of Alternative Pathway

### 7.1 Bi-Paratopic Engineering of Humanized VHH Sequence

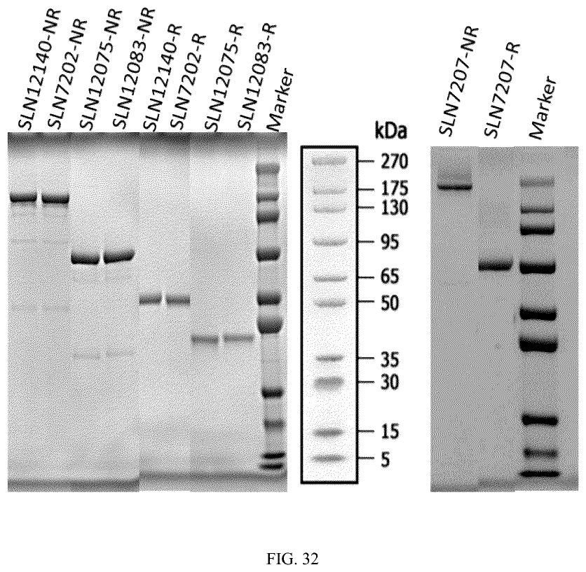

Bi-paratopic VHHs with humanized sequences through a G4S linker, by procedures as described above were created. Purified proteins were analyzed with 4-12% SDS-PAGE under non-reduced and reduced conditions (FIG. 32).

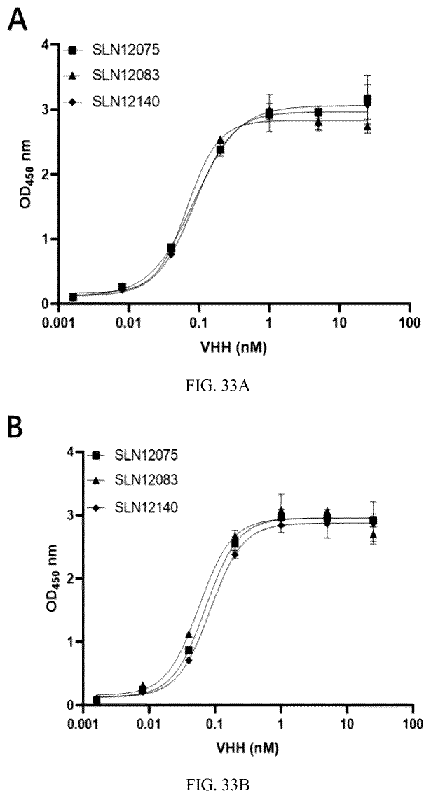

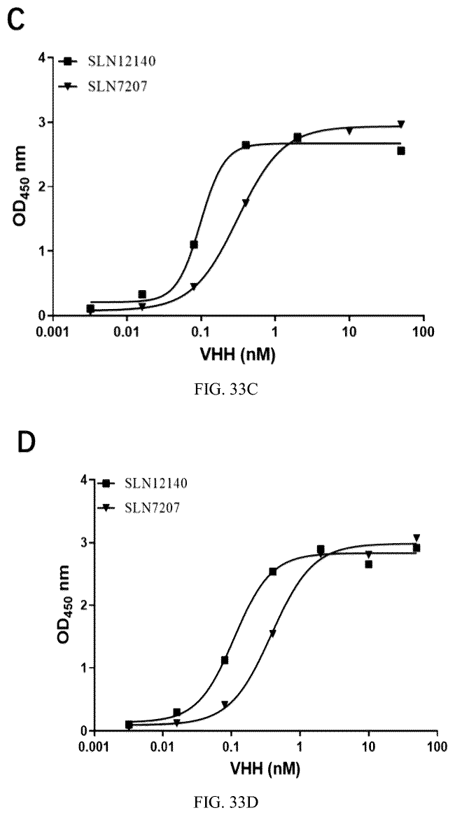

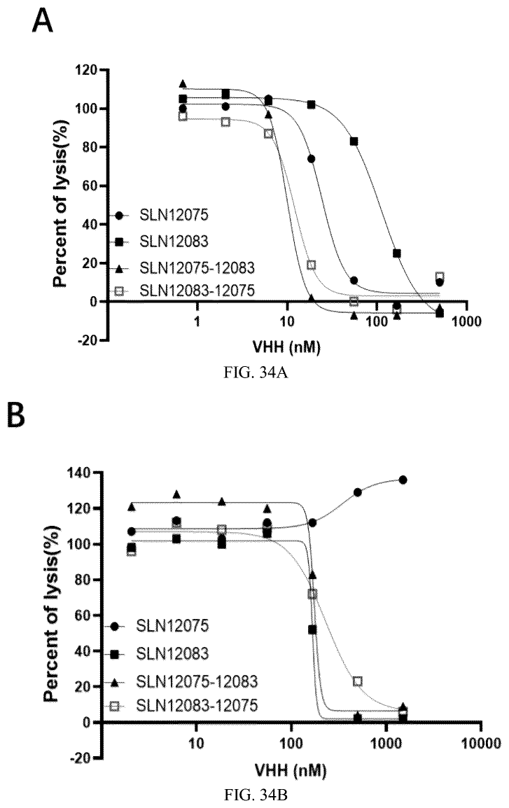

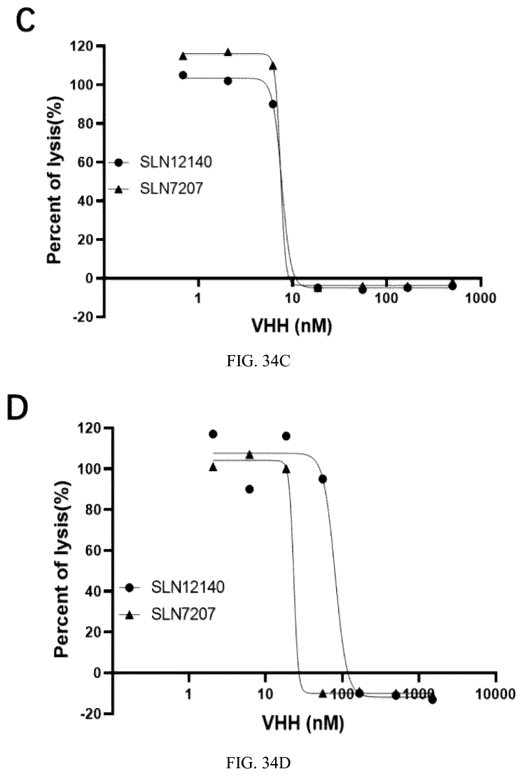

Target-binding ELISA shown in FIGS. 33A-33D indicated that such humanized bi-paratopic VHHs had negligible effect on the single VHH by human properdin binding (FIG. 33A) assay and mouse properdin binding assay (FIG. 33B). Alternative pathway activity indicated that the SLN12140 AP activity of bi-paratopic was better than that of single VHH (FIG. 34A & FIG. 34B).

### 7.2 Fusion Protein of VHH to Engineered Factor H

Truncated CFH (domain 1-4) was fused to the C-terminus of SLN12140 to form SLN7207 to make a dual functional recombinant protein inhibiting complement activation. The results were shown in FIGS. 33C-33D. FIG. 33C and FIG. 33D show binding activity of SLN7207 and SLN12140 to human properdin-biotin and mouse properdin-biotin. FIG. 34C and FIG. 34D show human and mouse alternative pathway activity of SLN12140 and SLN7207. Fusion with CFH (domain 1-4) at the C-terminus of SLN12140 showed negligible effect on that of SLN12140 by human serum, however it showed about three times biological activity increased by mouse serum. In any case, both SLN12140 and SLN7207 showed both human and mouse alternative pathway inhibition activity at nM level especially in humans with an IC50 of 17 nM. Such functional protein provides a potential therapeutical strategy for complement hyperactivation disease.

### Example 3.8 Pathway Selectivity and Species Cross Activity of SLN12140

### 8.1 Pathway Selectivity in Complement Inactivation

### 8.1.1 Complement Inactivation in Alternative Pathway

For alternative pathway assay, procedure refer to 2.3. The inhibition curves of single VHH SLN12075, SLN12083 and bi-paratopic SLN12140 exhibited consistent alternative pathway complement inhibitory activity, and SLN12140 showed superior activity compared to single VHH with IC50 of 17 nM. Eculizumab (Targetmol, T9915), a recombinant humanized monoclonal antibody against the complement protein C5 was as a control also showed inhibitory ability in the AP pathway, with IC50 of 50 nM.

### 8.1.2 Complement Inactivation in Classical Pathway

For classical pathway assay, all test samples were serially diluted 1:3 in PBS and added in duplicate (50 μl/well) to a U-bottom 96-well microtiter plate. Human complement-preserved serum (Quidel A113) was diluted to 20% vol/vol with GVB2+ buffer (0.1% gelatin, 141 mM NaCl, 0.5 mM MgCl₂, 0.15 mM CaCl₂, 1.8 mM sodium barbital) (Comp Tech B100) and added (50 μl/well) to the rows of the same 96-well plate such that the final concentration of human serum in each well was 10%. The plate was then incubated at RT for 30 min. Then chicken erythrocytes (1-4×10⁸) based on the samples were washed three times with 1 ml of GVBS2+ buffer and resuspended to a final concentration of 1×10⁸/ml in GVBS2+ buffer. After that, 1-6 ml of the chicken erythrocytes were sensitized by the addition of an anti-chicken red blood cell polyclonal antibody (Rockland, 103-4139) at 3% and the cells were incubated on ice for 15 min with frequent mixing. The cells were then washed twice with 1 ml of GVBS2+ buffer and resuspended to 1×10⁸/ml in GVBS2+ buffer. 30 μl aliquots of chicken erythrocytes (3×10⁶cells) were added to the plate as described above, mixed well, and incubated at 37° C. for 30 min. Then, each plate contained two wells of 50 μl of identically prepared chicken erythrocytes, one incubated with 50 μl PBS+50 μl GVBS 2+ buffer alone (negative control) as a control for spontaneous hemolysis, two wells containing 10 mM EDTA (Thermo 15575-038) as the serum blank and two wells normal NHS as 100% lysis. The plate was then centrifuged at 600 rpm for 2 min and 100 μl of the supernatant transferred to a new flat bottom 96-well plate. Hemoglobin release was determined at OD 405 nm using a microplate reader, and the percent hemolysis was determined using the following formula:

Hemolysis (%): 100×(OD sample−OD EDTA blank)/(OD 100% lysis−OD EDTA blank)

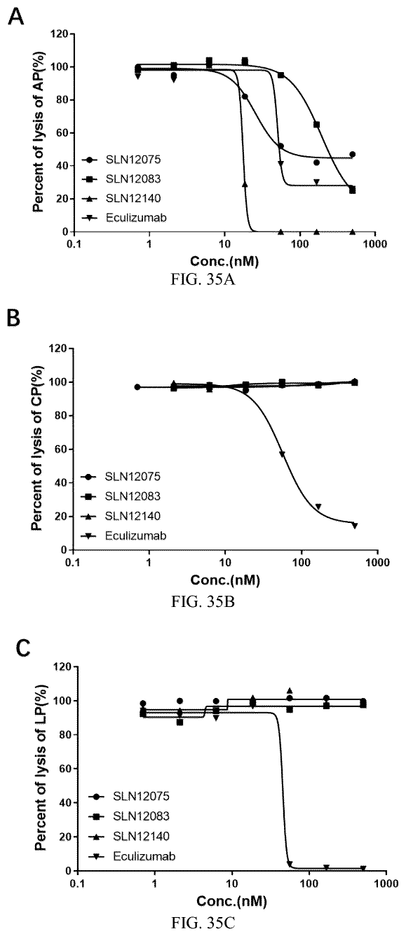

As shown in FIG. 35B, VHHs either single or bi-paratopic of the alternative pathway specific antibody, exhibited no inhibitory activity in the classical pathway. Eculizumab (Targetmol, T9915), a recombinant humanized monoclonal antibody against the complement protein C5 was as a control also showed inhibitory ability in the CP pathway, with IC50 of 57 nM.

### 8.1.3 Complement Inactivation in Lectin Pathway

For lectin pathway assay: 0.3 ml aliquots of mannan solution (0.5 mg/ml) were mixed with an equal volume of CrCl₃ solution (0.5 mg/ml) (Sigma 27096-100G-F, Lot #BCCB5331), an equal volume of the chicken erythrocyte suspension (1×10⁹ cells) was added, and the mixture was incubated with occasional mixing for 15 min at 25° C. Then wash with 1.0 ml of ice-cold GVBS2+. The erythrocytes coated with mannan (ME) (sigma M7604-100MG, Lot #SLOF4977) were washed three times by centrifugation with GVBS2+(gelatin-Veronal-buffered saline, 5 mM Veronal buffer, pH 7.4, containing 0.145 M NaCl, 0.1% gelatin, 0.15 M CaCl₂ and 0.5 mM MgCl₂) (Comp Tech, B100), resuspended to a final concentration of 5×10⁷ cells/ml in GVBS2+ and store on ice. All test samples were serially diluted 1:3 (from 500 nM to 0.2 nM) in PBS and added in duplicate (50 μl/well) to a U-bottom 96-well microtiter plate. Human complement-preserved serum was diluted to 20% vol/vol with GVBS2+ and added (50 l/well) to the rows of the same 96-well plate, such that the final concentration of human serum in each well was 10%, 100 μl ddH₂O or serum only and 50 μl PBS+50 μl GVBS2+ was used as 100% lysis and 0% controls, respectively, 10 mM EDTA was used as serum blank. 50 μl aliquots of chicken erythrocytes (2.5×10⁶ cells) was added to the plate as described above, mixed well, and incubated at 37° C. for 60 min. The plate was then centrifuged at 1,000 g for 2 min and 100 μl of the supernatant transferred to a new flat bottom 96-well plate. Hemoglobin release was determined at OD 405 nm using a microplate reader, and the percent hemolysis was determined using the following formula:

Percent hemolysis (%): 100×(OD sample−OD of EDTA)/(OD 100% lysis−OD of EDTA)

The inhibition curves of VHHs were shown in FIG. 35C, VHHs either single or bi-paratopic exhibited no inhibitory activity in the lectin pathway showing specificity in the alternative pathway. Eculizumab (Targetmol, T9915), a recombinant humanized monoclonal antibody against the complement protein C5 was as a control also showed inhibitory ability in the LP pathway, with IC50 of 45 nM.

### 8.2 Human, Cyno, Mouse and Rat Species Cross Activity of SLN12140

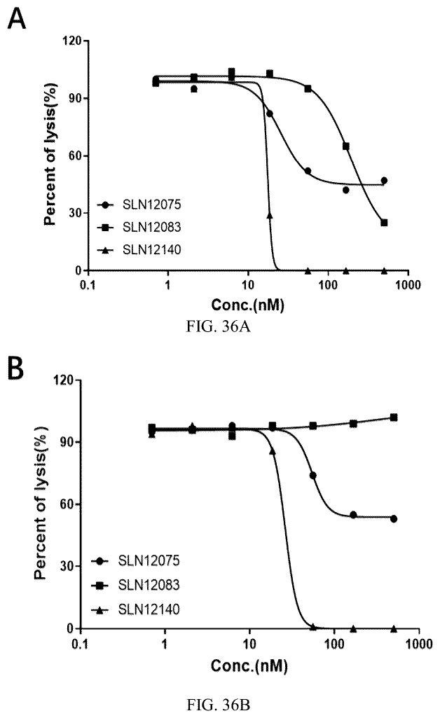

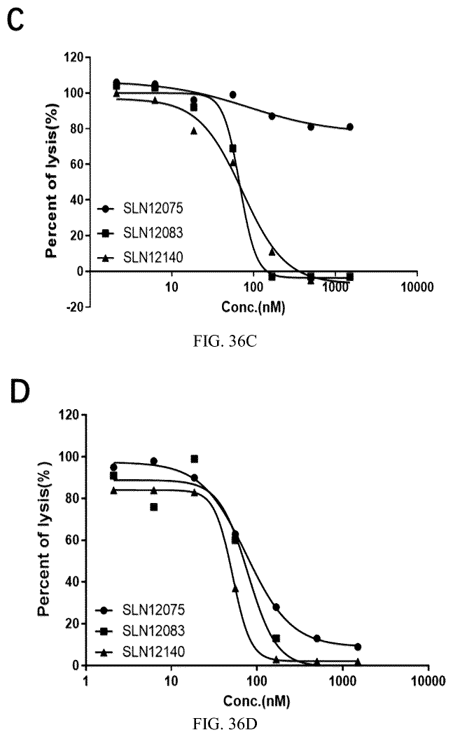

For alternative pathway assay, procedure refer to 2.3. The inhibition curves of VHHs shown in FIGS. 36A-36D, FIGS. 36A-36D show human, cyno, mouse and rat alternative pathway activity of SLN12075, SLN12083 and SLN12140 respectively. SLN12140 shows good species-crossing complement inhibitory activity in AP-specific pathways including human, mouse, monkey and rat. And all showed the characteristics of bivalent activity higher than single VHH.

### Example 3.9 Serum Stability of SLN12140 and It's PK Profiles in Mice

### 9.1 Serum Stability of SLN12140 in Mouse Plasma

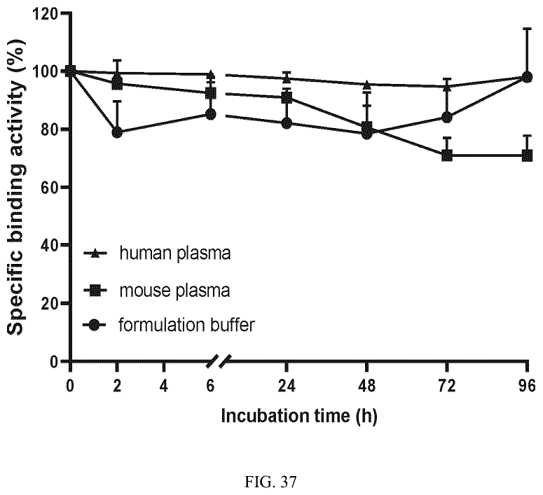

Test compound SLN12140 was prepared with C57BL/6 mouse plasma, human plasma and protein formulation buffer to a concentration of 10 μg/mL, and the samples were prepared and stored at −80° C. After that, samples were incubated at 37° C. with constant temperature and moisturizing conditions, and were collected at each time points (96, 72, 48, 24, 6, 2 and 0 h). Supernatant of each sample was obtained and analyzed for quantification by analytical Elisa as described below. All assays were performed in triplicate. FIG. 37 illustrates the buffer stability and plasma stability in human and mouse plasma. SLN12140 (SEQ ID NO: 229) maintain a stable concentration in formulation buffer within 96 h and maintain stable concentration until 96 h still 70% of SLN12140 can be detected.

### 9.2 Sample Analysis Method Establishment

### 9.2.1 Total Drug Pk Assay Development and QC Verification

Goat F(ab′)2 anti-human IgG-Fc (Abeam, CAT #Ab98587) was coated on a 96-well enzyme-linked plate, washed 3 times with PBST (Tween-20, 0.1%) and washed with 200 μl of PBST containing 1% BSA at 37° C. blocked for 1 h. After washing 3 times with PBST, serial dilutions of SLN12140 standard, serum samples and quality control samples in PBST containing 1% BSA were added. After incubation at 37° C. for 1 h. The cells were washed 3 times with PBST, horseradish peroxidase (HRP)-labeled goat anti-human IgG (FC-specific) antibody (Sigma, CAT #A0170) was added, and the cells were incubated at 37° C. for 1 h. After washing 3 times with PBST, 100 μl of TMB substrate solution was added to incubate for 15 min, and the absorbance at 450 nm was read after adding stop solution. The accuracy and accuracy of the standard curve and quality control materials were verified by SoftMax software, and the sample concentration was calculated. The validation of the PK method with three standard concentrations (high, medium, and low) showing the precision (CV %<20%) and accuracy (RE %+/−25%) of this method) meet the sample testing requirements.

### 9.2.2 Total mFP Assay Development and QC Verification

SLN12039 (SEQ ID NO: 241) was coated on a 96-well enzyme-linked plate, washed three times with PBST (Tween-20, 0.1%) and blocked with 200 μl of PBST containing 1% BSA for 1 h at 37° C. After washing 3 times with PBST, serially diluted mFP (Linno) with 1% BSA in PBST was added, incubated at 37° C. for 1 h, washed 3 times with PBST, and added with biotinylated SLN12030 antibody (Linno), incubate at 37° C. for 1 h, wash three times with PBST, add horseradish peroxidase (HRP)-labeled streptavidin antibody (Sigma, CAT #55512), and incubate at 37° C. for 1 h. After washing 3 times with PBST, 100 μl of TMB substrate solution was added to incubate for 15 minutes, and the absorbance at 450 nm was read after adding stop solution. The accuracy and accuracy of the standard curve and quality control materials were verified by SoftMax software, and the sample concentration was calculated. the validation of the PK method with three standard concentrations (high, medium, and low) showing the precision (CV %<20%) and accuracy (RE %+/−25%) of this method) meet the sample testing requirements.

### 9.3 Subcutaneous and Intravenous Single Dose Pharmacokinetic Studies in Mice

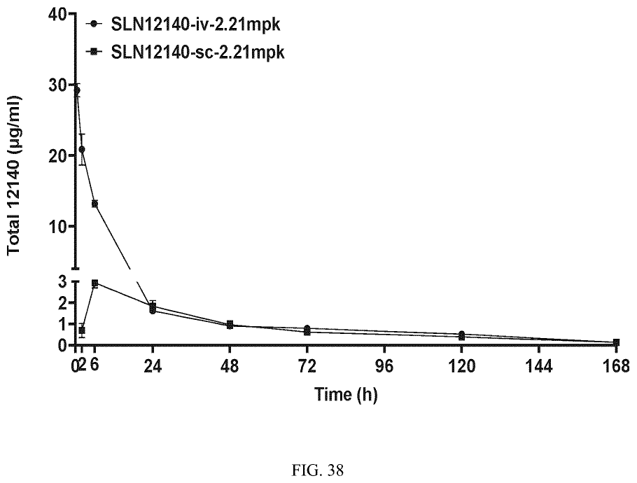

PK studies were performed in 8 weeks old male C57BL/6 mice. The study consisted of two groups, 4 animals each group and administered by a single-dose subcutaneous or intravenous injection with 2.21 mpk SLN12140 respectively. Collected at Pro-dose (0), 0.5 h, 2 h, 6 h, 24 h (D1), 48 h (D2), 72 h (D3), 120 h (D5), 144 h (D7) and 240 h (D10), serum was separated from blood samples, and the above-mentioned PK method was used for quantitative analysis of drugs in serum, and PK solver software was used to calculate PK parameters. As shown in FIG. 38, the terminal elimination half-lives of SLN12140 after i.v and s.c administration in mouse was 2.5d. SLN12140 was rapidly absorbed after s.c. administration and the bioavailability based on AUC₀₋ₜ was 40% for s.c. These results demonstrated that SLN12140 (SEQ ID NO: 229) exhibited a suitable half-time and favorable bioavailability.

### 9.4 Subcutaneous Dose Dependent Pharmacokinetic Studies in Mice

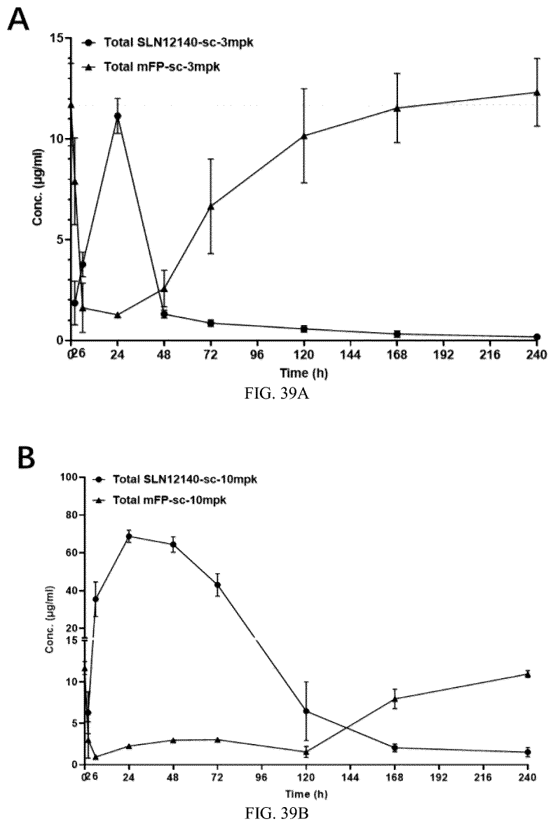

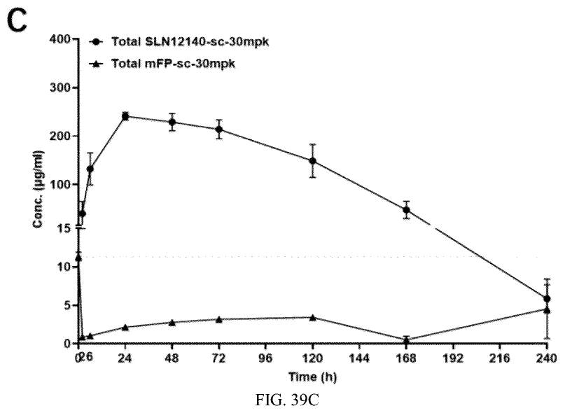

Dose dependent PK studies were performed in 8 weeks old male C57BL/6 mice. The study consisted of three groups, 5 animals each group and administered by a single-dose subcutaneous with 3, 10 and 30 mpk respectively. Blood samples were collected at pro-dose (0 h) and 8 sampling times post dosing: 2 h, 6 h, 24 h (D1), 48 h (D2), 72 h (D3), 120 h (D5), 144 h (D7) and 240 h (D10). Once collected, blood samples were centrifuged at 4° C. for 10 min at 1500 g and stored at −80° C. prior to analysis, and the above-mentioned PK method was used for quantitative analysis of drugs in serum, and PK solver software was used to calculate PK parameters. As results shown in FIGS. 39A-39C, the exposure of SLN12140 (SEQ ID NO: 229) in terms of AUC increased more than proportional with dose in the entire dose range between 3, 10 and 30 mpk. The concentration of the mFP also maintains a very consistent correspondence with the drug concentration. Low level FP concentration can be maintained for 48 h at 3 mpk, 120 h at 10 mpk, 168 h at 30 mpk respectively and showed good target inhibitory activity and good safety profile.

### 9.5 Multiple Dose Pharmacokinetic Studies in Mice

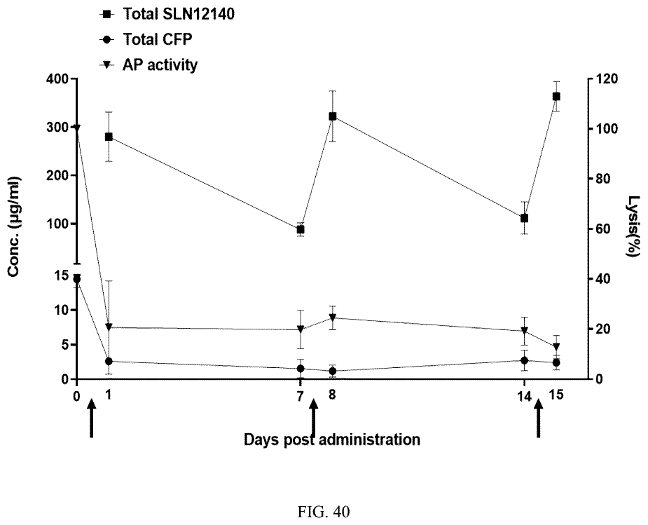

PK studies were performed in 8 weeks old male C57BL/6 mice. The subcutaneous doses of SLN12140 of 30 mpk, administered once every 7 days for four consecutive doses. Blood samples were collected at before each dose (0 h), 24 h post each dose. Once collected, after 2 hours of natural agglutination at room temperature, blood samples were centrifuged at 4° C. for 10 min at 1500 g and stored at −80° C. prior to analysis, and the above-mentioned PK method was used for quantitative analysis of drugs in serum, and PK solver software was used to calculate PK parameters. At the same time, AP activity was measured at different sampling time points using the method described above. As results shown in the Figure. 40, FP concentration decreased significantly 24 h after administration and remained below physiological levels 7 days after administration, an no risk of increased FP after multiple administrations was observed. The AP activity detected by the erythrocyte lysis method was highly consistent with the FP concentration, and the AP activity decreased significantly when the FP concentration decreased and SLN12140 (SEQ ID NO: 229) showed good and sustained AP pathway inhibitory activity after multiple dosing.

9.6 Multiple Dose Pharmacokinetic Studies in hCD89 Tg Mice

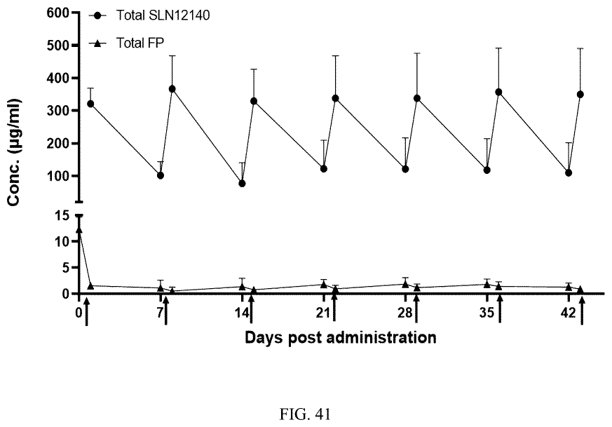

PK studies were performed in 13-14 weeks old male hCD89 Tg C57BL/6 mice. The hCD89 transgenic (Tg) mice expressed human CD89 on macrophage/monocytes and the key role of soluble CD89 in the pathogenesis of IgAN has been demonstrated in the literature. The subcutaneous doses of 30 mpk SLN12140, administered once every 7 days for seven consecutive doses. Blood samples were collected at before each dose (0 h), 24 h post each dose. Once collected, after 2 hours of natural agglutination at room temperature, blood samples were centrifuged at 4° C. for 10 min at 1500 g and stored at −80° C. prior to analysis, and the above-mentioned PK method was used for quantitative analysis of drugs in serum, and PK solver software was used to calculate PK parameters. At the same time, AP activity was measured at different sampling time points using the method described above. As results shown in FIG. 41, with 7 consecutive doses, the SLN12140 (SEQ ID NO: 229) serum concentration shows a continuous and stable pharmacokinetic character, and the weekly dosing can continuously decrease the target concentration at a low level, and there is no risk of immune complex formation according to blood concentration analysis and target concentration analysis. At the same time, mice are generally well observed.

### Example 3.10 SLN12140 Fusion with VEGF Inhibitors

10.1 Production of Recombinant Anti-Complement Protein VHH-G4Fc, Anti-VEGF Protein VHH-G4Fc, Bispecific Protein that Inhibited Both Complement and VEGF Pathways

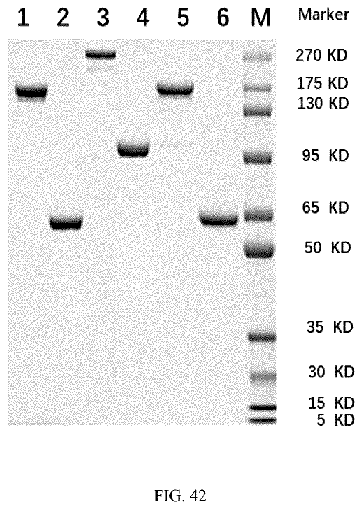

A nucleotide sequence corresponding to the amino acid of the bispecific fusion protein with VEGF inhibiting domain fused to N-terminal and complement inhibiting domain fused to C-terminal of Fc domain (SLN8284, SEQ ID NO: 262) and complement inhibiting protein (SLN12140, SEQ ID NO: 229) or VEGF inhibiting protein (SLN6073, SEQ ID NO: 261) with an G4Fc domain fused to C-terminal were constructed into the plasmid pCDNA3.4 or pCP. After these plasmids sequence were verified by sequencing, small scale production of recombinant proteins was performed using transient transfection of HEK293 cells with the recombinant plasmid using lipofectamine or PEI as transfection reagent. Cultures were grown in shaking flasks media at scales ranging from 30 ml to 100 ml for 5-7 days. Cells were removed by centrifugation and culture supernatants were used for protein purification via Protein A chromatography. The purified proteins were quantified by Coomassie Plus (Bradford) Assay Kit and analyzed with 4-12% gradient SDS-PAGE gel under non-reducing (lanes 1, lane3 and lane 5) or reducing conditions (lanes 2, lane 4 and lane 6) (FIG. 42).

### 10.2 In Vitro Binding of SLN8284, SLN12140, SLN6073 to Different Species' Properdin

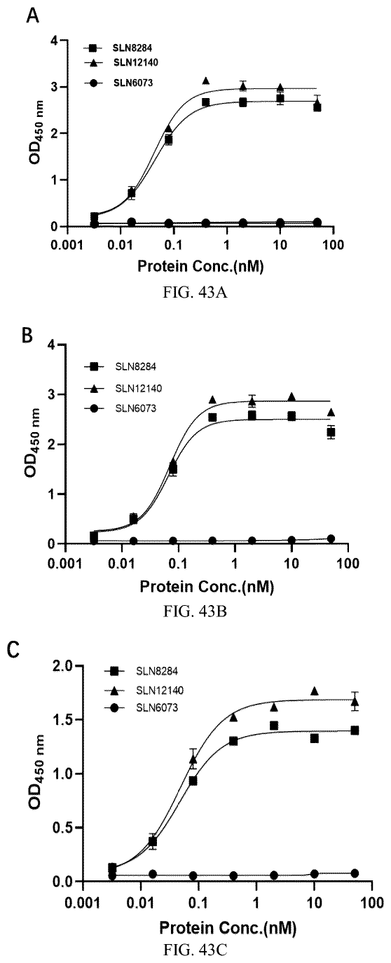

ELISAs were performed to determine whether proteins bind directly to properdin. Briefly, the wells of a 96-well ELISA plate were coated with streptavidin (1 μg/ml in CBS, 100 μl/well) and incubated at 4° C. overnight. After 3 times of washing with PBST, the plates were blocked with 1% BSA in PBST at RT for 1 hour. After 3 times of washing with PBST, biotinylated human or Cynomolgus macaques or mouse properdin protein (2 μg/ml in BSA, 100 μL/well) was added and incubate at RT for 1 hour. After 3 times of washing with PBST, the purified SLN8284 (SEQ ID NO: 262), SLN12140 (SEQ ID NO: 229), SLN6073 (SEQ ID NO: 261) (serially diluted in BSA, starting from 50 nM) were added and incubated at RT for 1 hour. After 3 times of washing with PBST, ANTI-HUMAN IGG (FC SPECIFIC) PEROXIDASE (Sigma Catalog No. A0170-1ML) were added to each well for incubation of 1 hour. After 3 times of washing with PBST, stop reagent for TMB Substrate was added to each well, and OD absorption at 450 nm was measured. The data was analyzed by sigmoidal curve fitting using GraphPad Prism 8.0. As shown in FIGS. 43A-43C. SLN8284, 12140 exhibited strong binding to human or Cynomolgus macaques or mouse properdin respectively, and SLN6073 (SEQ ID NO: 261) can't exhibit binding to human or Cynomolgus macaques properdin or mouse (FIGS. 43A-43C).

### 10.3 Inhibition of the Alternative Complement Pathway by SLN8284, SLN12140, SLN6073

In contrast to the classical and lectin complement pathways, which require both magnesium and calcium ions for activation, activation of the alternative complement pathway requires only magnesium ions. Thus, to quantify alternative complement activity in the presence of fusion proteins, the assay described above was modified such that rabbit erythrocytes (Er) were incubated with serum, 0-1500 nM fusion proteins, 5 mM Mg2+, and 5 mM EGTA, which preferentially chelates calcium ions.

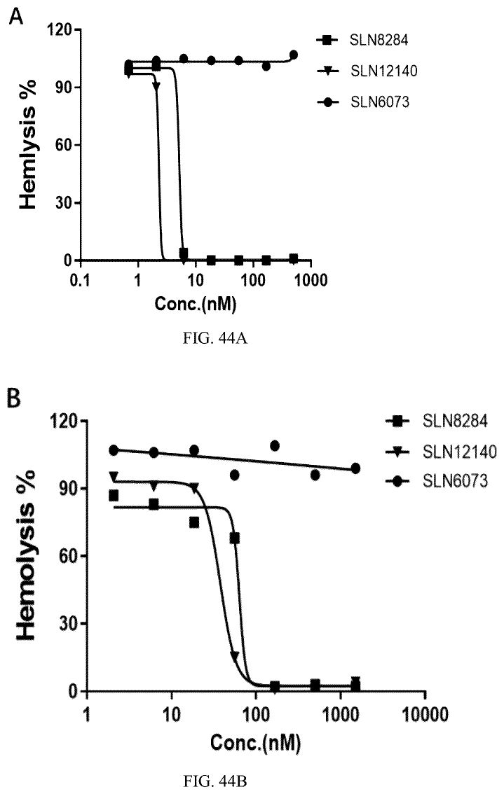

For this assay, all fusion proteins samples were serially diluted in PBS and added to a U-bottom 96-well microtiter plate, normal human serum (Complement Technology Catalog No. NHS A113) or complement-preserved mouse serum was diluted to the right concentration in assay buffer AP (20 mM Mg/EGTA in GVB0) and incubated on ice for 30 min. Then prepared of 5×10⁷ rabbit erythrocytes/ml (SenBeiJia Biological Technology Co., Ltd. Catalog No. SBJ-RBC-RAB003-10 mL) in assay buffer. Inhibition of the alternative complement pathway was initiated by mixing 0-1500 nM of SLN8284, SLN6073, SLN12140 with the dilution of normal human serum or mouse serum with 2.5×10⁶ rabbit erythrocytes for 30 min or 1 hour at 37° C. Hemolysis of Er was then assayed by measuring absorption at OD405 nm. The data was analyzed by sigmoidal curve fitting using Prism 8. Analysis of the percentage of hemolysis of the EA in the presence of the fusion proteins demonstrated that SLN8284 exhibited a high inhibitory activity with IC50 of 5.166 nM or 63.4 nM (FIGS. 44A, 44B, closed square). SLN12140 (SEQ ID NO: 229) exhibited an improved inhibitory effect (2.3 or 1.7-fold) to IC50 of 2.293 nM or 37.83 nM (FIGS. 44A, 44B, inverted triangle). SLN6073 (SEQ ID NO: 261) can't exhibit an inhibitory effect (FIGS. 44A, 44B, closed circle).

### 10.4 In Vitro Binding of SLN8284, SLN12140, SLN6073 to Human and Mouse VEGF

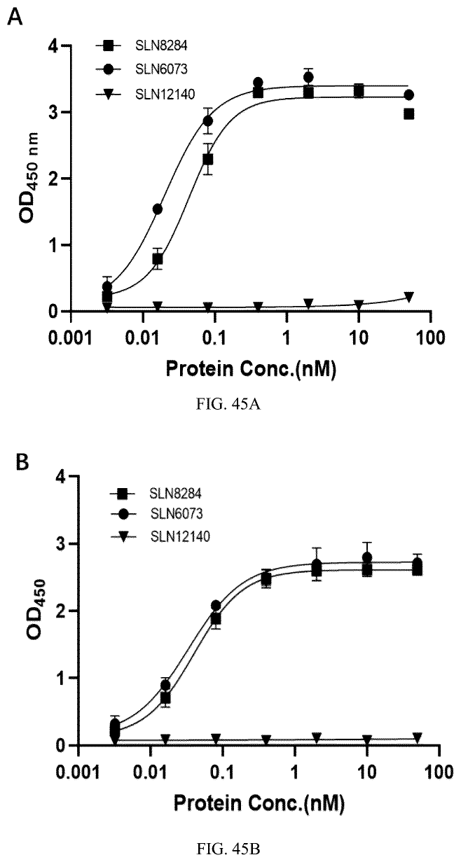

ELISAs were performed to determine whether proteins bind directly to VEGF. Briefly, the wells of a 96-well ELISA plate were coated with streptavidin (1 μg/mL in CBS, 100 μL/well) and incubated at 4° C. overnight. After 3 times of washing with PBST, the plates were blocked with 1% BSA in PBST at RT for 1 hr. After 3 times of washing with PBST, biotinylated human or mouse VEGF protein (0.5 μg/ml in BSA, 100 μL/well) was added and incubate at RT for 1 h. After 3 times of washing with PBST, the purified SLN8284 (SEQ ID NO: 262), SLN12140 (SEQ ID NO: 229), SLN6073 (SEQ ID NO: 261) proteins (serially diluted in BSA, starting from 50 nM) were added and incubated at RT for 1 h. After 3 times of washing with PBST, ANTI-HUMAN IGG (FC SPECIFIC) PEROXIDASE (Sigma Catalog No. A0170-1ML) were added to each well for incubation of 1 hour. After 3 times of washing with PBST, stop reagent for TMB Substrate was added to each well, and OD absorption at 450 nm was measured. The data was analyzed by sigmoidal curve fitting using GraphPad Prism 8.0. As shown in FIGS. 45A-45B. SLN8284 (SEQ ID NO: 262), SLN6073 (SEQ ID NO: 261) exhibited strong binding to human or mouse VEGF respectively, and SLN12140 (SEQ ID NO: 229) can't exhibit binding to human or Cynomolgus macaques properdin or mouse (FIGS. 45A, 45B).

(Human VEGF121: Accession #: P15692-9; VEGF120: Accession #: Q00731-3)

### 10.5 Blocking the Interaction Between VEGFA and VEGFR2 of SLN8284, SLN6073, SLN12140

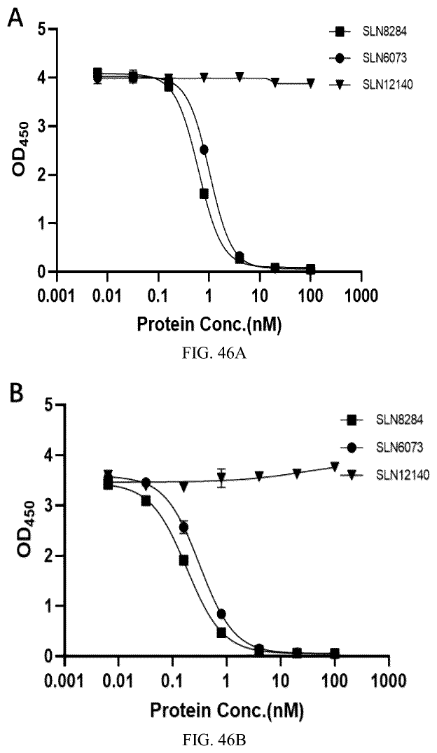

ELISAs were performed to determine whether proteins can block the interaction between VEGFA and VEGFR2. Briefly, the wells of a 96-well ELISA plate were coated with streptavidin (1 μg/ml in CBS, 100 μl/well) and incubated at 4° C. overnight. After 3 times of washing with PBST, the plates were blocked with 1% BSA in PBST at RT for 1 hour. After 3 times of washing with PBST, biotinylated human or mouse VEGF protein (0.07 μg/mL in BSA) was added and incubate at RT for 1 hour. After 3 times of washing with PBST, human or mouse VEGFR1 (0.14 μg/mL or 0.07 μg/mL in BSA) mixed with purified SLN8284, SLN12140, SLN6073 (a serially diluted in BSA, starting from 100 nM) was added and incubate at RT for 1 hour. After 3 times of washing with PBST, anti-mouse IgG Fc-HRP (Abcam, lot #GR3396448-2) were added to each well for incubation of 1 hour. After 3 times of washing with PBST, stop reagent for TMB Substrate was added to each well, and OD absorption at 450 nm was measured. The data was analyzed by sigmoidal curve fitting using GraphPad Prism 8.0. As shown in FIGS. 46A-46B. SLN8284, 6073 can block the interaction between human or mouse VEGFA and human or mouse VEGFR2 effectively. SLN12140 can't block the interaction between human or mouse VEGFA and human or mouse VEGFR2 (FIG. 46A and FIG. 46B).

(hVEGFR2: Accession #: P35968-1; mVEGFR2: Accession #: P35918-1)

### 10.6 Inhibition of VEGF-Dependent HUVEC Proliferation Assay by SLN8284, SLN12140, SLN6073

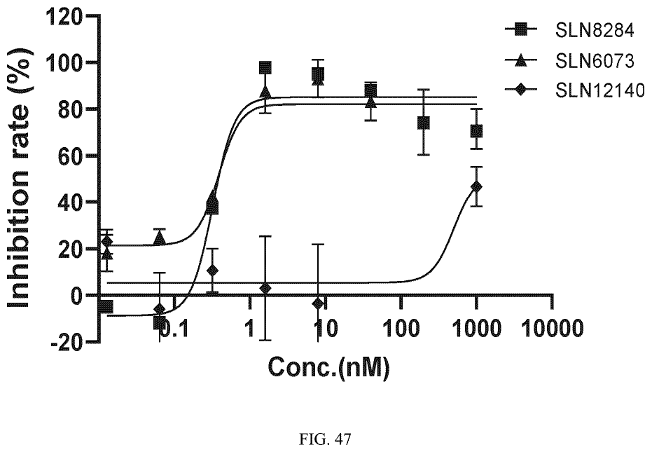

All proteins are tested for the ability to inhibit VEGF signaling pathway (e.g., inhibition of VEGF activity) in a cell-based assay. For example, SLN8284 was tested for the ability to inhibit VEGF signaling pathway (e.g, inhibition of VEGF activity) in this cell-based assay and compared to the VEGF inhibitory activity of SLN6073 and SLN12140. Human Umbilical Vein Endothelial Cells are often used to demonstrate VEGF-dependent cell proliferation which can be inhibited by binding of fusion proteins to VEGF. In this assay, HUVECs are maintained in Endothelial Cell Growth Medium with 1% FBS. A 96-well flat bottom microtiter plate is seeded 3000 HUVEC cells each well in complete medium (1% FBS), and culture overnight. Discard the medium from the cell plate and add 100 μL of various concentrations of fusion proteins (serially diluted in PBS, starting from 1000 nM) from mixed with 50 ng/mL VEGF-A in each well and incubate for 72 hours at 37° C. Cell proliferation is assayed by adding 10 μL of CCK-8 (Dojindo, Inc.) to each well and incubate for 2.5 hours at 37° C. Cell proliferation is measured at OD absorption of 450 nm. Results showed that SLN8284 and SLN6073 significantly inhibited VEGF-induced HUVEC proliferation as compared to the control (SLN12140) and the inhibitory effect of SLN8284 was similar to SLN6073 (FIG. 47).
SLN8284 (SEQ ID NO: 262)
SLN6073 (SEQ ID NO: 261)

### Example 3.11 SLN12140 Fusion with hTf Binding VHH (9056VHH)

11.1 Production of Anti-CFP Recombinant Antibodies with/without 9056VHH Domain

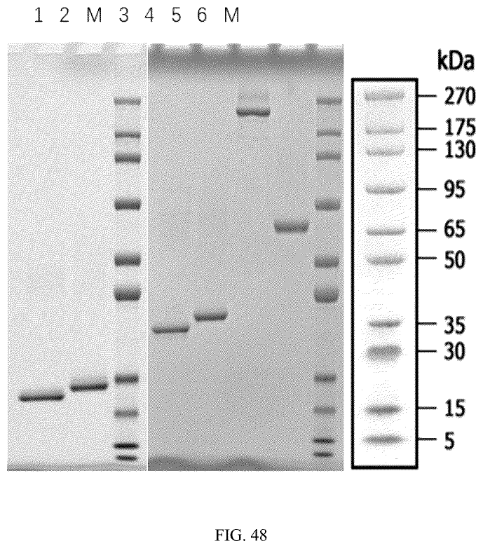

SLN12149 (12075-(G4S)3-12083-(G4S)3-9056-G4S-His) and SLN12150 (12075-(G4S)3-12083-(G4S)3-Fc-(G4S)3-9056) fusion proteins were constructed from SLN12147 (12075-(G4S)3-12083-(G4S)3-His) and SLN12140(12075-(G4S)3-12083-(G4S)3-Fc), respectively. Small scale production of recombinant proteins was performed using transient transfection of HEK293 cells with the recombinant plasmid using PEI as transfection reagent. Cultures were grown in 100 ml shaking flasks media for 5-7 days. Cells were removed by centrifugation and culture supernatants were used for protein purification by Ni-NTA or protein A sepharose. The purified proteins were analyzed with 4-12% gradient SDS-PAGE gel under non-reducing or reducing conditions (FIG. 48).

11.2 CFP Binding Assay with the Recombinant Antibodies in Different Species

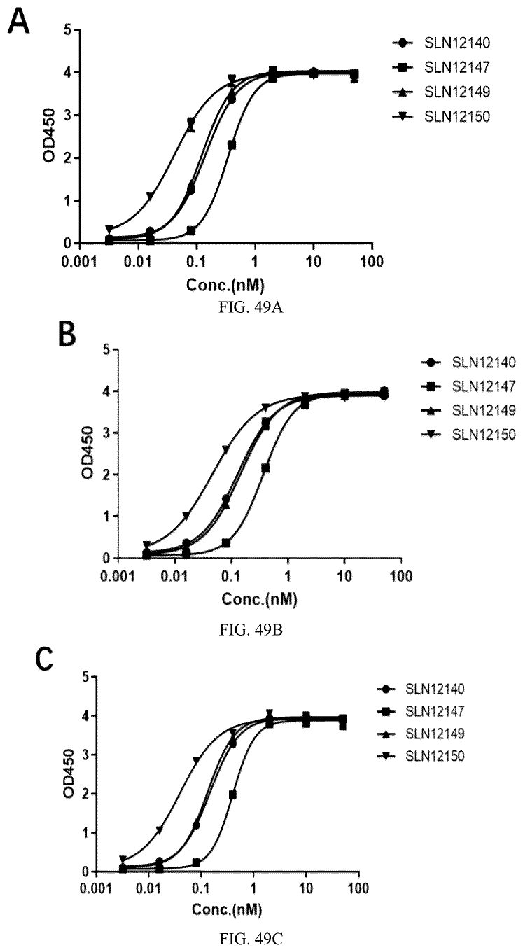

To test the binding activities of recombinant antibodies to CFP, 96-well microplates were coated with streptavidin (1 μg/ml in PBS, 100 μl/well) and incubated at 4° C. overnight. After 3 times of washing with PBST, the plates were blocked with 1% BSA in PBST at RT for 1 hr. After 3 times of washing with PBST, biotinylated human/rhesus/mouse CFP (10/5/2 μg/ml in 1% BSA, 100 μl/well) was added and incubate at RT for 1 h. After 3 times of washing with PBST, the recombinant antibodies were added and incubated at RT for 1 h (50 nM, 5× dilution, serially diluted in 1% BSA, 100 μl/well). SLN12143 (1 μg/ml, binding 12075/12083 humanized framework) and a secondary anti-mFc-HRP were incubated sequentially, before the plates were washed with PBST 3 times and incubated with substrate solution and stop solution as described above for ELISA (FIGS. 49A-49C).

11.3 Human Transferrin Binding Assay with the Recombinant Antibodies

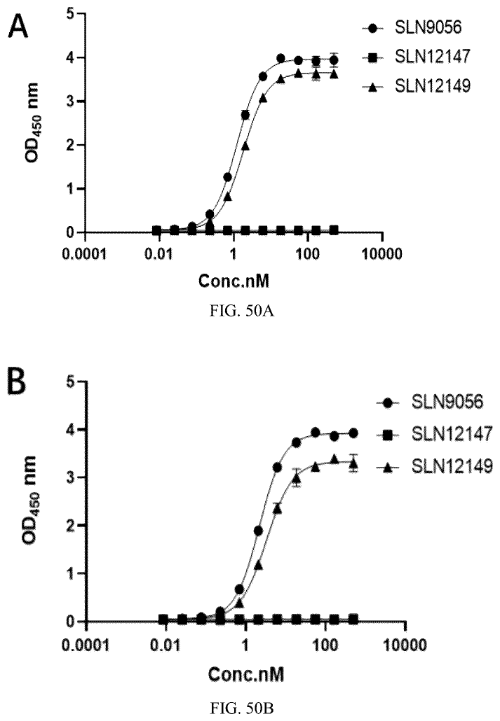

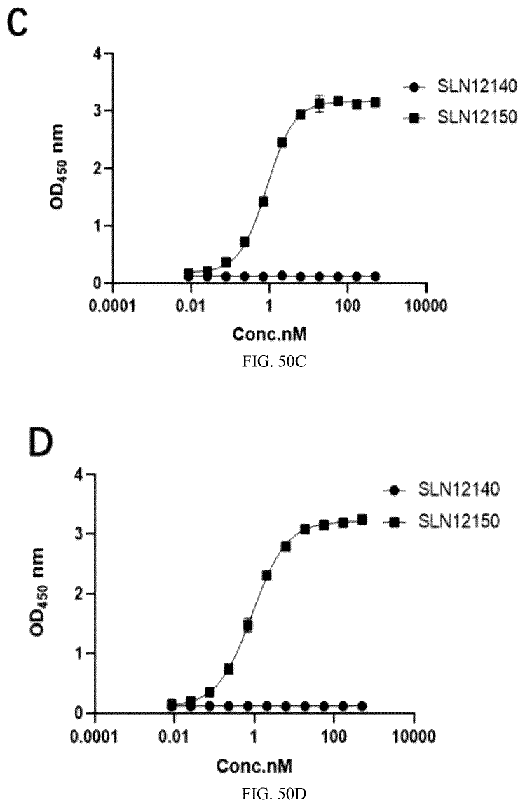

To test the binding activities of recombinant antibodies to human transferrin, 96-well microplates were coated with streptavidin (1 μg/ml in PBS, 100 μl/well) and incubated at 4° C. overnight. After 3 times of washing with PBST, the plates were blocked with 1% BSA in PBST at RT for 1 hr. After 3 times of washing with PBST, was added and incubate at RT for 1 h. After 3 times of washing with PBST, the recombinant antibodies (1000 nM, 5× dilution, serially diluted in 1% BSA) were premixed with biotinylated human transferrin (2 ug/ml in 1% BSA), added the mixture (100 μl/well) into the plates and incubated at RT for 1 h. Then, the secondary anti-hFc-HRP/anti-6×his-HRP were incubated sequentially, before the plates were washed with PBST 3 times and incubated with substrate solution and stop solution as described above for ELISA. (FIGS. 50A-50D).

11.4 Inhibiting Human/Cynomolgus/Mouse AP Activity with the Recombinant Antibodies

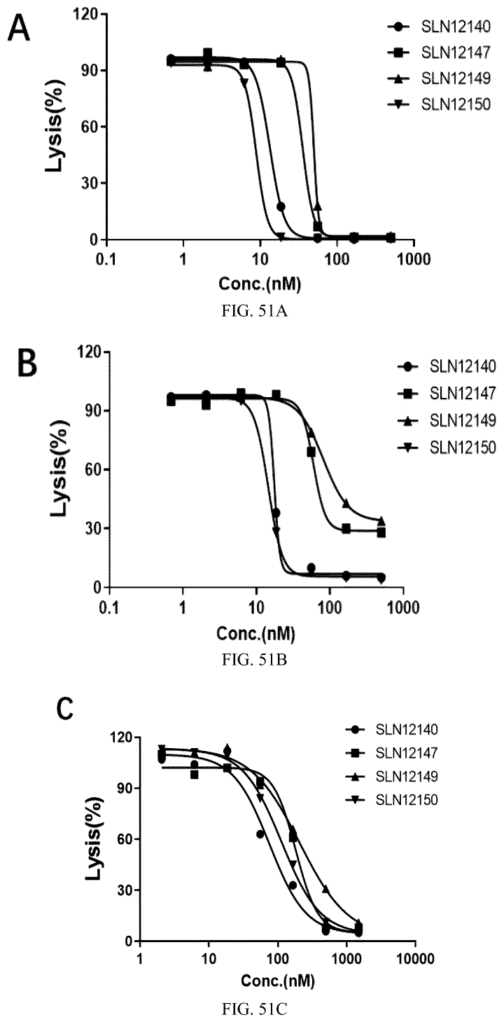

Recombinant antibodies were serially diluted with 1×PBS, then transfer to 96-well Plate with U bottom (50 μl/well). Prepare 20% human serum/20% cynomolgus serum with 1×AP buffer, 60% mouse serum with 2×AP buffer, incubate on ice for 30 minutes. Prepared the rabbit RBC during incubation of serum: 0.5 ml of rabbit erythrocytes (4×10⁸/ml) were washed three times with 1 ml of 1×AP buffer and resuspended to a final concentration of 5×10⁷/ml (4 ml) in 1×AP buffer. Then the activated serum (50 μl/well) and red cell (50 μl/well) were added to sample well. The plate with human/cynomolgus serum were incubated 30 mins in 37° C., the plate with mouse serum were incubated 60 mins in 37° C. The plate was centrifuged at 600 rpm for 2 mins and 100 μL of the supernatant transferred to a new flat bottom 96-well plate. Hemoglobin release was determined at OD 405 nm using a microplate reader (FIGS. 51A-51C).

11.5 Mouse BBB pK Assay in Mouse Tissues with the Recombinant Antibodies

### 11.5.1 Method Principle of PK Assay (SLN12140/SLN12150/SLN12147/SLN12149)

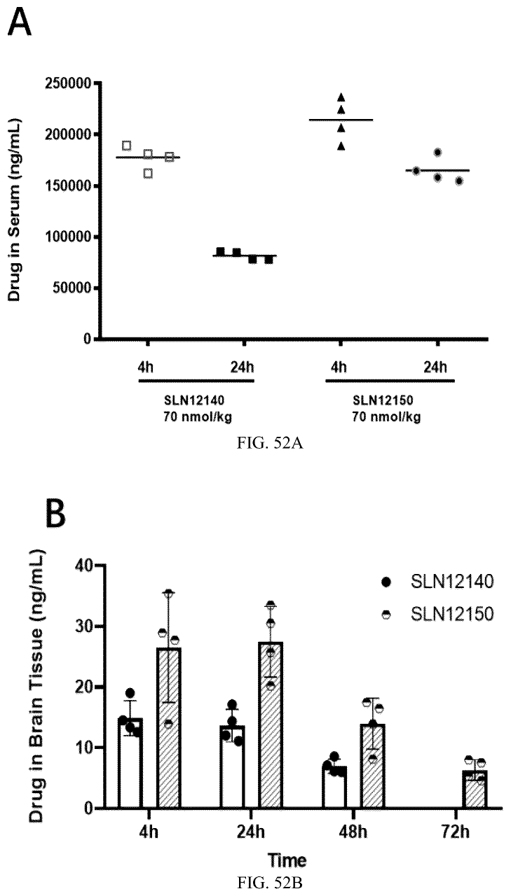

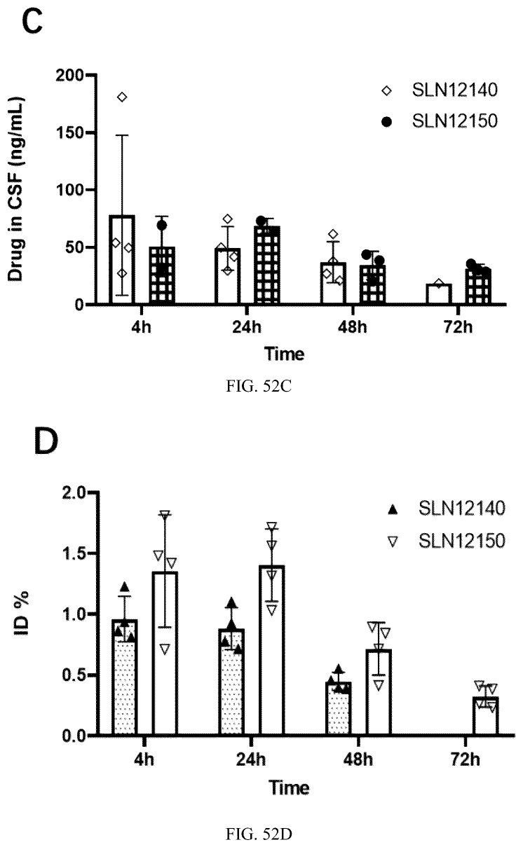

This method utilizes an antibody sandwich mode by ELISA to detect analyte in mouse serum/brain tissue homogenate/CSF. The assay plate coated with SLN2102 is incubated overnight. On the next day, analyte in samples and STD/QC will be captured on the plate by SLN2102. After the sample incubation completed, detection antibody which Biotin labeled will be added to the plate. The plate is washed to remove excess detection antibody (Bio-SLN2108). SA-HRP is added to bind Biotin. After completion, TMB is added and the plate is read on Micro-plate Reader. The resulting OD is proportional to the amount of analyte presents in the samples and STD/QC (FIGS. 52A-52D).
SLN12147 (SEQ ID NO: 264)
SLN12149 (SEQ ID NO: 265)
SLN12150 (SEQ ID NO: 266)

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents maybe considered to fall within the scope of the invention as defined by the claims that follow.

What is claimed is:

1. An isolated antibody or an antigen-binding fragment thereof, comprising a heavy chain variable region comprising HCDR1, HCDR2 and HCDR3, wherein
a) the HCDR1 comprises an amino acid sequence selected from the group consisting of: SEQ ID NOs: 3, 6, 9, 12, 39, 40, 41, 42, 43, 44, 45, 46, 47, 48, 49, 50, 51, 52, 53, 54, 55, 56, 57, 58, 59, 149, 150, 151, 152 and 153;
b) the HCDR2 comprises an amino acid sequence selected from the group consisting of: SEQ ID NOs: 2, 5, 8, 11, 60, 61, 62, 63, 64, 65, 66, 67, 68, 69, 70, 71, 72, 73, 74, 75, 76, 77, 78, 79, 80, 81, 82, 154, 155, 156, 157, 158 and 159; and
c) the HCDR3 comprises an amino acid sequence selected from the group consisting of: SEQ ID NOs: 1, 4, 7, 10, 83, 84, 85, 86, 87, 88, 89, 90, 91, 92, 93, 94, 95, 96, 97, 98, 99, 100, 101, 102, 103, 104, 105, 106, 107, 160, 161, 162, 163, 164 and 165.

2. The isolated antibody or antigen-binding fragment thereof of claim 1, comprising a heavy chain variable region comprising an amino acid sequence selected from the group consisting of: SEQ ID NOs: 13, 14, 15, 16, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 148, 208, 209, 210, 211, 212, 213, 214, 215, 216, 217, 218, 219, 220, 221, 222, 223, 224, 225, 226, 227, 228, 231, 232, 233, 234, 235, 236, 237, 238, 239, 240, 241, 242, 243, 244, 245 and 263.

3. The isolated antibody or antigen-binding fragment thereof of claim 1, having one or more properties selected from the group consisting of:
1) an ability of specifically binding to VEGFA (Vascular Endothelial Growth Factor A);
2) an ability of preventing VEGF from binding its corresponding receptor(s);
3) an ability of inhibiting VEGF-driven biological functions;
4) an ability of specifically binding to transferrin;
5) capable of extending in vivo half-life of an moiety linked to the isolated antigen-binding protein;
6) capable of crossing blood brain barriers;
7) an ability of specifically binding to properdin.

4. The isolated antibody or antigen-binding fragment thereof of claim 1, the antibody or the antigen-binding fragment thereof is a VHH.

5. A bi-paratopic antigen-binding protein, comprising a first antigen-binding domain, and a second antigen-binding domain, wherein said first antigen-binding domain and/or said second antigen binding domain comprises the isolated antibody or antigen-binding fragment thereof of claim 1.

6. The bi-paratopic antigen-binding protein of claim 5, wherein the first antigen-binding domain and the second antigen-binding domain target the same antigen.

7. The bi-paratopic antigen-binding protein of claim 5, wherein the first antigen-binding domain and the second antigen-binding domain target different antigens.

8. The bi-paratopic antigen-binding protein of claim 5, comprising an amino acid sequence selected from the group consisting of: SEQ ID NOs: 17, 18, 19, 21, 23, 229, 230, 246, 247, 248, 249, 250, 251, 252, 253, 254, 255, 261, 262, 264, 265 and 266.

9. A polypeptide comprising the isolated antibody or antigen-binding fragment thereof of claim 1.

10. The polypeptide of claim 9, further comprising one or more other functional domain(s), a therapeutic entity, or a functionally active protein.

11. The polypeptide of claim 10, wherein the functional domain is a Fc region or a VHH region.

12. The polypeptide of claim 10, wherein the functionally active protein is a factor H, a VEGF inhibiting protein or a transferrin binding protein.

13. The polypeptide of claim 9, comprising an amino acid sequence selected from the group consisting of: SEQ ID NOs: 17, 18, 19, 21, 23, 143, 144, 229, 230, 246, 247, 248, 249, 250, 251, 252, 253, 254, 255, 261, 262, 264, 265 and 266.

14. An isolated nucleic acid molecule encoding the isolated antibody or antigen-binding fragment thereof of claim 1.

15. A vector comprising the nucleic acid molecule of claim 14.

16. A cell comprising the nucleic acid molecule of claim 14.

17. A pharmaceutical composition comprising the isolated antibody or antigen-binding fragment thereof of claim 1.

18. A method of preparing the isolated antibody or antigen-binding fragment thereof of claim 1.

19. A method for preventing and/or treating diseases and/or disease-related symptoms, comprising administering to a subject in need thereof the isolated antibody or antigen-binding fragment thereof of claim 1.
